(19) United States
(12) Patent Application Publication
(10) Pub. No.: US 2024/0327375 A1
(43) Pub. Date: Oct. 3, 2024

(54) SUBSTITUTED HETEROBICYCLIC DERIVATIVES AS NEGATIVE ALLOSTERIC MODULATORS OF MGLU7 RECEPTOR

(71) Applicant: Addex Pharma S.A., Geneva (CH)

(72) Inventors: Jean-Laurent Paparin, Geneva (CH); Jean-Philippe Rocher, Geneva (CH); Patrick Mäder, Mannheim (DE); Tanja Stach, Ottweiler (DE); Guillaume Duvey, Thiez (FR); Lam Tang, Geneva (CH)

(21) Appl. No.: 18/560,011

(22) PCT Filed: May 13, 2022
(86) PCT No.: PCT/EP2022/063106

§ 371 Date: Nov. 9, 2023

(30) Foreign Application Priority Data
May 13, 2021 (GB) 2106871.3

(51) Int. Cl. C07D 401/04; A61K 31/502; A61K 31/506; A61K 31/5377; A61K 31/5386; A61K 31/541; C07D 237/32; C07D 401/14; C07D 403/04; C07D 403/10; C07D 403/14; C07D 405/04; C07D 491/107; C07D 498/08

(52) U.S. Cl. CPC C07D 401/04; A61K 31/502; A61K 31/506; A61K 31/5377; A61K 31/5386; A61K 31/541; C07D 237/32; C07D 401/14; C07D 403/04; C07D 403/10; C07D 403/14; C07D 405/04; C07D 491/107; C07D 498/08

(57) ABSTRACT

The present invention relates to novel compounds of Formula (I), wherein A, m, Q, R¹, R², R³, R⁴ and R⁵ are defined as in Formula (I); which are negative allosteric modulators of the metabotropic glutamate receptor subtype 7 (mGlu7) and which are useful for the treatment or prevention of neurological, ear and psychiatric disorders associated with glutamate dysfunction and diseases in which the mGlu7 subtype of metabotropic receptors is involved. The invention is also directed to pharmaceutical compositions comprising such compounds, to processes to prepare such compounds and such compositions, and to the use of such compounds for the prevention or treatment of neurological, ear and psychiatric disorders and diseases in which mGlu7 is involved.

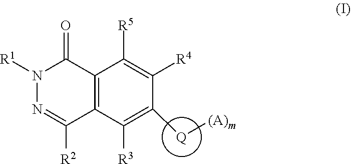

### SUMMARY OF THE INVENTION

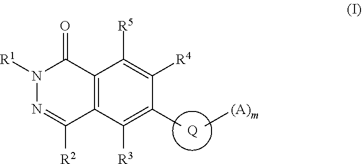

The present invention relates to novel compounds of Formula (I), wherein A, m, Q, R¹, R², R³, R⁴ and R⁵ are defined as in Formula (I); which are negative allosteric modulators of the metabotropic glutamate receptor subtype 7 (mGlu7) and which are useful for the treatment or prevention of neurological, ear and psychiatric disorders associated with glutamate dysfunction and diseases in which the mGlu7 subtype of metabotropic receptors is involved. The invention is also directed to pharmaceutical compositions comprising such compounds, to processes of preparing such compounds and such compositions, and to the use of such compounds for the prevention or treatment of neurological, ear and psychiatric disorders and diseases in which mGlu7 is involved.

### BACKGROUND OF THE INVENTION

Glutamate is the primary amino-acid transmitter in the mammalian central nervous system (CNS). Glutamate is associated with numerous physiological functions learning and memory, sensory perception, development of synaptic plasticity, motor control, respiration, and regulation of cardiovascular function. Furthermore, glutamate is at the centre of several different neurological and psychiatric diseases, where there is an imbalance in glutamatergic neurotransmission.

Glutamate mediates synaptic neurotransmission through the activation of ionotropic glutamate receptor channels (iGluRs), the NMDA, AMPA and kainate receptors which are responsible for fast excitatory transmission (Nakanishi et al. (1998) Brain Res. Rev., 26:230-235).

In addition, glutamate activates metabotropic glutamate receptors (mGluRs) which have a modulatory role that contributes to the fine-tuning of synaptic efficacy (Niswender & Conn (2010) Ann. Rev. Pharmacol. Toxicol. 50:295-322). As opposed to iGluRs, mGluRs do not mediate but rather “modulate” synaptic transmission acting at different levels of the tripartite synapse formed by the junction of axon terminals, dendritic spines, and astrocytes. The mGluRs are seven-transmembrane domain-containing G protein-coupled receptors (GPCRs) belonging to family 3 GPCRs along with the calcium-sensing, GABA_{B}, and pheromone receptors. Glutamate activates the mGluRs through binding to a site on the large extracellular amino-terminal domain of the receptor, herein called the orthosteric binding site. This activation induces a conformational change of the rest of the receptor which results in the activation of the G-protein and subsequently to a large variety of intracellular signalling pathways. The mGluR family is composed of eight members. They are classified into three groups (group I comprising mGlu1 and mGlu5; group II comprising mGlu2 and mGlu3; group III comprising mGlu4, mGlu6, mGlu7, and mGlu8) according to sequence homology, pharmacological profile, and nature of intracellular signalling cascades activated (Schoepp et al. (1999) Neuropharmacology, 38:1431-1476).

Among mGlu receptors, the mGlu7 subtype is the most widely distributed and is present pre-synaptically at a broad range of synapses that are postulated to be critical for both normal CNS functions and a range of psychiatric and neurological disorders (Ohishi et al. (1995) J. Comp. Neurol. 360(4):555-570; Kinzie et al. (1995) Neuroscience, 69(1):167-176; Corti et al. (1998) Eur. J. Neurosci, 10(12):3629-3641). mGlu7 is negatively coupled to adenylate cyclase via activation of Gαi-protein, and its activation as a pre-synaptic autoreceptor leads to inhibition of glutamate and GABA release in the synapse (Dalezios et al. (2002) Cereb. Cortex, 12(9):961-974; Cartmell and Schoepp (2000) J. Neurochem., 75:889-907; Somogyi et al. (2003) Eur. J. Neurosci. 17(12):2503-2520) therefore shaping the synaptic responses at glutamatergic synapses as well as being a key regulator of inhibitory GABAergic transmission with the final goal of fine tuning the overall excitability of the brain.

Previously, most available pharmacological tools targeting mGluRs were orthosteric ligands which cross react with several members of the family as they are structural analogs of glutamate (Schoepp et al. (1999) Neuropharmacology, 38:1431-1476). However, with new screening methods, it has become possible to identify molecules selective to individual mGluRs that act through allosteric mechanisms, modulating the receptor by binding to a site different from the highly conserved orthosteric binding site. These types of molecules have been discovered for several mGluRs (reviewed in Hellyer et al. (2017) Curr. Opin. Pharmacol. 32:49-55; Stansley & Conn (2019) Trends Pharmacol. Sci. 40(4):240-52; Dogra & Conn, (2022) Mol. 101(5):275-285). Several small molecules targeting mGlu7 receptors have been identified in recent years (reviewed in Vasquez-Villa & Trabanco (2019) Med. Chem. Comm. 10:193-9). AMN082 was described as being a potent, selective and systemically active mGlu7 allosteric agonist (Mitsukawa et al. (2005) Proc. Natl. Acad. Sci. USA, 102:18712-18717). 7-Hydroxy-3-(4-iodophenoxy)-4H-chromen-4-one (XAPO44), an allosteric antagonist of mGlu7 was also recently described (Gee et al. (2014) J. Biol. Chem. 18; 289(16):10975-10987), acting via a binding pocket localized in the receptor's extracellular Venus flytrap domain. Finally, several classes of compounds have been described, such as isoxazolopyridinone derivatives, phenylbenzamide derivatives, dihydrobenzoxazolone derivatives, tetrahydrophthalazinone derivatives and pharmacologically characterized as selective mGlu7 negative allosteric modulators (Suzuki et al. (2007) J. Pharmacol. Exp. Ther., 323:147-156; Kalinichev et al. (2013) J. Pharmacol. Exp. Ther. 344(3):624-636; Reed et al. (2017) ACS Med. Chem. Lett. (12):1326-1330 and Duvey et al (2019) WO2019063569).

Specifically, modulators of the mGlu7, and preferably antagonists, inverse agonists, and negative allosteric modulators (NAMs), are reported to hold potential for the treatment of neurological, psychiatric, mood disorders as well as pain and otic disorders, based on experimental studies on laboratory animals, deemed relevant to clinical syndromes.

Combined expression of mGlu7 in brain regions and pharmacological manipulations of mGlu7 in genetically modified mice and wild-type animals reveal an important role for mGlu7 in numerous CNS disorders, including depression, schizophrenia, anxiety, obsessive compulsive disorders and associated symptoms (reviewed by Pallazo et al. (2016) Curr. Neuropharmacol. 14(5): 504-513), and in particular in acute and chronic stress-related disorders (reviewed by Peterlik et al. (2016) Curr Neuropharmacol. 14(5):514-539).

mGlu7 has been shown to be located on limbic system nuclei such as the amygdala, hippocampus and the locus coerulus, regions that are known to be critical for the manifestation of anxiolysis and antidepressant actions (Kinoshita et al. (1998) J. Comp. Neurol., 393(3):332-352; Makoff et al. (1996) Brain Res. Mol. Brain Res., 40(1):165-170; Kinzie et al. (1995) Neuroscience, 69(1):167-176). Moreover, studies in several behavioral models (light-dark box test, elevated plus maze, staircase test, forced swim test and tail suspension test) have shown that mGlu7 knockout animals exhibit an anxiolytic and anti-depressant phenotype but also some deficits in amygdala-dependent behaviors (fear response and conditioned taste aversion) (Cryan et al. (2003) Eur. J. Neuroscience, 17:2409-2417). Therefore, a pharmacological agent aiming at modulating mGlu7 activity may represent a novel therapeutic approach for the treatment of neurological and psychiatric disorders such as anxiety and depression.

Activation of mGlu7 using the allosteric agonist AMN082 increase plasma levels of the stress hormones corticosterone and ACTH (Mitsukawa et al. (2005) PNAS, 102(51):18712-18717). This effect is totally absent in mGlu7 knock-out mice. Those results are in accordance with previous genetic studies showing that mGlu7 is an important regulator of stress response in vivo (Mitsukawa et al. (2006) Neuropsychopharm., 31(6):1112-1122). In this paper, Mitsukawa et al. demonstrated that mGlu7 ablation causes dysregulation of the HPA axis and increases hippocampal BDNF protein levels, indicating that this receptor might be implicated in stress-related psychiatric disorders such as anxiety, depression, post-traumatic stress syndrome, behaviours induced by innate fear such as acquisition and extinction of conditioned fear or conditioned taste aversion. These data also confirmed previous observations where mGlu7-deficient mice showed marked reduction in fear-mediated freezing responses during electric foot-shocks and impairment in the ability to associate between a taste stimulus and a malaise-evoking LiCl injection (conditioned taste aversion, CTA) (Masugi et al. (1999) J. Neurosc., 19(3):955-963). These mice also demonstrated a deficit in the acquisition and extinction learning of conditioned responses compared to wild type animals (Goddyn et al. (2008) Neurobiol. Learn. Mem., 90(1):103-111).

Contradictory effects observed with the allosteric agonist AMN082 may be explained by the rapid and long-lasting mGlu7 receptor internalization, coinciding with functional antagonism, and its scarce selectivity in vivo suggests a potential off-target involvement (Sukoff Rizzo et al. (2011) J. Pharmacol. Exp. Ther., 338(1):345-352; Pelkey et al. (2007) Neuropharmacology 52(1):108-117).

The recent discovery of several negative allosteric modulators has contributed to better understanding of functional role of mGlu7 in neural functioning. 6-(4-Methoxyphenyl)-5-methyl-3-pyridin-4-ylisoxazolo[4,5-c]pyridin-4(5H)-one (MMPIP) administered in vivo has demonstrated anxiolytic, anti-depressant like properties, as well as improved cognitive performance in rodent models (Palazzo et al. (2015) Pain, 156(6):1060-1073). 7-Hydroxy-3-(4-iodophenoxy)-4H-chromen-4-one (XAPO44) was shown to produce anti-stress, anti-depressant and anxiolytic-like effects and to reduce freezing in a fear-conditioning paradigm (Gee et al. (2014) J. Biol. Chem. 289(16):10975-10987). Furthermore, (S)-6-(2,4-dimethylphenyl)-2-ethyl-6,7-dihydrobenzo[d]oxazol-4(5H)-one (ADX71743) demonstrated anxiolytic-like effects in the elevated plus maze and marble burying tests, as well as reducing amphetamine-induced hyperactivity without altering baseline locomotor activity (Kalinichev et al. (2013) J. Pharmacol. Exp. Ther. 344(3):624-636). Taken together, these data indicate that inhibiting mGlu7 with a modulator would be useful for the treatment of mood disorders related to anxiety, depression and PTSD.

In addition, mGlu7 receptors have also been implicated in pathways affected during pain. Given its high and wide expression both in the peripheral and central nervous systems, mGlu7 was found to play a role in regulating pain behaviour. The role of mGlu7 in pain was also recently demonstrated using AMN082 injection directly into the central nucleus of the amygdala (CeA) or in the periaqueductal gray (PAG). Under normal conditions, activation of amygdala mGlu7 facilitates pain responses, as shown by a decrease in the spinal withdrawal reflex thresholds and increased audible and ultrasonic vocalizations evoked by brief compression of the knee (Palazzo et al. (2008) Neuropharmacol., 55(4):537-545). In a similar manner, activation of PAG mGlu7 decreased thermoceptive thresholds measured using the tail flick latency in rats (Marabese et al. (2007) J. Neurophysiol., 98:43-53). In rodent models of pain, AMNO82 inhibited hyperalgesia (Dolan et al. (2009) Behav. Pharmacol. 20(7):596-604); Osikowicz et al. (2008) Pain 139(1):117-126). In addition, the mGlu7 negative allosteric modulator ADX71743 was shown to reduce visceral pain in a stress-sensitive model of visceral hypersensitivity (Moloney et al. (2015) Neurobiol. Stress 2:28-33). Altogether these data suggest that activation of mGlu7 receptors worsen pain perception and mGlu7 inhibition reduces it, therefore suggesting negative allosteric modulators of this receptor might be useful in the treatment of pain and pain-related disorders.

Genome-wide studies have also shown an association of the mGlu7 receptor with age-related hearing impairment (ARHI), also called presbycusis. This resulted in the identification of a highly significant and replicated single nucleotide polymorphism (SNP) located in GRM7, the gene coding for the mGlu7 receptor (Van Laer et al. (2010) Eur. J. Hum. Genet., 18(6):685-693; Friedman et al. (2009) Hum. Mol. Genet., 18(4):785-796; Newman et al. (2012) Hear Res. 294:125-132; Luo et al. (2013) PLoS One, 8(10): e77153; Haider et al. (2017) Front. Aging Neurosci. 9:346; Matyas et al. (2019) Pathol. Oncol. Res. 25(4):1645-52; Chang et al. (2018) J. Int. Adv. Otol. 14(2):170-175). GRM7 variants were also identified to be in association of noise-induced hearing loss, as reported by Lu et al. (BMC Med. Genet. (2018), 19(1):4) and tinnitus, as reported by Haider et al. (Front. Aging Neurosci. (2017), 9:346). Finally, mGlu7 expression, studied by immunohistochemistry, is located in the neurons of the spiral ganglion, in the inner and outer hair cells of the organ of Corti, and the hair cells of the vestibular apparatus formed by the sacculus, the utriculus and the Crista ampullaris (Friedman et al. (2008) WO2008131439). These data suggest that mGlu7 receptor modulators are of potential use in the experimental treatment of otic disorders linked to the inner ear and auditory nervous system such as age-related hearing loss (presbycusis), noise-induced hearing loss, acute and chronic hearing loss, tinnitus, Meniere's disease and vestibular disorders.

Finally, on top of its wide distribution throughout the CNS, mGlu7 shows the highest degree of evolutionary conservation of all mGluRs (Flor et al. (1997) Neuropharmacol., 36:153-159), suggesting an important role for this receptor in CNS functioning. Moreover, it has a relatively low affinity for glutamate (Okamato et al. (1994) J. Biol. Chem., 269:1231-1236), thus it may remain inactive during normal transmission, only becoming active during times of excessive glutamate release (Ferraguti F. and Shigemoto R. (2006) Cell Tissue Res., 326:483-504). Taken together these data strongly highlight the potential of mGlu7 modulators in clinical indications such as neuroprotection (to treat stroke and head injury, ischemic damage and neurotoxicity).

Altogether, these pharmacological and genetic data strongly support the potential of mGlu7 modulators for the treatment of a wide range of disease and associated symptoms across psychiatric, neurological, neurodevelopmental, otic and pain disorders.

### SUMMARY OF THE INVENTION

The invention relates to compounds having metabotropic glutamate receptor 7 modulator activity. In its most general compound aspect, the present invention provides a compound according to Formula (I),

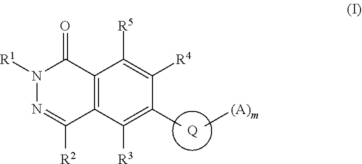

a pharmaceutically acceptable acid or base addition salt thereof, a stereochemically isomeric form thereof or an N-oxide form thereof, wherein:
Q is an optionally substituted aryl or heteroaryl which may further be substituted by 1 to 5 radicals (A)ₘ;
m is an integer ranging from 1 to 5;
the or each (A)ₘ is independently selected from the group of (for example the group consisting of) hydrogen, halogen, —CN, —OH, —NO₂, —CF₃, —OCF₃, —SH, —NH₂ and an optionally substituted radical selected from the group of (for example the group consisting of) —(C₁-C₆)alkyl, —(C₁-C₆)haloalkyl, —(C₂-C₆)alkynyl, —(C₂-C₆)alkenyl, —(C₃-C₇)cycloalkyl, —(C₁-C₆)alkylene-(C₃-C₇)cycloalkyl, —(C₃-C₅)cycloalkenyl, —(C₁-C₆)cyanoalkyl, —(C₁-C₆)alkylene-aryl, aryl, —(C₁-C₆)alkylene-heteroaryl, heteroaryl, —(C₁-C₆)alkylene-heterocycle, heterocycle, —(C₀-C₆)alkylene-OR⁶, —O—(C₂-C₆)alkylene-OR⁶, —NR⁶(C₂-C₆)alkylene-OR⁷, —(C₃-C₆)alkynylene-OR⁶, —(C₃-C₆)alkynylene-NR⁶R⁷, —(C₃-C₆)alkenylene-OR⁶, —(C₃-C₆)alkenylene-NR⁶R⁷, —(C₀-C₆)alkylene-S—R⁶, —O—(C₂-C₆)alkylene-S—R⁶, —NR⁶—(C₂-C₆)alkylene-S—R⁷, —(C₀-C₆)alkylene-S(═O)—R⁶, —O—(C₁-C₆)alkylene-S(═O)—R⁶, —NR⁶—(C₁-C₆)alkylene-S(═O)—R⁷, —(C₀-C₆)alkylene-S(═O)₂—R⁶, —O—(C₁-C₆)alkylene-S(═O)₂—R⁶, —NR⁶—(C₁-C₆)alkylene-S(═O)₂—R⁷, —(C₀-C₆)alkylene-NR⁶R⁷, —O—(C₂-C₆)alkylene-NR⁶R⁷, —NR⁶—(C₂-C₆)alkylene-NR⁷R⁸, —(C₀-C₆)alkylene-S(═O)₂NR⁶R⁷, —O—(C₁-C₆)alkylene-S(═O)₂NR⁶R⁷, —NR⁶—(C₁-C₆)alkylene-S(═O)₂NR⁷R⁸, —(C₀-C₆)alkylene-NR⁶—S(═O)₂R⁷, —O—(C₂-C₆)alkylene-NR⁶—S(═O)₂R⁷, —NR⁶—(C₂-C₆)alkylene-NR⁷—S(═O)₂R⁸, —(C₀-C₆)alkylene-C(═O)—NR⁶R⁷, —O—(C₁-C₆)alkylene-C(═O)—NR⁶R⁷, —NR⁶—(C₁-C₆)alkylene-C(═O)—NR⁷R⁸, —(C₀-C₆)alkylene-NR⁶C(═O)—R⁷, —O—(C₂-C₆)alkylene-NR⁶C(═O)—R⁷, —NR⁶—(C₂-C₆)alkylene-NR⁷C(═O)—R⁸, —NR⁶C(═O)—(C₁-C₆)alkylene-OR⁷, —NR⁶C(═O)—(C₁-C₆)alkylene-NR⁷R⁸, —(C₀-C₆)alkylene-OC(═O)—R⁶, —O—(C₂-C₆)alkylene-OC(═O)—R⁶, —NR⁶—(C₂-C₆)alkylene-OC(═O)—R⁷, —(C₀-C₆)alkylene-C(═O)—OR⁶, —O—(C₁-C₆)alkylene-C(═O)—OR⁶, —NR⁶—(C₁-C₆)alkylene-C(═O)—OR⁷, —(C₀-C₆)alkylene-C(═O)—R⁶, —O—(C₁-C₆)alkylene-C(═O)—R⁶, —NR⁶—(C₁-C₆)alkylene-C(═O)—R⁷, —C(═O)—(C₁-C₆)alkylene-OR⁶, —C(═O)—(C₁-C₆)alkylene-NR⁶R⁷, —(C₀-C₆)alkylene-NR⁶—C(═O)—OR⁷, —C(═O)—(C₁-C₆)alkylene-NR⁶—C(═O)—OR⁷, —(C₀-C₆)alkylene-O—C(═O)—NR⁶R⁷, —(C₀-C₆)alkylene-NR⁶—C(═O)—NR⁷R⁸, —O—(C₂-C₆)alkylene-NR⁶—C(═O)—NR⁷R⁸, —NR⁶—(C₂-C₆)alkylene-NR⁷—C(═O)—NR⁸R⁹ and —(C₀-C₆)alkylene-NR⁶—C(═NR⁷)—NR⁸R⁹; R¹ is an optionally substituted —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₂-C₆)alkylene-O—(C₀-C₆)alkyl, aryl, —(C₁)alkylene-aryl, heterocycle, —(C₁-C₆)alkylene-heterocycle, heteroaryl or —(C₁)alkylene-heteroaryl, wherein the aryl, heterocycle or heteroaryl ring can be substituted by 1 to 5 independent (B)ₙ radicals;
n is an integer ranging from 1 to 5;
the or each (B)ₙ is independently selected from the group of (for example the group consisting of) hydrogen, halogen, —CN, —OH, —NO₂, —CF₃, —OCF₃, —SH, —NH₂ and an optionally substituted radical selected from the group of (for example the group consisting of) —(C₁-C₆)alkyl, —(C₁-C₆)haloalkyl, —(C₂-C₆)alkynyl, —(C₂-C₆)alkenyl, —(C₃-C₇)cycloalkyl, —(C₁-C₆)alkylene-(C₃-C₇)cycloalkyl, —(C₃-C₅)cycloalkenyl, —(C₁-C₆)cyanoalkyl, —(C₁-C₆)alkylene-aryl, aryl, —(C₁-C₆)alkylene-heteroaryl, heteroaryl, —(C₁-C₆)alkylene-heterocycle, heterocycle, —(C₀-C₆)alkylene-OR¹⁰, —O—(C₂-C₆)alkylene-OR¹⁰, —NR¹⁰ (C₂-C₆)alkylene-OR¹¹, —(C₃-C₆)alkynylene-OR¹⁰, —(C₃-C₆)alkynylene-NR¹⁰R¹¹, —(C₃-C₆)alkenylene-OR¹⁰, —(C₃-C₆)alkenylene-NR¹⁰R¹¹, —(C₀-C₆)alkylene-S—R¹⁰, —O—(C₂-C₆)alkylene-S—R¹⁰—NR¹⁰—(C₂-C₆)alkylene-S—R¹¹, —(C₀-C₆)alkylene-S(═O)—R¹⁰, —O—(C₁-C₆)alkylene-S(═O)—R¹⁰, —NR¹⁰—(C₁-C₆)alkylene-S(═O)—R¹, —(C₀-C₆)alkylene-S(═O)₂—R¹⁰, —O—(C₁-C₆)alkylene-S(═O)₂—R¹⁰, —NR¹⁰—(C₁-C₆)alkylene-S(═O)₂—R¹¹, —S(═O)(═NH)—R¹⁰, —(C₀-C₆)alkylene-NR¹⁰R¹¹, —O—(C₂-C₆)alkylene-NR¹⁰R¹¹, —NR¹⁰—(C₂-C₆)alkylene-NR¹¹R¹², —(C₀-C₆)alkylene-S(═O)₂NR¹⁰R¹¹, —O—(C₁-C₆)alkylene-S(═O)₂NR¹⁰R¹¹, —NR¹⁰—(C₁-C₆)alkylene-S(═O)₂NR¹¹R¹², —(C₀-C₆)alkylene-NR¹⁰—S(═O)₂R¹¹, —O—(C₂-C₆)alkylene-NR¹⁰—S(═O)₂R¹¹, —NR¹⁰—(C₂-C₆)alkylene-NR¹¹—S(═O)₂R¹², —(C₀-C₆)alkylene-C(═O)—NR¹⁰R¹¹, —O—(C₁-C₆)alkylene-C(═O)—NR¹⁰R¹¹, —NR¹⁰—(C₁-C₆)alkylene-C(═O)—NR¹¹R¹², —(C₀-C₆)alkylene-NR¹⁰C(═O)—R¹, —O—(C₂-C₆)alkylene-NR¹⁰C(═O)—R¹¹, —NR¹⁰—(C₂-C₆)alkylene-NR¹¹C(═O)—R¹², —NR¹⁰C(═O)—(C₁. C₆)alkylene-OR¹¹, —NR¹⁰C(═O)—(C₁-C₆)alkylene-NR¹¹R¹², —(C₀-C₆)alkylene-OC(═O)—R¹⁰, —O—(C₂-C₆)alkylene-OC(═O)—R¹⁰, —NR¹⁰—(C₂-C₆)alkylene-OC(═O)—R¹¹, —(C₀-C₆)alkylene-C(═O)—OR¹⁰, —O—(C₁-C₆)alkylene-C(═O)—OR¹⁰, —NR¹⁰—(C₁-C₆)alkylene-C(═O)—OR¹¹, —(C₀-C₆)alkylene-C(═O)—R¹⁰, —O—(C₁-C₆)alkylene-C(═O)—R¹⁰, —NR¹⁰—(C₁-C₆)alkylene-C(═O)—R¹¹, —C(═O)—(C₁-C₆)alkylene-OR¹⁰, —C(═O)—(C₁-C₆)alkylene-NR¹⁰R¹¹, —(C₀-C₆)alkylene-NR¹⁰—C(═O)—OR¹¹, —C(═O)—(C₁-C₆)alkylene-NR¹⁰—C(═O)—OR¹¹, —(C₀-C₆)alkylene-O—C(═O)—NR¹⁰R¹¹, —(C₀-C₆)alkylene-NR¹⁰—C(═O)—NR¹¹R¹², —O—(C₂-C₆)alkylene-NR¹⁰—C(═O)—NR¹¹R¹², —NR¹⁰—(C₂-C₆)alkylene-NR¹¹—C(═O)—NR¹²R¹³ and —(C₀-C₆)alkylene-NR¹⁰—C(═NR¹¹)—NR¹²R¹³;
wherein optionally two radicals B are combined with the intervening atoms to form a 3 to 10 membered bicyclic heterocycle ring; wherein each ring is optionally further substituted with 1 to 5 radicals independently selected from the group of (for example the group consisting of) halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂;
R² is selected from the group of (for example the group consisting of) hydrogen, halogen, —CN, —NO₂, —CF₃ and an optionally substituted radical selected from the group of (for example the group consisting of) —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₂-C₆)alkylene-heterocycle, —(C₁-C₆)alkylene-OR¹⁴, —NR¹⁴(C₂-C₆)alkylene-OR¹⁵, —(C₀-C₆)alkylene-S—R¹⁴, —(C₀-C₆)alkylene-S(═O)—R¹⁴, —(C₀-C₆)alkylene-S(═O)₂—R¹⁴, —(C₀-C₆)alkylene-NR¹⁴R¹⁵, —NR¹⁴—(C₂-C₆)alkylene-NR¹⁵R¹⁶, —(C₀-C₆)alkylene-S(═O)₂NR¹⁴R¹⁵, —(C₀-C₆)alkylene-NR¹⁴—S(═O)₂R¹⁵, —(C₀-C₆)alkylene-C(═O)—NR¹⁴R¹⁵, —(C₀-C₆)alkylene-NR¹⁴C(═O)—R¹⁵, —(C₁-C₆)alkylene-OC(═O)—R¹⁴, —(C₀-C₆)alkylene-C(═O)—OR¹⁴, —(C₀-C₆)alkylene-C(═O)—R¹⁴, —(C₀-C₆)alkylene-NR¹⁴—C(═O)—OR¹⁵, —(C₀-C₆)alkylene-O—C(═O)—NR¹⁴R¹⁵, —(C₀-C₆)alkylene-NR¹⁴—C(═O)—NR¹⁵R¹⁶ and —(C₀-C₆)alkylene-NR¹⁴—C(═NR¹⁵)—NR¹⁶R¹⁷;
R³ and R⁴ are each independently selected from the group of (for example the group consisting of) hydrogen, halogen, —CN, —OH, —NO₂, —CF₃, —OCF₃, —SH, —NH₂ and an optionally substituted radical selected from the group of (for example the group consisting of) —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, —(C₁-C₆)alkylene-heteroaryl, heteroaryl, heterocycle, —(C₁-C₆)alkylene-heterocycle, —(C₀-C₆)alkylene-OR¹⁸, —O—(C₂-C₆)alkylene-OR¹⁹, —NR¹⁸(C₂-C₆)alkylene-OR¹⁹, —(C₀-C₆)alkylene-S—R¹⁸, —(C₀-C₆)alkylene-S(═O)—R¹⁸, —(C₀-C₆)alkylene-S(═O)₂—R¹⁸, —(C₀-C₆)alkylene-NR¹⁸R¹⁹, —O—(C₂-C₆)alkylene-NR¹⁸R¹⁹, —NR¹⁸—(C₂-C₆)alkylene-NR¹⁹R²⁰, —(C₀-C₆)alkylene-S(═O)₂NR¹⁸R¹⁹, —(C₀-C₆)alkylene-NR¹⁸—S(═O)₂R¹⁹, —(C₀-C₆)alkylene-C(═O)—NR¹⁸R¹⁹, —(C₀-C₆)alkylene-NR¹⁸C(═O)—R¹⁹, —(C₀-C₆)alkylene-OC(═O)—R¹⁹, —(C₀-C₆)alkylene-C(═O)—OR¹⁹, —(C₀-C₆)alkylene-C(═O)—R¹⁸, —(C₀-C₆)alkylene-NR¹⁸—C(═O)—OR¹⁹, —(C₀-C₆)alkylene-O—C(═O)—NR¹⁸R¹⁹, —(C₀-C₆)alkylene-NR¹⁸—C(═O)—NR¹⁹R²⁰ and —(C₀-C₆)alkylene-NR¹⁸—C(═NR¹⁹)—NR²⁰R²¹;
R⁵ is independently selected from the group of (for example the group consisting of) hydrogen, halogen, —CN, —OH, —NO₂, —CF₃, —OCF₃, —SH and an optionally substituted radical selected from the group of (for example the group consisting of) —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₁-C₆)alkylene-heterocycle, —(C₀-C₆)alkylene-OR²², —O—(C₂-C₆)alkylene-OR²², —NR²²(C₂-C₆)alkylene-OR²³, —(C₀-C₆)alkylene-S—R²², —(C₀-C₆)alkylene-S(═O)—R²², —(C₀-C₆)alkylene-S(═O)₂—R²², —(C₁-C₆)alkylene-NR²²R²³, —O—(C₂-C₆)alkylene-NR²²R²³—NR²²—(C₂-C₆)alkylene-NR²³R²⁴, —(C₀-C₆)alkylene-S(═O)₂NR²²R²³, —(C₀-C₆)alkylene-NR²²—S(═O)₂R²³, —(C₀-C₆)alkylene-C(═O)—NR²²R²³, —(C₀-C₆)alkylene-NR²²C(═O)—R²³, —(C₀-C₆)alkylene-OC(═O)—R²², —(C₀-C₆)alkylene-C(═O)—OR²², —(C₀-C₆)alkylene-C(═O)—R²², —(C₀-C₆)alkylene-NR²²—C(═O)—OR²³, —(C₀-C₆)alkylene-O—C(═O)—NR²²R²³, —(C₀-C₆)alkylene-NR²²—C(═O)—NR²³R²⁴ and —(C₀-C₆)alkylene-NR²²—C(═NR²³)—NR²⁴R²⁵; and
R⁶, R⁷, R¹, R⁹, R¹⁰, R¹¹, R², R¹³, R¹⁴, R¹⁵, R¹⁶, R¹⁷, Rig, R¹⁹R²⁰, R²¹, R²², R²³, R²⁴ and R²⁵ are each independently hydrogen or an optionally substituted radical selected from the group of (for example the group consisting of) —(C₁-C₆)haloalkyl, —(C₁-C₆)alkyl, —(C₁-C₆)cyanoalkyl, —(C₃-C₇)cycloalkyl, —(C₁-C₆)alkylene-(C₃-C₇)cycloalkyl, heteroaryl, —(C₁-C₆)alkylene-heteroaryl, aryl, —(C₁-C₆)alkylene-aryl, —(C₁-C₆)alkylene-heterocycle, heterocycle, —(C₀-C₆)alkylene-O—(C₀-C₆)alkyl and (C₀-C₆)alkylene-N—((C₀-C₆)alkyl)₂.

The compound of formula (I), a pharmaceutically acceptable acid or base addition salt thereof, a stereochemically isomeric form thereof or an N-oxide form thereof may be as defined above provided that:

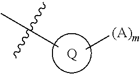

is not

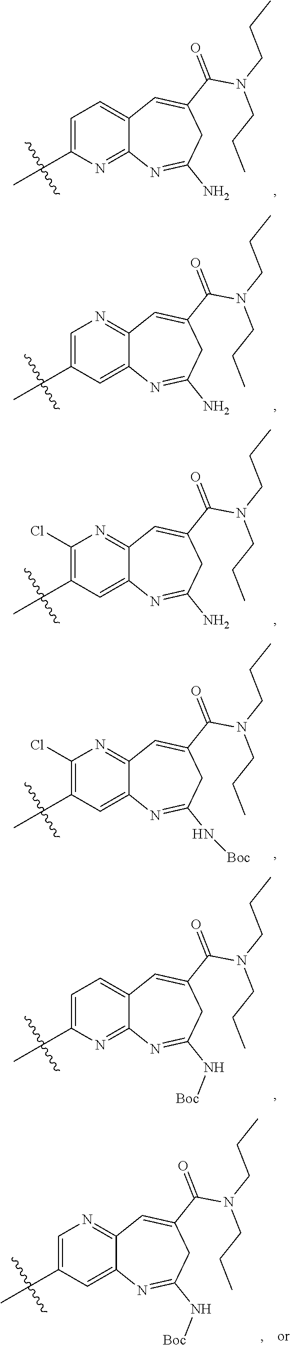

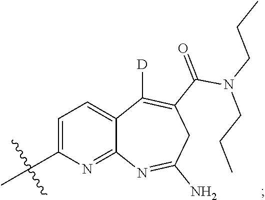 [SMILES: *c1ccc2c(n1)N=C(N)CC(C(=O)N(CCC)CCC)=C2[2H]]

is not

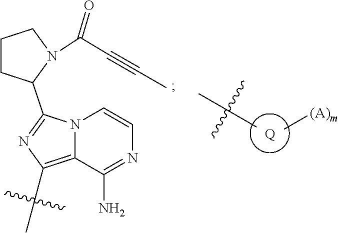

is not

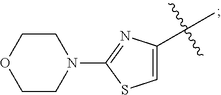 [SMILES: *c1csc(N2CCOCC2)n1]

when

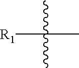

is pyridyl, B is not a substituted pyrrolidinyl radical, for example a pyrrolidinyl substituted by a secondary or tertiary aminyl radical, and when

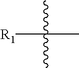

is pyridyl, the pyridyl may not be substituted by methyl and a substituted pyrrolidinyl radical, for example a pyrrolidinyl substituted by a secondary or tertiary aminyl radical.

An example of a substituted pyrrolidinyl radical is

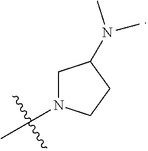 [SMILES: *N1CCC(N(C)C)C1]

For example, when

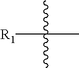

is pyridyl, B is not

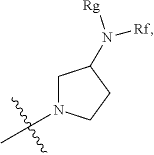

and when

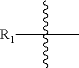

is pyridyl, the pyridyl may not be substituted by

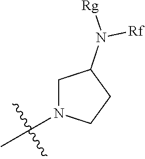

and methyl, wherein R_{g} and R_{f} are each independently selected from (C₁-C₆)alkyl, (C₁-C₆)alkyl-O—(C₁-C₆)alkyl, (C₁-C₆)alkyl-(C═O)— (C₁-C₆)alkyl, and hydrogen, or R_{g} and R_{f}, together with the nitrogen to which they are attached, form a morpholinyl group or a pyrrolidinyl group;
and provided that R² is not

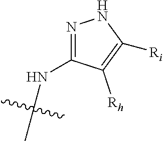

wherein Rₕ is hydrogen, (C₁-C₆)alkyl, cyano or halogen, and Rᵢ is hydrogen, (C₁-C₆)alkyl or (C₃-C₇)cycloalkyl; and provided that R² is not

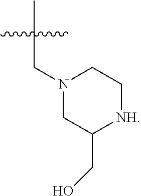 [SMILES: *CN1CCNC(CO)C1]

The compound of formula I, a pharmaceutically acceptable acid or base addition salt thereof, a stereochemically isomeric form thereof or an N-oxide form thereof may be as defined above provided that: Q is not

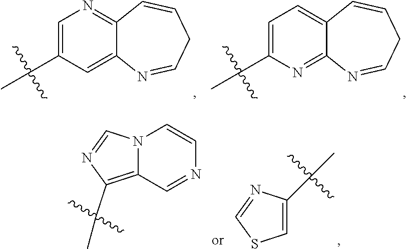 [SMILES: *c1cnc2c(c1)N=CCC=C2 ; *c1ccc2c(n1)N=CCC=C2 ; *c1ncn2ccncc12 ; *c1cscn1]

and when

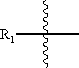

is pyridyl, B is not a substituted pyrrolidinyl radical, and when

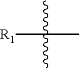

is pyridyl, the pyridyl may not be substituted by methyl and a substituted pyrrolidinyl radical, for example a pyrrolidinyl substituted by a secondary or tertiary aminyl radical. An example of a substituted pyrrolidinyl radical is

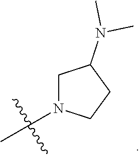 [SMILES: *N1CCC(N(C)C)C1]

For example, when

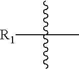

is pyridyl, B is not

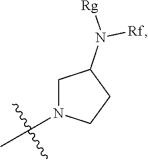

and when

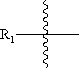

is pyridyl, the pyridyl may not be substituted by methyl and

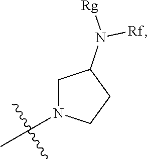

wherein R_{g} and R_{f} are each independently selected from (C₁-C₆)alkyl, (C₁-C₆)alkyl-O—(C₁-C₆)alkyl, (C₁-C₆)alkyl-(C═O)—(C₁-C₆)alkyl, and hydrogen, or R_{g} and R_{f}, together with the nitrogen to which they are attached, form a morpholinyl group or a pyrrolidinyl group;
and provided that R² is not

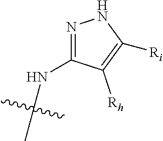

Rₕ wherein Rₕ is hydrogen, (C₁-C₆)alkyl, cyano or halogen, and Rᵢ is hydrogen, (C₁-C₆)alkyl or (C₃-C₇)cycloalkyl, for example Rₕ is hydrogen, (C₁-C₁₀)alkyl, cyano or halogen; and Rᵢ is hydrogen, (C₁-C₁₀)alkyl or cycloalkyl;
and provided that R² is not

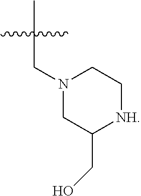 [SMILES: *CN1CCNC(CO)C1]

The compound of Formula (I), a pharmaceutically acceptable acid or base addition salt thereof, a stereochemically isomeric form thereof or an N-oxide form thereof may be as defined above provided that:

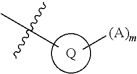

is not

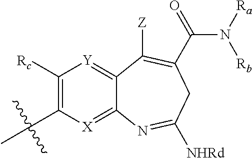

wherein Rₐ and R_{b} are each independently (C₁-C₆)alkyl, R_{c} is hydrogen or halogen; R_{d} is hydrogen or tert-butyloxycarbonyl (BOC), and Z is hydrogen or deuterium;
provided that:

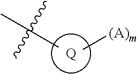

is not

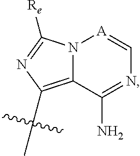

wherein A is CH or N, and Rₑ is

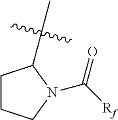

R_{f} is selected from the group of (for example the group consisting of) (C₁-C₆)alkyl, (C₂-C₆)alkenyl, (C₂-C₆) alkynyl, (C₃-C₆)cycloalkyl, (C₃-C₆)heterocycloalkyl, (C₆-C₁₀)aryl, and (C₆-C₁₀) heteroaryl;
provided that:

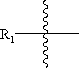

is not

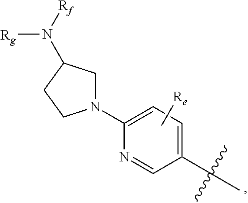

wherein Rₑ is hydrogen or (C₁-C₆)alkyl, R_{g} and R_{f} are each independently selected from the group of (for example the group consisting of) hydrogen, (C₁-C₆)alkyl, (C₁-C₆)alkyl-O—(C₁-C₆)alkyl, and (C₁-C₆)alkyl-(C═O)—(C₁-C₆)alkyl, or R_{g} and R_{f}, together with the nitrogen to which they are attached, form a morpholinyl group or a pyrrolidinyl group, for example provided that if R¹ is pyridyl, the pyridyl ring is bonded to the phthalazinone nitrogen at the 2 position with respect to the pyridyl nitrogen;
provided that R² is not

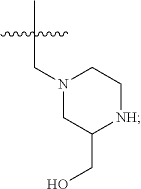 [SMILES: *CN1CCNC(CO)C1]

and
provided that R² is not

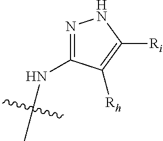

wherein Rₕ is hydrogen, (C₁-C₁₀)alkyl, cyano or halogen; and Rᵢ is hydrogen, (C₁-C₁₀)alkyl or cycloalkyl.

The compound of Formula (I), a pharmaceutically acceptable acid or base addition salt thereof, a stereochemically isomeric form thereof or an N-oxide form thereof may be as defined above provided that R¹ is not

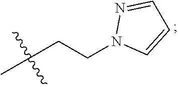 [SMILES: *CCn1cccn1]

Q is not naphthyl, benzothiophenyl or quinolinyl; and Q is not

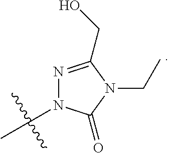 [SMILES: *n1nc(CO)n(CC)c1=O]

For example, Q may not be

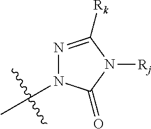

wherein Rⱼ is selected from the group consisting of C₁₋₆ alkyl, C₁₋₆ haloalkyl and C₃₋₆ cycloakyl; and Rk is C₁₋₆ alkyl or C₁₋₆ alkyl substituted with a member selected from the group consisting of: OH, halo, CN, OC₁₋₆ alkyl, OC₁₋₆ haloalkyl and OC₃₋₆ cycloalkyl.

It has surprisingly been found that the compounds of general Formula (I) show potent activity and selectivity on mGlu7 receptor. The compounds of the invention demonstrate advantageous properties over compounds of the prior art. Improvements have been observed in one or more of the following characteristics of the compounds of the invention: the potency on the target, the selectivity for the target, the bioavailability, the brain penetration, and the pharmacodynamics.

R² may be selected from the group of (for example the group consisting of) hydrogen, halogen, —CN, —NO₂, —CF₃ and a radical selected from the group of (for example the group consisting of) —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₂-C₆)alkylene-heterocycle, —(C₁-C₆)alkylene-OR¹⁴, —NR¹⁴(C₂-C₆)alkylene-OR¹⁵, —(C₀-C₆)alkylene-S—R¹⁴, —(C₀-C₆)alkylene-S(═O)—R¹⁴, —(C₀-C₆)alkylene-S(═O)₂—R¹⁴, —NR¹⁴—(C₂-C₆)alkylene-NR¹⁵R¹⁶, —(C₀-C₆)alkylene-S(═O)₂NR¹⁴R¹⁵, —(C₀-C₆)alkylene-NR¹⁴—S(═O)₂R¹⁵, —(C₀-C₆)alkylene-C(═O)—NR¹⁴R¹⁵, —(C₀-C₆)alkylene-NR¹⁴C(═O)—R¹5, —(C₁-C₆)alkylene-OC(═O)—R¹⁴, —(C₀-C₆)alkylene-C(═O)—OR¹⁴, —(C₀-C₆)alkylene-C(═O)—R¹⁴, —(C₀-C₆)alkylene-NR¹⁴, —C(═O)—OR¹⁵, —(C₀-C₆)alkylene-O—C(═O)—NR¹⁴R¹⁵, —(C₀-C₆)alkylene-NR¹⁴—C(═O)—NR¹⁵R¹⁶ and —(C₀-C₆)alkylene-NR¹⁴—C(═NR¹⁵)—NR¹⁶R¹⁷.

R² may be selected from the group of (for example the group consisting of) hydrogen, halogen, —CN, —NO₂, —CF₃ and an optionally substituted radical selected from the group of —(C₂-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₂-C₆)alkylene-heterocycle, —(C₁-C₆)alkylene-OR¹⁴, —NR¹⁴(C₂-C₆)alkylene-OR¹⁵, —(C₀-C₆)alkylene-S—R¹⁴, —(C₀-C₆)alkylene-S(═O)—R¹⁴, —(C₀-C₆)alkylene-S(═O)₂—R¹⁴, —NR¹⁴—(C₂-C₆)alkylene-NR¹⁵R¹⁶, —(C₀-C₆)alkylene-S(═O)₂NR¹⁴R¹⁵, —(C₀-C₆)alkylene-NR¹⁴—S(═O)₂R¹⁵, —(C₀-C₆)alkylene-C(═O)—NR¹⁴R¹⁵, —(C₀-C₆)alkylene-NR¹⁴C(═O)—R¹⁵, —(C₁-C₆)alkylene-OC(═O)—R¹⁴, —(C₀-C₆)alkylene-C(═O)—OR¹⁴, —(C₀-C₆)alkylene-C(═O)—R¹⁴, —(C₀-C₆)alkylene-NR¹⁴—C(═O)—OR¹⁵, —(C₀-C₆)alkylene-O—C(═O)—NR¹⁴R¹⁵, —(C₀-C₆)alkylene-NR¹⁴—C(═O)—NR¹⁵R¹⁶ and —(C₀-C₆)alkylene-NR¹⁴—C(═NR¹⁵)—NR¹⁶R¹⁷.

R², R³, R⁴ and R⁵ may each be independently selected from hydrogen or (C₁-C₆)alkyl.

R² may be hydrogen or (C₁-C₆)alkyl, for example (C₂-C₆)alkyl.

R¹ may be —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₂-C₆)alkylene-O—(C₀-C₆)alkyl, aryl, —(C₁)alkylene-aryl, heterocycle, —(C₁-C₆)alkylene-heterocycle, heteroaryl or —(C₁)alkylene-heteroaryl, wherein the aryl, heterocycle or heteroaryl ring can be substituted by 1 to 5 independent (B)ₙ radicals; n may be an integer ranging from 1 to 5.

For example, R¹ may be —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₂-C₆)alkylene-O—(C₀-C₆)alkyl, aryl, —(C₁)alkylene-aryl, heterocycle, heteroaryl or —(C₁)alkylene-heteroaryl, wherein the aryl, heterocycle or heteroaryl ring can be substituted by 1 to 5 independent (B)ₙ radicals; n may be an integer ranging from 1 to 5.

Preferably, Q represents an aryl or heteroaryl group of formula:

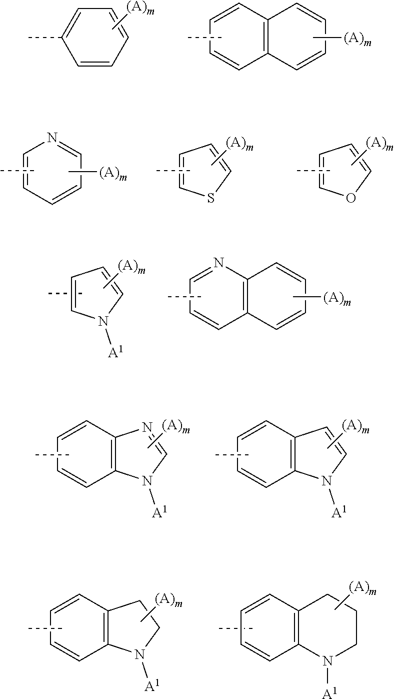

wherein each radical is optionally substituted with m radicals A, wherein m is an integer equal to zero, 1, 2, 3, 4 or 5, and A¹ is a radical A as described above. For example, A¹ may be hydrogen, —(C₁-C₆)alkyl or —(C₃-C₇)cycloalkyl.

Q may be an optionally substituted phenyl or heteroaryl which may further be substituted by 1 to 5 radicals (A)ₘ, wherein m is an integer ranging from 1 to 5.

Q may represent an aryl or heteroaryl group of formula:

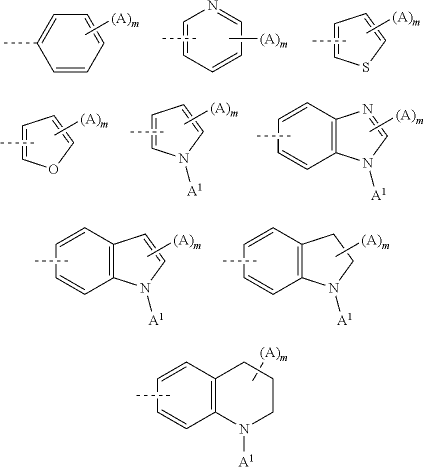

wherein each radical is optionally substituted with m radicals A, wherein m is an integer equal to zero, 1, 2, 3, 4 or 5, and A¹ is a radical A as described above. For example, A¹ may be hydrogen —(C₁-C₆)alkyl or —(C₃-C₇)cycloalkyl.

For the case in which Q is as defined above, when

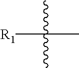

is pyridyl, B may not be a substituted pyrrolidinyl radical, for example a pyrrolidinyl substituted by a secondary or tertiary aminyl radical, and when

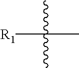

is pyridyl, the pyridyl may not be substituted by methyl and a substituted pyrrolidinyl radical, for example a pyrrolidinyl substituted by a secondary or tertiary aminyl radical. An example of a substituted pyrrolidinyl radical is

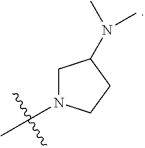 [SMILES: *N1CCC(N(C)C)C1]

For example, when

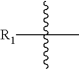

is pyridyl, B may not be

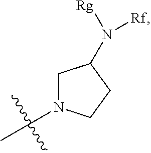

and when

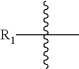

is pyridyl, the pyridyl may not be substituted by

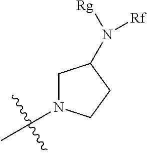

and methyl, wherein R_{g} and R_{f} are each independently selected from (C₁-C₆)alkyl, (C₁-C₆)alkyl-O—(C₁-C₆)alkyl, (C₁-C₆)alkyl-(C═O)—(C₁-C₆)alkyl, and hydrogen, or R_{g} and R_{f}, together with the nitrogen to which they are attached, form a morpholinyl group or a pyrrolidinyl group.

R² may not be

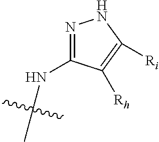

wherein Rₕ is hydrogen, (C₁-C₆)alkyl, cyano or halogen, and Rᵢ is hydrogen, (C₁-C₆)alkyl or (C₃-C₇)cycloalkyl; and
R² may not be

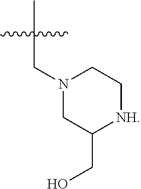 [SMILES: *CN1CCNC(CO)C1]

For the case in which Q is as defined above, R¹ may not be

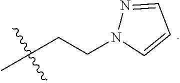 [SMILES: *CCn1cccn1]

The cycloalkyl, heterocycle, aryl and heteroaryl ring systems of (A)ₘ may be selected from the group of (for example the group consisting of) azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl, and each ring of said ring system may be optionally substituted independently with 1 to 4 substituents R⁶, R⁷, R⁸ or R⁹.

For example, the or each (A)ₘ may independently be selected from the group of (for example the group consisting of) hydrogen, halogen, —CF₂CH₃, —OCHF₂ and an optionally substituted radical selected from the group of —(C₁-C₆)alkyl, —(C₃-C₇)cycloalkyl, heterocycle and —(C₀-C₆)alkylene-OR⁶; wherein R⁶ may be selected from the group of hydrogen, —(C₁-C₆)alkyl and —(C₃-C₇)cycloalkyl.

The cycloalkyl, heterocycle, aryl and heteroaryl ring systems of (B)ₙ may be selected from the group of (for example the group consisting of) azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl, cyclooctenyl, 2-oxa-6-azaspiro[3.3]heptan-6-yl and 2-oxa-5-azabicyclo[2.2.1]heptan-5-yl, and each ring of said ring system may be optionally substituted independently with 1 to 4 substituents R¹⁰, R¹¹, R¹² or R¹³.

For example, the cycloalkyl, heterocycle, aryl and heteroaryl ring systems of (B)ₙ may be selected from the group of (for example the group consisting of) azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl, cyclooctenyl, 2-oxa-6-azaspiro[3.3]heptan-6-yl and 2-oxa-5-azabicyclo[2.2.1]heptan-5-yl, and each ring of said ring system may be optionally substituted independently with 1 to 4 substituents R¹⁰, R¹¹, R¹² or R¹³.

For example, the or each (B)ₙ may be independently selected from the group of (for example the group consisting of) hydrogen, halogen and an optionally substituted radical selected from the group of —(C₁-C₆)alkyl, heterocycle, —(C₀-C₆)alkylene-OR¹⁰,
—O—(C₂-C₆)alkylene-OR¹⁰, —NR¹⁰ (C₂-C₆)alkylene-OR¹¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-NR¹⁰R¹¹, —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹;
optionally two radicals B are combined with the intervening atoms to form a 3 to 10 membered bicyclic heterocycle ring; wherein each ring is optionally further substituted with 1 to 5 radicals independently selected from the group of (for example the group consisting of) halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and R¹⁰, R¹¹ and R¹² may be each independently selected from the group of hydrogen, —(C₁-C₆)alkyl, —(C₃-C₇)cycloalkyl, heterocycle, —(C₁-C₆)alkylene-heterocycle, —(C₁-C₆)alkylene-heteroaryl and —(C₀-C₆)alkylene-O—(C₀-C₆)alkyl.

The cycloalkyl, heterocycle, aryl and heteroaryl ring systems of R¹, R², R³, R⁴ or R⁵ may be selected from the group of (for example the group consisting of) azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl, and each ring of said ring system may be optionally substituted with 1-5 radicals independently selected from the group of (for example the group consisting of) hydrogen, halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂.

For example, the heteroaryl ring systems of R¹ may be as defined above, provided that if R¹ is pyridyl, the pyridyl ring is bonded to the phthalazinone nitrogen at the 2 position with respect to the pyridyl nitrogen.

R¹ may be heteroaryl optionally substituted by 1 to 5 independent (B)ₙ radicals; and Q may represent an aryl or heteroaryl group of formula

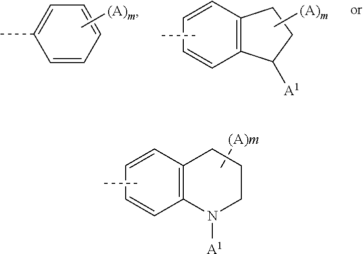

optionally substituted by 1 to 5 radicals (A)ₘ, wherein (A)ₘ and A¹ may be as defined in any statement herein.

Preferably, the compounds of Formula (I), are the compounds according to Formula (II):

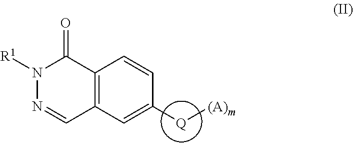

a pharmaceutically acceptable acid or base addition salt thereof, a stereochemically isomeric form thereof or an N-oxide form thereof, wherein R¹, Q and (A)ₘ are as defined in any statement set out above.

The compounds of Formula (II) a pharmaceutically acceptable acid or base addition salt thereof, a stereochemically isomeric form thereof or an N-oxide form thereof may be defined as above, provided that:

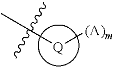

is

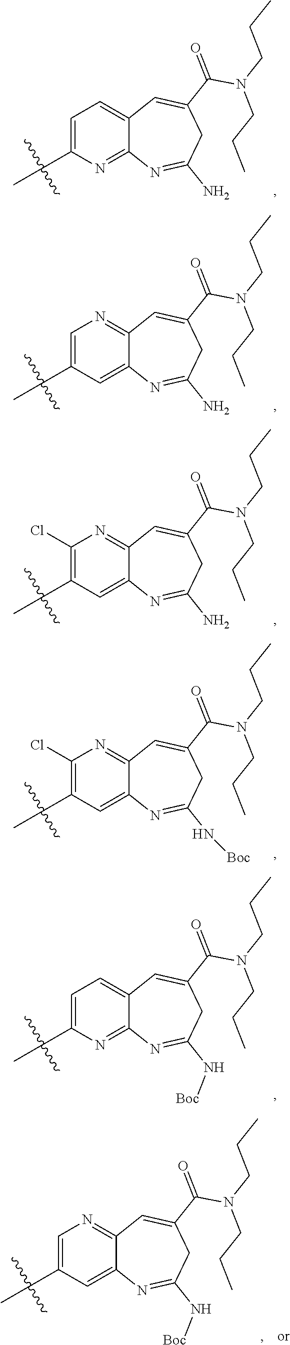

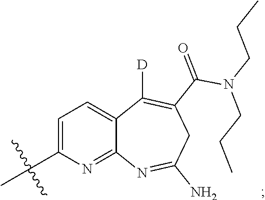 [SMILES: *c1ccc2c(n1)N=C(N)CC(C(=O)N(CCC)CCC)=C2[2H]]

is not

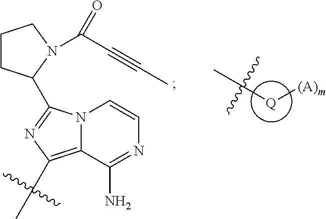

is not

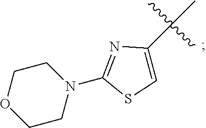 [SMILES: *c1csc(N2CCOCC2)n1]

when

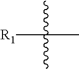

is pyridyl, B is not a substituted pyrrolidinyl radical, for example a pyrrolidinyl substituted by a secondary or tertiary aminyl radical, and when

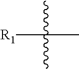

is pyridyl, the pyridyl may not be substituted by methyl and a substituted pyrrolidinyl radical, for example a pyrrolidinyl substituted by a secondary or tertiary aminyl radical.

An example of a substituted pyrrolidinyl radical is

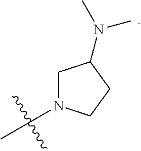 [SMILES: *N1CCC(N(C)C)C1]

For example, when

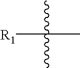

pyridyl, B is not

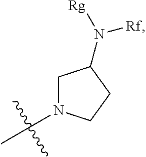

and when

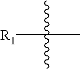

is pyridyl, the pyridyl may not be substituted

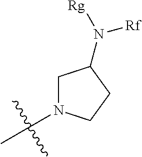

and methyl, wherein R_{g} and R_{f} are each independently selected from (C₁-C₆)alkyl, (C₁-C₆)alkyl-O—(C₁-C₆)alkyl, (C₁-C₆)alkyl-(C═O)—(C₁-C₆)alkyl, and hydrogen, or R_{g} and R_{f}, together with the nitrogen to which they are attached, form a morpholinyl group or a pyrrolidinyl group.

The compounds of Formula (II), a pharmaceutically acceptable acid or base addition salt thereof, a stereochemically isomeric form thereof or an N-oxide form thereof may be as defined above provided that:
Q is not

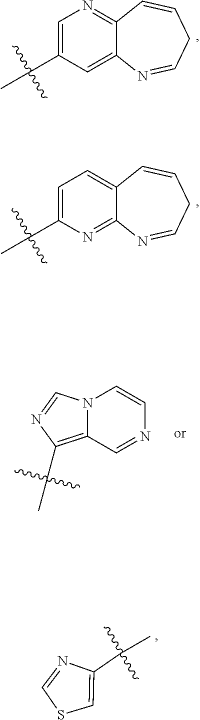 [SMILES: *c1cnc2c(c1)N=CCC=C2 ; *c1ccc2c(n1)N=CCC=C2 ; *c1ncn2ccncc12 ; *c1cscn1]

and when

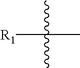

is pyridyl, B is not a substituted pyrrolidinyl radical, and when

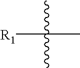

is pyridyl, the pyridyl may not be substituted by methyl and a substituted pyrrolidinyl radical, for example a pyrrolidinyl substituted by a secondary or tertiary aminyl radical. An example of a substituted pyrrolidinyl radical is

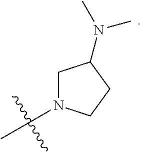 [SMILES: *N1CCC(N(C)C)C1]

For example, when

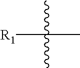

is pyridyl, B is not

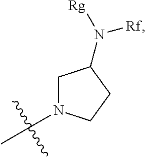

and when

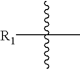

is pyridyl, the pyridyl may not be substituted by methyl and

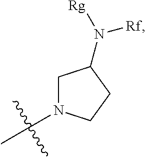

wherein R_{g} and R_{f} are each independently selected from (C₁-C₆)alkyl, (C₁-C₆)alkyl-O—(C₁-C₆)alkyl, (C₁-C₆)alkyl-(C═O)—(C₁-C₆)alkyl, and hydrogen, or R_{g} and R_{f}, together with the nitrogen to which they are attached, form a morpholinyl group or a pyrrolidinyl group.

The compounds of Formula (II), a pharmaceutically acceptable acid or base addition salt thereof, a stereochemically isomeric form thereof or an N-oxide form thereof may be as defined above provided:

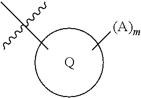

is not

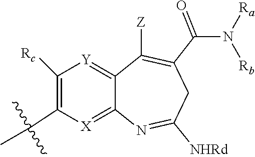

wherein Rₐ and R_{b} are each independently (C₁-C₆)alkyl, R_{c} is hydrogen or halogen; R_{d} is hydrogen or tert-butyloxycarbonyl, and Z is hydrogen or deuterium; provided that:

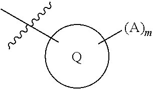

is not

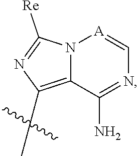

wherein A is CH or N, and Rₑ is

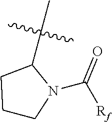

R_{f} is selected from the group of (for example the group consisting of) (C₁-C₆)alkyl, (C₂-C₆)alkenyl, (C₂-C₆) alkynyl, (C₃-C₆)cycloalkyl, (C₃-C₆)heterocycloalkyl, (C₆-C₁₀)aryl, and (C₆-C₁₀) heteroaryl;
provided that:

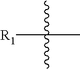

is not

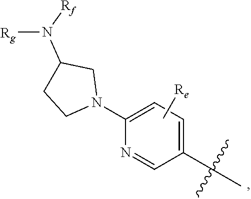

wherein Rₑ is hydrogen or (C₁-C₆)alkyl, R_{g} and R_{f} are each independently selected from the group of (for example the group consisting of) hydrogen, (C₁-C₆)alkyl, (C₁-C₆)alkyl-O—(C₁-C₆)alkyl, and (C₁-C₆)alkyl-(C═O)—(C₁-C₆)alkyl, or R_{g} and R_{f}, together with the nitrogen to which they are attached, form a morpholinyl group or a pyrrolidinyl group.

The compound of Formula (II), a pharmaceutically acceptable acid or base addition salt thereof, a stereochemically isomeric form thereof or an N-oxide form thereof may be as defined above provided that Q is not naphthyl, benzothiophenyl or quinolinyl.

Q may represent an aryl or heteroaryl group of formula:

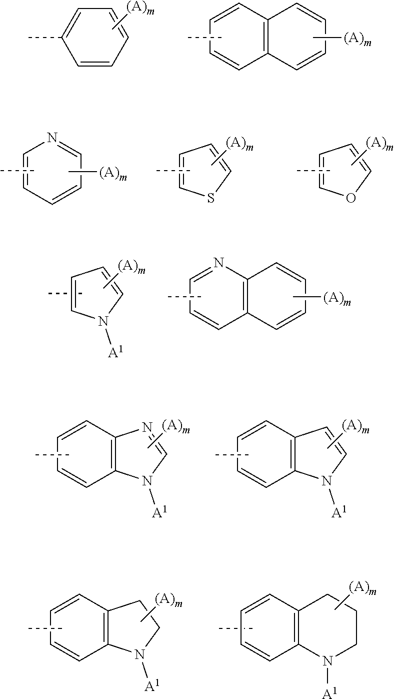

wherein each radical is optionally substituted with m radicals A, wherein m is an integer equal to zero, 1, 2, 3, 4 or 5, and A¹ is a radical A. For example, A¹ may be hydrogen, —(C₁-C₆)alkyl or —(C₃-C₇)cycloalkyl.

Q may represent an aryl or heteroaryl group of formula:

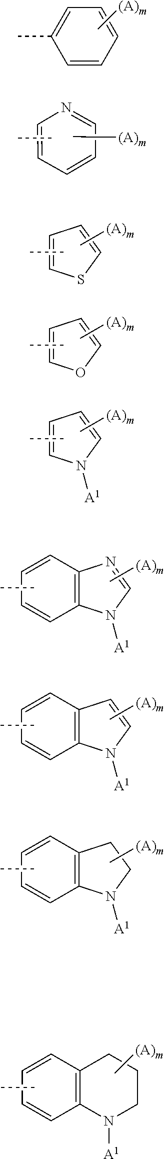

wherein each radical is optionally substituted with m radicals A, wherein m is an integer equal to zero, 1, 2, 3, 4 or 5, and A¹ is a radical A. For example, A¹ may be hydrogen, —(C₁-C₆)alkyl or —(C₃-C₇)cycloalkyl.

Preferably, the compounds of Formula (II), are the compounds according to Formula (III):

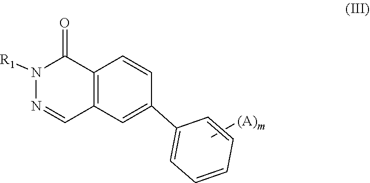

a pharmaceutically acceptable acid or base addition salt thereof, a stereochemically isomeric form thereof or an N-oxide form thereof; wherein (A)ₘ and R₁ are as defined in any statement set out above.

The compounds of Formula (III) a pharmaceutically acceptable acid or base addition salt thereof, a stereochemically isomeric form thereof or an N-oxide form thereof may be defined as above, provided that:
when

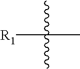

is pyridyl, B is not a substituted pyrrolidinyl radical, for example a pyrrolidinyl substituted by a secondary or tertiary aminyl radical, and when

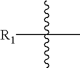

is pyridyl, the pyridyl may not be substituted by methyl and a substituted pyrrolidinyl radical, for example a pyrrolidinyl substituted by a secondary or tertiary aminyl radical.

An example of a substituted pyrrolidinyl radical is

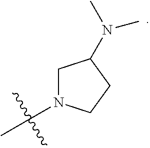 [SMILES: *N1CCC(N(C)C)C1]

For example, when

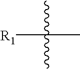

is pyridyl, B is not

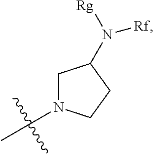

and when

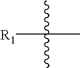

is pyridyl, the pyridyl may not be substituted by

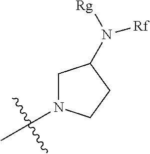

and methyl, wherein R_{g} and R_{f} are each independently selected from (C₁-C₆)alkyl, (C₁-C₆)alkyl-O—(C₁-C₆)alkyl, (C₁-C₆)alkyl-(C═O)—(C₁-C₆)alkyl, and hydrogen, or R_{g} and R_{f}, together with the nitrogen to which they are attached, form a morpholinyl group or a pyrrolidinyl group.

The compounds of Formula (III), a pharmaceutically acceptable acid or base addition salt thereof, a stereochemically isomeric form thereof or an N-oxide form thereof may be as defined above provided that:

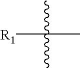

is not

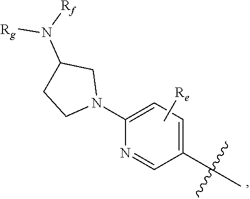

wherein Rₑ is hydrogen or (C₁-C₆)alkyl, R_{g} and R_{f} are each independently selected from the group of (for example the group consisting of) hydrogen, (C₁-C₆)alkyl, (C₁-C₆)alkyl-O—(C₁-C₆)alkyl, and (C₁-C₆)alkyl-(C═O)—(C₁-C₆)alkyl, or R_{g} and R_{f}, together with the nitrogen to which they are attached, form a morpholinyl group or a pyrrolidinyl group.

Preferably, the compounds of Formula (II), are the compounds according to Formula (IV):

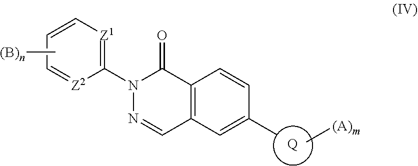

a pharmaceutically acceptable acid or base addition salt thereof, a stereochemically isomeric form thereof or an N-oxide form thereof, wherein Z¹ and Z² are each independently selected from C or N, and Q, (A)ₘ and (B)ₙ are as defined in any statement set out above.

The or each (A)ₘ may be independently selected from the group of (for example the group consisting of) hydrogen, halogen, —CF₂CH₃, —OCHF₂ and an optionally substituted radical selected from the group of —(C₁-C₆)alkyl, —(C₃-C₇)cycloalkyl, heterocycle and —(C₀-C₆)alkylene-OR⁶; R⁶ may be selected from the group of hydrogen, —(C₁-C₆)alkyl and —(C₃-C₇)cycloalkyl.

The or each (B)ₙ may be independently selected from the group of (for example the group consisting of) hydrogen, halogen and an optionally substituted radical selected from the group of —(C₁-C₆)alkyl, heterocycle, —(C₀-C₆)alkylene-OR¹⁰, —O—(C₂-C₆)alkylene-OR¹⁰, —NR¹⁰ (C₂-C₆)alkylene-OR¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-NR¹¹R¹², —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹.
optionally two radicals B are combined with the intervening atoms to form a 3 to 10 membered bicyclic heterocycle ring; wherein each ring is optionally further substituted with 1 to 5 radicals independently selected from the group of halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and
R¹⁰, R¹¹ and R¹² may each be independently selected from the group of (for example the group consisting of) hydrogen, —(C₁-C₆)alkyl, —(C₃-C₇)cycloalkyl, heterocycle, —(C₁-C₆)alkylene-heterocycle, —(C₁-C₆)alkylene-heteroaryl and —(C₀-C₆)alkylene-O—(C₀-C₆)alkyl.

The compounds of Formula (IV) a pharmaceutically acceptable acid or base addition salt thereof, a stereochemically isomeric form thereof or an N-oxide form thereof may be defined as above, provided that:

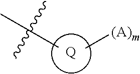

is

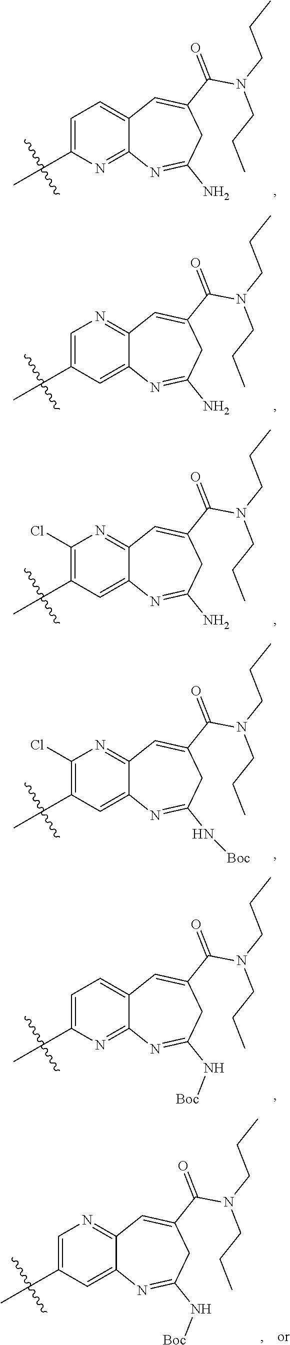

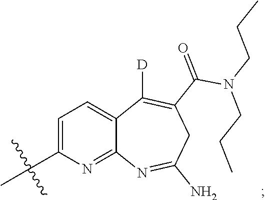 [SMILES: *c1ccc2c(n1)N=C(N)CC(C(=O)N(CCC)CCC)=C2[2H]]

is not

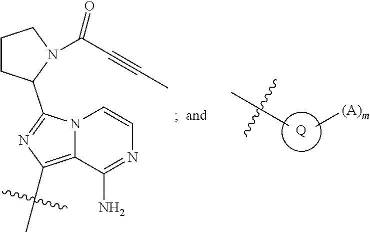

is not

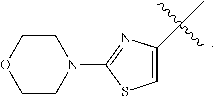 [SMILES: *c1csc(N2CCOCC2)n1]

The compounds of Formula (IV), a pharmaceutically acceptable acid or base addition salt thereof, a stereochemically isomeric form thereof or an N-oxide form thereof may be as defined above provided that: Q is not

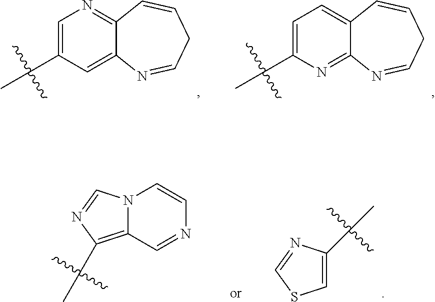 [SMILES: *c1cnc2c(c1)N=CCC=C2 ; *c1ccc2c(n1)N=CCC=C2 ; *c1ncn2ccncc12 ; *c1cscn1]

The compounds of Formula (IV), a pharmaceutically acceptable acid or base addition salt thereof, a stereochemically isomeric form thereof or an N-oxide form thereof may be as defined above provided that:

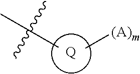

is not

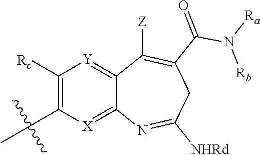

wherein Rₐ and R_{b} are each independently (C₁-C₆)alkyl, R_{c} is hydrogen or halogen; R_{d} is hydrogen or tert-butyloxycarbonyl, and Z is hydrogen or deuterium; provided that:

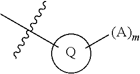

is not

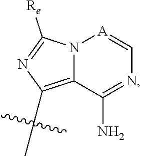

wherein A is CH or N, and Rₑ is

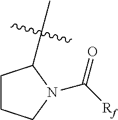

R_{f} is selected from the group of (for example the group consisting of) (C₁-C₆)alkyl, (C₂-C₆)alkenyl, (C₂-C₆) alkynyl, (C₃-C₆)cycloalkyl, (C₃-C₆)heterocycloalkyl, (C₆-C₁₀)aryl, and (C₆-C₁₀) heteroaryl.

The compound of Formula (IV), a pharmaceutically acceptable acid or base addition salt thereof, a stereochemically isomeric form thereof or an N-oxide form thereof may be as defined above provided that Q is not naphthyl, benzothiophenyl or quinolinyl.

Q may represent an aryl or heteroaryl group of formula:

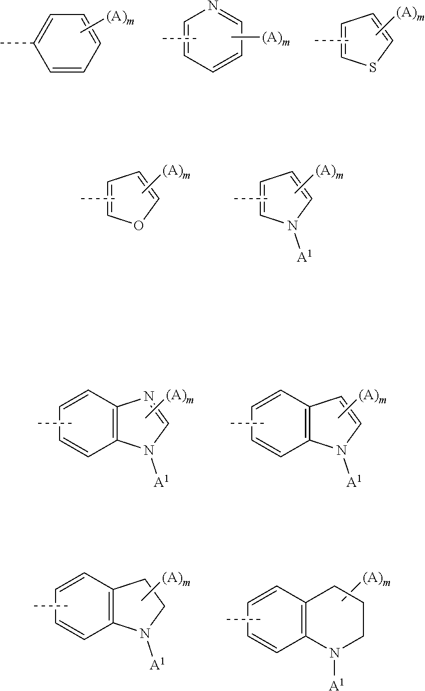

wherein each radical is optionally substituted with m radicals A, wherein m is an integer equal to zero, 1, 2, 3, 4 or 5, and A¹ is a radical A. For example, A¹ may be hydrogen —(C₁-C₆)alkyl or —(C₃-C₇)cycloalkyl.

The or each (A)ₘ may be independently selected from the group of (for example the group consisting of) hydrogen, halogen, —CF₂CH₃, —OCHF₂ and an optionally substituted radical selected from the group of —(C₁-C₆)alkyl, —(C₃-C₇)cycloalkyl, heterocycle and —(C₀-C₆)alkylene-OR⁶; R⁶ may be selected from the group of hydrogen, —(C₁-C₆)alkyl and —(C₃-C₇)cycloalkyl;
the or each (B)ₙ may be independently selected from the group of (for example the group consisting of) hydrogen, halogen and an optionally substituted radical selected from the group of —(C₁-C₆)alkyl, heterocycle, —(C₀-C₆)alkylene-OR¹⁰, —O—(C₂-C₆)alkylene-OR¹⁰, —NR¹⁰(C₂-C₆)alkylene-OR¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-NR¹⁰R¹¹, —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹.
optionally two radicals B are combined with the intervening atoms to form a 3 to 10 membered bicyclic heterocycle ring; wherein each ring is optionally further substituted with 1 to 5 radicals independently selected from the group of halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and
R¹⁰, R¹¹ and R¹² may each be independently selected from the group of hydrogen, —(C₁-C₆)alkyl, —(C₃-C₇)cycloalkyl, heterocycle, —(C₁-C₆)alkylene-heterocycle, —(C₁-C₆)alkylene-heteroaryl and —(C₀-C₆)alkylene-O—(C₀-C₆)alkyl.

Preferably, the compounds of Formula (II), are the compounds according to Formula (V):

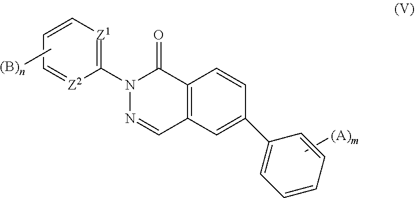

a pharmaceutically acceptable acid or base addition salt thereof, a stereochemically isomeric form thereof or an N-oxide form thereof; wherein Z¹ and Z² are each independently selected from C or N, and (A)ₘ and (B)ₙ are as defined in any statement set out above.

Preferably, the compounds of Formula (V), are the compounds according to Formula (VI):

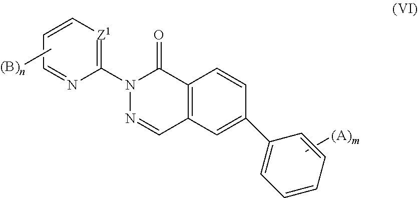

a pharmaceutically acceptable acid or base addition salt thereof, a stereochemically isomeric form thereof or an N-oxide form thereof; wherein Z¹ is selected from C or N;
the or each (A)ₘ is independently selected from the group of (for example the group consisting of) hydrogen, halogen, —CF₂CH₃, —OCHF₂ and an optionally substituted radical selected from the group of (for example the group consisting of) —(C₁-C₆)alkyl, —(C₃-C₇)cycloalkyl, heterocycle and —(C₀-C₆)alkylene-OR⁶;
R⁶ is selected from the group of (for example the group consisting of) hydrogen, —(C₁-C₆)alkyl or —(C₃-C₇)cycloalkyl;
the or each (B)ₙ is independently selected from the group of (for example the group consisting of) hydrogen, halogen and an optionally substituted radical selected from the group of —(C₁-C₆)alkyl, heterocycle, —(C₀-C₆)alkylene-OR¹⁰, —O—(C₂-C₆)alkylene-OR¹⁰, —NR¹⁰(C₂-C₆)alkylene-OR¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-NR¹⁰R¹¹, —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹;
two radicals B may be combined with the intervening atoms to form a 3 to 10 membered bicyclic heterocycle ring; wherein each ring is optionally further substituted with 1 to 5 radicals independently selected from halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and
R¹⁰, R¹¹ and R¹² are each independently hydrogen, —(C₁-C₆)alkyl, —(C₃-C₇)cycloalkyl, heterocycle, —(C₁-C₆)alkylene-heterocycle, —(C₁-C₆)alkylene-heteroaryl or —(C₀-C₆)alkylene-O—(C₀-C₆)alkyl.

Particular preferred compounds of the invention are compounds as mentioned in the following list, as well as a pharmaceutically acceptable acid or base addition salt thereof, a stereochemically isomeric form thereof or an N-oxide form thereof:
6-(2,4-Dimethylphenyl)-2-ethylphthalazin-1(2H)-one
2-Ethyl-6-mesitylphthalazin-1(2H)-one
2-Ethyl-6-(3-methoxyphenyl)phthalazin-1(2H)-one
6-(2,4-Dimethylphenyl)-2-(2-fluorophenyl)phthalazin-1(2H)-one
2-(2-Chlorophenyl)-6-(2,4-dimethylphenyl)phthalazin-1(2H)-one
6-(3-(Dimethylamino)phenyl)-2-ethylphthalazin-1(2H)-one
6-(3-Ethoxyphenyl)-2-ethylphthalazin-1(2H)-one
6-(2,4-Dimethylphenyl)-2-(4-(methylsulfonyl)phenyl)phthalazin-1(2H)-one
6-(2,4-Dimethylphenyl)-2-(4-(methoxymethyl)phenyl)phthalazin-1(2H)-one
6-(2,4-Dimethylphenyl)-2-(pyridin-2-yl)phthalazin-1(2H)-one
6-(3-(Azetidin-1-yl)phenyl)-2-ethylphthalazin-1(2H)-one
2-(4-Acetylphenyl)-6-(2,4-dimethylphenyl)phthalazin-1(2H)-one
2-Ethyl-6-(3-(pyrrolidin-1-yl)phenyl)phthalazin-1(2H)-one
6-(2,4-Dimethylphenyl)-2-(4-(1-hydroxyethyl)phenyl)phthalazin-1(2H)-one
6-(2-Methoxyphenyl)-2-(pyridin-2-yl)phthalazin-1(2H)-one
6-(3-Methoxyphenyl)-2-(pyridin-2-yl)phthalazin-1(2H)-one
6-(4-Methoxyphenyl)-2-(pyridin-2-yl)phthalazin-1(2H)-one
6-(2,4-Dimethylphenyl)-2-(pyridin-4-yl)phthalazin-1(2H)-one
6-(3-(Azetidin-1-yl)phenyl)-2-(2-methoxyphenyl)phthalazin-1(2H)-one
6-(3-(Azetidin-1-yl)phenyl)-2-benzylphthalazin-1(2H)-one
6-(3-Ethoxyphenyl)-2-(pyridin-2-yl)phthalazin-1(2H)-one
6-(3-Cyclopropylphenyl)-2-(pyridin-2-yl)phthalazin-1(2H)-one
2-(Pyridin-2-yl)-6-o-tolylphthalazin-1(2H)-one
6-(3-(Azetidin-1-yl)phenyl)-2-(5-fluoropyridin-2-yl)phthalazin-1(2H)-one
6-(3-(Methoxymethyl)phenyl)-2-(pyridin-2-yl)phthalazin-1(2H)-one
6-(4-Methoxy-2-methylphenyl)-2-(pyridin-2-yl)phthalazin-1(2H)-one
2-(Pyridin-2-yl)-6-(3-(pyrrolidin-1-yl)phenyl)phthalazin-1(2H)-one
6-(2,6-Dimethylphenyl)-2-(pyridin-2-yl)phthalazin-1(2H)-one
2-(3-Fluoropyridin-2-yl)-6-(3-methoxyphenyl)phthalazin-1(2H)-one
6-(3-Methoxyphenyl)-2-(4-methylpyridin-2-yl)phthalazin-1(2H)-one
6-(3-Methoxyphenyl)-2-(pyrimidin-2-yl)phthalazin-1(2H)-one
6-(3-Methoxyphenyl)-2-(6-methylpyridin-2-yl)phthalazin-1(2H)-one
6-(2-Chloro-3-methoxyphenyl)-2-(pyridin-2-yl)phthalazin-1(2H)-one
6-(2-Chloro-3-cyclopropoxyphenyl)-2-(pyridin-2-yl)phthalazin-1(2H)-one
6-(3-Methoxy-2-methylphenyl)-2-(pyridin-2-yl)phthalazin-1(2H)-one
6-(3-Cyclopropoxy-2-methylphenyl)-2-(pyridin-2-yl)phthalazin-1(2H)-one
6-(1-Methylindolin-4-yl)-2-(pyridin-2-yl)phthalazin-1(2H)-one
6-(3-(Difluoromethoxy)phenyl)-2-(pyridin-2-yl)phthalazin-1(2H)-one
6-(3-(1,1-Difluoroethyl)phenyl)-2-(pyridin-2-yl)phthalazin-1(2H)-one
6-(2-Chloro-3-methoxyphenyl)-2-(pyrimidin-2-yl)phthalazin-1(2H)-one
6-(2-Chloro-3-cyclopropoxyphenyl)-2-(pyrimidin-2-yl)phthalazin-1(2H)-one
6-(3-Methoxy-2-methylphenyl)-2-(pyrimidin-2-yl)phthalazin-1(2H)-one
6-(3-Cyclopropoxy-2-methylphenyl)-2-(pyrimidin-2-yl)phthalazin-1(2H)-one
6-(1-Methylindolin-4-yl)-2-(pyrimidin-2-yl)phthalazin-1(2H)-one
6-(3-(Azetidin-1-yl)phenyl)-2-(pyridin-2-yl)phthalazin-1(2H)-one
6-(3-(Azetidin-1-yl)phenyl)-2-(pyrimidin-2-yl)phthalazin-1(2H)-one
6-(1-Cyclopropyl-1H-indol-4-yl)-2-(pyridin-2-yl)phthalazin-1(2H)-one
6-(1-Cyclopropyl-1H-indol-4-yl)-2-(pyrimidin-2-yl)phthalazin-1(2H)-one
6-(3-(Azetidin-1-yl)-5-fluorophenyl)-2-(pyridin-2-yl)phthalazin-1(2H)-one
6-(4-(Azetidin-1-yl)pyrimidin-2-yl)-2-(pyridin-2-yl)phthalazin-1(2H)-one
6-(3-(Azetidin-1-yl)-4-fluorophenyl)-2-(pyridin-2-yl)phthalazin-1(2H)-one
6-(5-(Azetidin-1-yl)-2-fluorophenyl)-2-(pyridin-2-yl)phthalazin-1(2H)-one
6-(4-(Azetidin-1-yl)pyridin-2-yl)-2-(pyridin-2-yl)phthalazin-1(2H)-one
6-(3-Methoxy-2-methylphenyl)-2-(5-methylpyrimidin-2-yl)phthalazin-1(2H)-one
6-(3-Methoxy-2-methylphenyl)-2-(5-methoxypyrimidin-2-yl)phthalazin-1(2H)-one
6-(1-Methylindolin-4-yl)-2-(5-methylpyrimidin-2-yl)phthalazin-1(2H)-one
2-(5-Methoxypyrimidin-2-yl)-6-(1-methylindolin-4-yl)phthalazin-1(2H)-one
2-(3-Fluoropyridin-2-yl)-6-(3-methoxy-2-methylphenyl)phthalazin-1(2H)-one
2-(3-Fluoropyridin-2-yl)-6-(1-methylindolin-4-yl)phthalazin-1(2H)-one
6-(3-Methoxy-2-methylphenyl)-2-(tetrahydro-2H-pyran-4-yl)phthalazin-1(2H)-one
6-(1-Methyl-1,2,3,4-tetrahydroquinolin-5-yl)-2-(pyridin-2-yl)phthalazin-1(2H)-one
2-(5-Fluoropyrimidin-2-yl)-6-(3-methoxy-2-methylphenyl)phthalazin-1(2H)-one
6-(3-Methoxy-2-methylphenyl)-2-(5-(4-methylpiperazin-1-yl)pyrimidin-2-yl)phthalazin-1(2H)-one
6-(3-Methoxy-2-methylphenyl)-2-(5-morpholinopyrimidin-2-yl)phthalazin-1(2H)-one cis-2-(5-(3-Hydroxypyrrolidin-1-yl)pyrimidin-2-yl)-6-(3-methoxy-2-methylphenyl)phthalazin-1(2H)-one
trans-2-(5-(3-Hydroxypyrrolidin-1-yl)pyrimidin-2-yl)-6-(3-methoxy-2-methylphenyl)phthalazin-1(2H)-one
6-(3-Methoxy-2-methylphenyl)-2-(2-morpholinoethyl)phthalazin-1(2H)-one
6-(3-Methoxy-2-methylphenyl)-2-(5-(methylthio)pyridin-2-yl)phthalazin-1(2H)-one
6-(3-Methoxy-2-methylphenyl)-2-(5-(methylsulfonyl)pyridin-2-yl)phthalazin-1(2H)-one
2-(5-(Azetidin-3-yloxy)pyrimidin-2-yl)-6-(3-methoxy-2-methylphenyl)phthalazin-1(2H)-one
6-(3-Methoxy-2-methylphenyl)-2-(5-(pyrrolidin-1-yl)pyrimidin-2-yl)phthalazin-1(2H)-one
6-(3-Methoxy-2-methylphenyl)-2-(5-(piperidin-1-yl)pyrimidin-2-yl)phthalazin-1(2H)-one
2-(5-(4-Hydroxypiperidin-1-yl)pyrimidin-2-yl)-6-(3-methoxy-2-methylphenyl)phthalazin-1(2H)-one
cis-2-(5-(3-Hydroxypiperidin-1-yl)pyrimidin-2-yl)-6-(3-methoxy-2-methylphenyl)phthalazin-1(2H)-one
trans-2-(5-(3-Hydroxypiperidin-1-yl)pyrimidin-2-yl)-6-(3-methoxy-2-methylphenyl)phthalazin-1(2H)-one
2-(5-(4-Hydroxycyclohexylamino)pyrimidin-2-yl)-6-(3-methoxy-2-methylphenyl)phthalazin-1(2H)-one
cis-2-(5-(3-Hydroxycyclohexylamino)pyrimidin-2-yl)-6-(3-methoxy-2-methylphenyl)phthalazin-1(2H)-one
trans-2-(5-(3-Hydroxycyclohexylamino)pyrimidin-2-yl)-6-(3-methoxy-2-methylphenyl)phthalazin-1(2H)-one
6-(3-(Azetidin-1-yl)phenyl)-2-(5-fluoropyrimidin-2-yl)phthalazin-1(2H)-one
6-(3-(Azetidin-1-yl)-2-methylphenyl)-2-(pyridin-2-yl)phthalazin-1(2H)-one
6-(3-Methoxy-2-methylphenyl)-2-(5-thiomorpholinopyrimidin-2-yl)phthalazin-1(2H)-one
6-(3-Methoxy-2-methylphenyl)-2-(5-(2-methoxyethylamino)pyrimidin-2-yl)phthalazin-1(2H)-one
2-(5-(2-(Dimethylamino)ethylamino)pyrimidin-2-yl)-6-(3-methoxy-2-methylphenyl)phthalazin-1(2H)-one
6-(2,4-Dimethylphenyl)-2-(5-morpholinopyrimidin-2-yl)phthalazin-1(2H)-one
cis-6-(2,4-Dimethylphenyl)-2-(5-(3-hydroxypyrrolidin-1-yl)pyrimidin-2-yl)phthalazin-1(2H)-one
trans-6-(2,4-Dimethylphenyl)-2-(5-(3-hydroxypyrrolidin-1-yl)pyrimidin-2-yl)phthalazin-1(2H)-one
cis-6-(3-(Azetidin-1-yl)phenyl)-2-(5-(3-hydroxypyrrolidin-1-yl)pyrimidin-2-yl)phthalazin-1(2H)-one
trans-6-(3-(Azetidin-1-yl)phenyl)-2-(5-(3-hydroxypyrrolidin-1-yl)pyrimidin-2-yl)phthalazin-1(2H)-one
2-(5-(2-Oxa-6-azaspiro[3.3]heptan-6-yl)pyrimidin-2-yl)-6-(3-methoxy-2-methylphenyl)phthalazin-1(2H)-one
2-(5-Ethoxypyrimidin-2-yl)-6-(3-methoxy-2-methylphenyl)phthalazin-1(2H)-one
2-(5-(2-Oxa-5-azabicyclo[2.2.1]heptan-5-yl)pyrimidin-2-yl)-6-(3-methoxy-2-methylphenyl)phthalazin-1(2H)-one
cis-2-(5-(3-Hydroxycyclopentylamino)pyrimidin-2-yl)-6-(3-methoxy-2-methylphenyl)phthalazin-1(2H)-one
trans-2-(5-(3-Hydroxycyclopentylamino)pyrimidin-2-yl)-6-(3-methoxy-2-methylphenyl)phthalazin-1(2H)-one
2-(5-(2-Hydroxy-2-methylpropylamino)pyrimidin-2-yl)-6-(3-methoxy-2-methylphenyl)phthalazin-1(2H)-one
6-(3-Methoxy-2-methylphenyl)-2-(5-(2-methoxyethylamino)pyrimidin-2-yl)phthalazin-1(2H)-one
2-(5-Chloropyrimidin-2-yl)-6-(3-methoxy-2-methylphenyl)phthalazin-1(2H)-one
6-(3-Methoxy-2-methylphenyl)-2-(5-(S-methylsulfonimidoyl)pyridin-2-yl)phthalazin-1(2H)-one
N-(2-Hydroxy-2-methylpropyl)-6-(6-(3-methoxy-2-methylphenyl)-1-oxophthalazin-2(1H)-yl)nicotinamide
cis-2-(5-(3-Hydroxypyrrolidine-1-carbonyl)pyridin-2-yl)-6-(3-methoxy-2-methylphenyl)phthalazin-1(2H)-one
trans-2-(5-(3-Hydroxypyrrolidine-1-carbonyl)pyridin-2-yl)-6-(3-methoxy-2-methylphenyl)phthalazin-1(2H)-one
cis-N-(3-Hydroxycyclopentyl)-6-(6-(3-methoxy-2-methylphenyl)-1-oxophthalazin-2(1H)-yl)nicotinamide trans-N-(3-Hydroxycyclopentyl)-6-(6-(3-methoxy-2-methylphenyl)-1-oxophthalazin-2(1H)-yl)nicotinamide
6-(3-Methoxy-2-methylphenyl)-2-(5-(2-(pyrrolidin-1-yl)ethylamino)pyrimidin-2-yl)phthalazin-1(2H)-one
2-(5-(2-(Dimethylamino)-2-methylpropylamino)pyrimidin-2-yl)-6-(3-methoxy-2-methylphenyl)phthalazin-1(2H)-one
2-(5-(2-(1H-Imidazol-1-yl)ethylamino)pyrimidin-2-yl)-6-(3-methoxy-2-methylphenyl)phthalazin-1(2H)-one
(S)-2-Amino-N-(6-(6-(3-methoxy-2-methylphenyl)-1-oxophthalazin-2(1H)-yl)pyridin-3-yl)-3-methylbutanamide
(S)—N-(6-(6-(3-Methoxy-2-methylphenyl)-1-oxophthalazin-2(1H)-yl)pyridin-3-yl)pyrrolidine-2-carboxamide
2-(5-(1-Hydroxy-2-methylpropan-2-ylamino)pyrimidin-2-yl)-6-(3-methoxy-2-methylphenyl)phthalazin-1(2H)-one
2-(5-(3-(Dimethylamino)pyrrolidin-1-yl)pyrimidin-2-yl)-6-(3-methoxy-2-methylphenyl)phthalazin-1(2H)-one
(S)-2-Amino-3-hydroxy-N-(6-(6-(3-methoxy-2-methylphenyl)-1-oxophthalazin-2(1H)-yl)pyridin-3-yl)propenamide
2-Amino-3-hydroxy-N-(6-(6-(3-methoxy-2-methylphenyl)-1-oxophthalazin-2(1H)-yl)pyridin-3-yl)propanamide
2-(5-(3-(Dimethylamino)piperidin-1-yl)pyrimidin-2-yl)-6-(3-methoxy-2-methylphenyl)phthalazin-1(2H)-one
2-(5-(1-(Dimethylamino)-2-methylpropan-2-ylamino)pyrimidin-2-yl)-6-(3-methoxy-2-methylphenyl)phthalazin-1(2H)-one
6-(3-Methoxy-2-methylphenyl)-2-(5-(2-(methylamino)ethylamino)pyrimidin-2-yl)phthalazin-1(2H)-one
2-(5-(2-(1H-1,2,4-Triazol-1-yl)ethylamino)pyrimidin-2-yl)-6-(3-methoxy-2-methylphenyl)phthalazin-1(2H)-one
6-(3-Methoxy-2-methylphenyl)-2-(5-(1-methylpyrrolidin-3-ylamino)pyrimidin-2-yl)phthalazin-1(2H)-one
6-(1-Methyl-1,2,3,4-tetrahydroquinolin-5-yl)-2-(pyrimidin-2-yl)phthalazin-1(2H)-one
6-(3-Methoxy-2-methylphenyl)-2-(5-((1-methoxy-2-methylpropan-2-yl)amino)pyrimidin-2-yl)phthalazin-1(2H)-one
6-(3-Methoxy-2-methylphenyl)-2-(5-((2-methoxy-2-methylpropyl)amino)pyrimidin-2-yl)phthalazin-1(2H)-one
6-(2-Fluoro-3-methoxyphenyl)-2-(pyrimidin-2-yl)phthalazin-1(2H)-one
6-(3-Fluoro-2-methoxypyridin-4-yl)-2-(pyrimidin-2-yl)phthalazin-1(2H)-one
6-(1-Methyl-2,3-dihydro-1H-pyrrolo[2,3-b]pyridin-4-yl)-2-(pyridin-2-yl)phthalazin-1(2H)-one and
6-(1-methyl-2,3-dihydro-1H-pyrrolo[2,3-b]pyridin-4-yl)-2-(pyrimidin-2-yl)phthalazin-1(2H)-one.
   The above list of compounds can also be represented by the following skeletal formulae

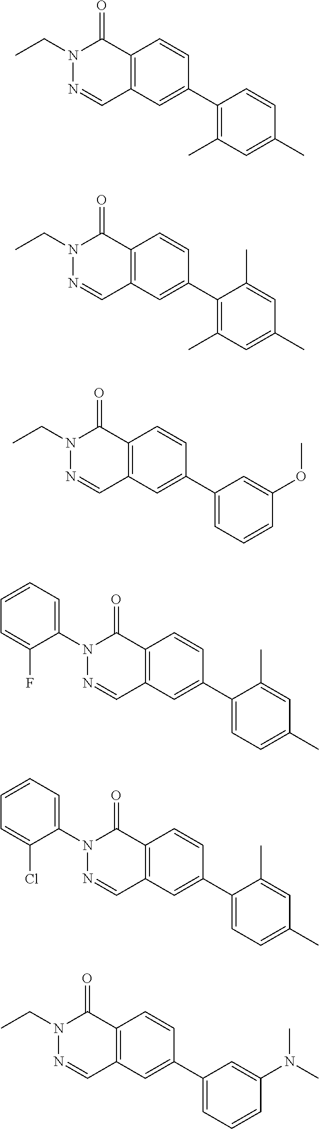

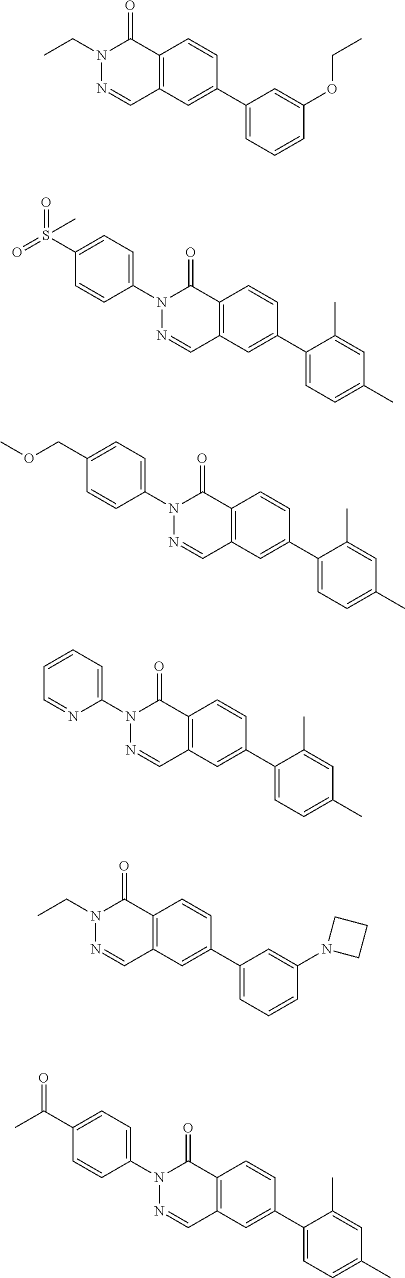

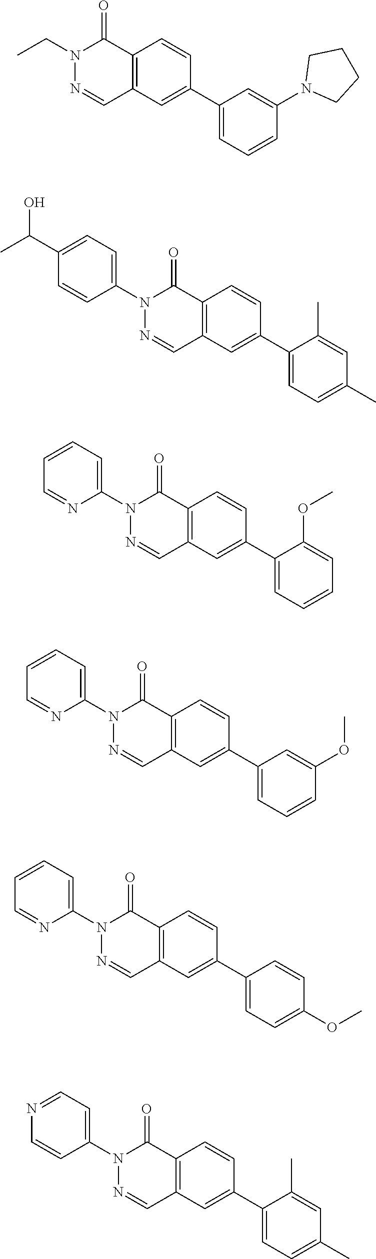 [SMILES: CCn1ncc2cc(-c3cccc(N4CCCC4)c3)ccc2c1=O ; Cc1ccc(-c2ccc3c(=O)n(-c4ccc(C(C)O)cc4)ncc3c2)c(C)c1 ; COc1ccccc1-c1ccc2c(=O)n(-c3ccccn3)ncc2c1 ; COc1cccc(-c2ccc3c(=O)n(-c4ccccn4)ncc3c2)c1 ; COc1ccc(-c2ccc3c(=O)n(-c4ccccn4)ncc3c2)cc1 ; Cc1ccc(-c2ccc3c(=O)n(-c4ccncc4)ncc3c2)c(C)c1]

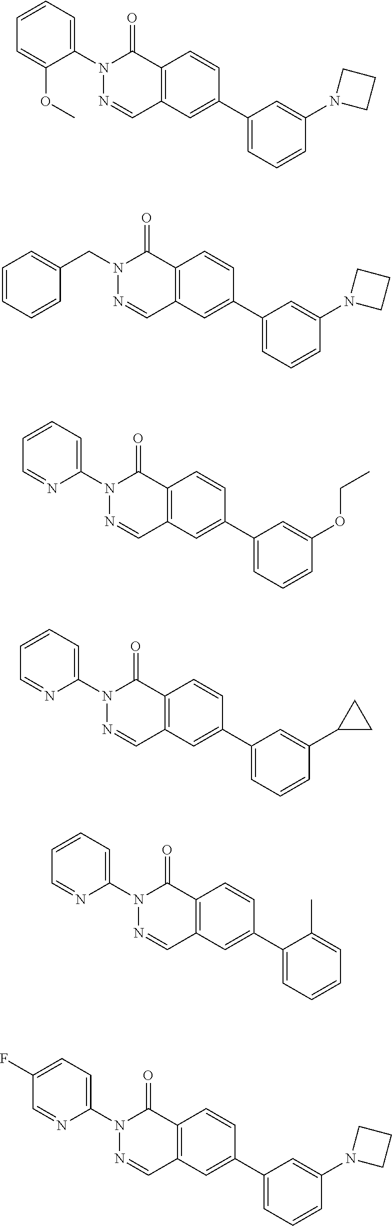 [SMILES: COc1ccccc1-n1ncc2cc(-c3cccc(N4CCC4)c3)ccc2c1=O ; O=c1c2ccc(-c3cccc(N4CCC4)c3)cc2cnn1Cc1ccccc1 ; CCOc1cccc(-c2ccc3c(=O)n(-c4ccccn4)ncc3c2)c1 ; O=c1c2ccc(-c3cccc(C4CC4)c3)cc2cnn1-c1ccccn1 ; Cc1ccccc1-c1ccc2c(=O)n(-c3ccccn3)ncc2c1 ; O=c1c2ccc(-c3cccc(N4CCC4)c3)cc2cnn1-c1ccc(F)cn1]

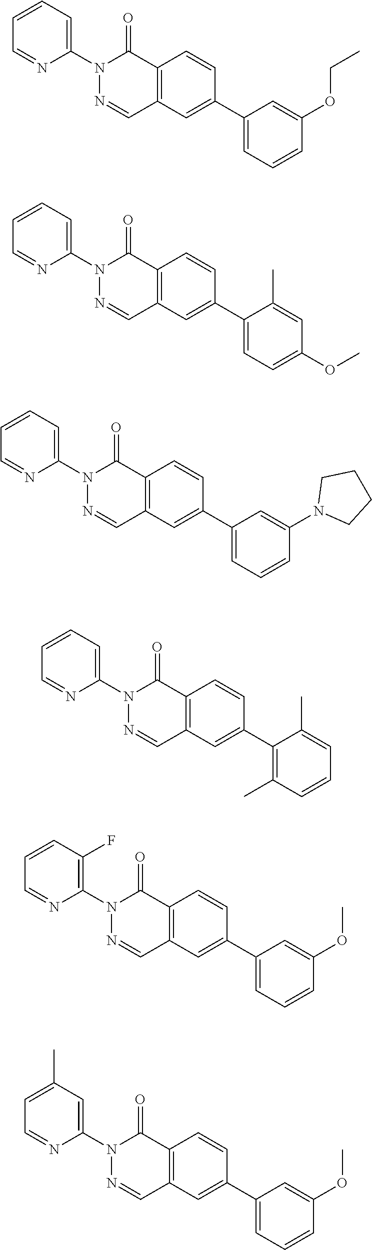 [SMILES: CCOc1cccc(-c2ccc3c(=O)n(-c4ccccn4)ncc3c2)c1 ; COc1ccc(-c2ccc3c(=O)n(-c4ccccn4)ncc3c2)c(C)c1 ; O=c1c2ccc(-c3cccc(N4CCCC4)c3)cc2cnn1-c1ccccn1 ; Cc1cccc(C)c1-c1ccc2c(=O)n(-c3ccccn3)ncc2c1 ; COc1cccc(-c2ccc3c(=O)n(-c4ncccc4F)ncc3c2)c1 ; COc1cccc(-c2ccc3c(=O)n(-c4cc(C)ccn4)ncc3c2)c1]

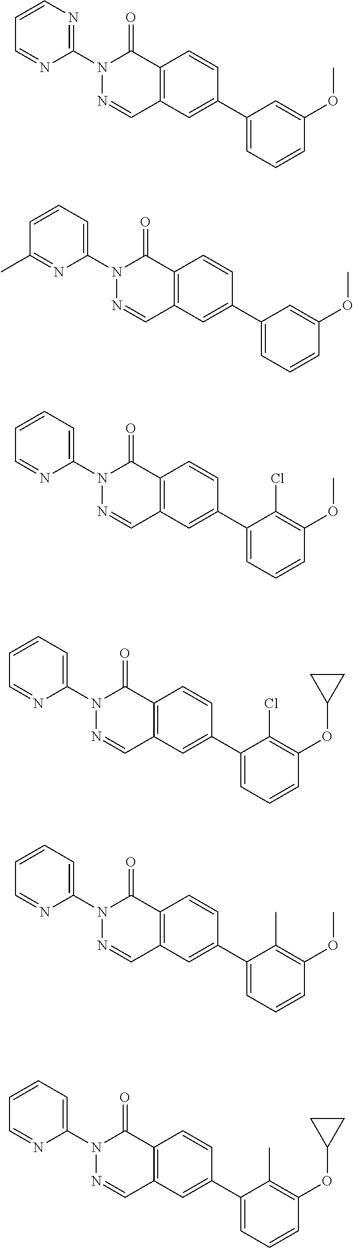

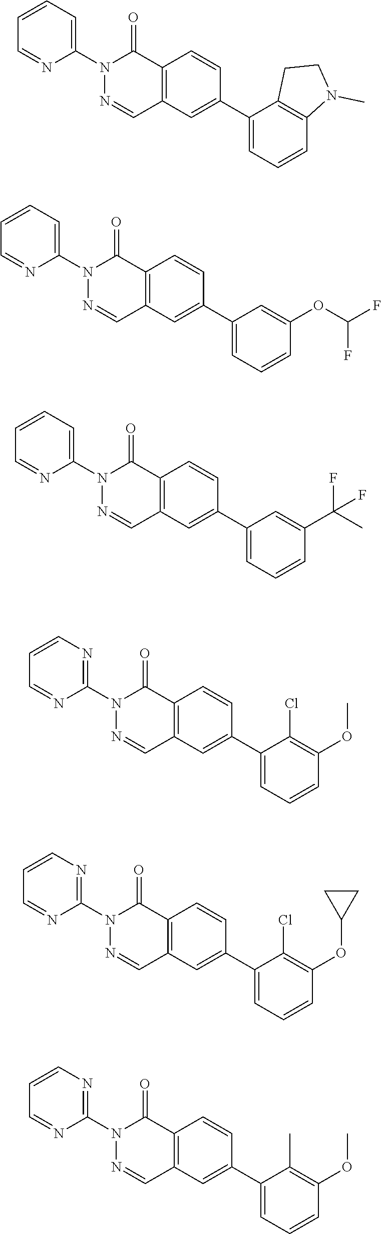 [SMILES: CN1CCc2c(-c3ccc4c(=O)n(-c5ccccn5)ncc4c3)cccc21 ; O=c1c2ccc(-c3cccc(OC(F)F)c3)cc2cnn1-c1ccccn1 ; CC(F)(F)c1cccc(-c2ccc3c(=O)n(-c4ccccn4)ncc3c2)c1 ; COc1cccc(-c2ccc3c(=O)n(-c4ncccn4)ncc3c2)c1Cl ; O=c1c2ccc(-c3cccc(OC4CC4)c3Cl)cc2cnn1-c1ncccn1 ; COc1cccc(-c2ccc3c(=O)n(-c4ncccn4)ncc3c2)c1C]

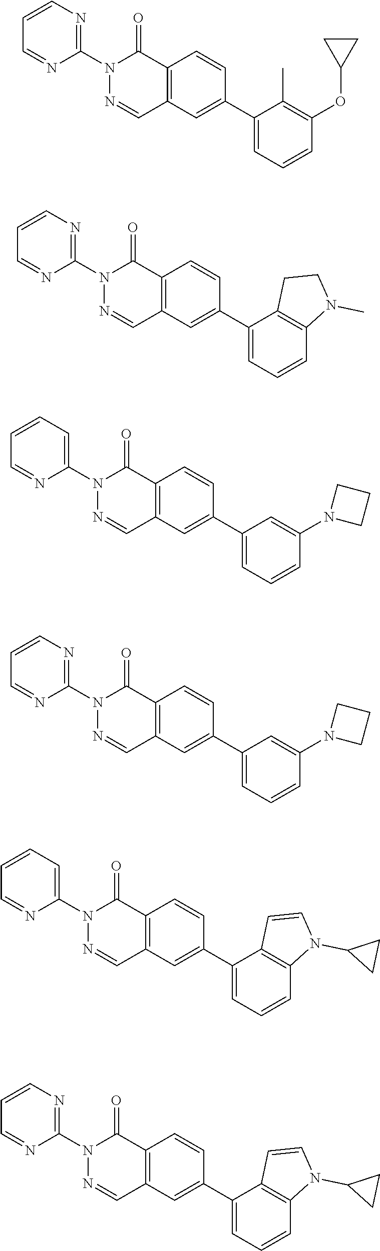 [SMILES: Cc1c(OC2CC2)cccc1-c1ccc2c(=O)n(-c3ncccn3)ncc2c1 ; CN1CCc2c(-c3ccc4c(=O)n(-c5ncccn5)ncc4c3)cccc21 ; O=c1c2ccc(-c3cccc(N4CCC4)c3)cc2cnn1-c1ccccn1 ; O=c1c2ccc(-c3cccc(N4CCC4)c3)cc2cnn1-c1ncccn1 ; O=c1c2ccc(-c3cccc4c3ccn4C3CC3)cc2cnn1-c1ccccn1 ; O=c1c2ccc(-c3cccc4c3ccn4C3CC3)cc2cnn1-c1ncccn1]

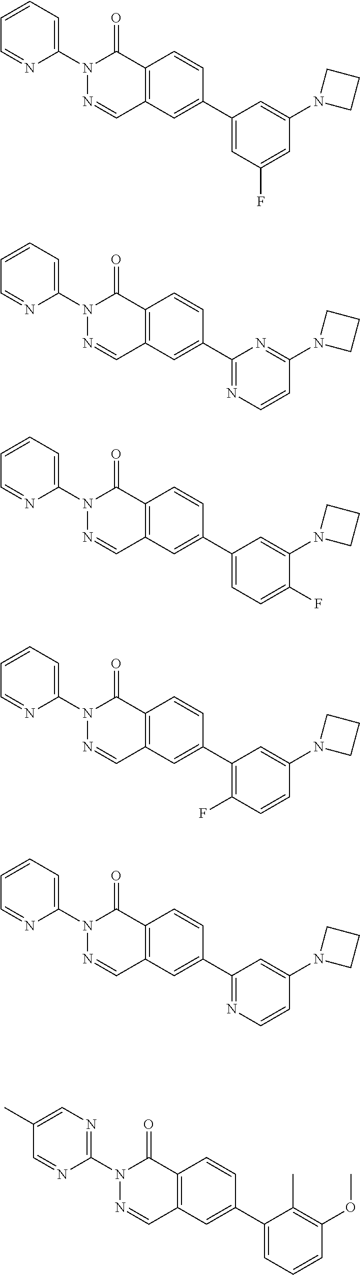

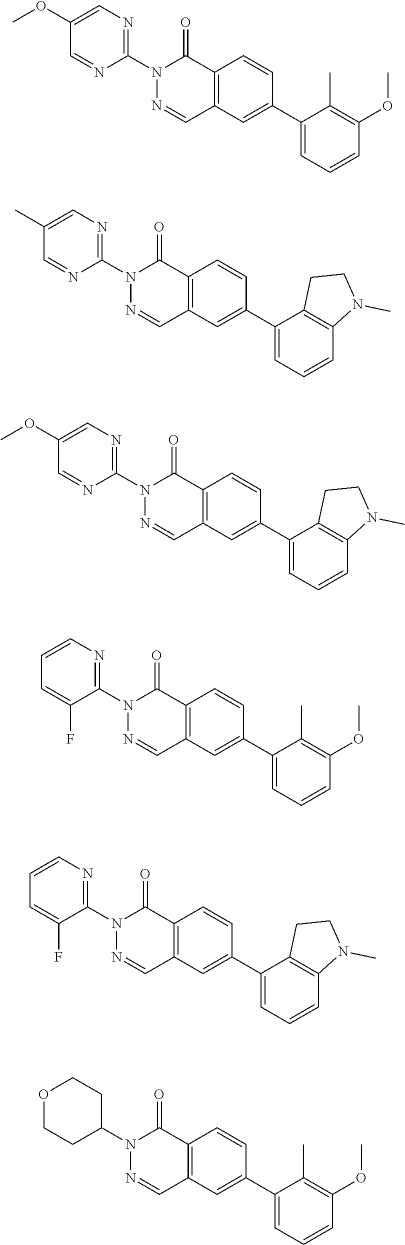

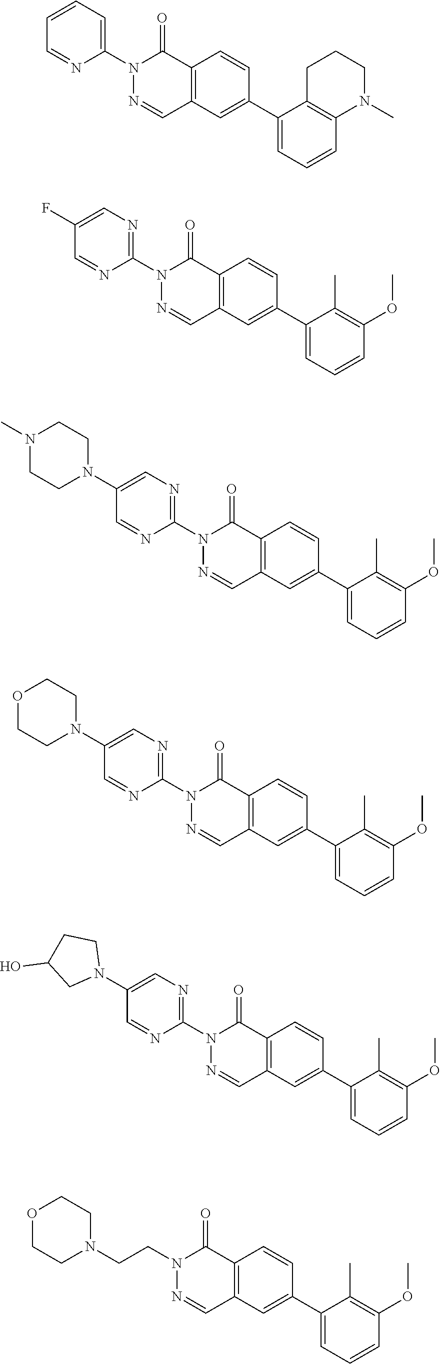

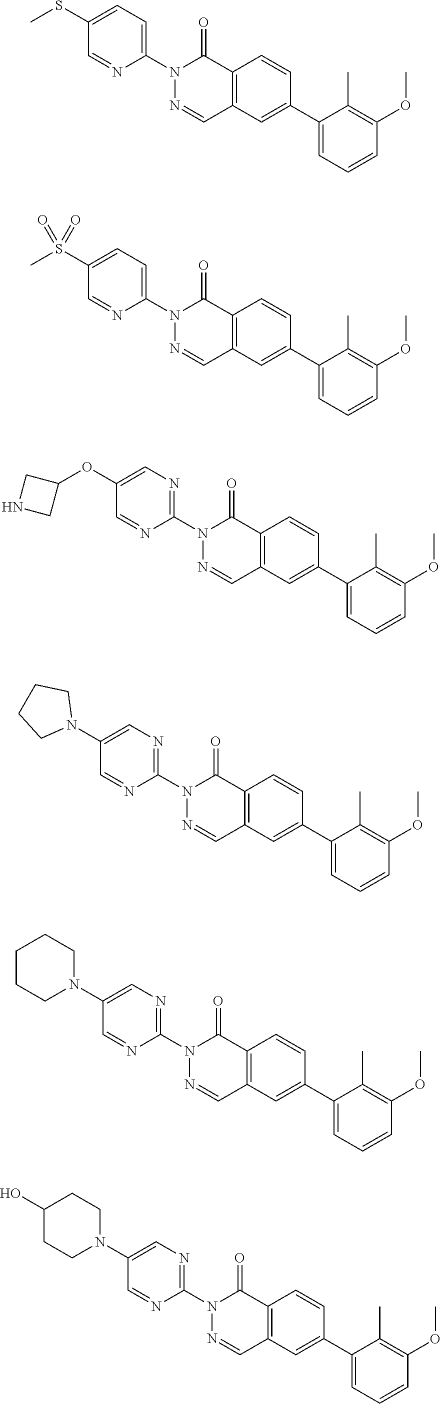

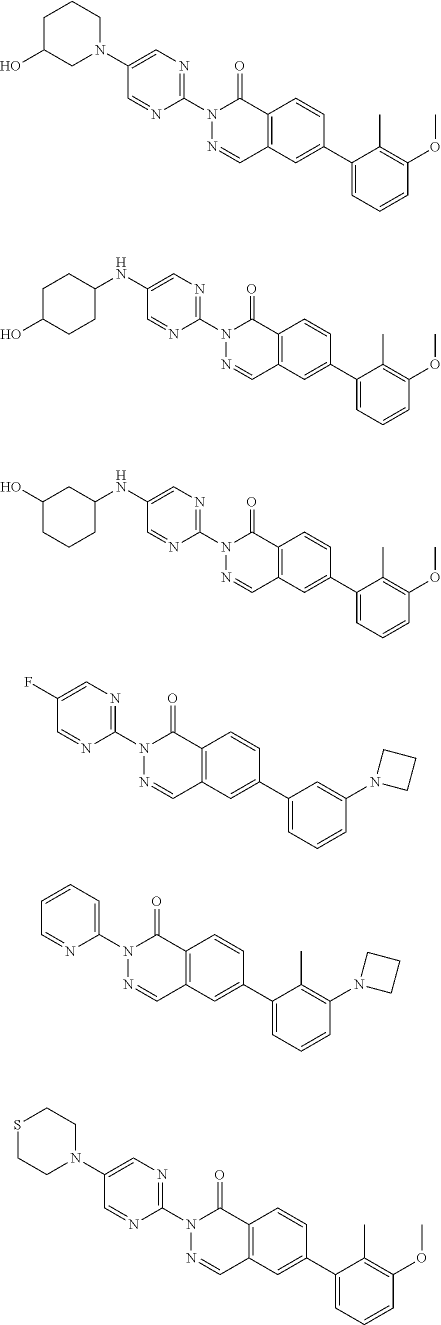 [SMILES: COc1cccc(-c2ccc3c(=O)n(-c4ncc(N5CCCC(O)C5)cn4)ncc3c2)c1C ; COc1cccc(-c2ccc3c(=O)n(-c4ncc(NC5CCC(O)CC5)cn4)ncc3c2)c1C ; COc1cccc(-c2ccc3c(=O)n(-c4ncc(NC5CCCC(O)C5)cn4)ncc3c2)c1C ; O=c1c2ccc(-c3cccc(N4CCC4)c3)cc2cnn1-c1ncc(F)cn1 ; Cc1c(-c2ccc3c(=O)n(-c4ccccn4)ncc3c2)cccc1N1CCC1 ; COc1cccc(-c2ccc3c(=O)n(-c4ncc(N5CCSCC5)cn4)ncc3c2)c1C]

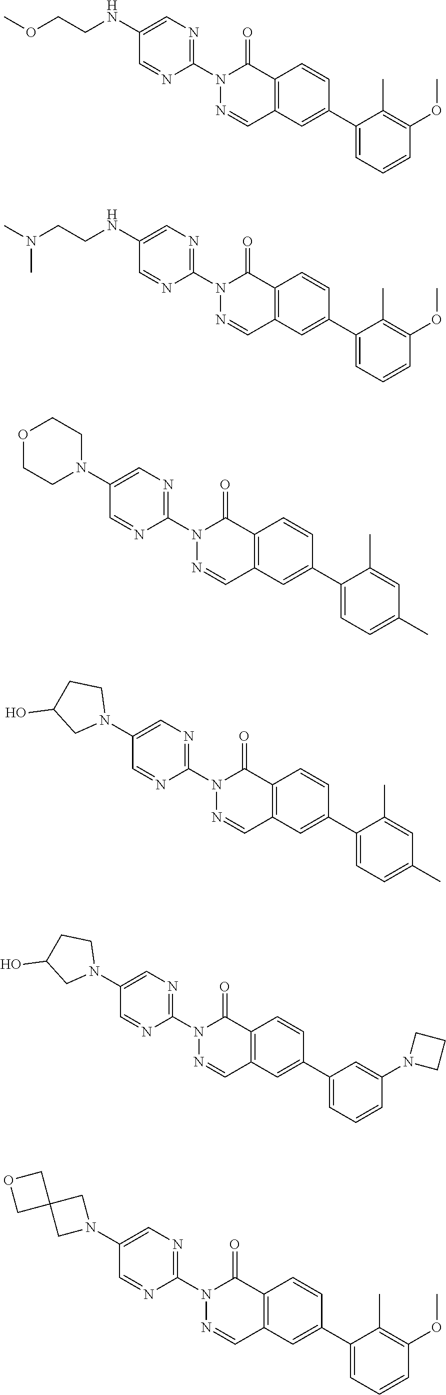

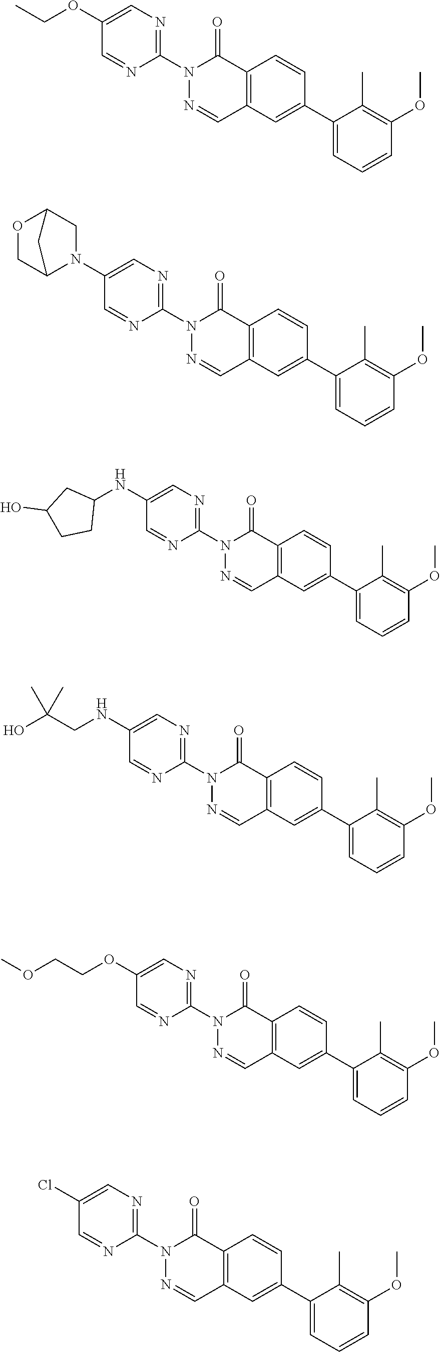

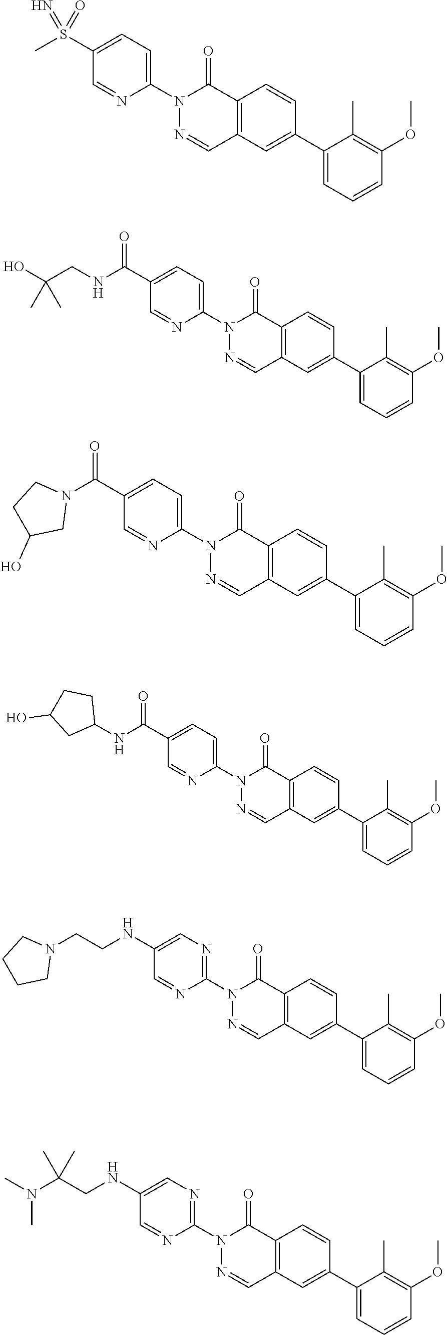

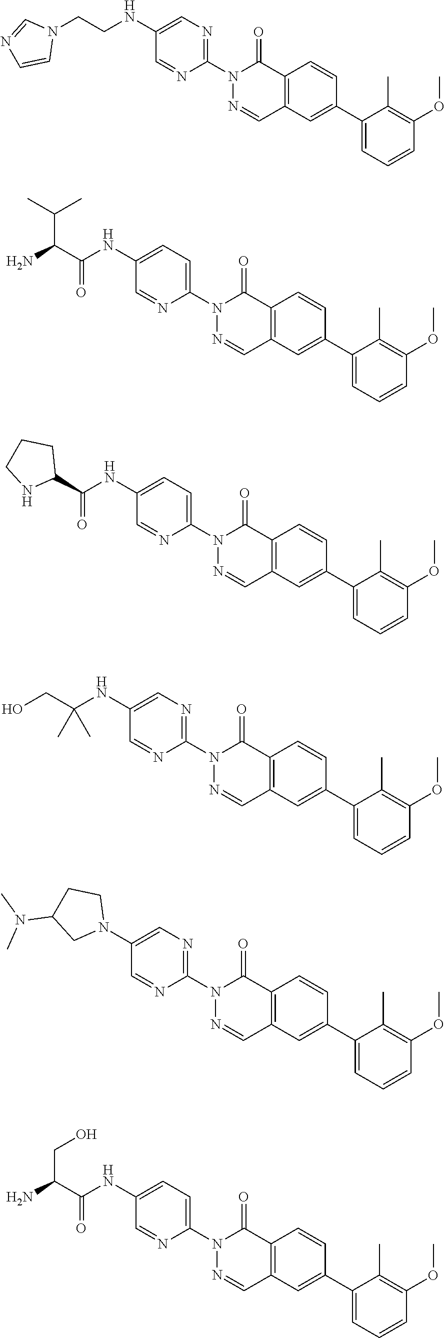

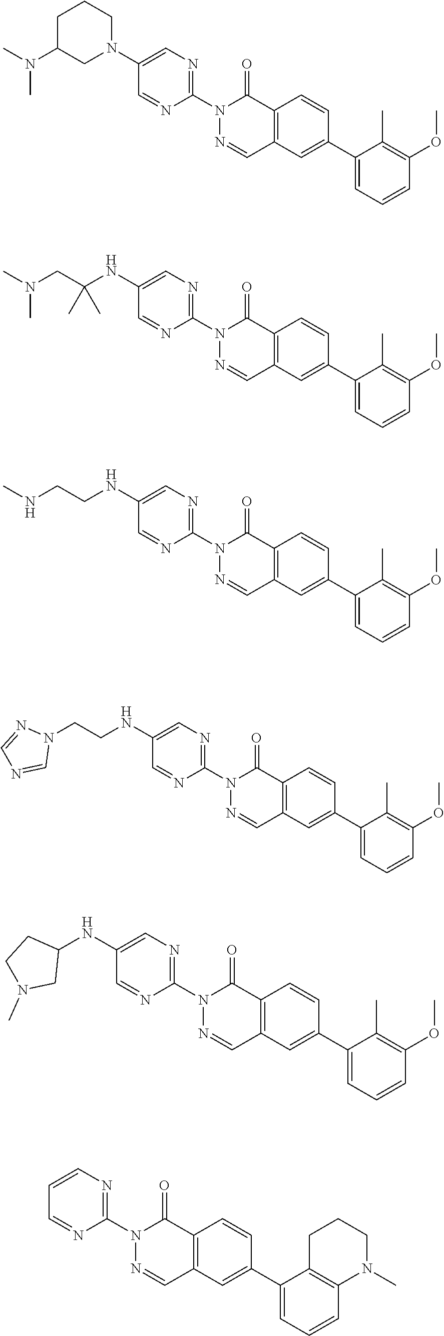

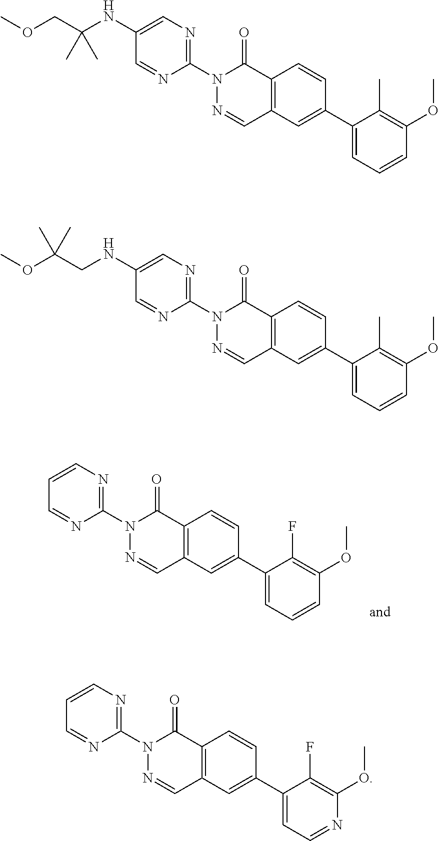

Preferably, the compounds of Formula (I) are one or more compounds as mentioned in the following list, as well as a pharmaceutically acceptable acid or base addition salt thereof, a stereochemically isomeric form thereof or an N-oxide form thereof:
6-(2,4-Dimethylphenyl)-2-(pyridin-2-yl)phthalazin-1(2H)-one
6-(2-Chloro-3-methoxyphenyl)-2-(pyridin-2-yl)phthalazin-1(2H)-one
6-(2-Chloro-3-cyclopropoxyphenyl)-2-(pyridin-2-yl)phthalazin-1(2H)-one
6-(3-Methoxy-2-methylphenyl)-2-(pyridin-2-yl)phthalazin-1(2H)-one
6-(3-Cyclopropoxy-2-methylphenyl)-2-(pyridin-2-yl)phthalazin-1(2H)-one
6-(1-Methylindolin-4-yl)-2-(pyridin-2-yl)phthalazin-1(2H)-one
6-(2-Chloro-3-methoxyphenyl)-2-(pyrimidin-2-yl)phthalazin-1(2H)-one
6-(3-Methoxy-2-methylphenyl)-2-(pyrimidin-2-yl)phthalazin-1(2H)-one
6-(5-(Azetidin-1-yl)-2-fluorophenyl)-2-(pyridin-2-yl)phthalazin-1(2H)-one
6-(3-Methoxy-2-methylphenyl)-2-(5-methoxypyrimidin-2-yl)phthalazin-1(2H)-one
2-(3-Fluoropyridin-2-yl)-6-(3-methoxy-2-methylphenyl)phthalazin-1(2H)-one
6-(1-Methyl-1,2,3,4-tetrahydroquinolin-5-yl)-2-(pyridin-2-yl)phthalazin-1(2H)-one
2-(5-Fluoropyrimidin-2-yl)-6-(3-methoxy-2-methylphenyl)phthalazin-1(2H)-one
6-(3-Methoxy-2-methylphenyl)-2-(5-morpholinopyrimidin-2-yl)phthalazin-1(2H)-one
cis-2-(5-(3-Hydroxypyrrolidin-1-yl)pyrimidin-2-yl)-6-(3-methoxy-2-methylphenyl)phthalazin-1(2H)-one
trans-2-(5-(3-Hydroxypyrrolidin-1-yl)pyrimidin-2-yl)-6-(3-methoxy-2-methylphenyl)phthalazin-1(2H)-one
2-(5-(4-Hydroxypiperidin-1-yl)pyrimidin-2-yl)-6-(3-methoxy-2-methylphenyl)phthalazin-1(2H)-one
cis-2-(5-(3-Hydroxypiperidin-1-yl)pyrimidin-2-yl)-6-(3-methoxy-2-methylphenyl)phthalazin-1(2H)-one
trans-2-(5-(3-Hydroxypiperidin-1-yl)pyrimidin-2-yl)-6-(3-methoxy-2-methylphenyl)phthalazin-1(2H)-one
6-(3-(Azetidin-1-yl)-2-methylphenyl)-2-(pyridin-2-yl)phthalazin-1(2H)-one
2-(5-(2-(Dimethylamino)ethylamino)pyrimidin-2-yl)-6-(3-methoxy-2-methylphenyl)phthalazin-1(2H)-one
2-(5-(2-Oxa-5-azabicyclo[2.2.1]heptan-5-yl)pyrimidin-2-yl)-6-(3-methoxy-2-methylphenyl)phthalazin-1(2H)-one
cis-2-(5-(3-Hydroxycyclopentylamino)pyrimidin-2-yl)-6-(3-methoxy-2-methylphenyl)phthalazin-1(2H)-one
trans-2-(5-(3-Hydroxycyclopentylamino)pyrimidin-2-yl)-6-(3-methoxy-2-methylphenyl)phthalazin-1(2H)-one
2-(5-(2-Hydroxy-2-methylpropylamino)pyrimidin-2-yl)-6-(3-methoxy-2-methylphenyl)phthalazin-1(2H)-one
6-(3-Methoxy-2-methylphenyl)-2-(5-(S-methylsulfonimidoyl)pyridin-2-yl)phthalazin-1(2H)-one
cis-2-(5-(3-Hydroxypyrrolidine-1-carbonyl)pyridin-2-yl)-6-(3-methoxy-2-methylphenyl)phthalazin-1(2H)-one and
trans-2-(5-(3-Hydroxypyrrolidine-1-carbonyl)pyridin-2-yl)-6-(3-methoxy-2-methylphenyl)phthalazin-1(2H)-one.

The above list of compounds can also be represented by the following skeletal formulae

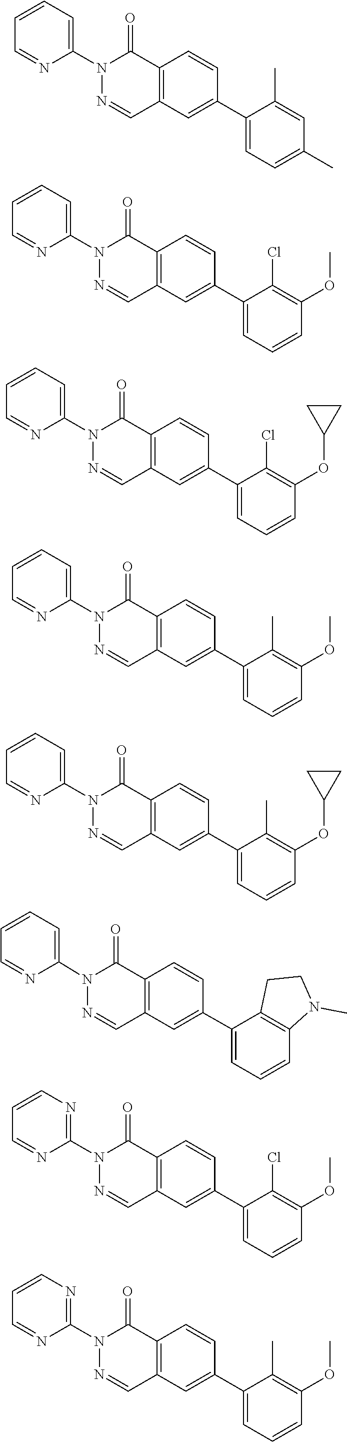

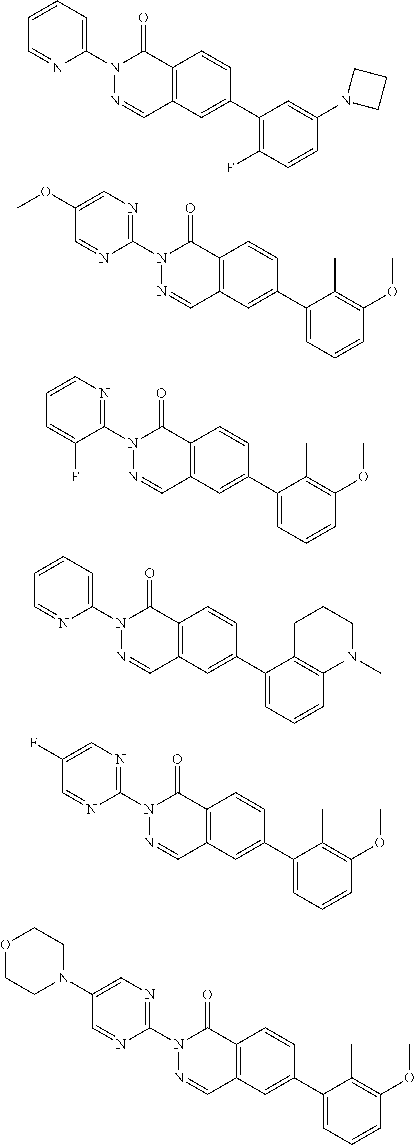

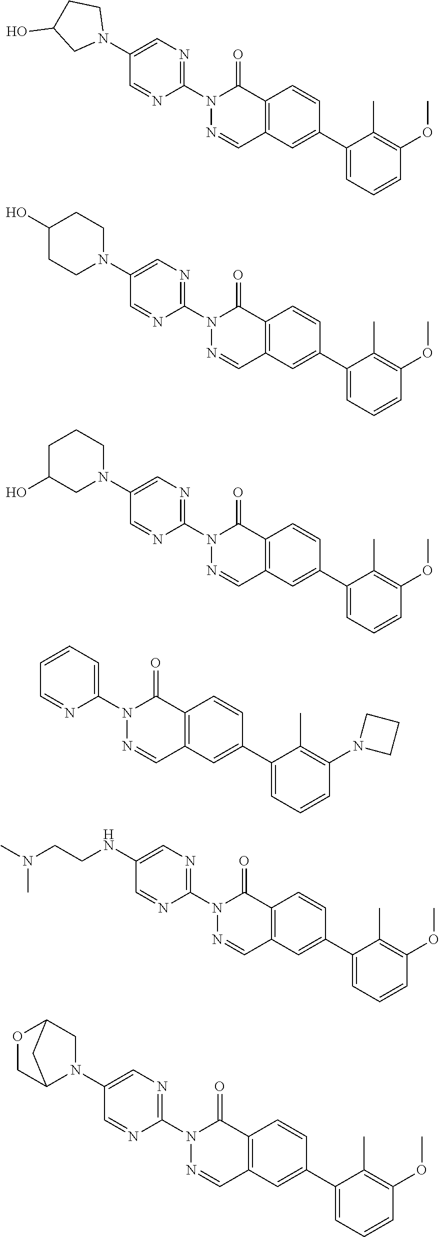

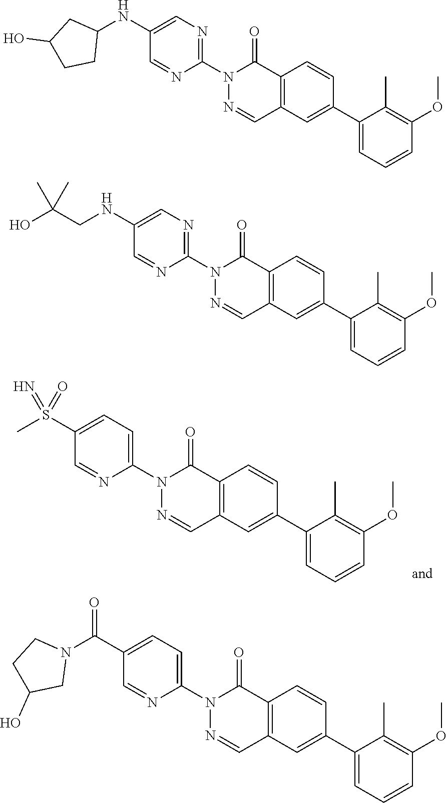

Preferably, the compounds of Formula (I) are one or more compounds as mentioned in the following list, as well as a pharmaceutically acceptable acid or base addition salt thereof, a stereochemically isomeric form thereof or an N-oxide form thereof:
6-(3-Methoxy-2-methylphenyl)-2-(pyrimidin-2-yl)phthalazin-1(2H)-one
cis-2-(5-(3-Hydroxypyrrolidine-1-carbonyl)pyridin-2-yl)-6-(3-methoxy-2-methylphenyl)phthalazin-1(2H)-one
trans-2-(5-(3-Hydroxypyrrolidine-1-carbonyl)pyridin-2-yl)-6-(3-methoxy-2-methylphenyl)phthalazin-1(2H)-one

The above compounds can also be represented by the following skeletal formulae:

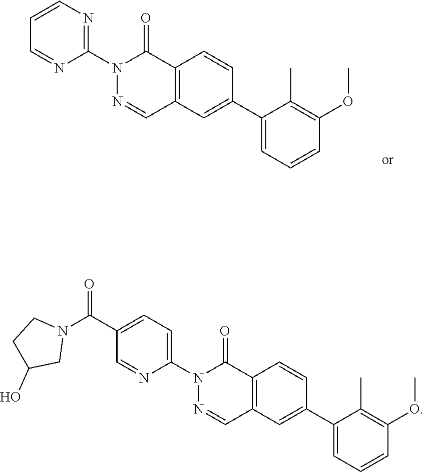 [SMILES: COc1cccc(-c2ccc3c(=O)n(-c4ncccn4)ncc3c2)c1C ; COc1cccc(-c2ccc3c(=O)n(-c4ccc(C(=O)N5CCC(O)C5)cn4)ncc3c2)c1C]

The compounds according to any statement above may exhibit metabotropic glutamate receptor 7 modulator activity.

The disclosed compounds also include all pharmaceutically acceptable isotopic variations, in which at least one atom is replaced by an atom having the same atomic number, but an atomic mass different from the atomic mass usually found in nature.

Examples of isotopes suitable for inclusion in the disclosed compounds include, without limitation, isotopes of hydrogen, such as ²H and ³H; isotopes of carbon, such as ¹¹C, ¹³C and ¹⁴C; isotopes of nitrogen, such as ¹⁵N; isotopes of oxygen, such as ¹⁷O and ¹⁸O; isotopes of phosphorus, such as ³¹P, ³²P and ³³P; isotopes of sulfur, such as ³⁵S; isotopes of fluorine, such as ¹⁸F; isotopes of chlorine, such as ³⁶Cl; and isotopes of iodine, such as ¹²⁵I. The invention includes various isotopically labelled compounds as defined herein, for example those into which radioactive isotopes, such as ³H and ¹⁴C, or those into which non-radioactive isotopes, such as ²H and ¹³C are present.

Such isotopically labelled compounds are useful in metabolic studies (with ¹⁴C), reaction kinetic studies (with for example ²H or ³H), detection or imaging techniques, such as positron emission tomography (PET) or single-photon emission computed tomography (SPECT) including drug or substrate tissue distribution assays, or in radioactive treatment of patients. In particular, ¹¹C, ¹⁸F, ¹⁵O and ¹³N or labelled compounds may be particularly desirable for PET studies for examining substrate receptor occupancy. Further, substitution with heavier isotopes, particularly deuterieum (e.g., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of Formula (I) to (VI). Isotopically-labelled compounds of Formula (I) to (VI) can generally be prepared by conventional techniques known to those skilled in the art or by processes analogous to those described in the accompanying Examples using appropriate isotopically-labelled reagents in place of the non-labelled reagent previously employed.

In an aspect of the present invention there is provided a pharmaceutical composition comprising a compound according to any statement set out above. The pharmaceutical composition may further comprise a pharmaceutically acceptable carrier and/or excipient. The pharmaceutical composition may comprise a therapeutically effective amount of the compound according to any statement set out above.

In an aspect of the present invention there is provided a method of treating or preventing a condition in a mammal comprising administering to a mammal in need of such treatment or prevention, an effective amount of a compound/composition according to any statement set out above.

The treatment or prevention may be affected or facilitated by the modulatory effect of a mGlu7 allosteric modulator such as a mGlu7 negative allosteric modulator.

The condition may be one or more of a central nervous system disorder or an otic disease or disorder or a pain disorder.

The central nervous system disorder may be post-traumatic stress disorder (PTSD).

The otic disease and disorder may be one or more of an inner ear impairment, age-related hearing impairment (presbycusis), Meniere's disease, sudden hearing loss, noise induced hearing loss, otitis media, autoimmune inner ear disease, acute tinnitus, chronic tinnitus, drug-induced hearing loss, hidden hearing loss, cisplatin-induced hearing loss, aminoglycosides-induced hearing loss, ototoxicity, central auditory processing disorder or vestibular disorder.

In a further aspect of the present invention, there is provided a method of treating, preventing, ameliorating, controlling or reducing the risk of various neurological and psychiatric disorders associated with glutamate dysfunction in a mammal, comprising administering to a mammal in need of such treatment or prevention, an effective amount of a compound/composition according to any statement set out above. The treatment or prevention may be affected or facilitated by the modulatory effect of mGlu7 negative allosteric modulators.

Preferably, the methods are for the treatment or prevention of a condition in a human.

In a further aspect of the present invention, there is provided the compounds or compositions as set out in any statement above for use as a medicament.

In a further aspect of the present invention there is provided the compounds or compositions as set out in any statement above for use in a method of treatment or prevention as defined in any statement set out above.

In a further aspect of the present invention there is provided a use of a compound according to any statement set out above in the manufacture of a medicament for the treatment or prevention of a condition as defined in any statement set out above.

### DEFINITION OF TERMS

Listed below are definitions of various terms used in the specification and claims to describe the present invention.

For the avoidance of doubt it is to be understood that in this specification “(C₁-C₆)” means a carbon radical having 1, 2, 3, 4, 5 or 6 carbon atoms. “(C₀-C₆)” means a carbon radical having 0, 1, 2, 3, 4, 5 or 6 carbon atoms. In this specification “C” means a carbon atom, “N” means a nitrogen atom, “O” means an oxygen atom and “S” means a sulphur atom.

In the case where a subscript is the integer 0 (zero) the radical to which the subscript refers, indicates that the radical is absent, i.e. there is a direct bond between the radicals.

In the case where a subscript is the integer 0 (zero) and the radical to which the subscript refers is alkyl, this indicates the radical is a hydrogen atom.

In this specification, unless stated otherwise, the term “bond” refers to a saturated covalent bond. When two or more bonds are adjacent to one another, they are assumed to be equal to one bond. For example, a radical -A-B-, wherein both A and B may be a bond, the radical is depicting a single bond.

In this specification, unless stated otherwise, the term “alkyl” includes both straight and branched chain alkyl radicals and may be methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term “(C₀-C₃)alkyl” refers to an alkyl radical having 0, 1, 2 or 3 carbon atoms and may be methyl, ethyl, n-propyl or i-propyl.

In this specification, unless stated otherwise, the term “alkylene” includes both straight and branched difunctional saturated hydrocarbon radicals and may be methylene (—CH₂—), ethylene (—CH₂—CH₂—), n-propylene (—CH₂—CH₂—CH₂—), i-propylene (—CH—(CH₃)—CH₂—), n-butylene (—CH₂—CH₂—CH₂—CH₂—), i-butylene (—CH₂—CH—(CH₃)—CH₂—), t-butylene (—CH₂—C—(CH₃)—CH₂—), n-pentylene (—CH₂—CH₂—CH₂—CH₂—CH₂—), i-pentylene (—CH₂—CH(CH₃)—CH₂—CH₂—), neo-pentylene (—CH₂—C(CH₃)₂—CH₂—), n-hexylene (—CH₂—CH₂—CH₂—CH₂—CH₂—CH₂—), i-hexylene (—CH₂—CH—(CH₃)—CH₂—CH₂—CH₂—) or neo-hexylene (—CH₂—C(CH₃)₂—CH₂—CH₂—). The term “O—(C₁-C₆)alkylene-aryl” refers to a an alkyl chain having 0, 1, 2, 3, 4, 5 or 6 carbon atoms between an oxygen atom and an aryl group.

In this specification, unless stated otherwise, the term “cycloalkyl” refers to an optionally substituted carbocycle containing no heteroatoms, including mono-, bi-, and tricyclic saturated carbocycles, as well as fused ring systems. Such fused ring systems can include one ring that is partially or fully unsaturated such as a benzene ring to form fused ring systems such as benzo-fused carbocycles. Cycloalkyl includes such fused ring systems as spirofused ring systems. Examples of cycloalkyl include cyclopropyl, cyclobutyl, cyclopentyl, cyclohexyl, bicyclo[1.1.1]pentanyl, decahydronaphthalene, adamantane, indanyl, fluorenyl and 1,2,3,4-tetrahydronaphthalene and the like. The term “(C₃-C₇)cycloalkyl” may be cyclopropyl, cyclobutyl, cyclopentyl, cyclohexyl, cycloheptyl and the like.

In this specification, unless stated otherwise, the term “alkenyl” includes both straight and branched chain alkenyl radicals. The term “(C₂-C₆)alkenyl” refers to an alkenyl radical having 2 to 6 carbon atoms and one or two double bonds, and may be, but is not limited to vinyl, allyl, propenyl, i-propenyl, butenyl, i-butenyl, crotyl, pentenyl, i-pentenyl or hexenyl.

In this specification, unless stated otherwise, the term “alkenylene” includes both straight and branched chain disubstituted alkenyl radicals. The term “(C₂-C₆)alkenylene” refers to an alkenylene radical having 2 to 6 carbon atoms and one or two double bonds, and may be, but is not limited to vinylene, allylene, propenylene, i-propenylene, butenylene, i-butenylene, crotylene, pentenylene, i-pentenylene or hexenylene.

In this specification, unless stated otherwise, the term “alkynyl” includes both straight and branched chain alkynyl radicals. The term (C₂-C₆)alkynyl having 2 to 6 carbon atoms and one or two triple bonds, and may be, but is not limited to ethynyl, propargyl, butynyl, i-butynyl, pentynyl, i-pentynyl or hexynyl.

In this specification, unless stated otherwise, the term “alkynylene” includes both straight and branched chain disubstituted alkynylene radicals. The term (C₂-C₆)alkynylene having 2 to 6 carbon atoms and one or two triple bonds, and may be, but is not limited to ethynylene, propargylene, butynylene, i-butynylene, pentynylene, i-pentynylene or hexynylene.

The term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl, indenyl and the like.

In this specification, unless stated otherwise, the term “heteroaryl” refers to an optionally substituted monocyclic or bicyclic unsaturated, aromatic ring system containing at least one heteroatom selected independently from N, O or S. Examples of “heteroaryl” may be, but are not limited to benzimidazolyl, benzisothiazolyl benzisoxazolyl, benzofuryl, benzopyrazolyl, benzothiazolyl, benzothiophenyl, benzotriazolyl, benzoxazolyl, furazanyl, furyl, imidazolonyl, imidazolyl, imidazopyridazinyl, imidazopyridyl, indolyl, isoindolyl, isoquinolinyl, isothiazolyl, isoxazolyl, naphthyridinyl, oxadiazolyl, oxazolonyl, oxazolopyridazinyl, oxazolopyridyl, oxazolyl, phtalazinyl, pteridinyl, purinyl, pyrazinyl, pyrazolopyridinyl, pyrazolyl, pyridazinyl, pyridonyl, pyridyl, pyrimidyl, pyrrolyl, quinazolyl, quinolyl, quinoxalinyl, tetrahydrotriazolopyridyl, tetrahydrotriazolopyrimidinyl, tetrazolyl, thiadiazolyl, thiazolonyl, thiazolopyridazinyl, thiazolopyridyl, thienyl, thionaphthyl, triazinyl and triazolyl.

In this specification, unless stated otherwise, the term “heterocycle” refers to an optionally substituted, monocyclic, bicyclic or tricyclic saturated, partially saturated or unsaturated ring system containing at least one heteroatom selected independently from N, O and S. Bicyclic or tricyclic ring systems may be formed by annelation of two or more rings, by a bridging atom (e.g. O, S, N) or by a bridging group (e.g. alkylene).

Examples of heterocyclic moieties include, but are not limited to: azetidinyl, dihydrofuranyl, dihydrothienyl, dioxolanyl, 1,1-dioxo-thiomorpholinyl, imidazolidinyl, imidazolinyl, isothiazolinyl, isoxazolidinyl, isoxazolinyl, morpholinyl, oxazolidinyl, oxazolinyl, oxetanyl, piperazinonyl, piperazinyl, piperidinonyl, piperidinyl, pyranyl, pyrrolidinonyl, pyrrolidinyl, pyrrolinyl, tetrahydrofuranyl, tetrahydropyranyl, tetrahydrothiopyranyl, thiazolidinyl, thiazolinyl, thiomorpholinyl, thiopyranyl, triazolinyl, and the corresponding benzannulated heterocycles (e.g. dihydrobenzofuranyl, dihydrobenzothiophenyl, dihydrobenzoxazinyl, dihydrofuropyridinyl, dihydroquinolinyl, dihydrothienopyridinyl, indolinyl, pyrrolopyridinyl, tetrahydroquinolinyl, tetrahydroquinoxalinyl, and the like).

In this specification, unless stated otherwise, the term “alkylene-aryl”, “alkylene-heteroaryl”, “alkylene-heterocycle” and “alkylene-cycloalkyl” refers respectively to a substituent that is attached via the alkyl radical to an aryl, heteroaryl or cycloalkyl radical, respectively. The term “(C₁-C₆)alkylene-aryl” includes aryl-C₁-C₆-alkyl radicals such as benzyl, 1-phenylethyl, 2-phenylethyl, 1-phenylpropyl, 2-phenylpropyl, 3-phenylpropyl, 1-naphthylmethyl and 2-naphthylmethyl. The term “(C₁-C₆)alkylene-heteroaryl”includes heteroaryl-C₁-C₆-alkyl radicals, wherein examples of heteroaryl are the same as those illustrated in the above definition, such as 2-furylmethyl, 3-furylmethyl, 2-thienylmethyl, 3-thienylmethyl, 1-imidazolylmethyl, 2-imidazolylmethyl, 3-imidazolylmethyl, 2-oxazolylmethyl, 3-oxazolylmethyl, 2-thiazolylmethyl, 3-thiazolylmethyl, 2-pyridinylmethyl, 3-pyridinylmethyl, 4-pyridinylmethyl, 1-quinolylmethyl and the like.

In this specification, unless stated otherwise, a 5- or 6-membered ring containing one or more atoms independently selected from C, N, O and S, includes aromatic and heteroaromatic rings as well as carbocyclic and heterocyclic rings which may be saturated or unsaturated. Such rings include spirocyclic and bridged bicyclic systems. Examples of such rings may be, but are not limited to dihydrofuranyl, dihydrothienyl, dioxolanyl, 1,1-dioxo-thiomorpholinyl, furazanyl, furyl, imidazolidinyl, imidazolinyl, imidazolonyl, imidazolyl, isothiazolinyl, isothiazolyl, isoxazolidinyl, isoxazolinyl, isoxazolyl, morpholinyl, oxadiazolyl, oxazolidinyl, oxazolinyl, oxazolonyl, oxazolyl, phenyl, piperazinonyl, piperazinyl, piperidinonyl, piperidinyl, pyranyl, pyrazinyl, pyrazolyl, pyridazinyl, pyridonyl, pyridyl, pyrimidyl, pyrrolidinonyl, pyrrolidinyl, pyrrolinyl, pyrrolyl, tetrahydrofuranyl, tetrahydropyranyl, tetrahydrothiopyranyl, tetrazolyl, thiadiazolyl, thiazolidinyl, thiazolinyl, thiazolonyl, thiazolyl, thienyl, thiomorpholinyl, thiopyranyl, triazolinyl, triazinyl, triazolyl, cyclopentyl, cyclopentenyl, cyclohexyl and cyclohexenyl.

In this specification, unless stated otherwise, a 3- to 10-membered ring containing one or more atoms independently selected from C, N, O and S, includes aromatic and heteroaromatic rings as well as carbocyclic and heterocyclic rings which may be saturated or unsaturated. Examples of such rings may be, but are not limited to azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl.

In this specification, unless stated otherwise, the term “halo” or “halogen” may be fluoro, chloro, bromo or iodo.

In this specification, unless stated otherwise, the term “haloalkyl” means an alkyl radical as defined above, substituted with one or more halo radicals. The term “(C₁-C₆)haloalkyl” may include, but is not limited to, fluoromethyl, difluoromethyl, trifluoromethyl, fluoroethyl and difluoroethyl. The term “O—C₁-C₆-haloalkyl” may include, but is not limited to, fluoromethoxy, difluoromethoxy, trifluoromethoxy and fluoroethoxy.

In this specification, unless stated otherwise, the term “cyanoalkyl” means an alkyl radical as defined above, substituted with one or more cyano groups.

In this specification, unless stated otherwise, the term “optionally substituted” refers to radicals further bearing one or more substituents which may be, acyl, (C₁-C₆)alkyl, —(C₁-C₆)haloalkyl, —(C₃-C₇)cycloalkyl, —(C₁-C₆)alkylene-(C₃-C₇)cycloalkyl, —(C₃-C₇)cycloalkyl-(C₁-C₆)alkylene, —(C₀-C₆)alkylene-(C₃-C₇)spiroalkyl-(C₀-C₆)alkylene, hydroxy, (C₁-C₆)alkylene-oxy, dimethylamino(C₁-C₃)alkyl, mercapto, aryl, heterocycle, heteroaryl, (C₁-C₆)alkylene-aryl, (C₁-C₆)alkylene-heterocycle, (C₁-C₆)alkylene-heteroaryl, halogen, haloalkyl, trifluoromethyl, pentafluoroethyl, haloalkoxy, cyano, cyanomethyl, nitro, amino, amido, amidinyl, oxo, carboxyl, carboxamide, (C₁-C₆)alkylene-oxycarbonyl, carbamate, sulfonamide, ester or sulfonyl.

In this specification, unless stated otherwise, the term “independently” means that where more than one substituent is selected from a number of possible substituents, those substituents may be the same or different.

In this specification, unless stated otherwise, the term “solvate” refers to a complex of variable stoichiometry formed by a solute (e.g. a compound of Formula (I)) and a solvent. The solvent is a pharmaceutically acceptable solvent such as water; such solvent may not interfere with the biological activity of the solute.

In this specification, unless stated otherwise, the term “salt” refers to an acid addition or base addition salt of a compound of the invention. “Salts” include in particular “pharmaceutically acceptable salts”.

The pharmaceutically acceptable salts of the invention can be synthesized from a basic or acidic moiety, by conventional chemical methods.

When both a basic and an acid group are present in the same molecule, the compounds of the invention may also form internal salts, e.g., zwitterionic molecules.

In this specification, unless stated otherwise, certain compounds may exist in one or more particular geometric, optical, enantiomeric, diastereoisomeric, epimeric, stereoisomeric, tautomeric, conformational, or anomeric forms, including, but not limited to, cis- and trans-forms; E- and Z-forms; endo- and exo-forms, R-, S-, and meso-forms; D- and L-forms; d- and i-forms; (+) and (−) forms; keto-, enol-, and enolate-forms; α- and β-forms; axial and equatorial forms; and combinations thereof, collectively referred to as “isomers” or “isomeric forms”.

The term “isomer” includes compounds with one or more isotopic substitutions. For example, H may be in any isotopic form, including, but not limited to, ¹H, ²H (D), and 3H (T); C may be in any isotopic form, including, but not limited to, ¹²C, ¹³C, ¹⁴C; O may be in any isotopic form, including, but not limited to, ¹⁶O and ¹⁸O; and the like. F may be in any isotopic form, including, but not limited to, ¹⁹F and ¹⁸F; and the like.

In this specification, unless stated otherwise, the term “negative allosteric modulator of mGlu7” or “allosteric modulator of mGlu7” refers also to a pharmaceutically acceptable acid or base addition salt thereof, a stereochemically isomeric form thereof or an N-oxide form thereof.

In this specification, unless stated otherwise, the abbreviation BOC means tert-butyloxycarbonyl.

### Pharmaceutical Compositions

Allosteric modulators of mGlu7 described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The allosteric modulators of mGlu7 will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, PA (1995).

The amount of allosteric modulators of mGlu7, administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used CNS drugs are well known to the skilled person. The total daily dose usually ranges from about 0.05-2000 mg.

The present invention relates to pharmaceutical compositions which provide from about 0.01 to 1000 mg of the active ingredient per unit dose. The compositions may be administered by any suitable route. For example, orally in the form of capsules and the like, parenterally in the form of solutions for injection, topically in the form of onguents or lotions, ocularly in the form of eye-drops, rectally in the form of suppositories, intranasally or transcutaneously in the form of a delivery system like patches.

For oral administration, the allosteric modulators of mGlu7 thereof can be combined with a suitable solid or liquid carrier or diluent to form capsules, tablets, pills, powders, syrups, solutions, suspensions and the like.

The tablets, pills, capsules, and the like contain from about 0.01 to about 99 weight percent of the active ingredient and a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parenteral administration the disclosed allosteric modulators of mGlu7, or salts thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations described previously, the compounds may also be formulated as a depot preparation. Such long acting formulations may be administered for example, by subcutaneously implantation or by intramuscular injection. Thus, for example, the compounds may be formulated as an emulsion in an acceptable oil, or ion exchange resins, or as sparingly soluble derivatives, for example, as sparingly soluble salts.

Preferably disclosed allosteric modulators of mGlu7 or pharmaceutical formulations containing these compounds are in unit dosage form for administration to a mammal.

The unit dosage form can be any unit dosage form known in the art including, for example, a capsule, an IV bag, a tablet, or a vial. The quantity of active ingredient in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

### Methods of Synthesis

The compounds according to the invention, in particular the compounds according to the Formula (I) to (VI), may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M., (1991) Protecting Groups in Organic Synthesis, John Wiley & Sons). These groups are removed at a convenient stage of the compound synthesis using methods that are readily apparent to those skilled in the art. The selection of process as well as the reaction conditions and order of their execution shall be consistent with the preparation of compounds of Formula (I) to (VI).

The compounds according to the invention may be represented as a mixture of enantiomers, which may be resolved into the individual pure R- or S-enantiomers. If for instance, a particular enantiomer is required, it may be prepared by asymmetric synthesis or by derivation with a chiral auxiliary, where the resulting diastereomeric mixture is separated and the auxiliary group cleaved to provide the pure desired enantiomers. Alternatively, where the molecule contains a basic functional group such as an amino or an acidic functional group such as carboxyl, this resolution may be conveniently performed by fractional crystallization from various solvents as the salts of an optical active acid or by other methods known in the literature (e.g. chiral column chromatography).

Resolution of the final product, an intermediate or a starting material may be performed by any suitable method known in the art (Eliel E. L., Wilen S. H. and Mander L. N. (1984) Stereochemistry of Organic Compounds, Wiley-Interscience).

Many of the heterocyclic compounds of the invention can be prepared using synthetic routes well known in the art (Katrizky A. R. and. Rees C. W. (1984) Comprehensive Heterocyclic Chemistry, Pergamon Press).

The product from the reaction can be isolated and purified by employing standard techniques, such as extraction, chromatography, recrystallization and distillation.

The compounds of the invention may be prepared by general route of synthesis as disclosed in the following methods.

In one embodiment of the present invention, compounds of Formula (V) may be prepared according to the synthetic sequence illustrated in Scheme 1. 5-Bromoisobenzofuran-1(3H)-one g1 may be oxidized in the presence of N-bromosuccinimide, in an appropriate solvent such as carbon tetrachloride, at an appropriate temperature, to afford the intermediate 5-bromo-3-hydroxyisobenzofuran-1(3H)-one g2. Intermediate g2 can then be converted into bromophthalazinone derivatives g4 by condensation with selected hydrazine derivatives g3, in an appropriate solvent such as ethanol, at an appropriate temperature. Intermediates g4 may be converted into final compounds g6 by suitable reactions known by people skilled in the art of organic synthesis, for example by Suzuki cross coupling reaction, mediated by palladium-complex catalyst such as Pd(PPh₃)₄, PdCl₂(dppf), in the presence of a base such as potassium carbonate or cesium carbonate, in an appropriate solvent such as a mixture of DME/water, at an appropriate temperature.

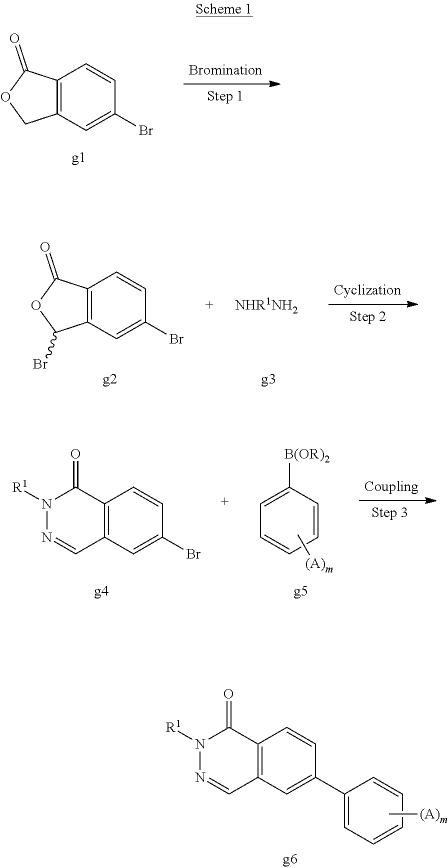

Similarly, final compounds g6 may be prepared according to the synthetic sequence illustrated in Scheme 2. 5-Bromoisoindoline-1,3-dione g7 may be converted into 5-bromo-hydroxyisoindolinone derivative g8 under reductive conditions for example, with zinc and copper (II) sulfate, in the presence of a base such as sodium hydroxide. Intermediate g8 may be converted into intermediate compound g9 by reaction with hydrazine hydrate, in an appropriate solvent such as ethanol, at an appropriate temperature. The corresponding bromo-phthalazinone derivative g9 may be converted into intermediates g10 by Suzuki coupling reaction with aryl boronic acids g5′, mediated by palladium-complex catalyst such as Pd(PPh₃)₄, in the presence of a base such as potassium carbonate, and in an appropriate solvent such as a mixture of DME/water, at an appropriate temperature. Intermediates g10 may then be converted into final compounds g6 by suitable reactions known by people skilled in the art of organic synthesis, such as Chan-Lam or Ullmann cross coupling reactions, mediated by copper-complex catalysts such as CuI and Cu(OAc)₂, in the presence of a base such as potassium carbonate, in an appropriate solvent such as DMF and at an appropriate temperature.

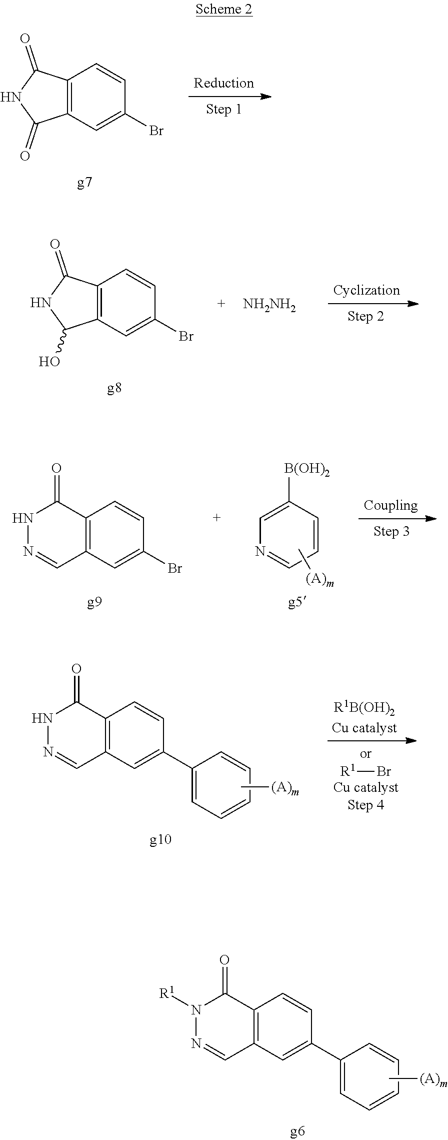

In another specific aspect of Formula (V), final compounds g6 may be prepared according to the synthetic sequence illustrated in Scheme 3. Intermediate g4, prepared according to Scheme 1 Step 2, may be converted into boronate-phthalizinone derivatives g1l by reaction with diboron reageant, in an appropriate solvent such as 1,4-dioxane, and at an appropriate temperature. Suzuki cross coupling of g1l with an appropriate aryl bromide g12, mediated by palladium-complex such as PdCl₂(dppf), in the presence of a base such as potassium carbonate give the final compounds g6.

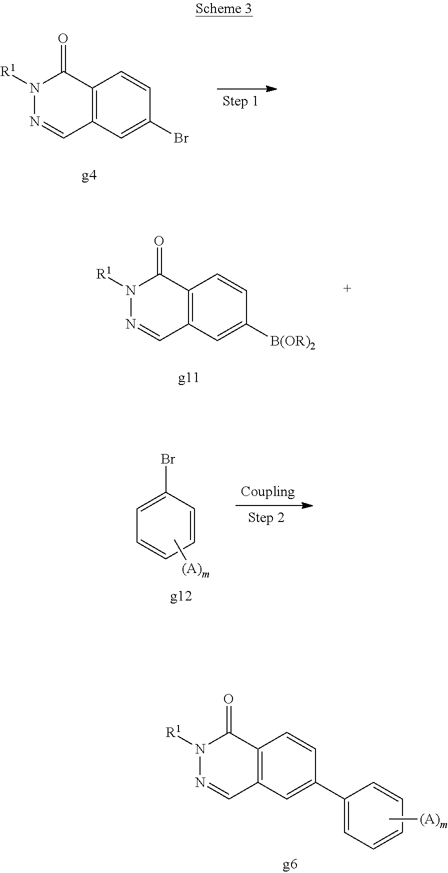

Final compounds g6 may also be prepared according to the synthetic sequence illustrated in Scheme 4. Suzuki cross coupling of g2 with an appropriate aryl boronic acid g5′, mediated by palladium-complex such as Pd(PPh₃)₄, in the presence of a base such as potassium carbonate provides the intermediate compounds g13. The 3-hydroxy-isobenzofuran-1(3H)-one derivatives g13 may be converted into final compounds g6 by reaction with substituted hydrazines g3, in an appropriate solvent such as ethanol, at an appropriate temperature.

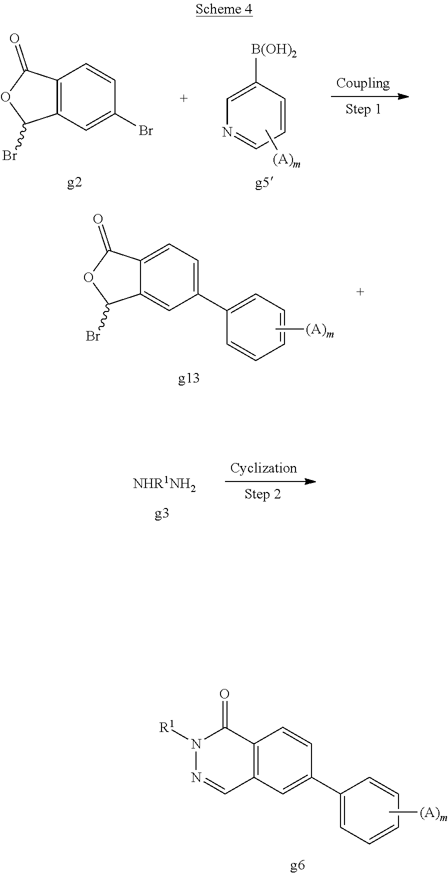

Final compounds g6 may also be prepared according to the synthetic sequence illustrated in Scheme 5. Intermediate compounds g13, prepared according to Scheme 4 Step 1, may be converted into compounds g10 by reaction with hydrazine hydrate, in an appropriate solvent such as ethanol, at an appropriate temperature. Chan-Lam cross coupling of g10 with appropriate aryl boronic acids (R¹=aryl), mediated by copper-complex such as Cu(OAc)₂, in the presence of a base such as potassium carbonate, in an appropriate solvent such as DMF, afford the final compounds g6.

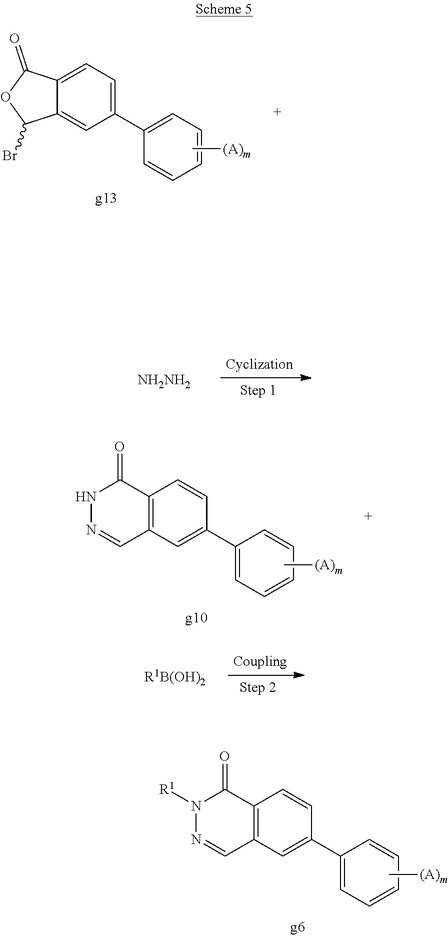

In another specific aspect of Formula (V), final compounds of structure g15 may be prepared according to the synthetic sequence illustrated in Scheme 6. Derivatives g14, prepared according to Scheme 1, where X is a halogen such as bromine, may be transformed into final compounds g15 by reaction with an acyclic or cyclic amine, catalyzed by palladium reagents such as PdCl₂(dppf) or Pd(OAc)₂, in the presence of a base such as Cs₂CO₃ or K₃PO₄, in an appropriate solvent such as DMF or dioxane at the appropriate temperature.

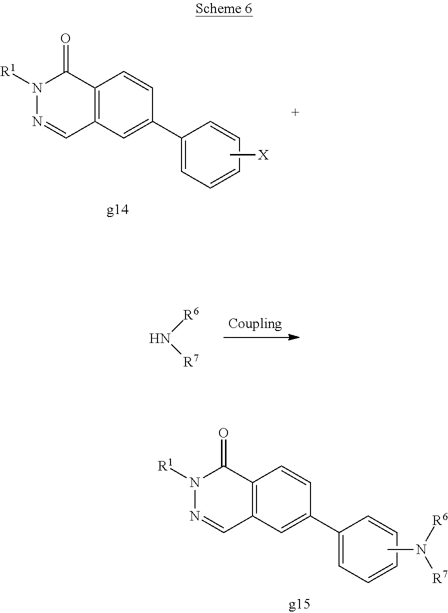

In another specific aspect of Formula (V), final compounds of structure g17 may be prepared according to the synthetic sequence illustrated in Scheme 7. Intermediate compounds g16, prepared according to Scheme 5, may be converted into final compounds g17 by reaction with NaBH₄, in an appropriate solvent such as DCM at the appropriate temperature.

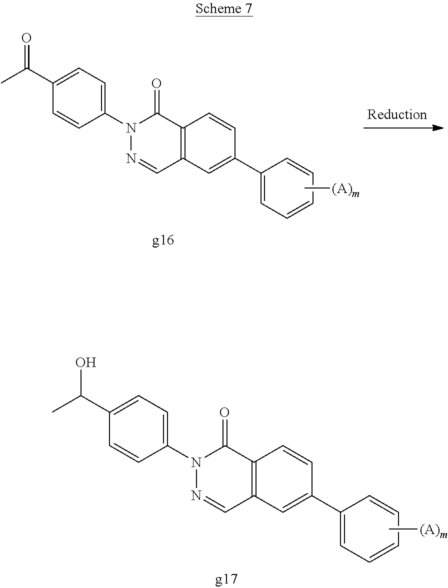

In another specific aspect of Formula (VI), final compounds g19 may be prepared according to the synthetic sequence illustrated in Scheme 8. Intermediate derivatives g18, prepared according to Scheme 1, may be converted into final compounds g19 by reaction with a nucleophile (such as amines) in an appropriate solvent such as DMF at the appropriate temperature.

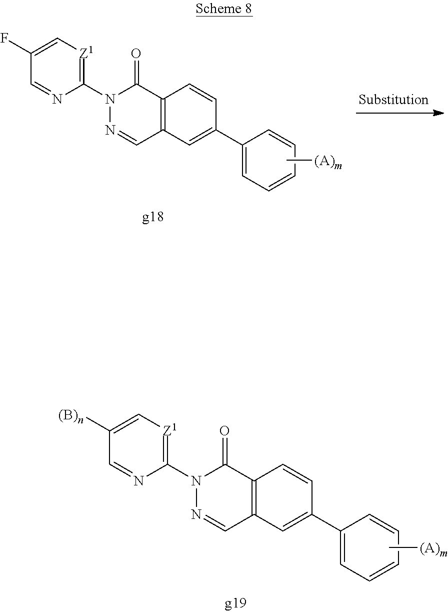

In another specific aspect of Formula (VI), final compounds g19 may be prepared according to the synthetic sequence illustrated in Scheme 9. Intermediate derivatives g20, prepared according to Scheme 1, may be converted into final compounds g19 by reduction followed by coupling reaction in an appropriate solvent such as DMF at the appropriate temperature.

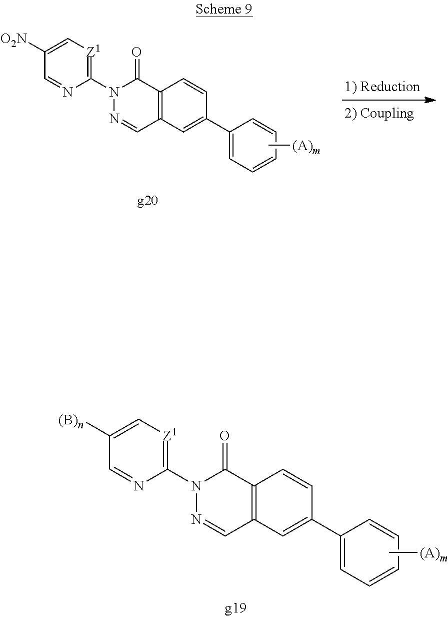

Finally, in another specific aspect of Formula (VI), final compounds g22 may be prepared according to the synthetic sequence illustrated in Scheme 10. Intermediate derivatives g21, prepared according to Scheme 8, may be converted into final compounds g22 by methylation of the alcohol group using methyl iodide in a presence of a base such as sodium hydride.

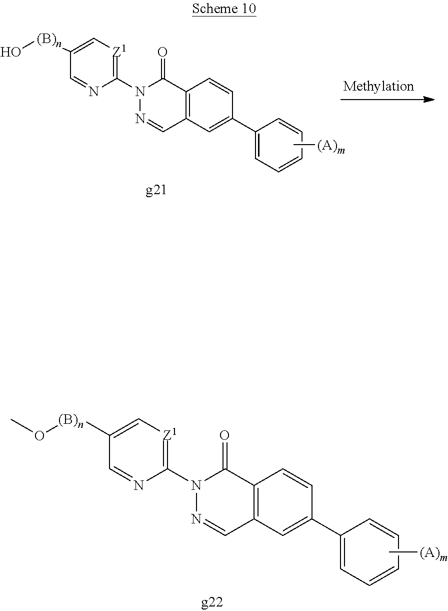

### EXPERIMENTAL

Unless otherwise noted, all starting materials were obtained from commercial suppliers and used without further purification.

Specifically, the following abbreviations may be used in the examples and throughout the specification.

| | |
|---|---|
| ACN (Acetonitrile) | MgSO₄ (Magnesium sulfate) |
| CCl₄ (Carbon tetrachloride) | MHz (Megahertz) |
| CDCl₃ (Deuterated chloroform) | min (Minutes) |
| Cs₂CO₃ (Cesium carbonate) | mL (Milliliters) |
| CuI (Copper (I) iodide) | mmol (Millimoles) |
| Cu(OAc)₂ (Copper (II) acetate) | M.p. (Melting point) |
| DCM (Dichloromethane) | μl (Microliters) |
| DME (1, 2-Dimethoxyethane) | μm (Micrometers) |
| DMF (Dimethylformamide) | μmol (Micromoles) |
| EtOAc (Ethyl acetate) | NMR (Nuclear Magnetic Resonance) |
| EtOH (Ethanol) | NH₄Cl (Ammonium chloride) |
| Et₂O (Diethyl ether) | NaBH₄ (Sodium borohydride) |
| g (Grams) | NaHCO₃ (Sodium bicarbonate) |
| h (Hours) | NaOH (Sodium hydroxide) |
| ¹H (Proton) | Na₂CO₃ (Sodium carbonate) |
| HCl (Hydrochloric acid) | Na₂SO₄ (Sodium sulfate) |
| HPLC (High Performance Liquid | nd (Not determined) |
| Chromatography) | |
| Hz (Hertz) | PdCl₂(dppf) [1, 1′-Bis(diphenylphosphino)ferrocene] |
| | dichloropalladium(II) |
| H₂O (Water) | Pd(OAc)₂ (Palladium (II) acetate) |
| K₂CO₃ (Potassium carbonate) | Pd(Ph₃)₄ (Tetrakis(triphenylphosphine)palladium(0)) |
| K₃PO₄ (Potassium phosphate) | psi (Pounds per square inch) |
| LC-MS (Liquid Chromatography | rt (Room temperature) |
| Mass Spectrometry) | |
| M (Molar) | RT (Retention Time) |
| MeOH (Methanol) | UPLC-MS (Ultra Performance Liquid |
| | Chromatography-Mass Spectrometry) |
| mg (Milligrams) | Xantphos (4, 5-Bis(diphenylphosphino)-9, 9-dimethyl |
| | xanthene) |

All references to brine refer to a saturated aqueous solution of NaCl. Unless otherwise indicated, all temperatures are expressed in ° C. (degrees Celsius). All reactions are conducted under an inert atmosphere at room temperature unless otherwise noted.

Most of the reactions were monitored by thin-layer chromatography on 0.25 mm Merck silica gel plates (60F-254), visualized with UV light. Flash column chromatography was performed on prepacked silica gel cartridges (15-40 μM, Merck).

### EXAMPLES

### Example 1: 6-(2,4-Dimethylphenyl)-2-ethylphthalazin-1(2H)-one (Final Compound 1-1)

### 5-Bromo-3-hydroxyisobenzofuran-1 (3H)-one

According to Scheme 1 Step 1: To a solution of 5-bromoisobenzofuran-1(3H)-one (3.00 g, 14.1 mmol) in CCl₄ (30 mL) was added 1-bromopyrrolidine-2,5-dione (3.26 g, 18.3 mmol) and the reaction mixture was stirred at 90° C. for 5 h. After evaporation of the solvent, water was added to the residue, then the reaction mixture was stirred for 1 h at rt and stored in the fridge overnight. The resulting precipitate was filtered, washed with water and dried under reduced pressure to afford the title compound in quantitative yield. The crude product was used in the next step without any further purification.

UPLC-MS: RT=1.07 min; MS m/z ES⁺=no ionisation. 6-Bromo-2-ethylphthalazin-1(2H)-one According to Scheme 1 Step 2: A mixture of 5-bromo-3-hydroxyisobenzofuran-1(3H)-one (3.30 g, 14.1 mmol) and ethylhydrazine oxalate (2.38 g, 15.8 mmol) in EtOH (60 mL) was stirred at 120° C. in the microwave for 20 min. After cooling to rt, the mixture was diluted with EtOAc and washed with water. The organic layer was separated, dried over MgSO₄, filtered and concentrated under reduced pressure to afford the title compound (2.8 g, 77%). The crude product was used in the next step without any further purification.

UPLC-MS: RT=0.91 min; MS m/z ES⁺=253.

### 6-(2,4-Dimethylphenyl)-2-ethylphthalazin-1(2H)-one

According to Scheme 1 Step 3: To a solution of 6-bromo-2-ethylphthalazin-1(2H)-one (200 mg, 790 μmol) in DME (2 mL) was added 2,4-dimethylphenylboronic acid (119 mg, 790 μmol), K₂CO₃ (218 mg, 1.58 mmol) and then Pd(PPh₃)₄(46 mg, 40 μmol). The mixture was stirred in the microwave at 100° C. for 30 min. After cooling to rt, the reaction mixture was diluted with EtOAc and was washed with water. The organic layer was separated, dried over MgSO₄, filtered and concentrated under reduced pressure. The crude residue was purified by preparative HPLC to afford the title compound (47 mg, 21%) as a white foam.

UPLC-MS: RT=1.20 min; MS m/z ES⁺=279; ¹H-NMR (300 MHz, DMSO-d₆) δ: 8.47 (1H, s), 8.30 (1H, d), 7.90 (1H, s), 7.81 (1H, d), 7.18 (3H, q), 4.16 (2H, q), 2.34 (3H, s), 2.23 (3H, s), 1.31 (3H, t).

### Example 2: 2-Ethyl-6-(3-(pyrrolidin-1-yl)phenyl)phthalazin-1(2H)-one (Final Compound 1-13)

According to Scheme 1 Step 3: To a solution of 6-bromo-2-ethylphthalazin-1(2H)-one (100 mg, 395 μmol) in dioxane/water (5:1, 1.2 mL) was added 1-(3-(4,4,5,5-tetramethyl-1,3,2-dioxaborolan-2-yl)phenyl)pyrrolidine (162 mg, 593 μmol), Cs₂CO₃ (257 mg, 790 μmol) and PdCl₂(dppf) (29 mg, 40 μmol). The mixture was stirred in the microwave at 90° C. for 30 min. After cooling to rt, the reaction mixture was then diluted with EtOAc and was washed with water. The organic layer was separated, dried over MgSO₄, filtered and concentrated under reduced pressure. The crude residue was purified by preparative HPLC to afford the title compound (9 mg, 7%) as a white solid.

UPLC-MS: RT=1.17 min; MS m/z ES⁺=329; ¹H-NMR (300 MHz, DMSO-d₆) δ: 8.49 (1H, s), 8.31-8.14 (4H, m), 7.35 (1H, t), 7.02 (1H, d), 6.89 (1H, s), 6.63 (1H, d), 4.19 (3H, q), 1.99 (5H, s), 1.31 (4H, t).

### Example 3: 6-(4-(Azetidin-1-yl)pyridin-2-yl)-2-(pyridin-2-yl)phthalazin-1(2H)-one (Final Compound 1-53)

### 6-Bromo-2-(pyridin-2-yl)phthalazin-1(2H)-one

According to Scheme 3 Step 1: Under an inert atmosphere, a mixture of 5-bromo-3-hydroxyisobenzofuran-1(3H)-one (10.0 g, 43.9 mmol, 1.0 equiv.) and 2-hydrazinopyridine (5.75 g, 52.6 mmol, 1.2 equiv.) in EtOH (100 mL) was stirred at 120° C. overnight. After cooling to room temperature, the resulting precipitate was filtered, washed with little amounts of EtOH, and dried under high vacuum to give the title compound (9.2 g, 30.6 mmol, 70%) as an off-white solid.

UPLC-MS: RT=0.50 min; MS m/z ES⁺=229; ¹H-NMR (500 MHz, CDCl₃): δ=8.68 (ddd, J=4.9, 1.9, 0.8 Hz, 1H), 8.35 (dt, J=8.3, 0.7 Hz, 1H), 8.27 (d, J=0.7 Hz, 1H), 7.95-7.84 (m, 3H), 7.75 (dt, J=8.1, 1.0 Hz, 1H), 7.37 (ddd, J=7.4, 4.9, 1.1 Hz, 1H).

### 2-(Pyridin-2-yl)-6-(4,4,5,5-tetramethyl-1, 3,2-dioxaborolan-2-yl)phthalazin-1(2H)-one

According to Scheme 3 Step 2: A stirred mixture of 6-bromo-2-(pyridin-2-yl)phthalazin-1(2H)-one (1.0 g, 3.3 mmol, 1.0 equiv.), bis(pinacolato)diboron (1.0 g, 4.0 mmol, 1.2 equiv.) and KOAc (978 mg, 9.97 mmol, 3.0 equiv.) in anhydrous 1,4-dioxane (50 mL) was degassed with argon for 10 min. Pd(dppf)C₁₂ (122 mg, 0.17 mmol, 0.05 equiv.) was added and the resulting mixture was stirred for 24 h under argon atmosphere in a closed vial at 100° C. The reaction mixture was diluted with DCM (200 mL), and filtered over a short plug of Celite. Water (75 mL) was added and the layers were separated. The aqueous layer was extracted with DCM (2×200 mL). The combined organic layers were washed with brine (2×50 mL), dried over MgSO₄, filtered, and solvents were removed in vacuo. Silica gel chromatography (dcm/EtOAc 1:1 to 1:2) gave the title compound (832 mg, 2.38 mmol, 72%) as a colorless solid. ¹H-NMR (500 MHz, CDCl₃): δ=8.68 (ddd, J=4.9, 1.9, 0.9 Hz, 1H), 8.46 (dt, J=7.9, 0.7 Hz, 1H), 8.36 (d, J=0.8 Hz, 1H), 8.24-8.16 (m, 2H), 7.87 (ddd, J=8.1, 7.4, 1.8 Hz, 1H), 7.78 (dt, J=8.1, 0.9 Hz, 1H), 7.35 (ddd, J=7.4, 4.9, 1.1 Hz, 1H), 1.38 (s, 12H).

### 6-(4-(Azetidin-1-yl)pyridin-2-yl)-2-(pyridin-2-yl)phthalazin-1(2H)-one

According to Scheme 3 Step 3: To a stirred solution of 4-(azetidin-1-yl)-2-bromopyridine (125 mg, 0.59 mmol, 1.0 equiv.) in a pressure tube were added 2-(pyridin-2-yl)-6-(4,4,5,5-tetramethyl-1,3,2-dioxaborolan-2-yl)phthalazin-1(2H)-one (248 mg, 0.71 mmol, 1.2 equiv.), K₂CO₃ (106 mg, 0.77 mmol, 1.3 equiv.), DME (4 mL), and water (6 mL). Pd(PPh₃)₄(34 mg, 0.03 mmol, 0.05 equiv.) was added in one portion, and the solution was stirred at 90° C. for 36 h. After TLC indicated full conversion, the reaction mixture was partitioned between sat. aqueous NaHCO₃ solution (50 mL) and DCM (50 mL). The layers were separated, and the aqueous layer was extracted with DCM (3×75 mL). The combined organic layers were dried over MgSO₄, filtered, and concentrated under reduced pressure. Silica gel chromatography (DCM/EtOAc 1:1 TO 3:7) gave the title compound (9 mg, 0.03 mmol, 4%) as a pale yellow solid.

LC-MS: RT=3.99 min; MS m/z=356.18 [M+H]+; ¹H-NMR (500 MHz, CDCl₃): δ=8.70 (ddd, J=4.9, 1.9, 0.9 Hz, 1H), 8.59-8.55 (m, 1H), 8.42 (d, J=0.7 Hz, 1H), 8.27 (dd, J=5.3, 0.8 Hz, 1H), 8.01 (dd, J=8.3, 1.7 Hz, 1H), 7.95 (dd, J=1.7, 0.6 Hz, 1H), 7.90 (ddd, J=8.1, 7.4, 1.9 Hz, 1H), 7.79 (dt, J=8.0, 1.0 Hz, 1H), 7.37 (ddd, J=7.5, 4.9, 1.0 Hz, 1H), 6.87 (dd, J=5.3, 1.6 Hz, 1H), 6.49 (dd, J=1.7, 0.8 Hz, 1H), 4.15 (t, J=7.4 Hz, 4H), 2.46 (tt, J=8.2, 7.0 Hz, 2H).

### Example 4: 6-(3-Cyclopropylphenyl)-2-(pyridin-2-yl)phthalazin-1(2H)-one (Final Compound 1-22)

### 6-(3-Cyclopropylphenyl)phthalazin-1(2H)-one

According to Scheme 2 Step 3: In a sealed tube, a mixture of 6-bromophthalazin-1(2H)-one (250 mg, 1.11 mmol), 3-cyclopropylphenylboronic acid (198 mg, 1.22 mmol), K₂CO₃ (307 mg, 2.22 mmol) and Pd(PPh₃)₄(64.2 mg, 55.6 μmol) in DME/water (2:1, 3 mL) was stirred at 100° C. overnight. After cooling to rt, the reaction mixture was diluted with EtOAc and water. The organic layer was separated, dried over MgSO₄, filtered and concentrated under reduced pressure to afford the title compound (250 mg, 86%) as a yellow solid. The crude solid was used in the next step without any further purification.

UPLC-MS: RT=0.99 min; MS m/z ES⁺=263.

### 6-(3-Cyclopropylphenyl)-2-(pyridin-2-yl)phthalazin-1(2H)-one

According to Scheme 2 Step 4: A mixture of 2-bromopyridine (113 mg, 715 μmol), 6-(3-cyclopropylphenyl)phthalazin-1(2H)-one (125 mg, 477 μmol), CuI (9.08 mg, 47.7 μmol) and K₂CO₃ (65.9 mg, 477 μmol) in DMF (1 mL) was stirred at 150° C. for 3 h. EtOAc and water were added to the residue. The aqueous layer was extracted with EtOAc. The organic layers were combined, dried over MgSO₄, filtered and concentrated under reduced pressure to afford a red oil. The crude oil was purified by flash chromatography on silica gel using DCM/MeOH (99:1) as eluent. The resulting solid was triturated in Et₂O, filtered and dried under reduced pressure to afford the title compound (58 mg, 36%) as a light pink solid. M.p.: 181-184° C.; UPLC-MS: RT=1.09 min; MS m/z ES⁺=341; ¹H-NMR (300 MHz, CDCl₃) δ: 8.76-8.69 (1H, m), 8.58 (1H, d), 8.42 (1H, s), 8.09-8.01 (1H, m), 7.98-7.79 (3H, m), 7.53-7.35 (4H, m), 7.20-7.13 (1H, m), 2.09-1.97 (1H, m), 1.10-1.02 (2H, m), 0.85-0.77 (2H, m).

### Example 5: 6-(2,4-Dimethylphenyl)-2-(2-fluorophenyl)phthalazin-1(2H)-one (Final Compound 1-4)

### 6-Bromo-2-(2-fluorophenyl)phthalazin-1(2H)-one

According to Scheme 3 Step 1: To a solution of 5-bromo-3-hydroxyisoindolin-1-one (200 mg, 877 μmol) in water (3 mL) was added (2-fluorophenyl)hydrazine hydrochloride (157 mg, 965 μmol) and NaOH iN (1 mL). The mixture was stirred at 100° C. for 2 h. After cooling to rt, the solid was filtered, washed with water, triturated with hot EtOAc and dried under reduced pressure to afford the title compound (82 mg, 29%) as a yellow solid.

UPLC-MS: RT=1.05 min; MS m/z ES⁺=319.

### 6-(2,4-Dimethylphenyl)-2-(2-fluorophenyl)phthalazin-1(2H)-one

According to Scheme 3 Step 2: To a mixture of 6-bromo-2-(2-fluorophenyl)phthalazin-1(2H)-one (82.0 mg, 257 μmol), K₂CO₃ (71.0 mg, 514 μmol) and 2,4-dimethylphenylboronic acid (48.2 mg, 321 μmol) in DMF/water (10:1, 4.4 mL), previously degassed with nitrogen, was added PdCl₂(dppf) (18.8 mg, 26.0 μmol). The reaction mixture was stirred in the microwave at 100° C. for 25 min. After cooling to rt, the reaction mixture was filtered through Celite and washed with EtOAc. The organic layer was separated, washed with brine, dried over MgSO₄, filtered and concentrated under reduced pressure. The crude was purified by preparative HPLC to afford the title compound (10 mg, 11%) as a brown solid.

UPLC-MS: RT=1.27 min; MS m/z ES⁺=345; ¹H-NMR (300 MHz, CDCl₃) δ: 8.53 (1H, d), 8.32 (1H, s), 7.83-7.77 (2H, m), 7.56-7.41 (2H, m), 7.33-7.26 (3H, m), 7.20-7.15 (2H, m), 2.51 (3H, s), 2.36 (3H, s).

### Example 6: 2-(2-Chlorophenyl)-6-(2,4-dimethylphenyl)phthalazin-1(2H)-one (Final Compound 1-5)

### 5-(2,4-Dimethylphenyl)-3-hydroxyisobenzofuran-1(3H)-one

According to Scheme 4 Step 1: Prepared as per example 1 in Scheme 1 Step 3, from 2,4-dimethylphenylboronic acid (2.16 g, 14.4 mmol), 5-bromo-3-hydroxyisobenzofuran-1(3H)-one (2.20 g, 9.61 mmol), Pd(PPh₃)₄ (333 mg, 288 μmol) and K₂CO₃ (2.66 g, 19.2 mmol) in DME/water (2:1, 32 mL) to afford the title compound (1.4 g, 57%) as a brown foam. The crude was used in the next step without any further purification.

UPLC-MS: RT=1.01 min; MS m/z ES⁺=255.

### 2-(2-Chlorophenyl)-6-(2,4-dimethylphenyl)phthalazin-1(2H)-one

According to Scheme 4 Step 2: To a solution of 5-(2,4-dimethylphenyl)-3-hydroxyisobenzofuran-1(3H)-one (50.0 mg, 197 μmol) in EtOH (2 mL) was added (2-chlorophenyl)hydrazine hydrochloride (38.7 mg, 216 μmol). The reaction mixture was stirred in the microwave at 100° C. for 20 min. After cooling to rt, a precipitate appeared. The solid was filtered, washed with cold EtOH and Et₂O, dried under reduced pressure to afford the title compound (10 mg, 14%) as a white solid.

UPLC-MS: RT=1.29 min; MS m/z ES⁺=361; ¹H-NMR (300 MHz, DMSO-d₆) δ: 8.61 (1H, s), 8.34 (1H, d), 8.02 (1H, s), 7.90 (1H, d), 7.72-7.53 (4H, m), 7.25-7.15 (3H, m), 2.36 (3H, s), 2.27 (3H, s).

### Example 7: 6-(2,4-Dimethylphenyl)-2-(4-(methylsulfonyl)phenyl)phthalazin-1(2H)-one (Final Compound 1-8)

### 6-(2,4-Dimethylphenyl)phthalazin-1(2H)-one

According to Scheme 5 Step 1: Prepared as per example 6 in Scheme 4 Step 2, from 5-(2,4-dimethylphenyl)-3-hydroxyisobenzofuran-1(3H)-one (940 mg, 3.70 mmol) and hydrazine hydrate (308 μL, 4.07 mmol) in EtOH (2 mL) to afford the title compound (420 mg, 45%) as a grey solid.

UPLC-MS: RT=0.99 min; MS m/z ES⁺=251.

### 6-(2,4-Dimethylphenyl)-2-(4-(methylsulfonyl)phenyl)phthalazin-1(2H)-one

According to Scheme 5 Step 2: Prepared as per example 3 in Scheme 2 Step 4 from 6-(2,4-dimethylphenyl)phthalazin-1(2H)-one (100 mg, 400 μmol), K₂CO₃ (166 mg, 1.20 mmol), Cu(OAc)₂ (145 mg, 799 μmol) and 4-(methylsulfonyl)phenylboronic acid (80.0 mg, 400 μmol) in DMF (1.3 mL) to afford the title compound (4 mg, 2%) as a white solid.

UPLC-MS: RT=1.17 min; MS m/z ES⁺=405; ¹H-NMR (300 MHz, DMSO-d₆) δ: 8.68 (1H, s), 8.38 (1H, d), 8.10-7.90 (6H, m), 7.25-7.15 (3H, m), 3.31 (3H, s), 2.36 (3H, s), 2.27 (3H, s).

### Example 8: 6-(3-(Azetidin-1-yl)phenyl)-2-ethylphthalazin-1(2H)-one (Final Compound 1-11)

According to Scheme 6: To a solution of 6-(3-bromophenyl)-2-ethylphthalazin-1(2H)-one (20 mg, 61 μmol) in DMF (0.5 mL) was added azetidine (8.19 μl, 122 μmol), PdCl₂(dppf) (4.45 mg, 6.08 μmol), Cs₂CO₃ (30 mg, 91 μmol) and Xantphos (5.27 mg, 9.11 μmol). The reaction mixture was stirred at 130° C. for 3 h. The reaction mixture was diluted with EtOAc and washed with brine. The organic layer was separated, dried over MgSO₄, filtered and concentrated under reduced pressure. The crude residue was purified by preparative HPLC to afford the title compound (2.2 mg, 12%) as a beige solid.

UPLC-MS: RT=1.11 min; MS m/z ES⁺=306; ¹H-NMR (300 MHz, DMSO-d₆) δ: 8.50 (1H, s), 8.31-8.12 (3H, m), 7.40 (2H, q), 7.15 (1H, d), 6.82 (1H, s), 6.45 (1H, d), 4.19 (2H, d), 3.89 (4H, t), 1.36 (3H, t), 1.08 (1H, t).

### Example 9: 6-(3-(Azetidin-1-yl)phenyl)-2-(5-fluoropyridin-2-yl)phthalazin-1(2H)-one (Final Compound 1-24)

According to Scheme 6: To a solution of 6-(3-bromophenyl)-2-(5-fluoropyridin-2-yl)phthalazin-1(2H)-one (70.0 mg, 177 μmol) in dioxane (1 mL) was added azetidine (24.0 μl, 353 μmol), Pd(OAc)₂ (2.78 mg, 12.0 μmol), K₃PO₄ (94.0 mg, 442 μmol) and Xantphos (15.3 mg, 27.0 μmol). The reaction mixture was purged under nitrogen for 10 min and was stirred at 85° C. for 72 h. The reaction mixture was diluted with EtOAc and washed with brine. The organic layer was separated, dried over MgSO₄, filtered and concentrated under reduced pressure. The crude residue was purified by preparative HPLC to afford the title compound (1.9 mg, 3%) as a yellow oil.

UPLC-MS: RT=1.05 min; MS m/z ES⁺=373; ¹H-NMR (300 MHz, CDCl₃) δ: 8.59-8.50 (2H, m), 8.42 (1H, s), 8.09-8.02 (1H, m), 7.95 (1H, s), 7.87-7.80 (1H, m), 7.67-7.58 (1H, m), 7.41-7.33 (1H, m), 7.08-7.02 (1H, m), 6.71 (1H, s), 6.59-6.53 (1H, m), 3.99 (4H, t), 2.45 (2H, q).

### Example 10: 6-(2,4-Dimethylphenyl)-2-(4-(1-hydroxyethyl)phenyl)phthalazin-1(2H)-one (Final Compound 1-14)

According to Scheme 7: To a solution of 2-(4-acetylphenyl)-6-(2,4-dimethylphenyl)phthalazin-1(2H)-one (50.0 mg, 136 μmol) (prepared according to Scheme 5), under nitrogen, in DCM (2 mL) was added NaBH₄ (5.13 mg, 136 μmol). The reaction mixture was stirred at rt for 2 h and was quenched with an aqueous solution of NH₄Cl. The organic layer was separated and washed with a saturated aqueous solution of NaHCO₃. The organic layer was dried over MgSO₄, filtered and concentrated under reduced pressure. The crude residue was purified by preparative HPLC to afford the title compound (20 mg, 40%) as a white solid.

UPLC-MS: RT=1.16 min; MS m/z ES⁺=371; ¹H-NMR (300 MHz, CDCl₃) δ: 8.54 (1H, d), 8.30 (1H, s), 7.75 (1H, d), 7.66 (2H, d), 7.52 (2H, d), 7.19-7.12 (3H, m), 4.98 (1H, brs), 2.40 (3H, s), 2.28 (3H, s), 1.88-1.87 (1H, m), 1.55 (3H, d).

### Example 11: 2-(5-(2-Oxa-6-azaspiro[3.3]heptan-6-yl)pyrimidin-2-yl)-6-(3-methoxy-2-methylphenyl)phthalazin-1(2H)-one (Final Compound 1-84)

According to Scheme 8: To a stirred solution of 6-(3-methoxy-2-methylphenyl)-2-(5-fluoropyrimidin-2-yl)phthalazin-1(2H)-one (75 mg, 0.21 mmol, 1.0 equiv.) in a pressure tube were added DMF (8 mL), K₂CO₃ (57 mg, 0.42 mmol, 2.0 equiv.), and 2-oxa-6-aza-spiro[3.3]heptane (23 mg, 0.23 mmol, 1.1 equiv.), and the solution was stirred at 100° C. for 4 h. After cooling to room temperature, solvents were removed under reduced pressure. The residue was taken up in DCM (25 mL), and the reaction mixture was partitioned between sat. aqueous NaHCO₃ solution (50 mL) and DCM (50 mL). The layers were separated, and the aqueous layer was extracted with DCM (3×50 mL). The combined organic layers were dried over MgSO₄, filtered, and concentrated under reduced pressure. Silica gel chromatography (DCM/MeOH 95:5) gave the side product (18 mg, 0.05 mmol, 22%) as a colorless solid.

LC-MS: m/z=442.06 [M+H]⁺, RT=4.06 min; ¹H-NMR (500 MHz, CDCl₃): δ=8.51 (d, J=8.2 Hz, 1H), 8.27 (s, 1H), 8.08 (s, 2H), 7.74 (dd, J=8.2, 1.7 Hz, 1H), 7.66 (d, J=1.7 Hz, 1H), 7.30-7.23 (m, 1H), 6.93 (d, J=8.2 Hz, 1H), 6.91-6.88 (m, 1H), 4.88 (s, 4H), 4.23 (s, 4H), 3.90 (s, 3H), 2.13 (s, 3H).

### Example 12: (S)-2-Amino-N-(6-(6-(3-methoxy-2-methylphenyl)-1-oxophthalazin-2(1H)-yl)pyridin-3-yl)-3-methylbutanamide hydrochloride (Final Compound 1-98) 6-(3-Methoxy-2-methylphenyl)-2-(5-nitropyridin-2-yl)phthalazin-1(2H)-one

According to Scheme 1 Step 3: To a stirred solution of 6-bromo-2-(5-nitropyridin-2-yl)phthalazin-1(2H)-one (4.00 g, 11.6 mmol, 1.0 equiv.) in a pressure flask were added 2-(3-methoxy-2-methylphenyl)-4,4,5,5-tetramethyl-1,3,2-dioxaborolane (3.44 g, 13.9 mmol, 1.2 equiv.), K₂CO₃ (2.08 mg, 15.0 mmol, 1.3 equiv.), DMF (200 mL), and water (40 mL). Pd(PPh₃)₄(670 mg, 0.578 mmol, 0.05 equiv.) was added in one portion, and the solution was stirred at 120° C. for 20 h. After TLC indicated full conversion, the reaction mixture was partitioned between sat. aqueous NaHCO₃ solution (200 mL) and DCM (200 mL). The layers were separated, and the aqueous layer was extracted with DCM (3×200 mL). The combined organic layers were dried over MgSO₄, filtered, and concentrated under reduced pressure. Silica gel chromatography (DCM/MeOH 100:0 to 95:5) gave the title compound (769 mg, 1.98 mmol, 17%) as a brown solid. ¹H-NMR (500 MHz, CDCl₃): δ=8.51 (dd, J=8.2, 0.7 Hz, 1H), 8.32 (d, J=0.7 Hz, 1H), 8.11 (dd, J=2.9, 0.6 Hz, 1H), 7.75 (dd, J=8.2, 1.7 Hz, 1H), 7.67 (dd, J=1.7, 0.6 Hz, 1H), 7.51 (dd, J=8.5, 0.6 Hz, 1H), 7.29-7.25 (m, 1H), 7.14 (dd, J=8.5, 2.9 Hz, 1H), 6.95-6.92 (m, 1H), 6.90 (dd, J=7.6, 1.1 Hz, 1H), 3.90 (s, 3H), 2.14 (s, 3H).

### 6-(3-Methoxy-2-methylphenyl)-2-(5-aminopyridin-2-yl)phthalazin-1(2H)-one

According to Scheme 9: To a stirred suspension of 6-(3-methoxy-2-methylphenyl)-2-(5-nitropyridin-2-yl)phthalazin-1(2H)-one (750 mg, 2.08 mmol, 1.0 equiv.) in EtOH (100 mL) was added SnCl₂ dihydrate (2.34 g, 10.38 mmol, 5.0 equiv.). The mixture was stirred at 50° C. overnight. After TLC indicated full conversion, the reaction mixture was brought to pH=12 by adding aqueous NaOH solution, and was stirred for 30 min. DCM (200 mL) was added, and the layers were separated. The aqueous layer was extracted with DCM (3×200 mL), the organic layers were combined, dried over MgSO₄, filtered, and concentrated under reduced pressure. Silica gel chromatography (DCM/MeOH 100:0 to 95:5) gave the title compound (509 mg, 1.55 mmol, 75%) as a pale yellow solid.

¹H-NMR (500 MHz, DMSO-d₆): δ=8.50 (d, J=0.7 Hz, 1H), 8.36-8.28 (m, 1H), 7.94 (d, J=1.7 Hz, 1H), 7.88 (dd, J=2.8, 0.6 Hz, 1H), 7.83 (dd, J=8.2, 1.7 Hz, 1H), 7.31 (t, J=7.9 Hz, 1H), 7.22 (d, J=8.4 Hz, 1H), 7.08 (ddd, J=7.9, 6.0, 2.0 Hz, 2H), 6.93 (dd, J=7.7, 1.1 Hz, 1H), 5.60 (s, 2H), 3.86 (s, 3H), 2.09 (s, 3H).

### tert-Butyl (S)-(1-((6-(6-(3-methoxy-2-methylphenyl)-1-oxophthalazin-2(1H)-yl)pyridin-3-yl)amino)-3-methyl-1-oxobutan-2-yl)carbamate

According to Scheme 9: To a solution of 2-(5-aminopyridin-2-yl)-6-(3-methoxy-2-methylphenyl)phthalazin-1(2H)-one (100 mg, 0.28 mmol, 1.0 equiv.), (tert-butoxycarbonyl)-L-valine (91 mg, 0.42 mmol, 1.5 equiv.), DMAP (17 mg, 0.14 mmol, 0.5 equiv.) and DIPEA (0.15 mL, 0.84 mmol, 3.0 equiv.) in anhydrous DMF (10 mL) was added HATU (127 mg, 0.34 mmol, 1.2 equiv.), and the reaction was stirred at 50° C. overnight. TLC indicated no full conversion, and an additional aliquot of HATU (62 mg, 0.17 mmol, 0.6 equiv.) was added. The reaction mixture was stirred for further 72 h at 50° C. The solvents were removed under reduced pressure, the residue was taken up in EtOAc (100 mL), was washed with sat. aqueous NaHCO₃ solution (100 mL) and brine (100 mL), dried over MgSO₄, filtered, and concentrated under reduced pressure. Silica gel chromatography (DCM/MeOH 95:5) gave the title compound (95 mg, 0.17 mmol, 61%) as a colorless solid.

¹H-NMR (500 MHz, DMSO-d₆): δ=10.45 (s, 1H), 8.79 (dd, J=2.7, 0.6 Hz, 1H), 8.57 (d, J=0.7 Hz, 1H), 8.39-8.31 (m, 1H), 8.26 (dd, J=8.7, 2.7 Hz, 1H), 8.01-7.95 (m, 1H), 7.86 (dd, J=8.2, 1.7 Hz, 1H), 7.63 (d, J=8.7 Hz, 1H), 7.32 (t, J=7.8 Hz, 1H), 7.08 (dd, J=8.3, 1.1 Hz, 1H), 7.01 (d, J=8.4 Hz, 1H), 6.93 (dd, J=7.9, 1.1 Hz, 1H), 3.98 (t, J=7.9 Hz, 1H), 3.87 (s, 3H), 2.09 (s, 3H), 2.05 (q, J=6.9 Hz, 1H), 1.39 (d, J=19.8 Hz, 9H), 0.94 (dd, J=6.7, 2.4 Hz, 6H).

### (S)-2-Amino-N-(6-(6-(3-methoxy-2-methylphenyl)-1-oxophthalazin-2(1H)-yl)pyridin-3-yl)-3-methylbutanamide hydrochloride

To a solution of tert-butyl (S)-(1-((6-(6-(3-methoxy-2-methylphenyl)-1-oxophthalazin-2(1H)-yl)pyridin-3-yl)amino)-3-methyl-1-oxobutan-2-yl)carbamate (90 mg, 0.162 mmol, 1.0 equiv.) in DCM (10 mL) was added HCl in 1,4-dioxane (4M, 0.41 mL) at 0° C. The reaction mixture was allowed to warm to room temperature, and was stirred for 5 h. After TLC indicated full conversion, the reaction mixture was concentrated under reduced pressure to give the title compound (49 mg, 0.10 mmol, 62%) as a pale yellow solid.

LC/MS: m/z=458.22 [M+H]⁺, RT=3.65 min; ¹H-NMR (500 MHz, DMSO-d₆): δ=11.44 (s, 1H), 8.89 (d, J=2.7 Hz, 1H), 8.59 (s, 1H), 8.43 (d, J=5.4 Hz, 3H), 8.35 (d, J=8.1 Hz, 1H), 8.30 (dd, J=8.6, 2.6 Hz, 1H), 7.99 (s, 1H), 7.87 (d, J=8.2 Hz, 1H), 7.69 (d, J=8.6 Hz, 1H), 7.32 (t, J=7.9 Hz, 1H), 7.08 (d, J=8.2 Hz, 1H), 6.93 (d, J=7.6 Hz, 1H), 4.00-3.93 (m, 1H), 3.87 (s, 3H), 2.28 (q, J=6.7 Hz, 1H), 2.09 (s, 3H), 1.04 (t, J=6.5 Hz, 6H).

### Example 13: 6-(3-Methoxy-2-methylphenyl)-2-(5-((2-methoxy-2-methylpropyl)amino)pyrimidin-2-yl)phthalazin-1(2H)-one (Final Compound 1-110)

### 6-(3-Methoxy-2-methylphenyl)-2-(5-((2-methoxy-2-methylpropyl)amino)pyrimidin-2-yl)phthalazin-1(2H)-one

According to Scheme 10: To a stirred suspension of sodium hydride (60% dispersion in minimum oil, 12.0 mg, 0.302 mmol) in dry THE (1 mL) was added dropwise a solution of 2-(5-(2-hydroxy-2-methylpropylamino)pyrimidin-2-yl)-6-(3-methoxy-2-methylphenyl)phthalazin-1(2H)-one (100 mg, 0.231 mmol) (prepared according to Scheme 8) in dry DMF (2 mL) at 0° C. After 10 min, a solution of methyl iodide (43 mg, 0.30 mmol) in dry THE (1 mL) was added, and the mixture was stirred overnight under a nitrogen atmosphere while slowly warming to rt. After 19 h, the reaction mixture was quenched with MeOH (0.3 mL). After stirring for further 30 min at rt, the mixture was partitioned between EtOAc (24 mL) and water (4 mL). The aqueous phase was extracted with EtOAc (1×4 mL), and the combined organic phases were dried over MgSO₄, filtered and concentrated in vacuo. Silica gel chromatography (DCM:MeOH 98:2) was performed to afford the title compound (20 mg, 0.045 mmol, 19%) as an off-white solid.

LC/MS: m/z=446.3 [M+H]⁺, RT=4.08 min; ¹H-NMR (500 MHz, CDCl₃): δ=8.52 (d, J=8.1 Hz, 1H), 8.45 (s, 2H), 8.28 (d, J=0.7 Hz, 1H), 7.74 (dd, J=8.1, 1.7 Hz, 1H), 7.66 (d, J=1.6 Hz, 1H), 7.27 (t, J=7.9 Hz, 1H), 6.93 (dd, J=8.3, 1.1 Hz, 1H), 6.90 (dd, J=7.7, 1.1 Hz, 1H), 3.90 (s, 3H), 3.41 (s, 2H), 3.17 (s, 3H), 2.13 (s, 3H), 1.64 (s, 2H), 1.33 (s, 6H).

The compounds in the following Table have been synthezised according to the same methods as previous Examples 1 to 12, as denoted in the column denoted as “Exp. nr”.

The compounds denoted with the asterisk have been exemplified in the Examples.

TABLE 1
Compounds prepared according to the Examples.

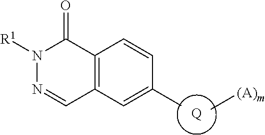

| Co.nr. | Exp nr. | R¹ | 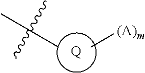 |
|---|---|---|---|
| 1-1 | 1* | 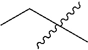 [SMILES: *CC] | 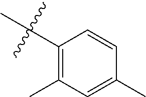 [SMILES: *c1ccc(C)cc1C] |
| 1-2 | 1 | 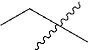 [SMILES: *CC] | 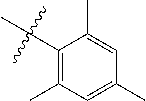 [SMILES: *c1c(C)cc(C)cc1C] |
| 1-3 | 1 | 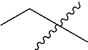 [SMILES: *CC] | 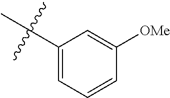 [SMILES: *c1cccc(OC)c1] |
| 1-4 | 5* | 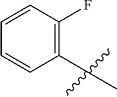 [SMILES: *c1ccccc1F] | 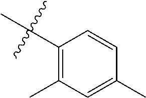 [SMILES: *c1ccc(C)cc1C] |
| 1-5 | 6* | 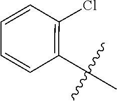 [SMILES: *c1ccccc1Cl] | 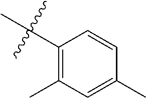 [SMILES: *c1ccc(C)cc1C] |
| 1-6 | 1 | 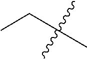 [SMILES: *CC] | 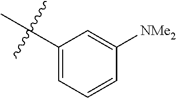 [SMILES: *c1cccc(N(C)C)c1] |
| 1-7 | 1 | 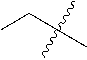 [SMILES: *CC] | 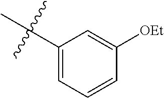 [SMILES: *c1cccc(OCC)c1] |
| 1-8 | 7* | 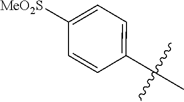 [SMILES: *c1ccc(S(C)(=O)=O)cc1] | 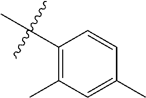 [SMILES: *c1ccc(C)cc1C] |
| 1-9 | 7 | 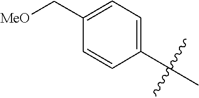 [SMILES: *c1ccc(COC)cc1] | 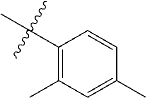 [SMILES: *c1ccc(C)cc1C] |
| 1-10 | 1 | 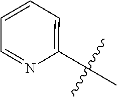 [SMILES: *c1ccccn1] | 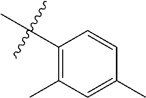 [SMILES: *c1ccc(C)cc1C] |
| 1-11 | 8* | 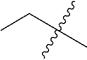 [SMILES: *CC] | 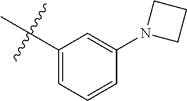 [SMILES: *c1cccc(N2CCC2)c1] |
| 1-12 | 7 | 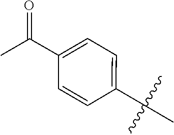 [SMILES: *c1ccc(C(C)=O)cc1] | 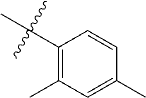 [SMILES: *c1ccc(C)cc1C] |
| 1-13 | 2* | 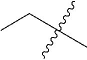 [SMILES: *CC] | 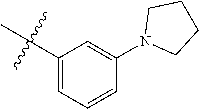 [SMILES: *c1cccc(N2CCCC2)c1] |
| 1-14 | 10* | 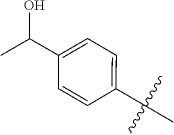 [SMILES: *c1ccc(C(C)O)cc1] | 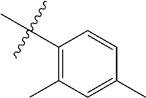 [SMILES: *c1ccc(C)cc1C] |
| 1-15 | 2 | 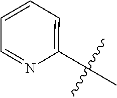 [SMILES: *c1ccccn1] | 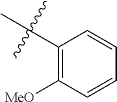 [SMILES: *c1ccccc1OC] |
| 1-16 | 2 | 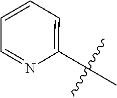 [SMILES: *c1ccccn1] | 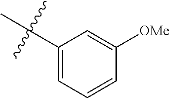 [SMILES: *c1cccc(OC)c1] |
| 1-17 | 2 | 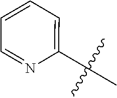 [SMILES: *c1ccccn1] | 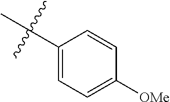 [SMILES: *c1ccc(OC)cc1] |
| 1-18 | 2 | 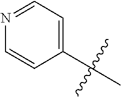 [SMILES: *c1ccncc1] | 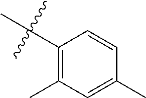 [SMILES: *c1ccc(C)cc1C] |
| 1-19 | 8 | 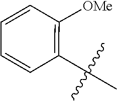 [SMILES: *c1ccccc1OC] | 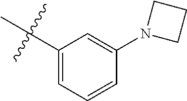 [SMILES: *c1cccc(N2CCC2)c1] |
| 1-20 | 8 | 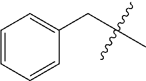 [SMILES: *Cc1ccccc1] | 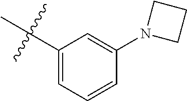 [SMILES: *c1cccc(N2CCC2)c1] |
| 1-21 | 2 | 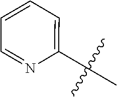 [SMILES: *c1ccccn1] | 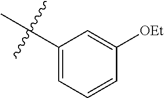 [SMILES: *c1cccc(OCC)c1] |
| 1-22 | 4* | 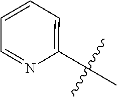 [SMILES: *c1ccccn1] | 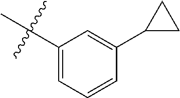 [SMILES: *c1cccc(C2CC2)c1] |
| 1-23 | 4 | 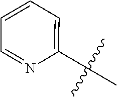 [SMILES: *c1ccccn1] | 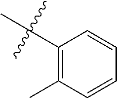 [SMILES: *c1ccccc1C] |
| 1-24 | 9* | 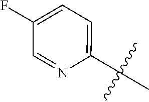 [SMILES: *c1ccc(F)cn1] | 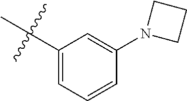 [SMILES: *c1cccc(N2CCC2)c1] |
| 1-25 | 4 | 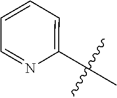 [SMILES: *c1ccccn1] | 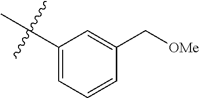 [SMILES: *c1cccc(COC)c1] |
| 1-26 | 4 | 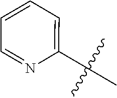 [SMILES: *c1ccccn1] | 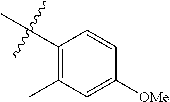 [SMILES: *c1ccc(OC)cc1C] |
| 1-27 | 1 | 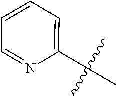 [SMILES: *c1ccccn1] | 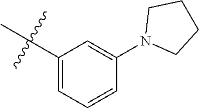 [SMILES: *c1cccc(N2CCCC2)c1] |
| 1-28 | 1 | 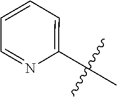 [SMILES: *c1ccccn1] | 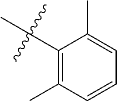 [SMILES: *c1c(C)cccc1C] |
| 1-29 | 1 | 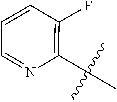 [SMILES: *c1ncccc1F] | 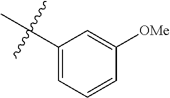 [SMILES: *c1cccc(OC)c1] |
| 1-30 | 4 | 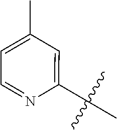 [SMILES: *c1cc(C)ccn1] | 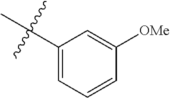 [SMILES: *c1cccc(OC)c1] |
| 1-31 | 4 | 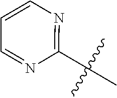 [SMILES: *c1ncccn1] | 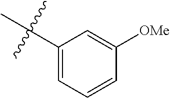 [SMILES: *c1cccc(OC)c1] |
| 1-32 | 4 | 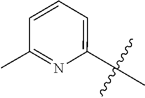 [SMILES: *c1cccc(C)n1] | 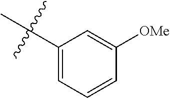 [SMILES: *c1cccc(OC)c1] |
| 1-33 | 1 | 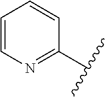 [SMILES: *c1ccccn1] | 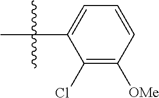 [SMILES: *c1cccc(OC)c1Cl] |
| 1-34 | 1 | 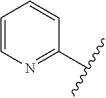 [SMILES: *c1ccccn1] | 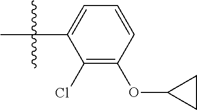 [SMILES: *c1cccc(OC2CC2)c1Cl] |
| 1-35 | 1 | 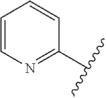 [SMILES: *c1ccccn1] | 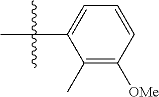 [SMILES: *c1cccc(OC)c1C] |
| 1-36 | 1 | 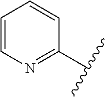 [SMILES: *c1ccccn1] | 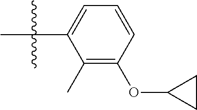 [SMILES: *c1cccc(OC2CC2)c1C] |
| 1-37 | 1 | 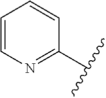 [SMILES: *c1ccccn1] | 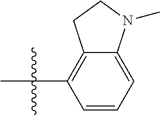 [SMILES: *c1cccc2c1CCN2C] |
| 1-38 | 1 | 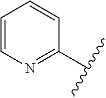 [SMILES: *c1ccccn1] | 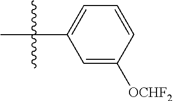 [SMILES: *c1cccc(OC(F)F)c1] |
| 1-39 | 1 | 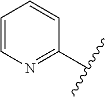 [SMILES: *c1ccccn1] | 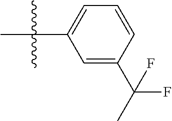 [SMILES: *c1cccc(C(C)(F)F)c1] |
| 1-40 | 1 | 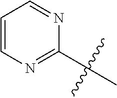 [SMILES: *c1ncccn1] | 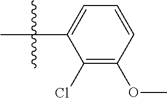 [SMILES: *c1cccc(OC)c1Cl] |
| 1-41 | 1 | 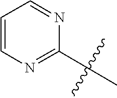 [SMILES: *c1ncccn1] | 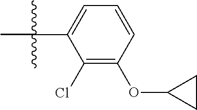 [SMILES: *c1cccc(OC2CC2)c1Cl] |
| 1-42 | 1 | 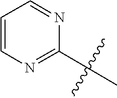 [SMILES: *c1ncccn1] | 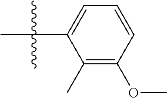 [SMILES: *c1cccc(OC)c1C] |
| 1-43 | 1 | 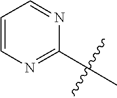 [SMILES: *c1ncccn1] | 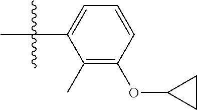 [SMILES: *c1cccc(OC2CC2)c1C] |
| 1-44 | 1 | 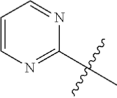 [SMILES: *c1ncccn1] | 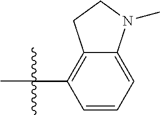 [SMILES: *c1cccc2c1CCN2C] |
| 1-45 | 1 | 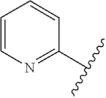 [SMILES: *c1ccccn1] | 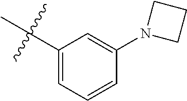 [SMILES: *c1cccc(N2CCC2)c1] |
| 1-46 | 1 | 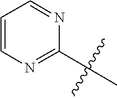 [SMILES: *c1ncccn1] | 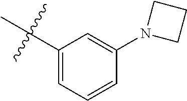 [SMILES: *c1cccc(N2CCC2)c1] |
| 1-47 | 4 | 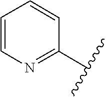 [SMILES: *c1ccccn1] | 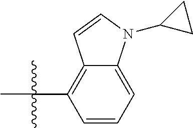 [SMILES: *c1cccc2c1ccn2C1CC1] |
| 1-48 | 1 | 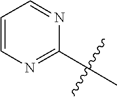 [SMILES: *c1ncccn1] | 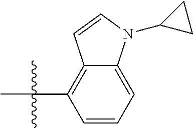 [SMILES: *c1cccc2c1ccn2C1CC1] |
| 1-49 | 1 | 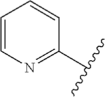 [SMILES: *c1ccccn1] | 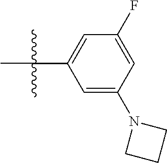 [SMILES: *c1cc(F)cc(N2CCC2)c1] |
| 1-50 | 3 | 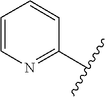 [SMILES: *c1ccccn1] | 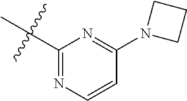 [SMILES: *c1nccc(N2CCC2)n1] |
| 1-51 | 1 | 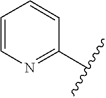 [SMILES: *c1ccccn1] | 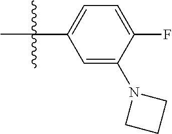 [SMILES: *c1ccc(F)c(N2CCC2)c1] |
| 1-52 | 1 | 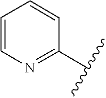 [SMILES: *c1ccccn1] | 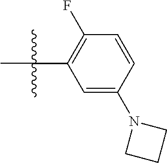 [SMILES: *c1cc(N2CCC2)ccc1F] |
| 1-53 | 3* | 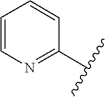 [SMILES: *c1ccccn1] | 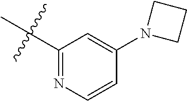 [SMILES: *c1cc(N2CCC2)ccn1] |
| 1-54 | 1 | 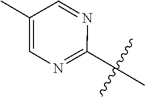 [SMILES: *c1ncc(C)cn1] | 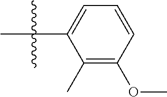 [SMILES: *c1cccc(OC)c1C] |
| 1-55 | 1 | 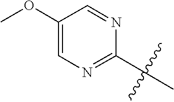 [SMILES: *c1ncc(OC)cn1] | 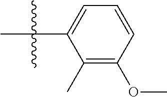 [SMILES: *c1cccc(OC)c1C] |
| 1-56 | 1 | 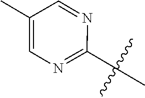 [SMILES: *c1ncc(C)cn1] | 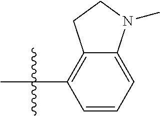 [SMILES: *c1cccc2c1CCN2C] |
| 1-57 | 1 | 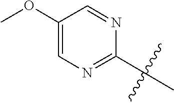 [SMILES: *c1ncc(OC)cn1] | 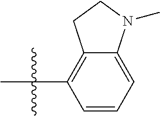 [SMILES: *c1cccc2c1CCN2C] |
| 1-58 | 1 | 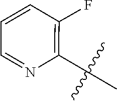 [SMILES: *c1ncccc1F] | 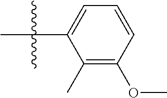 [SMILES: *c1cccc(OC)c1C] |
| 1-59 | 1 | 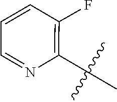 [SMILES: *c1ncccc1F] | 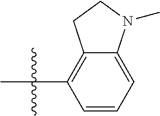 [SMILES: *c1cccc2c1CCN2C] |
| 1-60 | 2 | 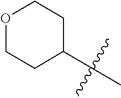 [SMILES: *C1CCOCC1] | 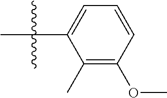 [SMILES: *c1cccc(OC)c1C] |
| 1-61 | 1 | 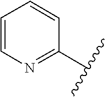 [SMILES: *c1ccccn1] | 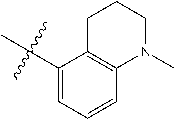 [SMILES: *c1cccc2c1CCCN2C] |
| 1-62 | 1 | 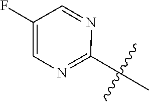 [SMILES: *c1ncc(F)cn1] | 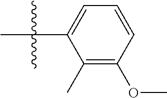 [SMILES: *c1cccc(OC)c1C] |
| 1-63 | 11 | 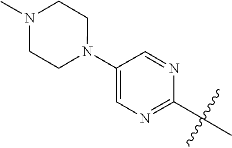 [SMILES: *c1ncc(N2CCN(C)CC2)cn1] | 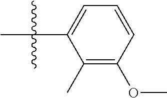 [SMILES: *c1cccc(OC)c1C] |
| 1-64 | 11 | 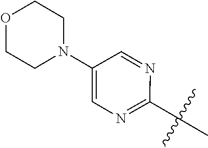 [SMILES: *c1ncc(N2CCOCC2)cn1] | 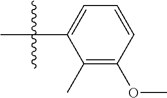 [SMILES: *c1cccc(OC)c1C] |
| 1-65 | 11 | 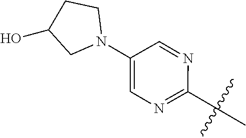 [SMILES: *c1ncc(N2CCC(O)C2)cn1] | 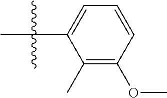 [SMILES: *c1cccc(OC)c1C] |
| 1-66 | 2 | 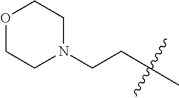 [SMILES: *CCN1CCOCC1] | 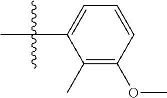 [SMILES: *c1cccc(OC)c1C] |
| 1-67 | 1 | 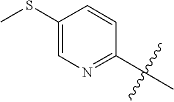 [SMILES: *c1ccc(SC)cn1] | 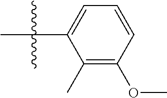 [SMILES: *c1cccc(OC)c1C] |
| 1-68 | 1 | 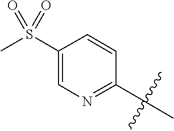 [SMILES: *c1ccc(S(C)(=O)=O)cn1] | 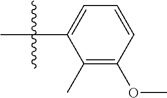 [SMILES: *c1cccc(OC)c1C] |
| 1-69 | 11 | 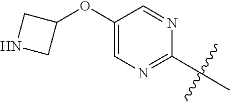 [SMILES: *c1ncc(OC2CNC2)cn1] | 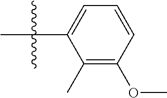 [SMILES: *c1cccc(OC)c1C] |
| 1-70 | 11 | 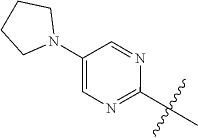 [SMILES: *c1ncc(N2CCCC2)cn1] | 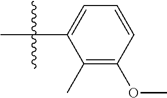 [SMILES: *c1cccc(OC)c1C] |
| 1-71 | 11 | 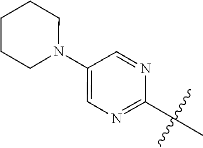 [SMILES: *c1ncc(N2CCCCC2)cn1] | 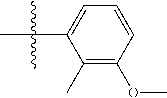 [SMILES: *c1cccc(OC)c1C] |
| 1-72 | 11 | 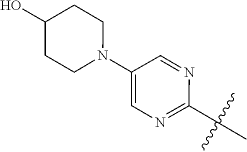 [SMILES: *c1ncc(N2CCC(O)CC2)cn1] | 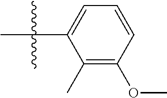 [SMILES: *c1cccc(OC)c1C] |
| 1-73 | 11 | 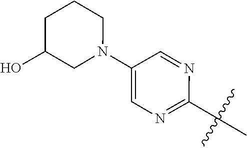 [SMILES: *c1ncc(N2CCCC(O)C2)cn1] | 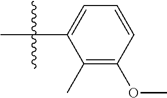 [SMILES: *c1cccc(OC)c1C] |
| 1-74 | 11 | 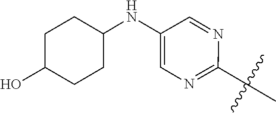 [SMILES: *c1ncc(NC2CCC(O)CC2)cn1] | 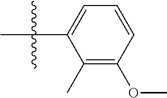 [SMILES: *c1cccc(OC)c1C] |
| 1-75 | 11 | 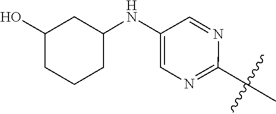 [SMILES: *c1ncc(NC2CCCC(O)C2)cn1] | 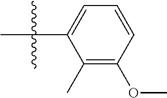 [SMILES: *c1cccc(OC)c1C] |
| 1-76 | 4 | 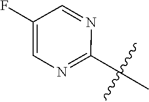 [SMILES: *c1ncc(F)cn1] | 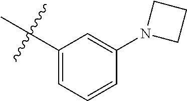 [SMILES: *c1cccc(N2CCC2)c1] |
| 1-77 | 1 | 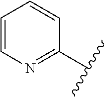 [SMILES: *c1ccccn1] | 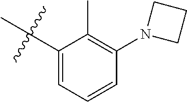 [SMILES: *c1cccc(N2CCC2)c1C] |
| 1-78 | 11 | 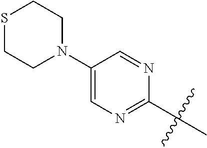 [SMILES: *c1ncc(N2CCSCC2)cn1] | 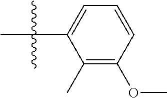 [SMILES: *c1cccc(OC)c1C] |
| 1-79 | 11 | 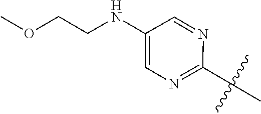 [SMILES: *c1ncc(NCCOC)cn1] | 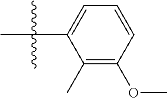 [SMILES: *c1cccc(OC)c1C] |
| 1-80 | 11 | 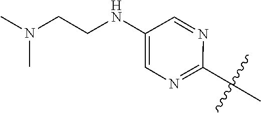 [SMILES: *c1ncc(NCCN(C)C)cn1] | 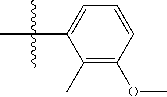 [SMILES: *c1cccc(OC)c1C] |
| 1-81 | 11 | 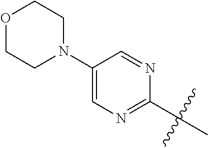 [SMILES: *c1ncc(N2CCOCC2)cn1] | 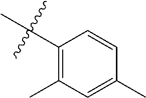 [SMILES: *c1ccc(C)cc1C] |
| 1-82 | 11 | 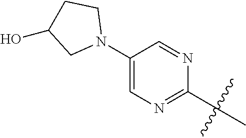 [SMILES: *c1ncc(N2CCC(O)C2)cn1] | 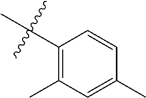 [SMILES: *c1ccc(C)cc1C] |
| 1-83 | 11 | 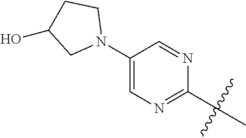 [SMILES: *c1ncc(N2CCC(O)C2)cn1] | 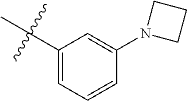 [SMILES: *c1cccc(N2CCC2)c1] |
| 1-84 | 11* | 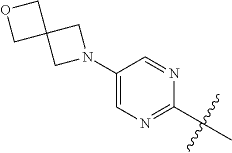 [SMILES: *c1ncc(N2CC3(COC3)C2)cn1] | 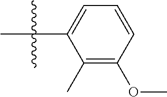 [SMILES: *c1cccc(OC)c1C] |
| 1-85 | 11 | 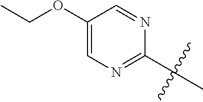 [SMILES: *c1ncc(OCC)cn1] | 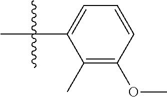 [SMILES: *c1cccc(OC)c1C] |
| 1-86 | 11 | 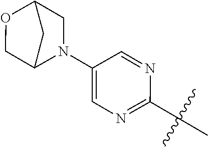 [SMILES: *c1ncc(N2CC3CC2CO3)cn1] | 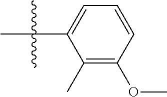 [SMILES: *c1cccc(OC)c1C] |
| 1-87 | 11 | 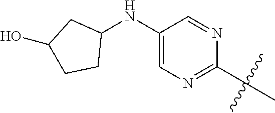 [SMILES: *c1ncc(NC2CCC(O)C2)cn1] | 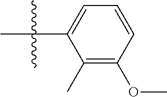 [SMILES: *c1cccc(OC)c1C] |
| 1-88 | 11 | 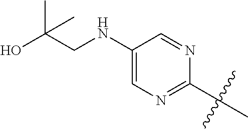 [SMILES: *c1ncc(NCC(C)(C)O)cn1] | 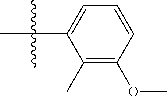 [SMILES: *c1cccc(OC)c1C] |
| 1-89 | 11 | 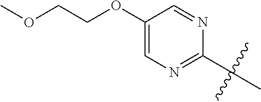 [SMILES: *c1ncc(OCCOC)cn1] | 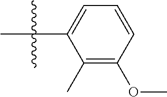 [SMILES: *c1cccc(OC)c1C] |
| 1-90 | 4 | 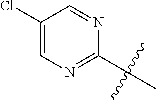 [SMILES: *c1ncc(Cl)cn1] | 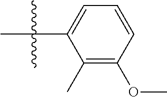 [SMILES: *c1cccc(OC)c1C] |
| 1-91 | 1 | 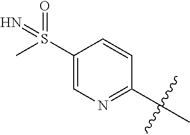 [SMILES: *c1ccc(S(C)(=N)=O)cn1] | 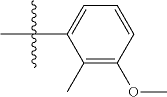 [SMILES: *c1cccc(OC)c1C] |
| 1-92 | 1 | 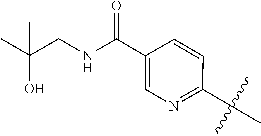 [SMILES: *c1ccc(C(=O)NCC(C)(C)O)cn1] | 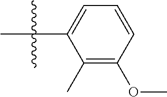 [SMILES: *c1cccc(OC)c1C] |
| 1-93 | 1 | 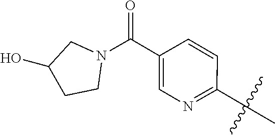 [SMILES: *c1ccc(C(=O)N2CCC(O)C2)cn1] | 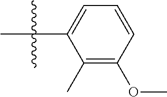 [SMILES: *c1cccc(OC)c1C] |
| 1-94 | 1 | 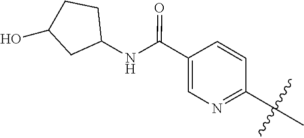 [SMILES: *c1ccc(C(=O)NC2CCC(O)C2)cn1] | 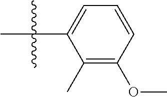 [SMILES: *c1cccc(OC)c1C] |
| 1-95 | 11 | 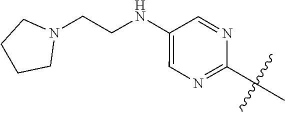 [SMILES: *c1ncc(NCCN2CCCC2)cn1] | 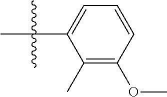 [SMILES: *c1cccc(OC)c1C] |
| 1-96 | 11 | 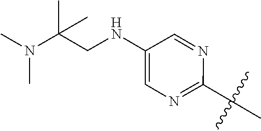 [SMILES: *c1ncc(NCC(C)(C)N(C)C)cn1] | 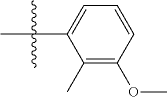 [SMILES: *c1cccc(OC)c1C] |
| 1-97 | 11 | 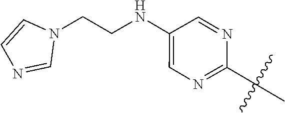 [SMILES: *c1ncc(NCCn2ccnc2)cn1] | 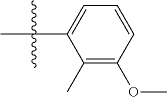 [SMILES: *c1cccc(OC)c1C] |
| 1-98 | 12* | 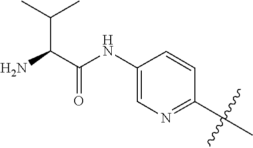 [SMILES: *c1ccc(NC(=O)[C@@H](N)C(C)C)cn1] | 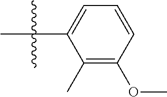 [SMILES: *c1cccc(OC)c1C] |
| 1-99 | 12 | 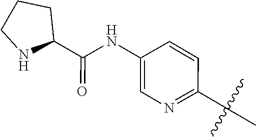 [SMILES: *c1ccc(NC(=O)[C@@H]2CCCN2)cn1] | 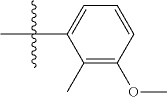 [SMILES: *c1cccc(OC)c1C] |
| 1-100 | 11 | 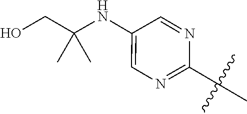 [SMILES: *c1ncc(NC(C)(C)CO)cn1] | 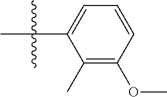 [SMILES: *c1cccc(OC)c1C] |
| 1-101 | 11 | 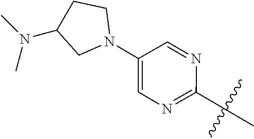 [SMILES: *c1ncc(N2CCC(N(C)C)C2)cn1] | 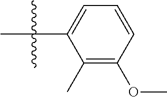 [SMILES: *c1cccc(OC)c1C] |
| 1-102 | 12 | 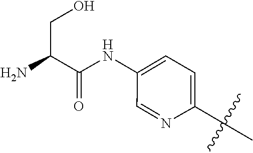 [SMILES: *c1ccc(NC(=O)[C@@H](N)CO)cn1] | 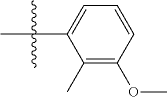 [SMILES: *c1cccc(OC)c1C] |
| 1-103 | 11 | 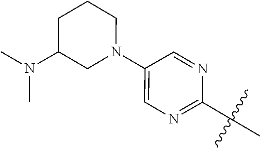 [SMILES: *c1ncc(N2CCCC(N(C)C)C2)cn1] | 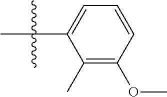 [SMILES: *c1cccc(OC)c1C] |
| 1-104 | 11 | 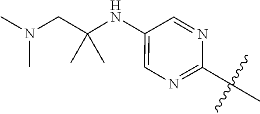 [SMILES: *c1ncc(NC(C)(C)CN(C)C)cn1] | 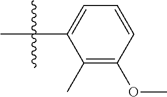 [SMILES: *c1cccc(OC)c1C] |
| 1-105 | 11 | 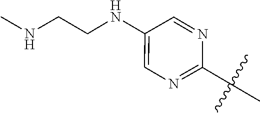 [SMILES: *c1ncc(NCCNC)cn1] | 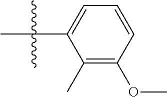 [SMILES: *c1cccc(OC)c1C] |
| 1-106 | 11 | 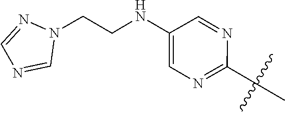 [SMILES: *c1ncc(NCCn2cncn2)cn1] | 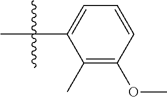 [SMILES: *c1cccc(OC)c1C] |
| 1-107 | 11 | 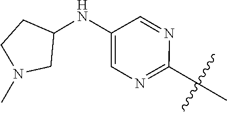 [SMILES: *c1ncc(NC2CCN(C)C2)cn1] | 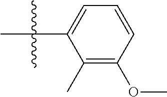 [SMILES: *c1cccc(OC)c1C] |
| 1-108 | 1 | 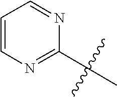 [SMILES: *c1ncccn1] | 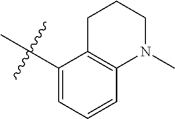 [SMILES: *c1cccc2c1CCCN2C] |
| 1-109 | 13 | 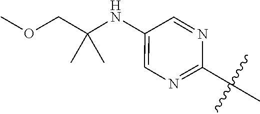 [SMILES: *c1ncc(NC(C)(C)COC)cn1] | 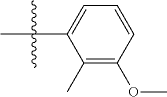 [SMILES: *c1cccc(OC)c1C] |
| 1-110 | 13* | 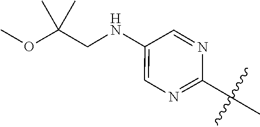 [SMILES: *c1ncc(NCC(C)(C)OC)cn1] | 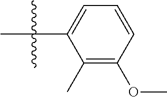 [SMILES: *c1cccc(OC)c1C] |
| 1-111 | 1 | 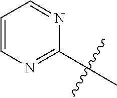 [SMILES: *c1ncccn1] | 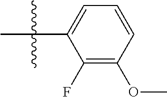 [SMILES: *c1cccc(OC)c1F] |
| 1-112 | 1 | 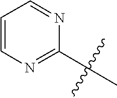 [SMILES: *c1ncccn1] | 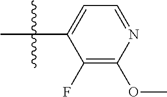 [SMILES: *c1ccnc(OC)c1F] |

### Physico-Chemical Data

### Melting Points:

Melting point determination was performed on a Buchi B-540 apparatus.

### UPLC-MS Method:

UPLC-MS were recorded on Waters ACQUITY UPLC with the following conditions:

### Method 1:

Reverse phase HPLC was carried out on BEH-C₁₈ cartridge (1.7 μm, 2.1×50 mm) from Waters, with a flow rate of 0.8 mL/min. The gradient conditions used are: 90% A (water+0.1% of formic acid), 10% B (ACN+0.1% of formic acid) to 100% B at 1.3 min, kept till 1.7 min and equilibrated to initial conditions at 1.8 min until 2.0 min. Injection volume 5 μL. ES MS detector was used, acquiring both in positive and negative ionization modes.

### Lc-Ms Method:

### Method 2:

Liquid chromatography-mass spectrometry (LC-MS) was performed on a LC-MS system, consisting of a Dionex UltiMate 3000 pump, autosampler, column compartment, and detector (Thermo Fisher Scientific, Dreieich, Germany) and ESI quadrupole MS (MSQ Plus or ISQ EC, Thermo Fisher Scientific, Dreieich, Germany).

Reversed phase (C₁₈), full scan (positive and negative) 100-1000 m/z; eluents: H₂O+0.1 Formic Acid (A) and MeCN+0.1 Formic Acid (B): 0 min 5% B→1 min 5% B→6.8 min 100% B (linear gradient from 5-100% B within 5.8 min)→8 min 100% B (1.2 min 100% B). Purity of the final compounds was determined by LS-MS using the area percentage method on the UV trace recorded at a wavelength of 254 nm.

### Method 3:

LC-MS were recorded on an Agilent Technologies 1260 Infinity LC/MSD system with DADELSD Alltech 3300 and Agilent LCMSD G6120B mass-spectrometer by the following conditions: Reverse phase UHPLC was carried out on a Poroshell 120 SB—C₁₈ cartridge (2.7 μm, 4.6×30 mm) with an UHPLC Guard Infinity Lab Poroshell 120 SB—C₁₈ cartridge (2.7 μm, 4.6×5 mm) from Agilent, with a flow rate at 3 mL/min and a temperature at 60° C. The gradient conditions used are: 1% A (ACN:water (99:1%)+0.1% formic acid), 99% B (water+0.1% formic acid) to 100% A at 1.5 min, kept till 2.2 min and equilibrated to initial conditions at 2.21 min. Injection volume 0.5 μL. ES MS detector was used, acquiring both in positive and negative ionization modes.

### NMR:

¹H-NMR spectra were recorded on a Bruker 300 MHz spectrometer, a Bruker Avance I 500 (500 MHz) spectrometer or Varian Unity Plus 400 MHz spectrometer. Chemical shifts are expressed in parts per million (ppm, 6 units). Splitting patterns describe apparent multiplicities and are designated as s (singlet), d (doublet), t (triplet), q (quadruplet), m (multiplet), br (broad). Coupling constants (J) are given in Hertz (Hz).

TABLE 2
Physico-chemical data.

| | RT | | MS | |
|---|---|---|---|---|
| Co. Nr. | (min) | [MH]⁺ | method | ¹H-NMR |
| 1-1 | 1.20 | 279 | Method 1 | (300 MHz, DMSO-d₆) δ: 8.47 (1H, s), 8.30 (1H, d), 7.90 (1H, |
| | | | | s), 7.81 (1H, d), 7.18 (3H, q), 4.16 (2H, q), 2.34 (3H, s), 2.23 |
| | | | | (3H, s), 1.31 (3H, t) |
| 1-2 | 1.27 | 293 | Method 1 | (300 MHz, DMSO-d₆) δ: 8.44 (1H, s), 8.31 (1H, d), 7.73 (1H, |
| | | | | s), 7.63 (1H, d), 6.99 (2H, s), 4.16 (2H, q), 2.29 (3H, s), 1.93 |
| | | | | (6H, s), 1.31 (3H, t) |
| 1-3 | 1.05 | 281 | Method 1 | (300 MHz, DMSO-d₆) δ: 8.49 (1H, s), 8.32 (2H, d), 8.18 (1H, |
| | | | | d), 7.50-7.37 (3H, m), 7.05 (1H, d), 4.19 (2H, q), 3.86 (3H, s), |
| | | | | 1.31 (3H, t) |
| 1-4 | 1.27 | 345 | Method 1 | (300 MHz, CDCl₃) δ: 8.53 (1H, d), 8.32 (1H, s), 7.83-7.77 |
| | | | | (2H, m), 7.56-7.41 (2H, m), 7.33-7.26 (3H, m), 7.20-7.15 (2H, |
| | | | | m), 2.51 (3H, s), 2.36 (3H, s) |
| 1-5 | 1.29 | 361 | Method 1 | (300 MHz, DMSO-d₆) δ: 8.61 (1H, s), 8.34 (1H, d), 8.02 (1H, |
| | | | | s), 7.90 (1H, d), 7.72-7.53 (4H, m), 7.25-7.15 (3H, m), 2.36 |
| | | | | (3H, s), 2.27 (3H, s) |
| 1-6 | 1.00 | 294 | Method 1 | (300 MHz, DMSO-d₆) δ: 8.50 (1H, s), 8.31-8.15 (2H, m), 7.35 |
| | | | | (1H, t), 7.07 (2H, d), 6.83 (1H, d), 4.16 (2H, q), 2.99 (6H, s), |
| | | | | 1.31 (3H, t) |
| 1-7 | 1.13 | 295 | Method 1 | (300 MHz, DMSO-d₆) δ: 8.49 (1H, s), 8.32-8.17 (3H, m), 7.46- |
| | | | | 7.36 (3H, m), 7.02 (1H, d), 4.22-4.10 (4H, m), 1.39-1.29 (6H, |
| | | | | m) |
| 1-8 | 1.17 | 405 | Method 1 | (300 MHz, DMSO-d₆) δ: 8.68 (1H, s), 8.38 (1H, d), 8.10-7.90 |
| | | | | (6H, m), 7.25-7.15 (3H, m), 3.31 (3H, s), 2.36 (3H, s), 2.27 |
| | | | | (3H, s) |
| 1-9 | 1.27 | 370 | Method 1 | (300 MHz, CDCl₃) δ: 8.60 (1H, s), 8.36 (1H, d), 8.02-7.99 |
| | | | | (1H, m), 7.91-7.69 (1H, m), 7.66-7.60 (2H, m), 7.48-7.43 (2H, |
| | | | | m), 7.25-7.15 (3H, m), 4.50 (2H, s), 3.34 (3H, s), 2.35 (3H, s), |
| | | | | 2.27 (3H, s) |
| 1-10 | 1.10 | 328 | Method 1 | (300 MHz, DMSO-d₆) δ: 8.64 (1H, d), 8.59 (1H, s), 8.35 (1H, |
| | | | | d), 8.08-7.88 (3H, m), 7.67 (1H, d), 7.56-7.52 (1H, m), 7.26- |
| | | | | 7.16 (3H, m), 2.36 (3H, s), 2.27 (3H, s) |
| 1-11 | 1.11 | 306 | Method 1 | (300 MHz, DMSO-d₆) δ: 8.50 (1H, s), 8.31-8.12 (3H, m), 7.40 |
| | | | | (2H, q), 7.15 (1H, d), 6.82 (1H, s), 6.45 (1H, d), 4.19 (2H, d), |
| | | | | 3.89 (4H, t), 1.36 (3H, t), 1.08 (1H, t) |
| 1-12 | 1.25 | 369 | Method 1 | (300 MHz, DMSO-d₆) δ: 8.68-8.66 (1H, m), 8.41-8.37 (1H, |
| | | | | m), 8.12-7.86 (6H, m), 7.25-7.21 (3H, m), 2.65-2.64 (3H, m), |
| | | | | 2.35-2.27 (6H, m) |
| 1-13 | 1.17 | 329 | Method 1 | (300 MHz, DMSO-d₆) δ: 8.49 (1H, s), 8.31-8.14 (4H, m), 7.35 |
| | | | | (1H, t), 7.02 (1H, d), 6.89 (1H, s), 6.63 (1H, d), 4.19 (3H, q), |
| | | | | 1.99 (5H, s), 1.31 (4H, t) |
| 1-14 | 1.16 | 371 | Method 1 | (300 MHz, CDCl₃) δ: 8.54 (1H, d), 8.30 (1H, s), 7.75 (1H, d), |
| | | | | 7.66 (2H, d), 7.52 (2H, d), 7.19-7.12 (3H, m), 4.98 (1H, brs), |
| | | | | 2.40 (3H, s), 2.28 (3H, s), 1.88-1.87 (1H, m), 1.55 (3H, d) |
| 1-15 | 0.94 | 331 | Method 1 | (300 MHz, CDCl₃) δ: 8.72 (1H, d), 8.53 (1H, d), 8.40 (1H, d), |
| | | | | 8.01 (1H, d), 7.96-7.78 (3H, m), 7.49-7.32 (3H, m), 7.17-7.01 |
| | | | | (2H, m), 3.87 (3H, s) |
| 1-16 | 0.94 | 331 | Method 1 | (300 MHz, CDCl₃) δ: 8.72 (1H, d), 8.58 (1H, d), 8.43 (1H, d), |
| | | | | 8.06 (1H, d), 7.99-7.79 (3H, m), 7.52-7.21 (4H, m), 7.03 (1H, |
| | | | | d), 3.87 (3H, s) |
| 1-17 | 0.93 | 330 | Method 1 | (300 MHz, CDCl₃) δ: 8.72 (1H, d), 8.55 (1H, d), 8.42 (1H, d), |
| | | | | 8.03 (1H, d), 7.97-7.79 (3H, m), 7.68 (2H, d), 7.39 (1H, m), |
| | | | | 7.07 (2H, d), 3.91 (3H, s) |
| 1-18 | 1.10 | 328 | Method 1 | (300 MHz, CDCl₃) δ: 8.79-8.71 (2H, m), 8.54 (1H, d), 8.35 |
| | | | | (1H, s), 7.93-7.86 (2H, m), 7.84-7.76 (1H, m), 7.69 (1H, s), |
| | | | | 7.22-7.10 (3H, m), 2.40 (3H, s), 2.28 (3H, s) |
| 1-19 | 1.15 | 384 | Method 1 | (300 MHz, DMSO-d₆) δ: 8.54 (1H, s), 8.33-8.28 (2H, m), 8.17 |
| | | | | (1H, d), 7.50-7.07 (6H, m), 6.81 (1H, s), 6.53 (1H, d), 3.93- |
| | | | | 3.88 (4H, m), 3.74 (3H, s), 2.37-2.33 (2H, m) |
| 1-20 | 1.27 | 368 | Method 1 | (300 MHz, DMSO-d₆) δ: 8.52-8.14 (4H, m), 7.33-7.28 (6H, |
| | | | | m), 7.09 (1H, d), 6.78 (1H, s), 6.51 (1H, d), 5.35 (2H, s), 3.91- |
| | | | | 3.86 (4H, m), 2.37-2.31 (2H, m) |
| 1-21 | 1.04 | 345 | Method 1 | (300 MHz, CDCl₃) δ: 8.76-8.67 (1H, m), 8.56 (1H, d), 8.43 |
| | | | | (1H, s), 8.08-8.01 (1H, m), 7.97-7.86 (2H, m), 7.85-7.78 (1H, |
| | | | | m), 7.50-7.34 (2H, m), 7.30 (1H, s), 7.26-7.20 (1H, m), 7.05- |
| | | | | 6.96 (1H, m), 4.15 (2H, q), 1.49 (3H, t) |
| 1-22 | 1.09 | 341 | Method 1 | (300 MHz, CDCl₃) δ: 8.76-8.69 (1H, m), 8.58 (1H, d), 8.42 |
| | | | | (1H, s), 8.09-8.01 (1H, m), 7.98-7.79 (3H, m), 7.53-7.35 (4H, |
| | | | | m), 7.20-7.13 (1H, m), 2.09-1.97 (1H, m), 1.10-1.02 (2H, m), |
| | | | | 0.85-0.77 (2H, m) |
| 1-23 | 1.00 | 315 | Method 1 | (300 MHz, CDCl₃) δ: 8.72 (1H, s), 8.57 (1H, d), 8.40 (1H, s), |
| | | | | 7.97-7.88 (1H, m), 7.87-7.77 (2H, m), 7.73 (1H, s), 7.43-7.29 |
| | | | | (5H, m), 2.32 (3H, s) |
| 1-24 | 1.05 | 373 | Method 1 | (300 MHz, CDCl₃) δ: 8.59-8.50 (2H, m), 8.42 (1H, s), 8.09- |
| | | | | 8.02 (1H, m), 7.95 (1H, s), 7.87-7.80 (1H, m), 7.67-7.58 (1H, |
| | | | | m), 7.41-7.33 (1H, m), 7.08-7.02 (1H, m), 6.71 (1H, s), 6.59- |
| | | | | 6.53 (1H, m), 3.99 (4H, t), 2.45 (2H, q) |
| 1-25 | 0.85 | 345 | Method 1 | (300 MHz, CDCl₃) δ: 8.76-8.70 (1H, m), 8.59 (1H, d), 8.44 |
| | | | | (1H, s), 8.08 (1H, dd), 8.01-7.97 (1H, m), 7.97-7.88 (1H, m), |
| | | | | 7.87-7.81 (1H, m), 7.72 (1H, s), 7.68-7.62 (1H, m), 7.54 (1H, |
| | | | | t), 7.48-7.42 (1H, m), 7.42-7.36 (1H, m), 4.59 (2H, s), 3.49 |
| | | | | (3H, s) |
| 1-26 | 0.96 | 345 | Method 1 | (300 MHz, CDCl₃) δ: 8.76-8.69 (1H, m), 8.54 (1H, d), 8.39 |
| | | | | (1H, s), 7.96-7.87 (1H, m), 7.87-7.76 (2H, m), 7.72-7.67 (1H, |
| | | | | m), 7.43-7.35 (2H, m), 6.92-6.84 (2H, m), 3.89 (3H, s), 2.33 |
| | | | | (3H, s) |
| 1-27 | 1.16 | 370 | Method 1 | (300 MHz, CDCl₃) δ: 8.75-8.70 (1H, m), 8.56 (1H, d), 8.43 |
| | | | | (1H, s), 8.11-8.04 (1H, m), 7.99-7.79 (3H, m), 7.43-7.34 (3H, |
| | | | | m), 6.99 (1H, d), 6.83 (1H, s), 6.72-6.65 (1H, m), 3.45-3.36 |
| | | | | (4H, m), 2.12-2.03 (4H, m) |
| 1-28 | 1.08 | 329 | Method 1 | (300 MHz, CDCl₃) δ: 8.75-8.69 (1H, m), 8.60 (1H, d), 8.38 |
| | | | | (1H, s), 7.97-7.88 (1H, m), 7.86-7.80 (1H, m), 7.66 (1H, d), |
| | | | | 7.59 (1H, s), 7.43-7.36 (1H, m), 7.27-7.22 (1H, m), 7.22-7.14 |
| | | | | (2H, m), 2.06 (6H, s) |
| 1-29 | 1.02 | 348 | Method 1 | (300 MHz, CDCl₃) δ: 8.57 (1H, d), 8.52 (1H, d), 8.42 (1H, s), |
| | | | | 8.07 (1H, d), 8.00-7.93 (1H, m), 7.72-7.63 (1H, m), 7.55-7.39 |
| | | | | (2H, m), 7.35-7.13 (2H, m), 7.07-6.97 (1H, m), 3.93 (3H, s) |
| 1-30 | 1.00 | 344 | Method 1 | (300 MHz, CDCl₃) δ: 8.57-8.54 (2H, m), 8.03 (1H, d), 7.94 |
| | | | | (1H, s), 7.61 (1H, s), 7.47-7.42 (1H, m), 7.30-7.20 (2H, m), |
| | | | | 7.02-7.00 (1H, m), 3.91 (3H, s), 2.47 (3H, s) |
| 1-31 | 0.86 | 331 | Method 1 | (300 MHz, CDCl₃) δ: 8.97 (2H, d), 8.57 (1H, d), 8.38 (1H, s), |
| | | | | 8.04 (1H, d), 7.94 (1H, s), 7.47-7.41 (2H, m), 7.30-7.21 (2H, |
| | | | | m), 7.03-7.00 (1H, m), 3.91 (3H, s) |
| 1-32 | 0.99 | 344 | Method 1 | (300 MHz, CDCl₃) δ: 8.54 (1H, d), 8.40 (1H, s), 8.02 (1H, d), |
| | | | | 7.93 (1H, s), 7.81-7.76 (1H, m), 7.30-7.21 (3H, m), 7.02-6.99 |
| | | | | (1H, m), 3.90 (3H, s) |
| 1-33 | 4.11 | 364.1 | Method 2 | (500 MHz, CDCl₃): δ = 8.70 (ddd, J = 4.8, 1.9, 0.9 Hz, 1H), |
| | | | | 8.55 (dt, J = 8.2, 0.7 Hz, 1H), 8.38 (d, J = 0.8 Hz, 1H), 7.93- |
| | | | | 7.85 (m, 2H), 7.83-7.77 (m, 2H), 7.39-7.31 (m, 2H), 7.01 |
| | | | | (ddd, J = 14.1, 8.0, 1.4 Hz, 2H), 3.97 (s, 3H) |
| 1-34 | 4.53 | 390.1 | Method 2 | (500 MHz, CDCl₃): δ = 8.70 (ddd, J = 4.9, 1.9, 0.9 Hz, 1H), |
| | | | | 8.54 (dt, J = 8.2, 0.7 Hz, 1H), 8.38 (d, J = 0.7 Hz, 1H), 7.93- |
| | | | | 7.85 (m, 2H), 7.84-7.76 (m, 2H), 7.44-7.30 (m, 3H), 7.01 |
| | | | | (dd, J = 7.5, 1.6 Hz, 1H), 3.93-3.82 (m, 1H), 0.89 (dddd, J = |
| | | | | 10.7, 5.0, 2.6, 1.3 Hz, 4H) |
| 1-35 | 4.30 | 344.1 | Method 2 | (500 MHz, CDCl₃): δ = 8.70 (ddd, J = 4.9, 1.9, 0.9 Hz, 1H), |
| | | | | 8.53 (dd, J = 8.2, 0.7 Hz, 1H), 8.36 (d, J = 0.7 Hz, 1H), 7.89 |
| | | | | (ddd, J = 8.2, 7.4, 1.9 Hz, 1H), 7.81 (dt, J = 8.1, 1.0 Hz, 1H), |
| | | | | 7.76 (dd, J = 8.2, 1.6 Hz, 1H), 7.68 (t, J = 0.8 Hz, 1H), 7.36 |
| | | | | (ddd, J = 7.4, 4.9, 1.1 Hz, 1H), 7.30-7.24 (m, 1H), 6.91 (ddd, |
| | | | | J = 19.2, 8.0, 1.1 Hz, 2H), 3.90 (s, 3H), 2.14 (s, 3H) |
| 1-36 | 4.71 | 370.1 | Method 2 | (500 MHz, CDCl₃): δ = 8.71 (ddd, J = 4.8, 2.0, 0.9 Hz, 1H), |
| | | | | 8.53 (dt, J = 8.0, 0.7 Hz, 1H), 8.37 (d, J = 0.7 Hz, 1H), 7.90 |
| | | | | (ddd, J = 8.1, 7.4, 1.9 Hz, 1H), 7.82 (dt, J = 8.1, 1.0 Hz, 1H), |
| | | | | 7.76 (dd, J = 8.2, 1.7 Hz, 1H), 7.70-7.67 (m, 1H), 7.37 (ddd, |
| | | | | J = 7.4, 4.9, 1.1 Hz, 1H), 7.34-7.25 (m, 2H), 6.91 (dd, J = |
| | | | | 7.4, 1.4 Hz, 1H), 3.86-3.76 (m, 1H), 2.08 (s, 3H), 0.90-0.77 |
| | | | | (m, 4H) |
| 1-37 | 3.84 | 355.1 | Method 2 | (500 MHz, CDCl₃): δ = 8.71-8.63 (m, 1H), 8.51 (d, J = 8.2 |
| | | | | Hz, 1H), 8.36 (s, 1H), 7.87 (ddd, J = 8.9, 7.1, 1.8 Hz, 2H), |
| | | | | 7.81-7.74 (m, 2H), 7.34 (ddd, J = 7.4, 4.9, 1.1 Hz, 1H), 7.28- |
| | | | | 7.20 (m, 1H), 6.82 (d, J = 7.7 Hz, 1H), 6.58 (d, J = 7.8 Hz, |
| | | | | 1H), 3.34 (t, J = 8.0 Hz, 2H), 3.04 (t, J = 8.0 Hz, 2H), 2.81 (s, |
| | | | | 3H) |
| 1-38 | 4.23 | 366.1 | Method 2 | (500 MHz, CDCl₃): δ = 8.69 (ddd, J = 4.9, 1.9, 0.8 Hz, 1H), |
| | | | | 8.56 (dt, J = 8.2, 0.7 Hz, 1H), 8.42 (d, J = 0.7 Hz, 1H), 8.00 |
| | | | | (dd, J = 8.3, 1.8 Hz, 1H), 7.93-7.87 (m, 2H), 7.80 (dt, J = |
| | | | | 8.0, 1.0 Hz, 1H), 7.57-7.49 (m, 2H), 7.45 (ddd, J = 2.4, 1.8, |
| | | | | 0.6 Hz, 1H), 7.37 (ddd, J = 7.4, 4.9, 1.1 Hz, 1H), 7.22 (dddd, |
| | | | | J = 7.0, 2.4, 1.6, 0.8 Hz, 1H), 6.61 (t, J_{C, F} = 73.5 Hz, 1H). |
| 1-39 | 4.35 | 364.1 | Method 2 | (500 MHz, CDCl₃): δ = 8.72 (ddd, J = 4.9, 1.9, 0.9 Hz, 1H), |
| | | | | 8.59 (dt, J = 8.3, 0.7 Hz, 1H), 8.44 (d, J = 0.7 Hz, 1H), 8.05 |
| | | | | (dd, J = 8.3, 1.8 Hz, 1H), 7.96 (dd, J = 1.8, 0.6 Hz, 1H), 7.92 |
| | | | | (ddd, J = 8.1, 7.4, 1.9 Hz, 1H), 7.86-7.82 (m, 2H), 7.78- |
| | | | | 7.74 (m, 1H), 7.62-7.59 (m, 2H), 7.39 (ddd, J = 7.4, 4.9, 1.1 |
| | | | | Hz, 1H), 2.00 (t, J_{C, F} = 18.2 Hz, 3H). |
| 1-40 | 3.82 | 365.1 | Method 2 | (500 MHz, CDCl₃): δ = 8.96 (d, J = 4.8 Hz, 2H), 8.57 (dd, J = |
| | | | | 8.2, 0.7 Hz, 1H), 8.35 (d, J = 0.7 Hz, 1H), 7.88 (dd, J = 8.2, |
| | | | | 1.6 Hz, 1H), 7.82 (dd, J = 1.7, 0.6 Hz, 1H), 7.41 (t, J = 4.9 Hz, |
| | | | | 1H), 7.35 (dd, J = 8.3, 7.6 Hz, 1H), 7.02 (ddd, J = 14.3, 8.0, |
| | | | | 1.4 Hz, 2H), 3.98 (s, 3H). |
| 1-41 | 4.27 | 391.0 | Method 2 | (500 MHz, CDCl₃): δ = 8.96 (d, J = 4.9 Hz, 2H), 8.56 (d, J = |
| | | | | 8.2 Hz, 1H), 8.34 (d, J = 0.7 Hz, 1H), 7.88 (dd, J = 8.2, 1.7 |
| | | | | Hz, 1H), 7.81 (dd, J = 1.7, 0.6 Hz, 1H), 7.43 7.39 (m, 2H), |
| | | | | 7.35 (dd, J = 8.3, 7.5 Hz, 1H), 7.01 (dd, J = 7.5, 1.6 Hz, 1H), |
| | | | | 3.88 (tt, J = 5.9, 2.9 Hz, 1H), 0.89 (dddt, J = 11.6, 4.8, 2.4, 1.3 |
| | | | | Hz, 4H). |
| 1-42 | 4.03 | 345.1 | Method 2 | (500 MHz, CDCl₃): δ = 8.95 (d, J = 4.9 Hz, 2H), 8.56-8.52 |
| | | | | (m, 1H), 8.32 (d, J = 0.7 Hz, 1H), 7.76 (dd, J = 8.1, 1.6 Hz, |
| | | | | 1H), 7.68 (dd, J = 1.7, 0.6 Hz, 1H), 7.41 (t, J = 4.9 Hz, 1H), |
| | | | | 7.30-7.23 (m, 1H), 6.91 (ddd, J = 18.5, 8.0, 1.1 Hz, 2H), |
| | | | | 3.90 (s, 3H), 2.14 (s, 3H). |
| 1-43 | 4.46 | 371.1 | Method 2 | (500 MHz, CDCl₃): δ = 8.96 (d, J = 4.9 Hz, 2H), 8.54 (dt, J = |
| | | | | 8.2, 0.7 Hz, 1H), 8.32 (d, J = 0.7 Hz, 1H), 7.76 (dd, J = 8.2, |
| | | | | 1.7 Hz, 1H), 7.68 (dd, J = 1.7, 0.6 Hz, 1H), 7.41 (t, J = 4.9 Hz, |
| | | | | 1H), 7.33-7.27 (m, 2H), 6.91 (dd, J = 7.4, 1.5 Hz, 1H), 3.85- |
| | | | | 3.76 (m, 1H), 2.08 (s, 3H), 0.83 (dddd, J = 6.8, 4.4, 2.4, 1.1 |
| | | | | Hz, 4H). |
| 1-44 | 3.51 | 356.1 | Method 2 | (500 MHz, CDCl₃): δ = 8.95 (d, J = 4.9 Hz, 2H), 8.54 (dt, J = |
| | | | | 8.2, 0.7 Hz, 1H), 8.33 (d, J = 0.8 Hz, 1H), 7.89 (dd, J = 8.2, |
| | | | | 1.7 Hz, 1H), 7.79 (dd, J = 1.7, 0.6 Hz, 1H), 7.41 (t, J = 4.9 Hz, |
| | | | | 1H), 7.27-7.20 (m, 1H), 6.82 (dd, J = 7.7, 0.9 Hz, 1H), 6.58 |
| | | | | (d, J = 7.8 Hz, 1H), 3.34 (t, J = 8.0 Hz, 2H), 3.05 (t, J = 8.1 |
| | | | | Hz, 2H), 2.81 (s, 3H) |
| 1-45 | 4.00 | 355.1 | Method 2 | (500 MHz, CDCl₃): δ = 8.69 (ddd, J = 4.8, 1.9, 0.8 Hz, 1H), |
| | | | | 8.55-8.48 (m, 1H), 8.40 (d, J = 0.7 Hz, 1H), 8.02 (dd, J = |
| | | | | 8.3, 1.7 Hz, 1H), 7.91 (d, J = 1.7 Hz, 1H), 7.88 (ddd, J = 8.1, |
| | | | | 7.4, 1.9 Hz, 1H), 7.80 (dt, J = 8.1, 1.0 Hz, 1H), 7.37-7.32 (m, |
| | | | | 2H), 7.03 (ddd, J = 7.7, 1.8, 1.0 Hz, 1H), 6.69 (t, J = 2.0 Hz, |
| | | | | 1H), 6.53 (ddd, J = 8.1, 2.4, 0.9 Hz, 1H), 3.96 (t, J = 7.2 Hz, |
| | | | | 4H), 2.41 (tt, J = 7.7, 6.8 Hz, 2H) |
| 1-46 | 3.69 | 356.1 | Method 2 | (500 MHz, CDCl₃): δ = 8.95 (d, J = 4.8 Hz, 2H), 8.54 (dt, J = |
| | | | | 8.3, 0.7 Hz, 1H), 8.37 (d, J = 0.7 Hz, 1H), 8.02 (dd, J = 8.2, |
| | | | | 1.8 Hz, 1H), 7.91 (dd, J = 1.8, 0.6 Hz, 1H), 7.41 (t, J = 4.8 Hz, |
| | | | | 1H), 7.34 (t, J = 7.8 Hz, 1H), 7.03 (ddd, J = 7.6, 1.7, 0.9 Hz, |
| | | | | 1H), 6.69 (t, J = 2.0 Hz, 1H), 6.53 (ddd, J = 8.0, 2.4, 0.9 Hz, |
| | | | | 1H), 3.96 (t, J = 7.2 Hz, 4H), 2.41 (tt, J = 7.7, 6.8 Hz, 2H). |
| 1-47 | 4.62 | 379.1 | Method 2 | (500 MHz, CDCl₃): δ = 8.74-8.68 (m, 1H), 8.62-8.55 (m, |
| | | | | 1H), 8.42 (s, 1H), 8.14 (dd, J = 8.2, 1.7 Hz, 1H), 8.03 (d, J = |
| | | | | 1.6 Hz, 1H), 7.90 (ddd, J = 8.1, 7.3, 1.9 Hz, 1H), 7.83 (d, J = |
| | | | | 8.1 Hz, 1H), 7.68 (dt, J = 8.1, 1.0 Hz, 1H), 7.41-7.34 (m, |
| | | | | 2H), 7.30 (dd, J = 7.3, 0.9 Hz, 1H), 7.26 (d, J = 3.4 Hz, 1H), |
| | | | | 6.60 (dd, J = 3.3, 0.9 Hz, 1H), 3.46-3.39 (m, 1H), 1.16- |
| | | | | 1.11 (m, 2H), 1.09-1.04 (m, 2H). |
| 1-48 | 4.23 | 380.1 | Method 2 | (500 MHz, CDCl₃): δ = 8.96 (d, J = 4.8 Hz, 2H), 8.62-8.55 |
| | | | | (m, 1H), 8.37 (s, 1H), 8.14 (dd, J = 8.2, 1.7 Hz, 1H), 8.02 (d, J = |
| | | | | 1.5 Hz, 1H), 7.68 (dt, J = 8.1, 1.0 Hz, 1H), 7.41 (t, J = 4.9 |
| | | | | Hz, 1H), 7.37 (dd, J = 8.2, 7.3 Hz, 1H), 7.30 (dd, J = 7.3, 1.0 |
| | | | | Hz, 1H), 7.25 (d, J = 3.3 Hz, 1H), 6.60 (dd, J = 3.3, 0.9 Hz, |
| | | | | 1H), 3.47-3.38 (m, 1H), 1.15-1.10 (m, 2H), 1.09-1.04 (m, |
| | | | | 2H) |
| 1-49 | 4.51 | 373.1 | Method 2 | (500 MHz, CDCl₃): δ = 8.69 (ddd, J = 4.8, 2.0, 0.9 Hz, 1H), |
| | | | | 8.53 (dt, J = 8.2, 0.7 Hz, 1H), 8.40 (d, J = 0.7 Hz, 1H), 7.98 |
| | | | | (dd, J = 8.3, 1.8 Hz, 1H), 7.92-7.85 (m, 2H), 7.79 (dt, J = |
| | | | | 8.1, 1.0 Hz, 1H), 7.36 (ddd, J = 7.4, 4.9, 1.1 Hz, 1H), 6.71 |
| | | | | (ddd, J = 9.6, 2.3, 1.5 Hz, 1H), 6.43 (t, J = 1.8 Hz, 1H), 6.19 |
| | | | | (dt, J = 10.7, 2.2 Hz, 1H), 3.96 (t, J = 7.3 Hz, 4H), 2.43 (tt, J = |
| | | | | 7.8, 6.9 Hz, 2H). |
| 1-50 | 2.50 | 357.1 | Method 2 | (500 MHz, CDCl₃): δ = 8.88-8.78 (m, 2H), 8.72-8.66 (m, |
| | | | | 1H), 8.55 (dd, J = 8.3, 0.7 Hz, 1H), 8.45 (s, 1H), 8.32 (d, J = |
| | | | | 5.9 Hz, 1H), 7.89 (ddd, J = 8.1, 7.4, 1.9 Hz, 1H), 7.80 (dt, J = |
| | | | | 8.2, 1.0 Hz, 1H), 7.36 (ddd, J = 7.4, 4.9, 1.1 Hz, 1H), 6.17 (d, |
| | | | | J = 5.9 Hz, 1H), 4.23 (s, 4H), 2.66 2.40 (m, 2H) |
| 1-51 | 4.43 | 373.1 | Method 2 | (500 MHz, CDCl₃): δ = 8.66 (ddd, J = 4.9, 1.9, 0.9 Hz, 1H), |
| | | | | 8.53-8.46 (m, 1H), 8.37 (d, J = 0.7 Hz, 1H), 7.94 (dd, J = |
| | | | | 8.3, 1.8 Hz, 1H), 7.88-7.82 (m, 2H), 7.77 (dt, J = 8.1, 1.0 Hz, |
| | | | | 1H), 7.33 (ddd, J = 7.4, 4.9, 1.1 Hz, 1H), 7.03 (dd, J = 12.0, |
| | | | | 8.3 Hz, 1H), 6.95 (ddd, J = 8.3, 4.2, 2.3 Hz, 1H), 6.67 (dd, J = |
| | | | | 8.5, 2.3 Hz, 1H), 4.04 (td, J = 7.3, 2.2 Hz, 4H), 2.37 (tt, J = |
| | | | | 7.7, 6.9 Hz, 2H) |
| 1-52 | 4.07 | 373.1 | Method 2 | (500 MHz, CDCl₃): δ = 8.69 (ddd, J = 5.0, 1.9, 0.9 Hz, 1H), |
| | | | | 8.54 (dt, J = 8.2, 0.7 Hz, 1H), 8.39 (d, J = 0.8 Hz, 1H), 7.97 |
| | | | | (dt, J = 8.3, 1.6 Hz, 1H), 7.92 (t, J = 1.5 Hz, 1H), 7.88 (ddd, |
| | | | | J = 8.2, 7.4, 2.0 Hz, 1H), 7.80 (dt, J = 8.2, 1.0 Hz, 1H), 7.36 |
| | | | | (ddd, J = 7.4, 4.9, 1.1 Hz, 1H), 7.07 (dd, J = 10.3, 8.7 Hz, 1H), |
| | | | | 6.51-6.44 (m, 2H), 3.90 (t, J = 7.2 Hz, 4H), 2.39 (tt, J = 7.6, |
| | | | | 6.8 Hz, 2H) |
| 1-53 | 3.99 | 356.2 | Method 2 | (500 MHz, CDCl₃): δ = 8.70 (ddd, J = 4.9, 1.9, 0.9 Hz, 1H), |
| | | | | 8.59-8.55 (m, 1H), 8.42 (d, J = 0.7 Hz, 1H), 8.27 (dd, J = |
| | | | | 5.3, 0.8 Hz, 1H), 8.01 (dd, J = 8.3, 1.7 Hz, 1H), 7.95 (dd, J = |
| | | | | 1.7, 0.6 Hz, 1H), 7.90 (ddd, J = 8.1, 7.4, 1.9 Hz, 1H), 7.79 (dt, |
| | | | | J = 8.0, 1.0 Hz, 1H), 7.37 (ddd, J = 7.5, 4.9, 1.0 Hz, 1H), 6.87 |
| | | | | (dd, J = 5.3, 1.6 Hz, 1H), 6.49 (dd, J = 1.7, 0.8 Hz, 1H), 4.15 |
| | | | | (t, J = 7.4 Hz, 4H), 2.46 (tt, J = 8.2, 7.0 Hz, 2H) |
| 1-54 | 4.20 | 359.1 | Method 2 | (500 MHz, CDCl₃): δ = 8.75 (d, J = 0.8 Hz, 2H), 8.55-8.50 |
| | | | | (m, 1H), 8.30 (d, J = 0.7 Hz, 1H), 7.75 (dd, J = 8.1, 1.6 Hz, |
| | | | | 1H), 7.67 (dd, J = 1.7, 0.6 Hz, 1H), 7.29-7.23 (m, 1H), 6.92 |
| | | | | (dd, J = 8.4, 1.1 Hz, 1H), 6.89 (dd, J = 7.6, 1.1 Hz, 1H), 3.89 |
| | | | | (s, 3H), 2.42 (d, J = 0.8 Hz, 3H), 2.13 (s, 3H) |
| 1-55 | 4.27 | 375.1 | Method 2 | (500 MHz, CDCl₃): δ = 8.56 (s, 2H), 8.55 8.51 (m, 1H), 8.30 |
| | | | | (d, J = 0.8 Hz, 1H), 7.76 (dd, J = 8.2, 1.6 Hz, 1H), 7.68 (dd, J = |
| | | | | 1.6, 0.6 Hz, 1H), 7.29-7.25 (m, 1H), 6.95-6.92 (m, 1H), |
| | | | | 6.90 (dd, J = 7.7, 1.1 Hz, 1H), 4.01 (s, 3H), 3.90 (s, 3H), 2.13 |
| | | | | (s, 3H) |
| 1-56 | 3.71 | 370.1 | Method 2 | (500 MHz, CDCl₃): δ = 8.75 (d, J = 0.9 Hz, 2H), 8.53 (dt, J = |
| | | | | 8.2, 0.7 Hz, 1H), 8.32 (d, J = 0.7 Hz, 1H), 7.88 (dd, J = 8.2, |
| | | | | 1.7 Hz, 1H), 7.78 (dd, J = 1.7, 0.6 Hz, 1H), 7.26-7.22 (m, |
| | | | | 1H), 6.82 (dd, J = 7.7, 0.9 Hz, 1H), 6.58 (d, J = 7.8 Hz, 1H), |
| | | | | 3.35 (t, J = 8.1 Hz, 2H), 3.05 (t, J = 8.0 Hz, 2H), 2.81 (s, 3H), |
| | | | | 2.42 (d, J = 0.8 Hz, 3H) |
| 1-57 | 3.8 | 386.1 | Method 2 | (500 MHz, CDCl₃): δ = 8.56 (s, 2H), 8.53 (dt, J = 8.2, 0.7 Hz, |
| | | | | 1H), 8.31 (d, J = 0.8 Hz, 1H), 7.88 (dd, J = 8.3, 1.7 Hz, 1H), |
| | | | | 7.78 (dd, J = 1.7, 0.5 Hz, 1H), 7.27-7.23 (m, 1H), 6.83 (d, J = |
| | | | | 7.6 Hz, 1H), 6.59 (d, J = 7.8 Hz, 1H), 4.01 (s, 3H), 3.36 (t, J = |
| | | | | 8.0 Hz, 2H), 3.06 (t, J = 8.0 Hz, 2H), 2.83 (s, 3H) |
| 1-58 | 4.59 | 362.1 | Method 2 | (500 MHz, CDCl₃): δ = 8.53 (dt, J = 8.2, 0.7 Hz, 1H), 8.50 |
| | | | | (ddd, J = 4.7, 1.5, 0.9 Hz, 1H), 8.35 (d, J = 0.7 Hz, 1H), 7.78 |
| | | | | (dd, J = 8.2, 1.6 Hz, 1H), 7.70 (dd, J = 1.6, 0.6 Hz, 1H), 7.65 |
| | | | | (ddd, J = 9.0, 8.3, 1.5 Hz, 1H), 7.47 (ddd, J = 8.3, 4.7, 3.8 Hz, |
| | | | | 1H), 7.30-7.26 (m, 1H), 6.94 (dd, J = 8.2, 1.1 Hz, 1H), 6.90 |
| | | | | (dd, J = 7.7, 1.1 Hz, 1H), 3.91 (s, 3H), 2.15 (s, 3H) |
| 1-59 | 4.45 | 373.1 | Method 2 | (500 MHz, CDCl₃): δ = 8.58 (dt, J = 8.3, 0.7 Hz, 1H), 8.51 |
| | | | | (ddd, J = 4.7, 1.4, 0.8 Hz, 1H), 8.40 (d, J = 0.7 Hz, 1H), 8.17 |
| | | | | (dd, J = 8.2, 1.7 Hz, 1H), 8.06 (dd, J = 1.7, 0.6 Hz, 1H), 7.66 |
| | | | | (ddd, J = 8.9, 8.2, 1.5 Hz, 1H), 7.48 (ddd, J = 8.3, 4.7, 3.7 Hz, |
| | | | | 1H), 7.44 (dt, J = 8.2, 1.0 Hz, 1H), 7.38 (dd, J = 8.2, 7.2 Hz, |
| | | | | 1H), 7.29 (dd, J = 7.2, 1.0 Hz, 1H), 7.19 (d, J = 3.2 Hz, 1H), |
| | | | | 6.65 (dd, J = 3.2, 0.9 Hz, 1H), 3.88 (s, 3H) |
| 1-60 | 4.74 | 351.2 | Method 2 | (500 MHz, CDCl₃): δ = 8.47 (dt, J = 8.2, 0.8 Hz, 1H), 8.23 (s, |
| | | | | 1H), 7.71 (dd, J = 8.2, 1.7 Hz, 1H), 7.61 (dd, J = 1.7, 0.6 Hz, |
| | | | | 1H), 7.28-7.24 (m, 1H), 6.94-6.91 (m, 1H), 6.87 (dd, J = |
| | | | | 7.7, 1.1 Hz, 1H), 5.36-5.25 (m, 1H), 4.17 4.08 (m, 2H), |
| | | | | 3.90 (s, 3H), 3.63 (td, J = 12.1, 2.0 Hz, 2H), 2.29-2.18 (m, |
| | | | | 2H), 2.11 (s, 3H), 1.82 (ddd, J = 12.4, 4.0, 1.8 Hz, 2H) |
| 1-61 | 4.10 | 369.1 | Method 2 | (500 MHz, CDCl₃): δ = 8.70 (ddd, J = 4.9, 1.9, 0.9 Hz, 1H), |
| | | | | 8.51 (dd, J = 8.2, 0.8 Hz, 1H), 8.35 (d, J = 0.7 Hz, 1H), 7.89 |
| | | | | (ddd, J = 8.1, 7.4, 1.9 Hz, 1H), 7.83-7.75 (m, 2H), 7.69 (d, J = |
| | | | | 1.6 Hz, 1H), 7.36 (ddd, J = 7.4, 4.9, 1.1 Hz, 1H), 7.18 (t, J = |
| | | | | 7.9 Hz, 1H), 6.70 (d, J = 8.2 Hz, 1H), 6.59 (d, J = 7.5 Hz, 1H), |
| | | | | 3.29 (t, J = 5.8 Hz, 2H), 2.97 (s, 3H), 2.60 (t, J = 6.3 Hz, 2H), |
| | | | | 1.94-1.86 (m, 2H) |
| 1-62 | 4.30 | 363.1 | Method 2 | (500 MHz, CDCl₃): δ = 8.79 (s, 2H), 8.54 (dt, J = 8.2, 0.7 Hz, |
| | | | | 1H), 8.32 (d, J = 0.7 Hz, 1H), 7.78 (dd, J = 8.1, 1.6 Hz, 1H), |
| | | | | 7.69 (dd, J = 1.6, 0.6 Hz, 1H), 7.30-7.26 (m, 1H), 6.94 (dd, J = |
| | | | | 8.3, 1.1 Hz, 1H), 6.90 (dd, J = 7.7, 1.1 Hz, 1H), 3.90 (s, 3H), |
| | | | | 2.14 (s, 3H) |
| 1-63 | 3.35 | 443.2 | Method 2 | (500 MHz, CDCl₃): δ = 8.54-8.51 (m, 1H), 8.49 (s, 2H), 8.29 |
| | | | | (d, J = 0.9 Hz, 1H), 7.74 (dd, J = 8.2, 1.7 Hz, 1H), 7.66 (d, J = |
| | | | | 1.6 Hz, 1H), 7.30-7.24 (m, 1H), 6.91 (ddd, J = 16.2, 8.0, 1.1 |
| | | | | Hz, 2H), 3.90 (s, 3H), 3.41-3.34 (m, 4H), 2.66-2.61 (m, |
| | | | | 4H), 2.39 (s, 3H), 2.13 (s, 3H) |
| 1-64 | 4.13 | 430.1 | Method 2 | (500 MHz, CDCl₃): δ = 8.51 (dd, J = 8.1, 0.7 Hz, 1H), 8.48 (s, |
| | | | | 2H), 8.28 (d, J = 0.7 Hz, 1H), 7.73 (dd, J = 8.2, 1.7 Hz, 1H), |
| | | | | 7.65 (d, J = 1.6 Hz, 1H), 7.27-7.24 (m, 1H), 6.91 (dd, J = |
| | | | | 8.2, 1.2 Hz, 1H), 6.88 (dd, J = 7.7, 1.1 Hz, 1H), 3.92-3.89 |
| | | | | (m, 4H), 3.88 (s, 3H), 3.32-3.28 (m, 4H), 2.11 (s, 3H) |
| 1-65 | 3.85 | 430.1 | Method 2 | (500 MHz, DMSO-d₆): δ = 8.53 (d, J = 0.7 Hz, 1H), 8.33 (dt, |
| | | | | J = 8.2, 0.7 Hz, 1H), 8.22 (s, 2H), 7.98 (dd, J = 1.7, 0.6 Hz, |
| | | | | 1H), 7.86 (dd, J = 8.1, 1.7 Hz, 1H), 7.32 (ddd, J = 8.3, 7.6, 0.7 |
| | | | | Hz, 1H), 7.08 (dd, J = 8.3, 1.1 Hz, 1H), 6.94 (dd, J = 7.6, 1.1 |
| | | | | Hz, 1H), 5.09 (d, J = 3.9 Hz, 1H), 4.50-4.43 (m, 1H), 3.86 (s, |
| | | | | 3H), 3.57-3.43 (m, 3H), 3.29-3.22 (m, 1H), 2.09 (s, 3H), |
| | | | | 2.06 (dt, J = 8.7, 4.4 Hz, 1H), 1.98 1.93 (m, 1H) |
| 1-66 | 3.28 | 380.2 | Method 2 | (500 MHz, CDCl₃): δ = 8.44 (dd, J = 8.2, 0.8 Hz, 1H), 8.17 (d, |
| | | | | J = 0.7 Hz, 1H), 7.71 (dd, J = 8.2, 1.6 Hz, 1H), 7.61 (d, J = 1.5 |
| | | | | Hz, 1H), 7.28-7.23 (m, 1H), 6.92 (dd, J = 8.3, 1.1 Hz, 1H), |
| | | | | 6.87 (dd, J = 7.6, 1.2 Hz, 1H), 4.41 (t, J = 6.9 Hz, 2H), 3.90 (s, |
| | | | | 3H), 3.72-3.67 (m, 4H), 2.85 (t, J = 6.9 Hz, 2H), 2.59 (t, J = |
| | | | | 4.6 Hz, 4H), 2.12 (s, 3H) |
| 1-67 | 4.75 | 390.1 | Method 2 | (500 MHz, CDCl₃): δ = 8.55 (dd, J = 2.3, 1.0 Hz, 1H), 8.54- |
| | | | | 8.51 (m, 1H), 8.36 (d, J = 0.7 Hz, 1H), 7.77 (dd, J = 8.2, 1.7 |
| | | | | Hz, 1H), 7.75-7.73 (m, 2H), 7.68 (dd, J = 1.7, 0.6 Hz, 1H), |
| | | | | 7.29-7.25 (m, 1H), 6.95-6.92 (m, 1H), 6.89 (dd, J = 7.7, 1.1 |
| | | | | Hz, 1H), 3.90 (s, 3H), 2.57 (s, 3H), 2.14 (s, 3H) |
| 1-68 | 4.38 | 422.0 | Method 2 | (500 MHz, CDCl₃): δ = 9.19 (d, J = 2.3 Hz, 1H), 8.52 (dd, J = |
| | | | | 8.2, 0.7 Hz, 1H), 8.41-8.35 (m, 2H), 8.11 (dd, J = 8.5, 0.7 |
| | | | | Hz, 1H), 7.79 (dd, J = 8.2, 1.6 Hz, 1H), 7.70 (dd, J = 1.6, 0.5 |
| | | | | Hz, 1H), 7.26 (ddd, J = 8.3, 7.6, 0.7 Hz, 1H), 6.93 (dd, J = 8.2, |
| | | | | 1.1 Hz, 1H), 6.87 (dd, J = 7.7, 1.1 Hz, 1H), 3.89 (s, 3H), 3.15 |
| | | | | (s, 3H), 2.12 (s, 3H) |
| 1-69 | 3.42 | 416.2 | Method 2 | (500 MHz, CDCl₃): δ = 8.53 (d, J = 8.2 Hz, 1H), 8.45 (s, 2H), |
| | | | | 8.30 (d, J = 0.7 Hz, 1H), 7.76 (dd, J = 8.2, 1.7 Hz, 1H), 7.68 |
| | | | | (dd, J = 1.6, 0.6 Hz, 1H), 7.29-7.25 (m, 1H), 6.95-6.92 (m, |
| | | | | 1H), 6.89 (dd, J = 7.7, 1.1 Hz, 1H), 4.95 (p, J = 5.6 Hz, 1H), |
| | | | | 3.90 (s, 3H), 3.88-3.84 (m, 2H), 3.23-3.16 (m, 2H), 2.59 |
| | | | | (q, J = 7.2 Hz, 1H), 2.13 (s, 3H) |
| 1-70 | 4.52 | 414.1 | Method 2 | (500 MHz, CDCl₃): δ = 8.52 (dd, J = 8.2, 0.8 Hz, 1H), 8.28 (d, |
| | | | | J = 0.7 Hz, 1H), 8.17 (s, 2H), 7.73 (dd, J = 8.2, 1.7 Hz, 1H), |
| | | | | 7.65 (dd, J = 1.7, 0.6 Hz, 1H), 7.29-7.25 (m, 1H), 6.93 (dd, J = |
| | | | | 8.2, 1.1 Hz, 1H), 6.90 (dd, J = 7.6, 1.1 Hz, 1H), 3.90 (s, 3H), |
| | | | | 3.45-3.36 (m, 4H), 2.13 (s, 3H), 2.12-2.08 (m, 4H) |
| 1-71 | 4.75 | 428.1 | Method 2 | (500 MHz, CDCl₃): δ = 8.53 (dd, J = 8.2, 0.7 Hz, 1H), 8.47 (s, |
| | | | | 2H), 8.28 (d, J = 0.7 Hz, 1H), 7.74 (dd, J = 8.2, 1.6 Hz, 1H), |
| | | | | 7.66 (dd, J = 1.7, 0.6 Hz, 1H), 7.29-7.25 (m, 1H), 6.93 (dd, J = |
| | | | | 8.4, 1.1 Hz, 1H), 6.90 (dd, J = 7.7, 1.1 Hz, 1H), 3.90 (s, 3H), |
| | | | | 3.36-3.30 (m, 4H), 2.13 (s, 3H), 1.78-1.73 (m, 4H), 1.69- |
| | | | | 1.65 (m, 2H) |
| 1-72 | 3.91 | 444.2 | Method 2 | (500 MHz, CDCl₃): δ = 8.53 (dd, J = 8.1, 0.7 Hz, 1H), 8.50 (s, |
| | | | | 2H), 8.29 (d, J = 0.7 Hz, 1H), 7.75 (dd, J = 8.2, 1.6 Hz, 1H), |
| | | | | 7.67 (dd, J = 1.6, 0.6 Hz, 1H), 7.29-7.25 (m, 1H), 6.93 (dd, J = |
| | | | | 8.4, 1.1 Hz, 1H), 6.90 (dd, J = 7.8, 1.1 Hz, 1H), 3.98 (td, J = |
| | | | | 8.2, 4.0 Hz, 1H), 3.90 (s, 3H), 3.68 (dt, J = 13.1, 4.8 Hz, 2H), |
| | | | | 3.16 (ddd, J = 12.6, 9.1, 3.4 Hz, 2H), 2.14 (s, 3H), 2.10-2.03 |
| | | | | (m, 2H), 1.75 (dtd, J = 12.8, 8.7, 3.9 Hz, 2H), 1.63-1.61 (m, |
| | | | | 1H) |
| 1-73 | 3.98 | 444.2 | Method 2 | (500 MHz, CDCl₃): δ = 8.52 (dt, J = 8.2, 0.7 Hz, 1H), 8.51 (s, |
| | | | | 2H), 8.29 (d, J = 0.7 Hz, 1H), 7.75 (dd, J = 8.1, 1.6 Hz, 1H), |
| | | | | 7.67 (dd, J = 1.7, 0.6 Hz, 1H), 7.29-7.25 (m, 1H), 6.93 (dd, J = |
| | | | | 8.3, 1.1 Hz, 1H), 6.90 (dd, J = 7.7, 1.0 Hz, 1H), 4.00-3.94 |
| | | | | (m, 1H), 3.90 (s, 3H), 3.63-3.57 (m, 1H), 3.44-3.37 (m, |
| | | | | 1H), 3.17 (ddd, J = 11.8, 8.4, 3.0 Hz, 1H), 3.11 (dd, J = 12.1, |
| | | | | 7.4 Hz, 1H), 2.13 (s, 3H), 1.99 (dp, J = 10.0, 3.7 Hz, 2H), 1.78- |
| | | | | 1.69 (m, 1H), 1.63 (dd, J = 16.9, 8.5 Hz, 1H) |
| 1-74 | 4.01 | 458.1 | Method 2 | (500 MHz, CDCl₃): δ = 8.52 (dt, J = 8.2, 0.7 Hz, 1H), 8.28 (d, |
| | | | | J = 0.7 Hz, 1H), 8.22 (s, 2H), 7.74 (dd, J = 8.2, 1.7 Hz, 1H), |
| | | | | 7.66 (dd, J = 1.7, 0.6 Hz, 1H), 7.29-7.25 (m, 1H), 6.93 (dd, J = |
| | | | | 8.3, 1.1 Hz, 1H), 6.90 (dd, J = 7.7, 1.1 Hz, 1H), 4.02 (d, J = |
| | | | | 7.8 Hz, 1H), 3.99 (s, 1H), 3.90 (s, 3H), 3.49-3.44 (m, 1H), |
| | | | | 2.13 (s, 3H), 1.85 (d, J = 4.9 Hz, 2H), 1.76 (dtd, J = 13.4, 9.8, |
| | | | | 5.1 Hz, 4H), 1.59 (s, 1H), 1.46 (s, 1H) |
| 1-75 | 4.0 | 458.1 | Method 2 | (500 MHz, CDCl₃): δ = 8.52 (dt, J = 8.2, 0.7 Hz, 1H), 8.28 (d, |
| | | | | J = 0.7 Hz, 1H), 8.22 (d, J = 19.9 Hz, 2H), 7.74 (dd, J = 8.2, |
| | | | | 1.7 Hz, 1H), 7.66 (d, J = 1.7 Hz, 1H), 7.26 (s, 1H), 6.95-6.92 |
| | | | | (m, 1H), 6.90 (dd, J = 7.7, 1.1 Hz, 1H), 3.90 (s, 3H), 3.60 (dd, |
| | | | | J = 10.5, 4.0 Hz, 1H), 3.54 (s, 1H), 3.27 (dd, J = 10.5, 7.9 Hz, |
| | | | | 1H), 2.79-2.71 (m, 1H), 2.24 (d, J = 12.8 Hz, 1H), 2.13 (s, |
| | | | | 3H), 1.91 (d, J = 5.6 Hz, 1H), 1.68 (d, J = 16.9 Hz, 2H), 1.48- |
| | | | | 1.40 (m, 2H), 1.33 1.23 (m, 2H) |
| 1-76 | 4.1 | 374.1 | Method 2 | (500 MHz, CDCl₃): δ = 8.79 (s, 2H), 8.54 (dd, J = 8.2, 0.7 Hz, |
| | | | | 1H), 8.35 (d, J = 0.7 Hz, 1H), 8.03 (dd, J = 8.3, 1.8 Hz, 1H), |
| | | | | 7.93-7.90 (m, 1H), 7.35 (t, J = 7.8 Hz, 1H), 7.03 (ddd, J = |
| | | | | 7.6, 1.8, 0.9 Hz, 1H), 6.69 (t, J = 2.0 Hz, 1H), 6.54 (ddd, J = |
| | | | | 8.1, 2.4, 0.9 Hz, 1H), 3.97 (t, J = 7.2 Hz, 4H), 2.43 (tt, J = 7.7, |
| | | | | 6.9 Hz, 2H) |
| 1-77 | 3.45 | 369.2 | Method 2 | (500 MHz, CDCl₃): δ = 8.70 (ddd, J = 4.9, 1.9, 0.9 Hz, 1H), |
| | | | | 8.52 (dt, J = 8.2, 0.7 Hz, 1H), 8.36 (d, J = 0.7 Hz, 1H), 7.89 |
| | | | | (ddd, J = 8.1, 7.4, 1.9 Hz, 1H), 7.83-7.76 (m, 2H), 7.70 (dd, |
| | | | | J = 1.7, 0.6 Hz, 1H), 7.36 (ddd, J = 7.4, 4.9, 1.1 Hz, 1H), 7.24 |
| | | | | 7.19 (m, 1H), 6.81 (dd, J = 7.4, 1.2 Hz, 1H), 6.62 (dd, J = |
| | | | | 8.0, 1.2 Hz, 1H), 3.97 (t, J = 7.2 Hz, 4H), 2.37 2.27 (m, 2H), |
| | | | | 2.09 (s, 3H) |
| 1-78 | 4.52 | 446.1 | Method 2 | (500 MHz, CDCl₃): δ = 8.53 (d, J = 8.2 Hz, 1H), 8.46 (s, 2H), |
| | | | | 8.29 (d, J = 0.7 Hz, 1H), 7.75 (dd, J = 8.1, 1.7 Hz, 1H), 7.67 |
| | | | | (dd, J = 1.7, 0.6 Hz, 1H), 7.29-7.26 (m, 1H), 6.94 (dd, J = |
| | | | | 8.3, 1.1 Hz, 1H), 6.90 (dd, J = 7.7, 1.1 Hz, 1H), 3.90 (s, 3H), |
| | | | | 3.77 3.70 (m, 4H), 2.82-2.77 (m, 4H), 2.13 (s, 3H) |
| 1-79 | 4.06 | 418.1 | Method 2 | (500 MHz, CDCl₃): δ = 8.53 (dt, J = 8.2, 0.7 Hz, 1H), 8.28 (d, |
| | | | | J = 0.7 Hz, 1H), 8.27 (s, 2H), 7.74 (dd, J = 8.1, 1.6 Hz, 1H), |
| | | | | 7.66 (dd, J = 1.6, 0.6 Hz, 1H), 7.29-7.25 (m, 1H), 6.95- |
| | | | | 6.92 (m, 1H), 6.90 (dd, J = 7.7, 1.1 Hz, 1H), 4.39 (t, J = 5.6 |
| | | | | Hz, 1H), 3.90 (s, 3H), 3.66 (dd, J = 5.6, 4.6 Hz, 2H), 3.43 (s, |
| | | | | 3H), 3.40 (q, J = 5.4 Hz, 2H), 2.13 (s, 3H) |
| 1-80 | 3.30 | 431.1 | Method 2 | (500 MHz, CDCl₃): δ = 8.53 (dd, J = 8.2, 0.7 Hz, 1H), 8.28 (d, |
| | | | | J = 0.8 Hz, 1H), 8.26 (s, 2H), 7.74 (dd, J = 8.2, 1.6 Hz, 1H), |
| | | | | 7.66 (dd, J = 1.6, 0.6 Hz, 1H), 7.29-7.25 (m, 1H), 6.93 (dd, J = |
| | | | | 8.4, 1.1 Hz, 1H), 6.90 (dd, J = 7.7, 1.1 Hz, 1H), 4.85 (s, 1H), |
| | | | | 3.90 (s, 3H), 3.27-3.20 (m, 2H), 2.65 (dd, J = 6.5, 5.0 Hz, |
| | | | | 2H), 2.30 (s, 6H), 2.13 (s, 3H) |
| 1-81 | 4.38 | 414.1 | Method 2 | (500 MHz, CDCl₃): δ = 8.55-8.49 (m, 1H), 8.49 (s, 2H), 8.29 |
| | | | | (d, J = 0.7 Hz, 1H), 7.75 (dd, J = 8.1, 1.6 Hz, 1H), 7.67 (d, J = |
| | | | | 1.6 Hz, 1H), 7.18 (d, J = 7.6 Hz, 1H), 7.15 (s, 1H), 7.14-7.11 |
| | | | | (m, 1H), 3.94-3.89 (m, 4H), 3.34-3.29 (m, 4H), 2.40 (s, |
| | | | | 3H), 2.27 (s, 3H) |
| 1-82 | 4.10 | 414.1 | Method 2 | (500 MHz, CDCl₃): δ = 8.51 (dt, J = 8.1, 0.7 Hz, 1H), 8.29 (d, |
| | | | | J = 0.7 Hz, 1H), 8.15 (s, 2H), 7.75 (dd, J = 8.2, 1.7 Hz, 1H), |
| | | | | 7.66 (dd, J = 1.7, 0.6 Hz, 1H), 7.19 (d, J = 7.7 Hz, 1H), 7.15 |
| | | | | (dq, J = 1.3, 0.7 Hz, 1H), 7.12 (ddt, J = 7.8, 1.9, 0.7 Hz, 1H), |
| | | | | 4.68 (s, 1H), 3.64-3.59 (m, 1H), 3.59-3.54 (m, 1H), 3.47 |
| | | | | (ddd, J = 9.1, 8.1, 3.5 Hz, 1H), 3.37 (dt, J = 10.5, 1.6 Hz, 1H), |
| | | | | 2.57 (d, J = 3.1 Hz, 1H), 2.39 (s, 3H), 2.27 (s, 3H), 2.22-2.13 |
| | | | | (m, 2H) |
| 1-83 | 3.54 | 441.2 | Method 2 | (500 MHz, CDCl₃): δ = 8.52 (dt, J = 8.3, 0.7 Hz, 1H), 8.33 (d, |
| | | | | J = 0.7 Hz, 1H), 8.17 (s, 2H), 8.00 (dd, J = 8.2, 1.7 Hz, 1H), |
| | | | | 7.90 (dd, J = 1.8, 0.6 Hz, 1H), 7.34 (t, J = 7.8 Hz, 1H), 7.03 |
| | | | | (ddd, J = 7.7, 1.8, 0.9 Hz, 1H), 6.69 (t, J = 2.0 Hz, 1H), 6.53 |
| | | | | (ddd, J = 8.0, 2.3, 0.9 Hz, 1H), 4.70 (s, 1H), 3.97 (t, J = 7.2 |
| | | | | Hz, 4H), 3.65-3.58 (m, 2H), 3.49 (td, J = 8.8, 3.3 Hz, 1H), |
| | | | | 3.38 (dt, J = 10.5, 1.6 Hz, 1H), 2.42 (tt, J = 7.7, 6.8 Hz, 2H), |
| | | | | 2.27 2.11 (m, 3H) |
| 1-84 | 4.0 | 442.0 | Method 2 | (500 MHz, CDCl₃): δ = 8.51 (d, J = 8.2 Hz, 1H), 8.27 (s, 1H), |
| | | | | 8.08 (s, 2H), 7.74 (dd, J = 8.2, 1.7 Hz, 1H), 7.66 (d, J = 1.7 |
| | | | | Hz, 1H), 7.30-7.23 (m, 1H), 6.93 (d, J = 8.2 Hz, 1H), 6.91- |
| | | | | 6.88 (m, 1H), 4.88 (s, 4H), 4.23 (s, 4H), 3.90 (s, 3H), 2.13 (s, |
| | | | | 3H) |
| 1-85 | 4.52 | 389.1 | Method 2 | (500 MHz, CDCl₃): δ = 8.54 (s, 2H), 8.53 (dt, J = 8.1, 0.7 Hz, |
| | | | | 1H), 8.30 (d, J = 0.7 Hz, 1H), 7.76 (dd, J = 8.1, 1.6 Hz, 1H), |
| | | | | 7.68 (dd, J = 1.7, 0.6 Hz, 1H), 7.30-7.25 (m, 1H), 6.95- |
| | | | | 6.92 (m, 1H), 6.90 (dd, J = 7.7, 1.0 Hz, 1H), 4.23 (q, J = 6.9 |
| | | | | Hz, 2H), 3.90 (s, 3H), 2.14 (s, 3H), 1.51 (t, J = 7.0 Hz, 3H) |
| 1-86 | 4.04 | 422.1 | Method 2 | (500 MHz, CDCl₃): δ = 8.53 (d, J = 8.1 Hz, 1H), 8.28 (s, 1H), |
| | | | | 8.22 (s, 2H), 7.74 (dd, J = 8.2, 1.6 Hz, 1H), 7.66 (d, J = 1.7 |
| | | | | Hz, 1H), 7.26 (t, J = 4.4 Hz, 1H), 6.95-6.92 (m, 1H), 6.90 |
| | | | | (dd, J = 7.7, 1.1 Hz, 1H), 4.75 (d, J = 1.9 Hz, 1H), 4.54 (d, J = |
| | | | | 1.8 Hz, 1H), 3.93 (s, 2H), 3.90 (s, 3H), 3.64 (dd, J = 9.1, 1.6 |
| | | | | Hz, 1H), 3.30 (d, J = 9.1 Hz, 1H), 2.13 (s, 3H), 2.08 (d, J = 1.9 |
| | | | | Hz, 2H) |
| 1-87 | 3.88 | 444.1 | Method 2 | (500 MHz, CDCl₃): δ = 8.52 (dt, J = 8.2, 0.7 Hz, 1H), 8.29 (d, |
| | | | | J = 0.7 Hz, 1H), 8.22 (s, 2H), 7.75 (dd, J = 8.2, 1.6 Hz, 1H), |
| | | | | 7.67 (dd, J = 1.6, 0.6 Hz, 1H), 7.29-7.25 (m, 1H), 6.95- |
| | | | | 6.92 (m, 1H), 6.90 (dd, J = 7.7, 1.1 Hz, 1H), 4.51 (dt, J = 5.6, |
| | | | | 2.8 Hz, 1H), 4.10 (d, J = 2.9 Hz, 1H), 3.90 (s, 3H), 2.39 (dq, |
| | | | | J = 14.7, 7.4 Hz, 1H), 2.22 (dd, J = 13.4, 5.9 Hz, 1H), 2.14 (s, |
| | | | | 3H), 2.09 (dddd, J = 14.1, 9.0, 5.3, 3.6 Hz, 1H), 1.79-1.69 |
| | | | | (m, 2H), 1.60 (s, 2H), 1.58-1.50 (m, 1H) |
| 1-88 | 3.94 | 432.1 | Method 2 | (500 MHz, CDCl₃): δ = 8.52 (dt, J = 8.2, 0.7 Hz, 1H), 8.29 (d, |
| | | | | J = 0.7 Hz, 1H), 8.25 (s, 2H), 7.75 (dd, J = 8.2, 1.7 Hz, 1H), |
| | | | | 7.67 (dd, J = 1.7, 0.6 Hz, 1H), 7.29-7.25 (m, 1H), 6.93 (dd, J = |
| | | | | 8.2, 1.1 Hz, 1H), 6.90 (dd, J = 7.7, 1.1 Hz, 1H), 4.60 (t, J = |
| | | | | 5.9 Hz, 1H), 3.90 (s, 3H), 3.17 (d, J = 5.9 Hz, 2H), 2.14 (s, |
| | | | | 3H), 1.92 (s, 1H), 1.36 (s, 6H) |
| 1-89 | 4.46 | 419.0 | Method 2 | (500 MHz, CDCl₃): δ = 8.49 (s, 2H), 8.13 (dd, J = 1.5, 0.7 Hz, |
| | | | | 1H), 7.90 (dd, J = 7.7, 0.7 Hz, 1H), 7.60 (dd, J = 7.7, 1.5 Hz, |
| | | | | 1H), 7.24 (ddd, J = 8.3, 7.6, 0.6 Hz, 1H), 6.91 (dd, J = 8.3, 1.1 |
| | | | | Hz, 1H), 6.87 (dd, J = 7.7, 1.1 Hz, 1H), 4.30-4.25 (m, 2H), |
| | | | | 3.89 (s, 3H), 3.82-3.77 (m, 2H), 3.47 (s, 3H), 2.14 (s, 3H) |
| 1-90 | 4.59 | 378.9 | Method 2 | (500 MHz, CDCl₃): δ = 8.88 (s, 2H), 8.54 (dt, J = 8.1, 0.7 Hz, |
| | | | | 1H), 8.32 (d, J = 0.7 Hz, 1H), 7.78 (dd, J = 8.1, 1.6 Hz, 1H), |
| | | | | 7.69 (dd, J = 1.7, 0.6 Hz, 1H), 7.30-7.25 (m, 1H), 6.97- |
| | | | | 6.92 (m, 1H), 6.89 (dd, J = 7.7, 1.1 Hz, 1H), 3.90 (s, 3H), 2.14 |
| | | | | (s, 3H) |
| 1-91 | 3.90 | 421.0 | Method 2 | (500 MHz, CDCl₃): δ = 9.26 (dd, J = 2.4, 0.9 Hz, 1H), 8.54 |
| | | | | (dt, J = 8.3, 0.7 Hz, 1H), 8.49-8.42 (m, 1H), 8.40 (d, J = 0.8 |
| | | | | Hz, 1H), 8.08 (dt, J = 8.5, 0.9 Hz, 1H), 7.81 (dt, J = 8.2, 1.3 |
| | | | | Hz, 1H), 7.71 (d, J = 1.3 Hz, 1H), 7.28 (t, J = 8.1 Hz, 1H), |
| | | | | 6.95 (dd, J = 8.4, 1.2 Hz, 1H), 6.89 (dd, J = 7.6, 1.2 Hz, 1H), |
| | | | | 3.91 (d, J = 1.0 Hz, 3H), 3.21 (t, J = 1.2 Hz, 3H), 2.92 (s, 1H), |
| | | | | 2.14 (s, 3H) |
| 1-92 | 3.93 | 459.1 | Method 2 | (500 MHz, CDCl₃): δ = 9.01 (dd, J = 2.5, 0.8 Hz, 1H), 8.51 |
| | | | | (dd, J = 8.2, 0.8 Hz, 1H), 8.37 (d, J = 0.7 Hz, 1H), 8.26 (dd, |
| | | | | J = 8.4, 2.4 Hz, 1H), 7.81 (dd, J = 8.4, 0.8 Hz, 1H), 7.79 (dd, |
| | | | | J = 8.2, 1.7 Hz, 1H), 7.70 (d, J = 1.6 Hz, 1H), 7.29-7.25 (m, |
| | | | | 2H), 6.94 (dd, J = 8.3, 1.1 Hz, 1H), 6.88 (dd, J = 7.7, 1.1 Hz, |
| | | | | 1H), 3.90 (s, 3H), 3.51 (d, J = 6.0 Hz, 2H), 3.00 (s, 1H), 2.13 |
| | | | | (s, 3H), 1.30 (s, 6H) |
| 1-93 | 3.77 | 457.1 | Method 2 | (500 MHz, CDCl₃): δ = 8.86 (dd, J = 22.5, 2.3 Hz, 1H), 8.53 |
| | | | | (dd, J = 8.1, 1.7 Hz, 1H), 8.39 (s, 1H), 8.08 (ddd, J = 10.4, |
| | | | | 8.3, 2.3 Hz, 1H), 7.96 7.88 (m, 1H), 7.78 (dd, J = 8.2, 1.7 |
| | | | | Hz, 1H), 7.70 (d, J = 1.6 Hz, 1H), 7.28 (d, J = 8.0 Hz, 1H), |
| | | | | 6.94 (dd, J = 8.3, 1.1 Hz, 1H), 6.89 (dd, J = 7.7, 1.1 Hz, 1H), |
| | | | | 4.57 (d, J = 57.0 Hz, 1H), 3.90 (s, 3H), 3.88-3.80 (m, 2H), |
| | | | | 3.74-3.67 (m, 1H), 3.64-3.50 (m, 1H), 2.24 (dd, J = 29.5, |
| | | | | 3.2 Hz, 1H), 2.14 (s, 3H), 2.04 (ddd, J = 8.7, 6.0, 3.5 Hz, 2H) |
| 1-94 | 3.91 | 471.1 | Method 2 | (500 MHz, CDCl₃): δ = 8.98 (dd, J = 2.4, 0.8 Hz, 1H), 8.52 |
| | | | | (dt, J = 8.2, 0.7 Hz, 1H), 8.38 (d, J = 0.7 Hz, 1H), 8.25 (dd, |
| | | | | J = 8.4, 2.4 Hz, 1H), 7.89 (dd, J = 8.4, 0.8 Hz, 1H), 7.79 (dd, |
| | | | | J = 8.2, 1.7 Hz, 1H), 7.70 (dd, J = 1.7, 0.6 Hz, 1H), 7.28 (ddd, |
| | | | | J = 8.4, 7.7, 0.8 Hz, 1H), 6.96-6.93 (m, 1H), 6.90 (dd, J = 7.7, |
| | | | | 1.1 Hz, 1H), 6.33 (d, J = 7.3 Hz, 1H), 4.69 (q, J = 7.4 Hz, 1H), |
| | | | | 4.50 (q, J = 3.0 Hz, 1H), 3.91 (s, 3H), 2.41 (dq, J = 14.4, 7.5 |
| | | | | Hz, 1H), 2.27-2.20 (m, 1H), 2.14 (s, 3H), 2.10 (dt, J = 9.0, |
| | | | | 5.7 Hz, 1H), 1.83 (ddd, J = 13.8, 7.9, 6.0 Hz, 1H), 1.71 |
| | | | | (ddddd, J = 13.5, 8.1, 6.6, 3.4, 1H), 1.63-1.60 (m, 1H), 1.57 |
| | | | | (d, J = 3.8 Hz, 1H) |
| 1-95 | 3.42 | 457.1 | Method 2 | (500 MHz, CDCl₃): δ = 8.52 (d, J = 8.1 Hz, 1H), 8.34-8.19 |
| | | | | (m, 3H), 7.74 (dd, J = 8.1, 1.7 Hz, 1H), 7.66 (d, J = 1.7 Hz, |
| | | | | 1H), 7.36-7.21 (m, 1H), 7.00 6.84 (m, 2H), 5.02 (s, 1H), |
| | | | | 3.90 (s, 3H), 3.31 (q, J = 5.5 Hz, 2H), 2.88 (t, J = 5.8 Hz, 2H), |
| | | | | 2.67 (s, 4H), 2.13 (s. 3H), 1.87 (q, J = 3.4 Hz, 4H) |
| 1-96 | 3.45 | 459.2 | Method 2 | (500 MHz, CDCl₃): δ = 8.52 (d, J = 8.1 Hz, 1H), 8.28 (d, J = |
| | | | | 0.7 Hz, 1H), 8.23 (s, 2H), 7.73 (dd, J = 8.1, 1.7 Hz, 1H), 7.66 |
| | | | | (d, J = 1.6 Hz, 1H), 7.29-7.25 (m, 1H), 6.93 (dd, J = 8.5, 1.2 |
| | | | | Hz, 1H), 6.90 (dd, J = 7.6, 1.1 Hz, 1H), 5.13 (s, 1H), 3.90 (s, |
| | | | | 3H), 3.03 (d, J = 4.5 Hz, 2H), 2.27 (s, 6H), 2.13 (s, 3H), 1.16 |
| | | | | (s, 6H) |
| 1-97 | 3.36 | 454.1 | Method 2 | (500 MHz, CDCl₃): δ = 8.51 (d, J = 8.2 Hz, 1H), 8.29 (s, 1H), |
| | | | | 8.24 (s, 2H), 7.75 (dd, J = 8.2, 1.6 Hz, 1H), 7.67 (d, J = 1.7 |
| | | | | Hz, 1H), 7.45 (s, 1H), 7.26 (s, 2H), 6.94 (d, J = 8.2 Hz, 1H), |
| | | | | 6.90 (dd, J = 7.7, 1.1 Hz, 1H), 6.81 (d, J = 2.4 Hz, 1H), 4.56 |
| | | | | (s, 1H), 4.26 (t, J = 5.7 Hz, 2H), 3.90 (s, 3H), 3.65 (q, J = 5.8 |
| | | | | Hz, 2H), 2.13 (s, 3H) |
| 1-98 | 3.65 | 458.2 | Method 2 | (500 MHz, DMSO-d₆): δ = 11.44 (s, 1H), 8.89 (d, J = 2.7 Hz, |
| | | | | 1H), 8.59 (s, 1H), 8.43 (d, J = 5.4 Hz, 3H), 8.35 (d, J = 8.1 Hz, |
| | | | | 1H), 8.30 (dd, J = 8.6, 2.6 Hz, 1H), 7.99 (s, 1H), 7.87 (d, J = |
| | | | | 8.2 Hz, 1H), 7.69 (d, J = 8.6 Hz, 1H), 7.32 (t, J = 7.9 Hz, 1H), |
| | | | | 7.08 (d, J = 8.2 Hz, 1H), 6.93 (d, J = 7.6 Hz, 1H), 4.00-3.93 |
| | | | | (m, 1H), 3.87 (s, 3H), 2.28 (q, J = 6.7 Hz, 1H), 2.09 (s, 3H), |
| | | | | 1.04 (t, J = 6.5 Hz, 6H) |
| 1-99 | 3.61 | 456.1 | Method 2 | (500 MHz, DMSO-d₆): δ = 11.57 (s, 1H), 10.19-10.07 (m, |
| | | | | 1H), 8.89 (d, J = 2.7 Hz, 1H), 8.78 (dd, J = 11.1, 5.5 Hz, 1H), |
| | | | | 8.58 (s, 1H), 8.35 (d, J = 8.1 Hz, 1H), 8.28 (dd, J = 8.7, 2.7 |
| | | | | Hz, 1H), 7.98 (d, J = 1.7 Hz, 1H), 7.86 (dd, J = 8.2, 1.7 Hz, |
| | | | | 1H), 7.69 (d, J = 8.6 Hz, 1H), 7.31 (t, J = 7.9 Hz, 1H), 7.08 (d, |
| | | | | J = 8.1 Hz, 1H), 6.96-6.87 (m, 1H), 4.54-4.47 (m, 1H), |
| | | | | 3.86 (s, 3H), 3.35-3.23 (m, 2H), 2.49-2.44 (m, 1H), 2.09 (s, |
| | | | | 3H), 2.08-2.02 (m, 1H), 2.00-1.94 (m, 2H) |
| 1-100 | 4.01 | 432.1 | Method 2 | (500 MHz, CDCl₃): δ = 8.50 (d, J = 8.1 Hz, 1H), 8.41 (s, 2H), |
| | | | | 8.29 (s, 1H), 7.74 (dd, J = 8.2, 1.6 Hz, 1H), 7.66 (d, J = 1.6 |
| | | | | Hz, 1H), 7.25 (t, J = 7.9 Hz, 1H), 6.94 6.85 (m, 2H), 3.88 (s, |
| | | | | 3H), 3.60 (s, 2H), 2.12 (s, 3H), 1.34 (s, 6H) |
| 1-101 | 3.44 | 457.2 | Method 2 | (500 MHz, DMSO-d₆): δ = 8.53 (s, 1H), 8.33 (d, J = 8.2 Hz, |
| | | | | 1H), 8.24 (s, 2H), 7.98 (d, J = 1.6 Hz, 1H), 7.86 (dd, J = 8.2, |
| | | | | 1.7 Hz, 1H), 7.32 (t, J = 7.9 Hz, 1H), 7.08 (dd, J = 8.4, 1.0 Hz, |
| | | | | 1H), 6.94 (dd, J = 7.7, 1.1 Hz, 1H), 4.10 (q, J = 5.2 Hz, 1H), |
| | | | | 3.86 (s, 3H), 3.62 (dd, J = 9.7, 7.1 Hz, 1H), 3.55 (td, J = 9.2, |
| | | | | 2.4 Hz, 1H), 3.41-3.34 (m, 1H), 2.90-2.81 (m, 1H), 2.23 (s, |
| | | | | 7H), 2.09 (s, 3H), 1.86 (dq, J = 11.9, 9.2 Hz, 1H) |
| 1-102 | 3.43 | 446.1 | Method 2 | (500 MHz, CDCl₃): δ = 9.87 (s, 0.75H), 9.64 (s, 0.25H), 8.72 |
| | | | | (d, J = 2.7 Hz, 0.75H), 8.67 (dd, J = 5.3, 2.7 Hz, 0.25H), 8.60 |
| | | | | (d, J = 9.0 Hz, 0.25H), 8.52 (d, J = 8.1 Hz, 0.75H), 8.42 (dd, |
| | | | | J = 8.7, 2.7 Hz, 0.75H), 8.36 (s, 0.75H), 8.06 (dt, J = 9.0, 3.2 |
| | | | | Hz, 0.25H), 7.91 (d, J = 7.8 Hz, 0.25H), 7.77 (dd, J = 9.5, 2.1 |
| | | | | Hz, 1.5H), 7.73-7.65 (m, 1H), 7.48 (dd, J = 7.8, 1.4 Hz, |
| | | | | 0.25H), 7.31-7.19 (m, 1H), 6.94 (d, J = 8.2 Hz, 0.75H), 6.90 |
| | | | | (dd, J = 7.8, 4.0 Hz, 1.25H), 6.33 (s, 0.25H), 4.14-4.06 (m, |
| | | | | 1H), 3.93-3.87 (m, 3H), 3.84 (dq, J = 10.2, 4.9 Hz, 1H), 3.64 |
| | | | | (t, J = 5.1 Hz, 1H), 2.14 (s, 3H), 1.88 (b.s, 3H) |
| 1-103 | 3.52 | 471.2 | Method 2 | (500 MHz, CDCl₃): δ = 8.51 (dd, J = 8.0, 0.7 Hz, 1H), 8.46 (s, |
| | | | | 2H), 8.27 (d, J = 0.7 Hz, 1H), 7.72 (dd, J = 8.1, 1.6 Hz, 1H), |
| | | | | 7.64 (d, J = 1.6 Hz, 1H), 7.25 (t, J = 7.9 Hz, 1H), 6.91 (dd, J = |
| | | | | 8.3, 1.1 Hz, 1H), 6.88 (dd, J = 7.7, 1.1 Hz, 1H), 3.88 (s, 4H), |
| | | | | 3.71 (dtt, J = 12.6, 3.3, 1.5 Hz, 1H), 2.91-2.79 (m, 2H), 2.48 |
| | | | | (tt, J = 10.7, 3.8 Hz, 1H), 2.36 (s, 6H), 2.11 (s, 3H), 2.05 (dd, |
| | | | | J = 12.2, 4.0 Hz, 1H), 1.94-1.86 (m, 1H), 1.74-1.61 (m, |
| | | | | 1H), 1.44 (tdd, J = 12.6, 11.1, 3.9 Hz, 1H) |
| 1-104 | 3.50 | 459.2 | Method 2 | (500 MHz, CDCl₃): δ = 8.53 (d, J = 8.2 Hz, 1H), 8.37 (s, 2H), |
| | | | | 8.28 (d, J = 0.7 Hz, 1H), 7.74 (dd, J = 8.1, 1.6 Hz, 1H), 7.66 |
| | | | | (d, J = 1.6 Hz, 1H), 7.30-7.26 (m, 1H), 6.93 (dd, J = 8.4, 1.1 |
| | | | | Hz, 1H), 6.90 (dd, J = 7.7, 1.1 Hz, 1H), 4.97 (s, 1H), 3.90 (s, |
| | | | | 3H), 2.44 (s, 2H), 2.39 (s, 6H), 2.14 (s, 3H), 1.38 (s, 6H) |
| 1-105 | 3.38 | 417.1 | Method 2 | (500 MHz, CDCl₃): δ = 8.53 (d, J = 8.1 Hz, 1H), 8.35 (s, 2H), |
| | | | | 8.28 (s, 1H), 7.74 (dd, J = 8.2, 1.6 Hz, 1H), 7.66 (d, J = 1.6 |
| | | | | Hz, 1H), 7.27 (t, J = 5.9 Hz, 4H), 6.96-6.89 (m, 2H), 3.90 (s, |
| | | | | 3H), 3.51 (t, J = 6.6 Hz, 2H), 3.12 (s, 3H), 3.01 (t, J = 6.5 Hz, |
| | | | | 2H), 2.13 (s, 3H) |
| 1-106 | 3.72 | 455.2 | Method 2 | (500 MHz, CDCl₃): δ = 8.50 (d, J = 8.2 Hz, 1H), 8.29 (d, J = |
| | | | | 0.7 Hz, 1H), 8.20 (s, 2H), 8.16 (s, 1H), 7.99 (s, 1H), 7.76 (dd, |
| | | | | J = 8.2, 1.7 Hz, 1H), 7.68 (d, J = 1.6 Hz, 1H), 7.27 (t, J = 7.9 |
| | | | | Hz, 1H), 6.93 (dd, J = 8.2, 1.1 Hz, 1H), 6.90 (dd, J = 7.7, 1.1 |
| | | | | Hz, 1H), 5.02 (t, J = 6.1 Hz, 1H), 4.48-4.43 (m, 2H), 3.90 (s, |
| | | | | 3H), 3.71 (q, J = 5.9 Hz, 2H), 2.13 (s, 3H) |
| 1-107 | 3.30 | 443.2 | Method 2 | (500 MHz, CDCl₃): δ = 8.53 (d, J = 8.2 Hz, 1H), 8.28 (s, 1H), |
| | | | | 8.21 (s, 2H), 7.74 (dd, J = 8.2, 1.6 Hz, 1H), 7.66 (d, J = 1.7 |
| | | | | Hz, 1H), 7.27 (t, J = 7.9 Hz, 1H), 6.95-6.92 (m, 1H), 6.90 |
| | | | | (dd, J = 7.6, 1.1 Hz, 1H), 4.38 (d, J = 7.8 Hz, 1H), 4.06 (tq, |
| | | | | J = 8.0, 3.1 Hz, 1H), 3.90 (s, 3H), 2.91 (td, J = 9.1, 8.4, 3.6 Hz, |
| | | | | 1H), 2.72 (dd, J = 9.8, 2.7 Hz, 1H), 2.65 (dd, J = 9.9, 6.0 Hz, |
| | | | | 1H), 2.46-2.32 (m, 5H), 2.13 (s, 3H), 1.81-1.71 (m, 1H) |
| 1-108 | 3.73 | 370.2 | Method 2 | (500 MHz, CDCl₃): δ = 8.96 (d, J = 4.8 Hz, 2H), 8.52 (d, J = |
| | | | | 8.1 Hz, 1H), 8.32 (s, 1H), 7.78 (dd, J = 8.2, 1.6 Hz, 1H), 7.69 |
| | | | | (d, J = 1.6 Hz, 1H), 7.41 (t, J = 4.9 Hz, 1H), 7.18 (t, J = 7.9 |
| | | | | Hz, 1H), 6.69 (dd, J = 8.3, 1.1 Hz, 1H), 6.58 (dd, J = 7.5, 1.1 |
| | | | | Hz, 1H), 3.31-3.25 (m, 2H), 2.97 (s, 3H), 2.60 (t, J = 6.4 Hz, |
| | | | | 2H), 1.94-1.86 (m, 2H) |
| 1-109 | 4.51 | 446.3 | Method 2 | (500 MHz, CDCl₃): δ = 8.53 (d, J = 8.2 Hz, 1H), 8.39 (s, 2H), |
| | | | | 8.29 (s, 1H), 7.75 (dd, J = 8.2, 1.7 Hz, 1H), 7.67 (d, J = 1.6 |
| | | | | Hz, 1H), 7.27 (t, J = 7.9 Hz, 1H), 6.94 (d, J = 8.2 Hz, 1H), |
| | | | | 6.90 (dd, J = 7.7, 1.1 Hz, 1H), 4.35 (s, 1H), 3.91 (s, 3H), 3.42 |
| | | | | (s, 3H), 3.34 (s, 2H), 2.14 (s, 3H), 1.39 (s, 6H). |
| 1-110 | 4.08 | 446.3 | Method 2 | (500 MHz, CDCl₃): δ = 8.52 (d, J = 8.1 Hz, 1H), 8.45 (s, 2H), |
| | | | | 8.28 (d, J = 0.7 Hz, 1H), 7.74 (dd, J = 8.1, 1.7 Hz, 1H), 7.66 |
| | | | | (d, J = 1.6 Hz, 1H), 7.27 (t, J = 7.9 Hz, 1H), 6.93 (dd, J = 8.3, |
| | | | | 1.1 Hz, 1H), 6.90 (dd, J = 7.7, 1.1 Hz, 1H), 3.90 (s, 3H), 3.41 |
| | | | | (s, 2H), 3.17 (s, 3H), 2.13 (s, 3H), 1.64 (s, 2H), 1.33 (s, 6H). |
| 1-111 | 1.10 | 349.0 | Method 3 | — |
| 1-112 | 1.21 | 350.1 | Method 3 | — |

(RT means retention time in minutes; [MH]⁺ means the protonated mass of the compound (free base); nd = not determined).

### Pharmacology

The compounds provided in the present invention are negative allosteric modulators of mGlu7. As such, these compounds are expected to have their effect at mGlu7 by virtue of their ability to block the function of the receptor after binding to a site that is not the orthosteric glutamate recognition site.

Some of the compounds of Formula (I) have been tested according to the following methods.

### Example A

mGlu7 Assay on HEK-Expressing Human mGlu7

### Transfection and Cell Culture

The cDNA encoding the human metabotropic glutamate 7 receptor (hmGlu7), (accession number NM_181874.2, NCBI Nucleotide database browser), was subcloned into an expression vector containing also the hygromycin resistance gene. In parallel, the cDNA encoding a G protein allowing redirection of the activation signal to intracellular calcium flux was subcloned into a different expression vector containing also the Puromycin resistance gene. Transfection of both these vectors into HEK293 cells with PolyFect reagent (Qiagen) according to supplier's protocol, and hygromycin and puromycin treatment allowed selection of antibiotic resistant cells which had integrated stably one or more copies of the plasmids. Positive cellular clones expressing hmGlu7 were identified in a functional assay measuring changes in calcium fluxes in response to glutamate and L-AP4 or known mGlu7 orthosteric antagonists.

HEK-293 cells expressing hmGlu7 were maintained in media containing DMEM, Fetal Bovine Serum (10%), Glutamax™ (2 mM), penicillin (100 units/mL), streptomycin (100 μg/mL), geneticin (100 μg/mL) and hygromycin-B (40 μg/mL) and puromycin (1 μg/mL) at 37° C. with 5% CO₂ in a humidified atmosphere.

Fluorescent Cell Based- Ca⁺ Mobilization Assay Human mGlu7 HEK-293 cells were plated out 24 hours prior to a fluorescent cell-based calcium mobilization assay using FLIPR³⁸⁴ assay (Molecular Device, Sunnyvale, CA, USA) in black-walled, clear-bottomed, poly-L-ornithine-coated 384-well plates at a density of 25,000 cells/well in a glutamine/glutamate free DMEM medium containing fetal bovine serum (10%), penicillin (100 units/mL), streptomycin (100 μg/mL) and doxycyline (1 μg/ml) at 37° C. with 5% CO₂ in a humidified atmosphere.

On the day of the assay, the medium was aspirated and the cells were loaded with a 3 μM solution of Fluo4-AM (LuBioScience, Lucerne, Switzerland) in 0.03% pluronic acid. After 1 hour at 37° C./5% CO₂, the non incorporated dye was removed by washing cell plate with the assay buffer. All assays were performed in a pH 7.4 buffered-solution containing 20 mM HEPES, 143 mM NaCl, 6 mM KCl, 1 mM MgSO₄, 1 mM CaCl₂, 0.125 mM sulfinpyrazone and 0.1% glucose.

After 10 s of basal fluorescence recording, various concentrations of the compounds of the invention were added to the cells. Changes in fluorescence levels were first monitored for 180 s in order to detect any agonist activity of the compounds. Then the cells were stimulated by an EC₈₀ L-AP4 concentration for an additional 110 s in order to measure inhibiting activities of the compounds of the invention. EC₈₀ L-AP4 concentration is the concentration giving 80% of the maximal glutamate response.

The concentration-response curves of L-AP4 or representative compounds of the present invention were generated using the Prism GraphPad software (Graph Pad Inc, San Diego, USA). The curves were fitted to a four-parameter logistic equation:

$$( Y = Bottom + ( Top - Bottom ) / ( 1 + 10 \hat{} ( ( Log I {C}_{50} - X ) * Hill Slope )$$

allowing determination of IC₅₀ values.

The compounds of this application have IC₅₀ values less than 10 μM.

The Table 3 below represents the mean IC₅₀ obtained from at least three independent experiments of selected molecules performed in duplicate.

TABLE 3
Activity data for selected compounds

| Co. Nr. | Ca²⁺ Flux* |
|---|---|
| 1-1 | ++ |
| 1-2 | ++ |
| 1-3 | ++ |
| 1-4 | ++ |
| 1-5 | ++ |
| 1-6 | ++ |
| 1-7 | ++ |
| 1-8 | ++ |
| 1-9 | ++ |
| 1-10 | +++ |
| 1-11 | +++ |
| 1-12 | ++ |
| 1-13 | + |
| 1-14 | ++ |
| 1-15 | ++ |
| 1-16 | ++ |
| 1-17 | ++ |
| 1-18 | ++ |
| 1-19 | ++ |
| 1-20 | ++ |
| 1-21 | ++ |
| 1-22 | ++ |
| 1-23 | ++ |
| 1-24 | ++ |
| 1-25 | ++ |
| 1-26 | +++ |
| 1-27 | ++ |
| 1-28 | ++ |
| 1-29 | ++ |
| 1-30 | ++ |
| 1-31 | ++ |
| 1-32 | ++ |
| 1-33 | +++ |
| 1-34 | +++ |
| 1-35 | +++ |
| 1-36 | +++ |
| 1-37 | +++ |
| 1-38 | +++ |
| 1-39 | ++ |
| 1-40 | +++ |
| 1-41 | ++ |
| 1-42 | +++ |
| 1-43 | ++ |
| 1-44 | +++ |
| 1-45 | +++ |
| 1-46 | +++ |
| 1-47 | + |
| 1-48 | ++ |
| 1-49 | ++ |
| 1-50 | + |
| 1-51 | + |
| 1-52 | +++ |
| 1-53 | + |
| 1-54 | +++ |
| 1-55 | +++ |
| 1-56 | +++ |
| 1-57 | +++ |
| 1-58 | +++ |
| 1-59 | ++ |
| 1-60 | ++ |
| 1-61 | +++ |
| 1-62 | +++ |
| 1-63 | ++ |
| 1-64 | +++ |
| 1-65 | +++ |
| 1-66 | ++ |
| 1-67 | ++ |
| 1-68 | ++ |
| 1-69 | ++ |
| 1-70 | ++ |
| 1-71 | ++ |
| 1-72 | +++ |
| 1-73 | +++ |
| 1-74 | +++ |
| 1-75 | +++ |
| 1-76 | +++ |
| 1-77 | +++ |
| 1-78 | ++ |
| 1-79 | +++ |
| 1-80 | +++ |
| 1-81 | ++ |
| 1-82 | +++ |
| 1-83 | ++ |
| 1-84 | ++ |
| 1-85 | +++ |
| 1-86 | +++ |
| 1-87 | +++ |
| 1-88 | +++ |
| 1-89 | ++ |
| 1-90 | +++ |
| 1-91 | +++ |
| 1-92 | +++ |
| 1-93 | +++ |
| 1-94 | +++ |
| 1-95 | ++ |
| 1-96 | ++ |
| 1-97 | +++ |
| 1-98 | ++ |
| 1-99 | ++ |
| 1-100 | +++ |
| 1-101 | ++ |
| 1-102 | +++ |
| 1-103 | ++ |
| 1-104 | ++ |
| 1-105 | ++ |
| 1-106 | +++ |
| 1-107 | +++ |
| 1-108 | +++ |
| 1-109 | +++ |
| 1-110 | ++ |
| 1-111 | +++ |
| 1-112 | + |

*Table legend:
(+): 1 μM < IC₅₀ < 10 μM
(++): 100 nM < IC₅₀ < 1 μM
(+++): IC₅₀ < 100 nM

The results shown in Table 3 demonstrate that the compounds of the present invention are negative allosteric modulators of human mGlu7 receptors.

### Example B

### Water Associated Zero Maze:

The procedure was performed as described previously by Ritov and Richter-Levin, 2014 with minor modifications. The apparatus consists of annular platform with two opposite, enclosed quadrants (with walls 35 cm height) and two open quadrants (with borders 5 mm height). The plastic tank that holds this platform is filled up with water (22±2° C., 50 cm deep), arising to 10 cm below the platform level. Thus, the annular platform and the plastic tank comprise one unified arena. For the tests, rats were first habituated to the room for 4 min and then were placed into one of the open quadrants facing a closed part of the apparatus. Rats were allowed to explore the arena for a 5 mins session. During this time rats behavior was tracked, recorded and analyzed by the Etho-Vision system (Noldus Information Technology, Wageningen, Netherlands). Behavioral measures that were analyzed include the time spent in the open quadrants, distance traveled in the open quadrants, distance travelled in the closed quadrants and total freezing behavior. The impact of exposure to various stressors and/or compounds were assessed using these parameters. Pre-treatment time and route of administration of the different tested compounds were defined based of their pharmacokinetic properties.

### Example C

### Elevated Plus Maze:

The elevated plus maze (EPM) test was conducted using Sprague-Dawley male rats. The EPM is made of plastic that has two open arms (50 cm×10 cm) and two closed arms of the same size with walls 40 cm high, elevated 86 cm above the ground. Both arms are made of black Plexiglas. The average illumination level on the open arms was 187 LUX and 100 LUX on the closed arms. At the beginning of the experiment, rats were brought into a holding room directly next to the testing room and allowed to habituate to the environment for 30 min. At the commencement of testing, rats were placed in the center of the maze, facing one of the open arms and observed for 5 min. During this time rats behavior was tracked, recorded and analyzed by the Etho-Vision system (Noldus Information Technology, Wageningen, Netherlands). Behavioral measures that were analyzed include the time spent in the open arms, number of entries in the open arms as well as the distance travelled. Pre-treatment time and route of administration of the different tested compounds were defined based on their pharmacokinetic properties.

### Example D

### Fear Conditioning Model of Post-Traumatic Stress Disorder in the Rat:

The fear-conditioning arena (30 cm×20 cm×25 cm, Med Associates) is made of Plexiglas in different contexts. The system is placed in a sound-proof ventilated box. The arena floor consists of grid floor (19 parallel 0.48 cm diameter stainless steel rods, 1.6 cm apart) above a stainless steel waste pan. All rods were wired to a shock generator and scrambler. A speaker was mounted in the chamber wall to provide the source of the auditory stimuli. Fear conditioning procedure was performed over two days. The first day (training), rats were placed in the training context (context A) and after a 120 s acclimation period, they received five pairings of the CS and US. The CS tone (78 dB, 2 kHz, 5 ms rise/fall time) was presented for 30 s and co-terminated with a brief US footshock (0.5 s, 0.66 mA). The inter-tone interval (tone onset to next tone onset) ranged from 60 s. The conditioning chambers were cleaned between subjects with 70% ethanol. The time-spent freezing during delivery of the CS tone was scored (CS freezing). The second day (test day), animals were placed in a new context (context B) and were exposed to the CS (120 s) after 60 s of acclimation. Time-spent in freezing was measured during both acclimation and CS. Tested compounds were administrated prior and/or after training phase as well as testing phase. Pre-treatment time and route of administration of the different tested compounds were adjusted based of their pharmacokinetic properties.

### Example E

### Noise-Induced Hearing Loss (NIHL) Model in the Mice:

Young adult males CBA/CaJ mice were used to assess the effect of tested compound on NIHL. Animals were exposed to octave band noise (8-16 khz) at a sound pressure level of 110 dB over 2 hours. Tested compounds were administrated prior and/or after noise exposure. Hearing function were measured using using auditory brainstrem response (ABR) audiograms or Distorsion Product of Autoacoustic Emissions (DPOAE) at different timepoint 24 hours, 2 and 4 weeks post acoustic trauma. Pre-treatment time and route of administration of the different tested compounds were adjusted based of their pharmacokinetic properties. The experimental groups were compared to the vehicle treated group through the measure of, for example, ABR Threshold, or ABR Threshold shift.

### Example F

### Colorectal Distension Test of Visceral Pain in Rat.

Male stress-sensitive Wistar Kyoto rats (250-300 g) were used in this study. Animals were fasted overnight (16 h) and on the day of testing, were anaesthetised using isoflurane. 6 cm latex balloon was inserted into the colorectal cavity, 1 cm from the anus. The animals were allowed to recover for 20 min before colorectal distension commenced. The paradigm used is an ascending phasic distension from 0 mmHg to 80 mmHg over 8 min using a computer-driven electronic barostat. The parameters measured were the threshold pressure (mmHg) that evoked visually identifiable visceral pain behaviour, and the total number of pain behaviours. Postures defined as visceral pain behaviours were abdominal retractions and/or abdominal withdrawal reflex.

Tested compounds were administrated prior colorectal distension. Pre-treatment time and route of administration of the different tested compounds were adjusted based of their pharmacokinetic properties.

### Formulation Examples

Typical examples of recipes for the formulation of the invention are as follows:

### 1. Tablets

| | | |
|---|---|---|
| Active ingredient | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this Example, active ingredient can be replaced by the same amount of any of the compounds according to the present invention, in particular by the same amount of any of the exemplified compounds.

### 2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 mL.

### 3. Injectable

A parenteral composition is prepared by stirring 1.5% by weight of active ingredient of the invention in 10% by volume propylene glycol and water.

### 4. Ointment

| | | |
|---|---|---|
| Active ingredient | 5 to 1000 | mg |
| Stearyl alcohol | 3 | g |
| Lanoline | 5 | g |
| White petroleum | 15 | g |
| Water | ad 100 | g |

In this Example, active ingredient can be replaced with the same amount of any of the compounds according to the present invention, in particular by the same amount of any of the exemplified compounds.

1. A compound having the Formula (I): 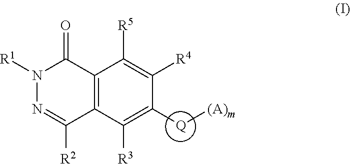
a pharmaceutically acceptable acid or base addition salt thereof, a stereochemically isomeric form thereof or an N-oxide form thereof, wherein:
Q is an optionally substituted aryl or heteroaryl which may further be substituted by 1 to 5 radicals (A)ₘ;
m is an integer ranging from 1 to 5;
the or each (A)ₘ is independently selected from the group of hydrogen, halogen, —CN, —OH, —NO₂, —CF₃, —OCF₃, —SH, —NH₂ and an optionally substituted radical selected from the group of —(C₁-C₆)alkyl, —(C₁-C₆)haloalkyl, —(C₂-C₆)alkynyl, —(C₂-C₆)alkenyl, —(C₃-C₇)cycloalkyl, —(C₁-C₆)alkylene-(C₃-C₇)cycloalkyl, —(C₃-C₈)cycloalkenyl, —(C₁-C₆)cyanoalkyl, —(C₁-C₆)alkylene-aryl, aryl, —(C₁-C₆)alkylene-heteroaryl, heteroaryl, —(C₁-C₆)alkylene-heterocycle, heterocycle, —(C₀-C₆)alkylene-OR⁶, —O—(C₂-C₆)alkylene-OR⁶, —NR⁶(C₂-C₆)alkylene-OR⁷, —(C₃-C₆)alkynylene-OR⁶, —(C₃-C₆)alkynylene-NR⁶R⁷, —(C₃-C₆)alkenylene-OR⁶, —(C₃-C₆)alkenylene-NR⁶R⁷, —(C₀-C₆)alkylene-S—R⁶, —O—(C₂-C₆)alkylene-S—R⁶, —NR⁶, —(C₂-C₆)alkylene-S—R⁷, —(C₀-C₆)alkylene-S(═O)—R⁶, —O—(C₁-C₆)alkylene-S(═O)—R⁶, —NR⁶—(C₁-C₆)alkylene-S(═O)—R⁷, —(C₀-C₆)alkylene-S(═O)₂—R⁶, —O—(C₁-C₆)alkylene-S(═O)₂—R⁶, —NR⁶—(C₁-C₆)alkylene-S(═O)₂—R⁷, —(C₀-C₆)alkylene-NR⁶R⁷, —O—(C₂-C₆)alkylene-NR⁶R⁷, —NR⁶—(C₂-C₆)alkylene-NR⁷R⁸, —(C₀-C₆)alkylene-S(═O)₂NR⁶R⁷, —O—(C₁-C₆)alkylene-S(═O)₂NR⁶R⁷, —NR⁶—(C₁-C₆)alkylene-S(═O)₂NR⁷R⁸, —(C₀-C₆)alkylene-NR⁶—S(═O)₂R⁷, —O—(C₂-C₆)alkylene-NR⁶—S(═O)₂R⁷, —NR⁶—(C₂-C₆)alkylene-NR⁷—S(═O)₂R⁸, —(C₀-C₆)alkylene-C(═O)—NR⁶R⁷, —O—(C₁-C₆)alkylene-C(═O)—NR⁶R⁷, —NR⁶—(C₁-C₆)alkylene-C(═O)—NR⁷R⁸, —(C₀-C₆)alkylene-NR⁶C(═O)—R⁷, —O—(C₂-C₆)alkylene-NR⁶C(═O)—R⁷, —NR⁶—(C₂-C₆)alkylene-NR⁷C(═O)—R⁸, —NR⁶C(═O)—(C₁-C₆)alkylene-OR⁷, —NR⁶C(═O)—(C₁-C₆)alkylene-NR⁷R⁸, —(C₀-C₆)alkylene-OC(═O)—R⁶, —O—(C₂-C₆)alkylene-OC(═O)—R⁶, —NR⁶—(C₂-C₆)alkylene-OC(═O)—R⁷, —(C₀-C₆)alkylene-C(═O)—OR⁶, —O—(C₁-C₆)alkylene-C(═O)—OR⁶, —NR⁶—(C₁-C₆)alkylene-C(═O)—OR⁷, —(C₀-C₆)alkylene-C(═O)—R⁶, —O—(C₁-C₆)alkylene-C(═O)—R⁶, —NR⁶—(C₁-C₆)alkylene-C(═O)—R⁷, —C(═O)—(C₁-C₆)alkylene-OR⁶, —C(═O)—(C₁-C₆)alkylene-NR⁶R⁷, —(C₀-C₆)alkylene-NR⁶—C(═O)—OR⁷, —C(═O)—(C₁-C₆)alkylene-NR⁶—C(═O)—OR⁷, —(C₀-C₆)alkylene-O—C(═O)—NR⁶R⁷, —(C₀-C₆)alkylene-NR⁶—C(═O)—NR⁷R⁸, —O—(C₂-C₆)alkylene-NR⁶, —C(═O)—NR⁷R⁸, —NR⁶—(C₂-C₆)alkylene-NR⁷—C(═O)—NR⁸R⁹ and —(C₀-C₆)alkylene-NR⁶—C(═NR⁷)—NR⁸R⁹;
R¹¹ is an optionally substituted —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₂-C₆)alkylene-O—(C₀-C₆)alkyl, aryl, —(C₁)alkylene-aryl, heterocycle, heteroaryl or —(C₁)alkylene-heteroaryl, wherein the aryl, heterocycle or heteroaryl ring can be substituted by 1 to 5 independent (B)ₙ radicals;
n is an integer ranging from 1 to 5;
the or each (B)ₙ is independently selected from the group of hydrogen, halogen, —CN, —OH, —NO₂, —CF₃, —OCF₃, —SH, —NH₂ and an optionally substituted radical selected from the group of —(C₁-C₆)alkyl, —(C₁-C₆)haloalkyl, —(C₂-C₆)alkynyl, —(C₂-C₆)alkenyl, —(C₃-C₇)cycloalkyl, —(C₁-C₆)alkylene-(C₃-C₇)cycloalkyl, —(C₃-C₈)cycloalkenyl, —(C₁-C₆)cyanoalkyl, —(C₁-C₆)alkylene-aryl, aryl, —(C₁-C₆)alkylene-heteroaryl, heteroaryl, —(C₁-C₆)alkylene-heterocycle, heterocycle, —(C₀-C₆)alkylene-OR¹⁰, —O—(C₂-C₆)alkylene-OR¹⁰, —NR¹⁰ (C₂-C₆)alkylene-OR¹¹, —(C₃-C₆)alkynylene-OR¹⁰, —(C₃-C₆)alkynylene-NR¹⁰R¹¹, —(C₃-C₆)alkenylene-OR¹⁰, —(C₃-C₆)alkenylene-NR¹⁰R¹¹, —(C₀-C₆)alkylene-S—R¹⁰, —O—(C₂-C₆)alkylene-S—R¹⁰, —NR¹⁰—(C₂-C₆)alkylene-S—R¹¹, —(C₀-C₆)alkylene-S(═O)—R¹⁰, —O—(C₁-C₆)alkylene-S(═O)—R¹⁰, —NR¹⁰—(C₁-C₆)alkylene-S(═O)—R¹¹, —(C₀-C₆)alkylene-S(═O)₂—R¹⁰, —O—(C₁-C₆)alkylene-S(═O)₂—R¹⁰, —NR¹⁰—(C₁-C₆)alkylene-S(═O)₂—R¹¹, —S(═O)(═NH)—R¹⁰, —(C₀-C₆)alkylene-NR¹⁰R¹¹, —O—(C₂-C₆)alkylene-NR¹⁰R¹¹, —NR¹⁰—(C₂-C₆)alkylene-NR¹¹R¹², —(C₀-C₆)alkylene-S(═O)₂NR¹⁰R¹¹, —O—(C₁-C₆)alkylene-S(═O)₂NR¹⁰R¹¹, —NR¹⁰—(C₁-C₆)alkylene-S(═O)₂NR¹¹R¹², —(C₀-C₆)alkylene-NR¹⁰, —S(═O)₂R¹¹, —O—(C₂-C₆)alkylene-NR¹⁰—S(═O)₂R¹¹, —NR¹⁰—(C₂-C₆)alkylene-NR¹¹—S(═O)₂R¹², —(C₀-C₆)alkylene-C(═O)—NR¹⁰R¹¹, —O—(C₁-C₆)alkylene-C(═O)—NR¹⁰R¹¹, —NR¹⁰—(C₁-C₆)alkylene-C(═O)—NR¹¹R¹², —(C₀-C₆)alkylene-NR¹⁰C(═O)—R¹¹, —O—(C₂-C₆)alkylene-NR¹⁰C(═O)—R¹¹, —NR¹⁰—(C₂-C₆)alkylene-NR¹¹C(═O)—R¹², —NR¹⁰C(═O)—(C₁-C₆)alkylene-OR¹¹, —NR¹⁰C(═O)—(C₁-C₆)alkylene-NR¹¹R¹², —(C₀-C₆)alkylene-OC(═O)—R¹⁰, —O—(C₂-C₆)alkylene-OC(═O)—R¹⁰, —NR¹⁰—(C₁-C₆)alkylene-OC(═O)—R¹¹, —(C₀-C₆)alkylene-C(═O)—OR¹⁰, —O—(C₁-C₆)alkylene-C(═O)—OR¹⁰, —NR¹⁰—(C₁-C₆)alkylene-C(═O)—OR¹¹, —(C₀-C₆)alkylene-C(═O)—R¹⁰, —O—(C₁-C₆)alkylene-C(═O)—R¹⁰, —NR¹⁰—(C₁-C₆)alkylene-C(═O)—R¹¹, —C(═O)—(C₁-C₆)alkylene-OR¹⁰, —C(═O)—(C₁-C₆)alkylene-NR¹⁰R¹¹, —(C₀-C₆)alkylene-NR¹⁰—C(═O)—OR¹¹, —C(═O)—(C₁-C₆)alkylene-NR¹⁰—C(═O)—OR¹¹, —(C₀-C₆)alkylene-O—C(═O)—NR¹⁰R¹¹, —(C₀-C₆)alkylene-NR¹⁰—C(═O)—NR¹¹R¹², —O—(C₂-C₆)alkylene-NR¹⁰—C(═O)—NR¹¹R¹², —NR¹⁰—(C₂-C₆)alkylene-NR¹⁰—C(═O)—NR¹²R¹³ and —(C₀-C₆)alkylene-NR¹⁰—C(═NR¹¹)—NR¹²R¹³;
optionally two radicals B are combined with the intervening atoms to form a 3 to 10 membered bicyclic heterocycle ring; wherein each ring is optionally further substituted with 1 to 5 radicals independently selected from the group of halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂;
R² is selected from the group of hydrogen, halogen, —CN, —NO₂, —CF₃ and an optionally substituted radical selected from the group of —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₂-C₆)alkylene-heterocycle, —(C₁-C₆)alkylene-OR¹⁴, —NR¹⁴(C₂-C₆)alkylene-OR¹⁵, —(C₀-C₆)alkylene-S—R¹⁴, —(C₀-C₆)alkylene-S(═O)—R¹⁴, —(C₀-C₆)alkylene-S(═O)₂—R¹⁴, —(C₀-C₆)alkylene-NR¹⁴R¹⁵, —NR¹⁴—(C₂-C₆)alkylene-NR¹⁵R¹⁶, —(C₀-C₆)alkylene-S(═O)₂NR¹⁴R¹⁵, —(C₀-C₆)alkylene-NR¹⁴—S(═O)₂R¹⁵, —(C₀-C₆)alkylene-C(═O)—N¹⁴R¹⁵, —(C₀-C₆)alkylene-NR¹⁴C(═O)—R¹⁵, —(C₁-C₆)alkylene-OC(═O)—R¹⁴, —(C₀-C₆)alkylene-C(═O)—OR¹⁴, —(C₀-C₆)alkylene-C(═O)—R¹⁴, —(C₀-C₆)alkylene-NR¹⁴—C(═O)—OR¹⁵, —(C₀-C₆)alkylene-O—C(═O)—NR¹⁴R¹⁵ —(C₀-C₆)alkylene-NR¹⁴—C(═O)—NR¹⁵R¹⁶ and —(C₀-C₆)alkylene-NR¹⁴—C(═NR¹⁵)—NR¹⁶R¹⁷;
R³ and R⁴ are each independently selected from the group of hydrogen, halogen, —CN, —OH, —NO₂, —CF₃, —OCF₃, —SH, —NH₂ and an optionally substituted radical selected from the group of —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, —(C₁-C₆)alkylene-heteroaryl, heteroaryl, heterocycle, —(C₁-C₆)alkylene-heterocycle, —(C₀-C₆)alkylene-OR¹⁸, —O—(C₂-C₆)alkylene-OR¹⁸, —NR¹(C₂-C₆)alkylene-OR¹⁹, —(C₀-C₆)alkylene-S—R¹⁸, —(C₀-C₆)alkylene-S(═O)—R¹⁹, —(C₀-C₆)alkylene-S(═O)₂—R¹⁸, —(C₀-C₆)alkylene-NR¹⁸R¹⁹, —O—(C₂-C₆)alkylene-NR¹⁸R¹⁹, —NR¹⁸(C₂-C₆)alkylene-NR¹⁹R²⁰, —(C₀-C₆)alkylene-S(═O)₂NR¹⁸R¹⁹, —(C₀-C₆)alkylene-NR¹⁸—S(═O)₂R¹⁹, —(C₀-C₆)alkylene-C(═O)—NR¹⁸R¹⁹, —(C₀-C₆)alkylene-NR¹⁸C(═O)—R¹⁹, —(C₀-C₆)alkylene-OC(═O)—R¹⁸, —(C₀-C₆)alkylene-C(═O)—OR¹⁹, —(C₀-C₆)alkylene-C(═O)—R¹¹, —(C₀-C₆)alkylene-NR¹⁸—C(═O)—OR¹⁹, —(C₀-C₆)alkylene-O—C(═O)—NR¹⁸R¹⁹, —(C₀-C₆)alkylene-NR¹⁸—C(═O)—NR¹⁹R²⁰ and —(C₀-C₆)alkylene-NR¹⁸—C(═NR¹⁹)—NR²⁰R²¹;
R⁵ is independently selected from the group of hydrogen, halogen, —CN, —OH, —NO₂, —CF₃, —OCF₃, —SH and an optionally substituted radical selected from the group of —(C₁-C₆)alkyl, —(C₁-C₆)haloalkyl, —(C₃-C₇)cycloalkyl, —(C₁-C₆)alkylene-(C₃-C₇)cycloalkyl, —(C₁-C₆)cyanoalkyl, —(C₁-C₆)alkylene-aryl, aryl, —(C₁-C₆)alkylene-heteroaryl, heteroaryl, heterocycle, —(C₁-C₆)alkylene-heterocycle, —(C₀-C₆)alkylene-OR²², —O—(C₂-C₆)alkylene-OR²², —NR²²(C₂-C₆)alkylene-OR²³, —(C₀-C₆)alkylene-S—R²², —(C₀-C₆)alkylene-S(═O)—R²², —(C₀-C₆)alkylene-S(═O)₂—R²², —(C₁-C₆)alkylene-NR²²R²³, —O—(C₂-C₆)alkylene-NR²²R²³, —NR²²—(C₂-C₆)alkylene-NR²³R²⁴, —(C₀-C₆)alkylene-S(═O)₂NR²²R²³, —(C₀-C₆)alkylene-NR²²—S(═O)₂R²³, —(C₀-C₆)alkylene-C(═O)—NR²²R²³, —(C₀-C₆)alkylene-NR²²C(═O)—R²³, —(C₀-C₆)alkylene-OC(═O)—R²², —(C₀-C₆)alkylene-C(═O)—OR²², —(C₀-C₆)alkylene-C(═O)—R²², —(C₀-C₆)alkylene-NR²²—C(═O)—OR²³, —(C₀-C₆)alkylene-O—C(═O)—NR²²R²³, —(C₀-C₆)alkylene-NR²²—C(═O)—NR²³R²⁴ and —(C₀-C₆)alkylene-NR²²—C(═NR²³)—NR²⁴R²⁵ and
R⁶, R⁷, R⁸, R⁹, R¹⁰, R¹¹, R¹², R¹³, R¹⁴, R¹⁵, R¹⁶, R¹⁷, R¹⁸, R¹⁹R²⁰, R²¹, R²², R²³, R²⁴ and R²⁵ are each independently hydrogen or an optionally substituted radical selected from the group of —(C₁-C₆)haloalkyl, —(C₁-C₆)alkyl, —(C₁-C₆)cyanoalkyl, —(C₃-C₇)cycloalkyl, —(C₁-C₆)alkylene-(C₃-C₇)cycloalkyl, heteroaryl, —(C₁-C₆)alkylene-heteroaryl, aryl, —(C₁-C₆)alkylene-aryl, —(C₁-C₆)alkylene-heterocycle, heterocycle, —(C₀-C₆)alkylene-O—(C₀-C₆)alkyl and (C₀-C₆)alkylene-N—((C₀-C₆)alkyl)₂.

2. The compound of claim 1 having the Formula (I),
provided that: 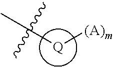
is not 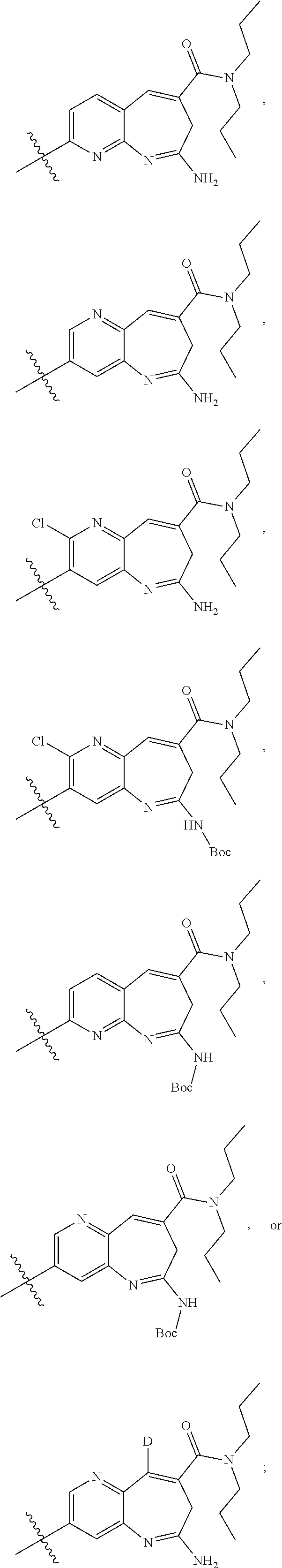 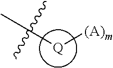
is not 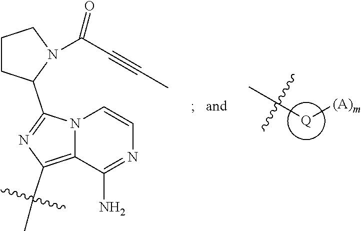
is not

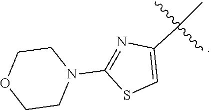 [SMILES: *c1csc(N2CCOCC2)n1]

3. The compound according to claim 1 or 2 having the Formula (I), provided that when 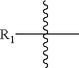
is pyridyl, B(n) is not a substituted pyrrolidinyl radical, and when 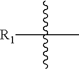
is pyridyl, the pyridyl is not substituted by methyl and a substituted pyrrolidinyl radical.

4. The compound according to claim 1 having the Formula (I),
provided that R² is not 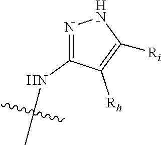
Rₕ wherein Rₕ is hydrogen, (C₁-C₆)alkyl, cyano or halogen, and Rᵢ is hydrogen, (C₁-C₆)alkyl or (C₃-C₇)cycloalkyl; and provided that R² is not

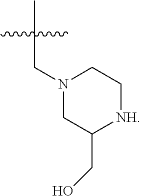 [SMILES: *CN1CCNC(CO)C1]

5. The compound according to claim 1 having Formula (I), provided that R¹ is not 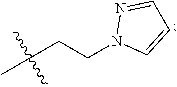 [SMILES: *CCn1cccn1]
Q is not naphthyl, benzothiophenyl or quinolinyl; and Q is not

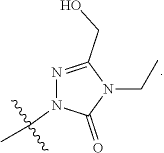 [SMILES: *n1nc(CO)n(CC)c1=O]

6. The compound according to claim 1 having the Formula (I) wherein:
Q represents an aryl or heteroaryl group of formula: 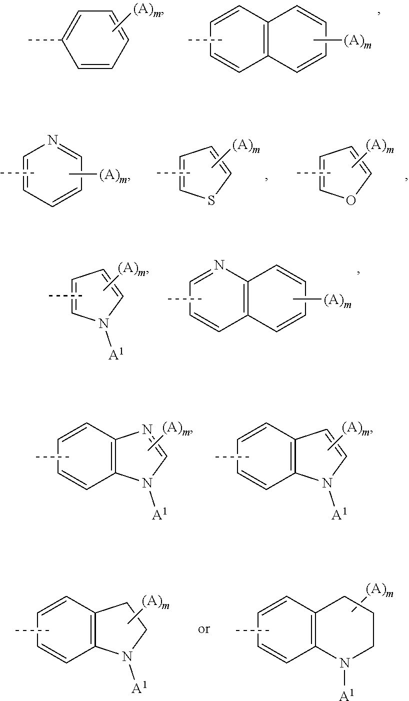
wherein each radical is optionally substituted with m radicals A, wherein m is an integer equal to zero, 1, 2, 3, 4 or 5, and A¹ is a radical A.

7. The compound according to claim 6 having the Formula (I) wherein:
Q represents an aryl or heteroaryl group of formula: 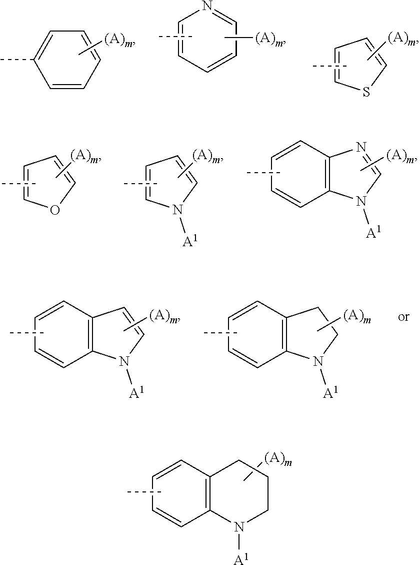
wherein each radical is optionally substituted with m radicals A, wherein m is an integer equal to zero, 1, 2, 3, 4 or 5, and A¹ is a radical A.

8. The compound according to claim 6 or 7 having the Formula (I) whererin A¹ is hydrogen, —(C₁-C₆)alkyl or —(C₃-C₇)cycloalkyl.

9. The compound according to claim 1 having the Formula (I) wherein:
the cycloalkyl, heterocycle, aryl and heteroaryl ring systems of (A)ₘ is selected from the group of azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl, and each ring of said ring system may be optionally substituted independently with 1 to 4 substituents R⁶, R⁷, R⁸ or R⁹.

10. The compound according to claim 1 having the Formula (I), wherein (A)ₘ are each independently selected from the group of hydrogen, halogen, —CF₂CH₃, —OCHF₂ and an optionally substituted radical selected from the group of —(C₁-C₆)alkyl, —(C₃-C₇)cycloalkyl, heterocycle and —(C₀-C₆)alkylene-OR⁶; wherein R⁶ is selected from the group of hydrogen, —(C₁-C₆)alkyl and —(C₃-C₇)cycloalkyl.

11. The compound according to claim 1 having the Formula (I) wherein:
the cycloalkyl, heterocycle, aryl and heteroaryl ring systems of (B)ₙ is selected from the group of azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl, and each ring of said ring system may be optionally substituted independently with 1 to 4 substituents R¹⁰, R¹¹, R¹² or R¹³.

12. The compound according to claim 1 having the Formula (I), wherein the or each (B)ₙ is independently selected from the group of hydrogen, halogen and an optionally substituted radical selected from the group of —(C₁-C₆)alkyl, heterocycle, —(C₀-C₆)alkylene-OR¹⁰, —O—(C₂-C₆)alkylene-OR¹⁰, —NR¹⁰ (C₂-C₆)alkylene-OR¹¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-NR¹⁰R¹¹, —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹;
optionally two radicals B are combined with the intervening atoms to form a 3 to 10 membered bicyclic heterocycle ring; wherein each ring is optionally further substituted with 1 to 5 radicals independently selected from the group of halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and R¹⁰, R¹¹ and R¹² are each independently selected from the group of hydrogen, —(C₁-C₆)alkyl, —(C₃-C₇)cycloalkyl, heterocycle, —(C₁-C₆)alkylene-heterocycle, —(C₁-C₆)alkylene-heteroaryl and —(C₀-C₆)alkylene-O—(C₀-C₆)alkyl.

13. The compound according to claim 1 having the Formula (I) wherein:
the cycloalkyl, heterocycle, aryl and heteroaryl ring systems of R¹, R², R³, R⁴ or R⁵ may be independently selected from the group of azetidinyl, benzimidazolyl, benzisothiazolyl benzisoxazolyl, benzofuryl, benzopyrazolyl, benzothiazolyl, benzothiophenyl, benzotriazolyl, benzoxazolyl, dihydrofuranyl, dihydrothienyl, dioxolanyl, 1,1-dioxo-thiomorpholinyl, furazanyl, furyl, imidazolidinyl, imidazolinyl, imidazolonyl, imidazolyl, imidazopyridazinyl, imidazopyridyl, indolyl, isoindolyl, isoquinolinyl, isothiazolinyl, isothiazolyl, isoxazolidinyl, isoxazolinyl, isoxazolyl, morpholinyl, naphthyl, naphthyridinyl, oxadiazolyl, oxazolidinyl, oxazolinyl, oxazolonyl, oxazolopyridazinyl, oxazolopyridyl, oxazolyl, oxetanyl, phenyl, piperazinonyl, piperazinyl, piperidinonyl, piperidinyl, phtalazinyl, pteridinyl, purinyl, pyranyl, pyrazinyl, pyrazolopyridinyl, pyrazolyl, pyridazinyl, pyridonyl, pyridyl, pyrimidyl, pyrrolidinonyl, pyrrolidinyl, pyrrolinyl, pyrrolyl, quinazolyl, quinolyl, quinoxalinyl, tetrahydrofuranyl, tetrahydropyranyl, tetrahydrothiopyranyl, tetrahydrotriazolopyridyl, tetrahydrotriazolopyrimidinyl, tetrazolyl, thiadiazolyl, thiazolidinyl, thiazolinyl, thiazolonyl, thiazolopyridazinyl, thiazolopyridyl, thiazolyl, thienyl, thiomorpholinyl, thionaphthyl, thiopyranyl, triazolinyl, triazinyl, triazolyl, cyclopropyl, cyclobutyl, cyclopentyl, cyclopentenyl, cyclohexyl, cyclohexenyl, cycloheptyl, cycloheptenyl, cyclooctyl and cyclooctenyl, and each ring of said ring system may be optionally substituted with 1-5 radicals independently selected from the group of hydrogen, halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂.

14. The compound according to claim 1 having Formula (I), wherein R², R³, R⁴ and R⁵ are each independently selected from hydrogen or (C₁-C₆)alkyl.

15. The compound according to claim 1 having Formula (I), wherein R¹ is heteroaryl optionally substituted by 1 to 5 independent (B)ₙ radicals; and Q represents an aryl or heteroaryl group of formula: 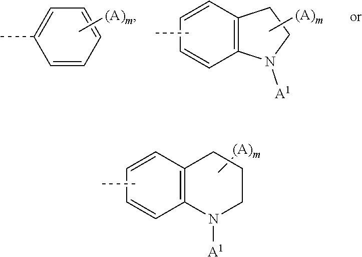
optionally substituted by 1 to 5 radicals (A)ₘ.

16. The compound according to claim 1 having the Formula (II): 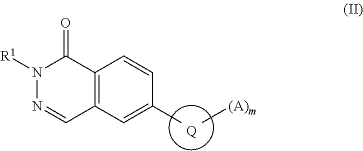
a pharmaceutically acceptable acid or base addition salt thereof, a stereochemically isomeric form thereof or an N-oxide form thereof.

17. The compound according to claim 16 having the Formula (III) 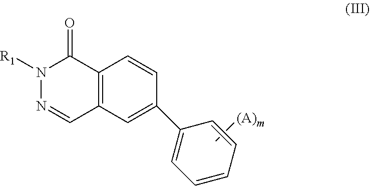
a pharmaceutically acceptable acid or base addition salt thereof, a stereochemically isomeric form thereof or an N-oxide form thereof.

18. The compound according to claim 16 having the Formula (IV) 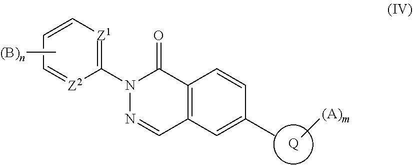
a pharmaceutically acceptable acid or base addition salt thereof, a stereochemically isomeric form thereof or an N-oxide form thereof; wherein Z¹ and Z² are each independently selected from C or N.

19. The compound according to claim 18 having the Formula (IV) wherein
Q represents an aryl or heteroaryl group of formula:

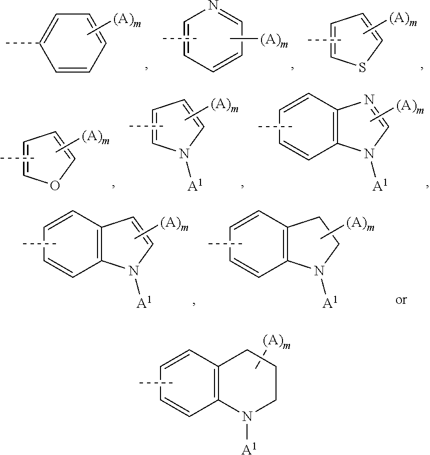

20. The compound according to claim 16 having the Formula (V): 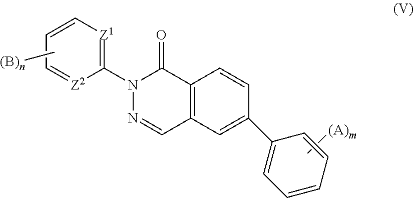
a pharmaceutically acceptable acid or base addition salt thereof, a stereochemically isomeric form thereof or an N-oxide form thereof, wherein:
Z¹ and Z² are each independently selected from C or N.

21. The compound according to claim 20 having the Formula (VI): 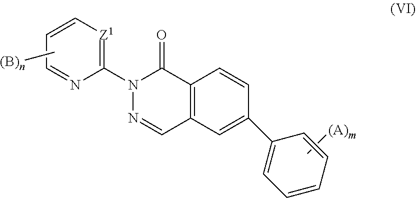
a pharmaceutically acceptable acid or base addition salt thereof, a stereochemically isomeric form thereof or an N-oxide form thereof, wherein:
Z¹ is selected from C or N;
the or each (A)ₘ is independently selected from the group of hydrogen, halogen, —CF₂CH₃, —OCHF₂ and an optionally substituted radical selected from the group of —(C₁-C₆)alkyl, —(C₃-C₇)cycloalkyl, heterocycle and —(C₀-C₆)alkylene-OR⁶;
R⁶ is selected from the group of hydrogen, —(C₁-C₆)alkyl and —(C₃-C₇)cycloalkyl;
the or each (B)ₙ is independently selected from the group of hydrogen, halogen and an optionally substituted radical selected from the group of —(C₁-C₆)alkyl, heterocycle, —(C₀-C₆)alkylene-OR¹⁰, —O—(C₂-C₆)alkylene-OR¹⁰, —NR¹⁰ (C₂-C₆)alkylene-OR¹¹, —(C₀-C₆)alkylene-S—R¹⁰, —(C₀-C₆)alkylene-S(═O)₂—R¹⁰, —S(═O)(═NH)—R¹⁰, —(C₀-C₆)alkylene-N¹⁰R¹¹, —NR¹⁰—(C₂-C₆)alkylene-NR¹¹R¹², —(C₀-C₆)alkylene-NR¹⁰C(═O)—R¹¹ and —(C₀-C₆)alkylene-C(═O)—NR¹⁰R¹¹;
optionally two radicals B are combined with the intervening atoms to form a 3 to 10 membered bicyclic heterocycle ring; wherein each ring is optionally further substituted with 1 to 5 radicals independently selected from the group of halogen, —CN, nitro, —(C₁-C₆)alkyl, —(C₀-C₆)alkylene-O—(C₀-C₆)alkyl and —(C₀-C₆)alkylene-N—((C₀-C₆)alkyl)₂; and
R¹⁰, R¹¹ and R¹² are each independently selected from the group of hydrogen, —(C₁-C₆)alkyl, —(C₃-C₇)cycloalkyl, heterocycle, —(C₁-C₆)alkylene-heterocycle, —(C₁-C₆)alkylene-heteroaryl and —(C₀-C₆)alkylene-O—(C₀-C₆)alkyl.

22. The compound according to claim 1, wherein the compound is an optical isomer, and wherein the compound is either a racemic mixture or one or both of the individual optical isomers.

23. The compound according to claim 1, wherein said compound is one or more selected from: 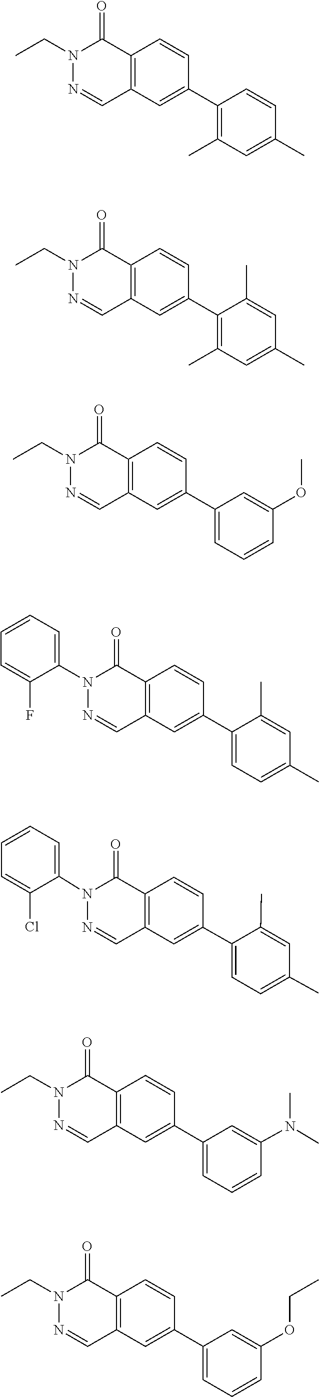 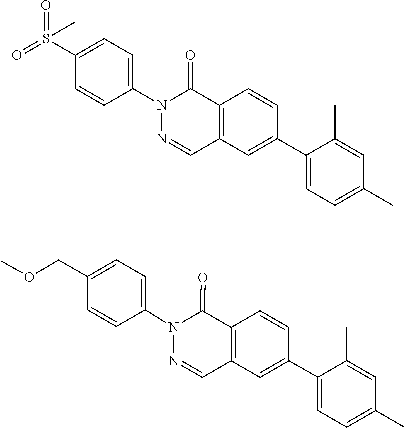 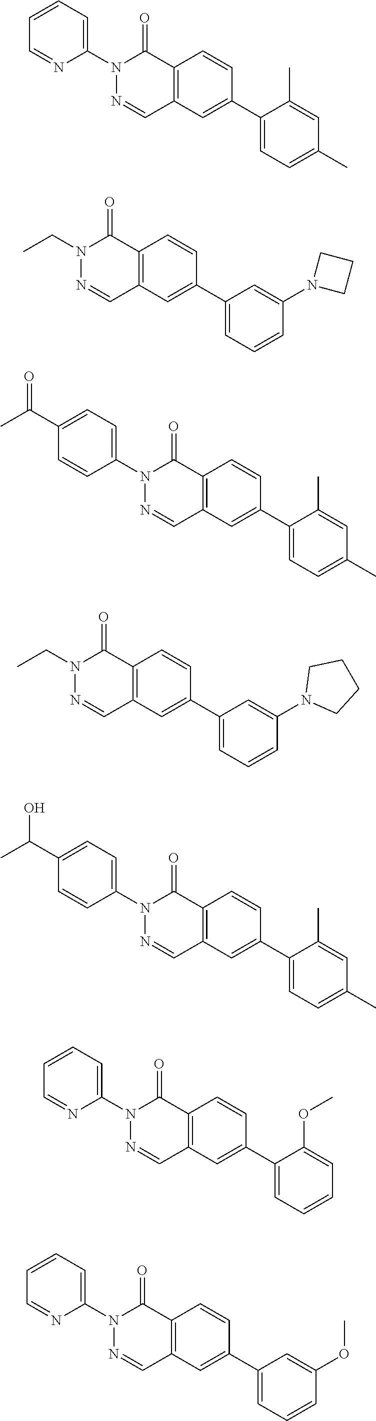 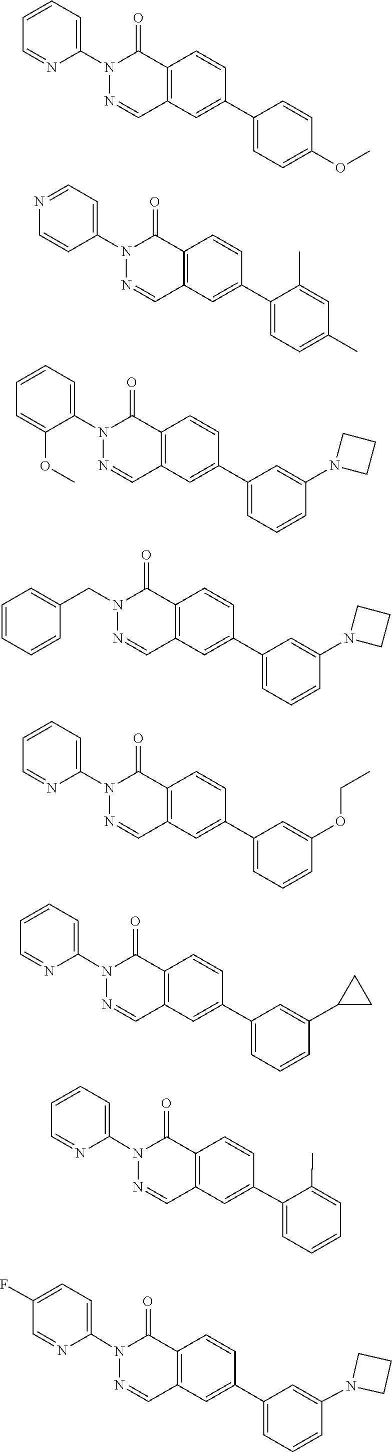 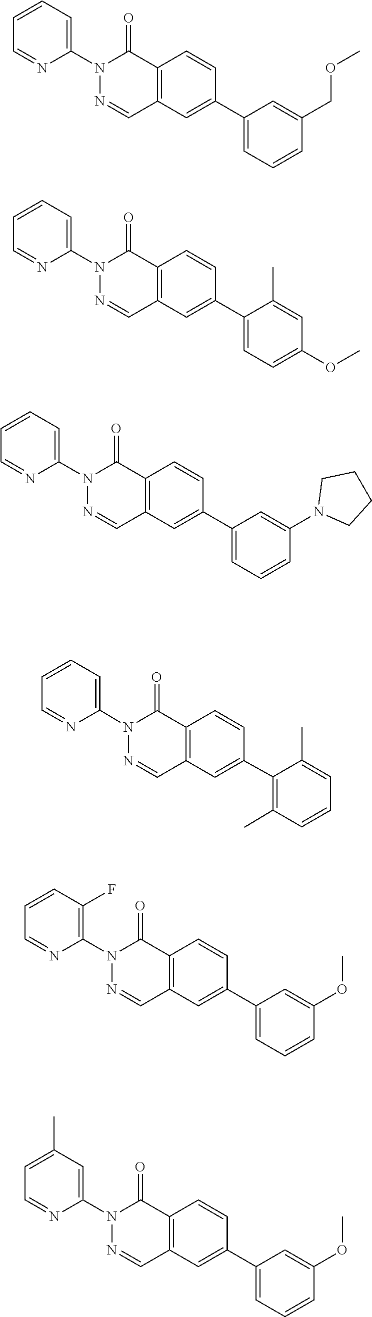 [SMILES: COCc1cccc(-c2ccc3c(=O)n(-c4ccccn4)ncc3c2)c1 ; COc1ccc(-c2ccc3c(=O)n(-c4ccccn4)ncc3c2)c(C)c1 ; O=c1c2ccc(-c3cccc(N4CCCC4)c3)cc2cnn1-c1ccccn1 ; Cc1cccc(C)c1-c1ccc2c(=O)n(-c3ccccn3)ncc2c1 ; COc1cccc(-c2ccc3c(=O)n(-c4ncccc4F)ncc3c2)c1 ; COc1cccc(-c2ccc3c(=O)n(-c4cc(C)ccn4)ncc3c2)c1] 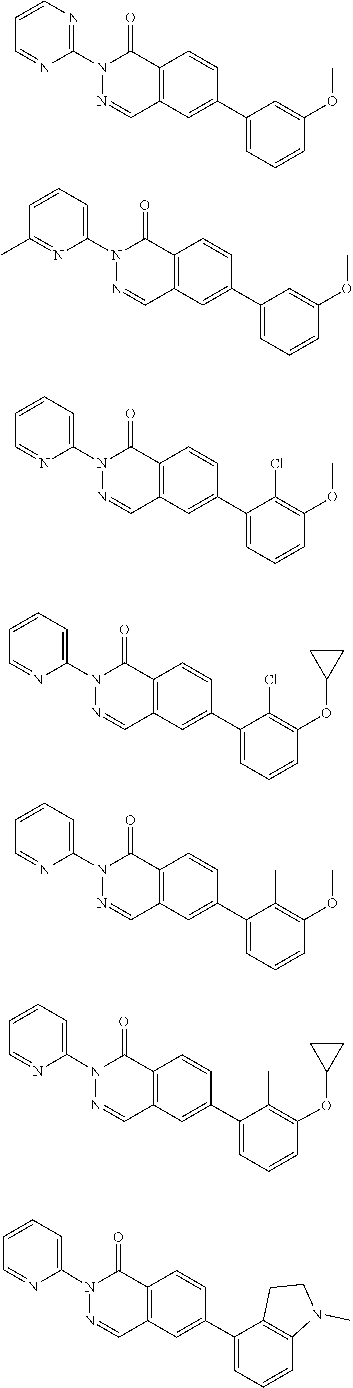 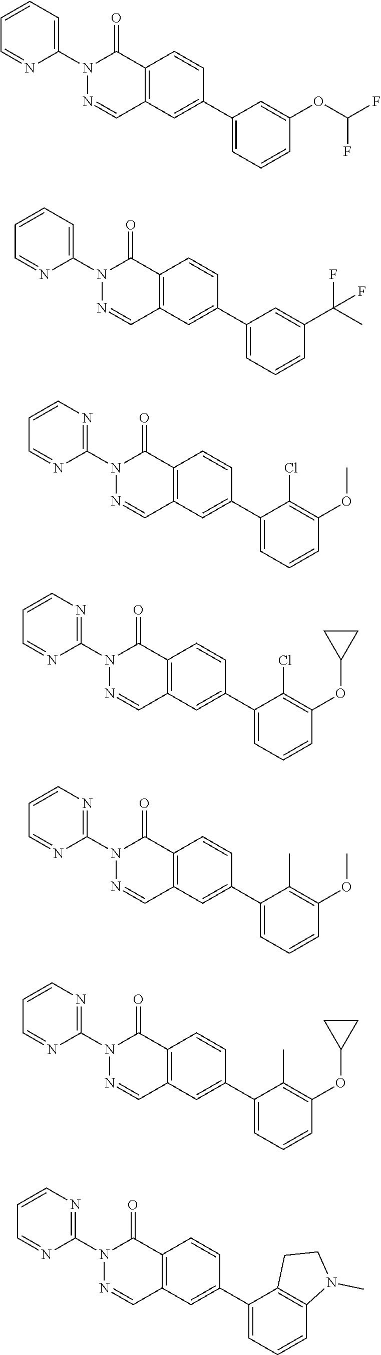 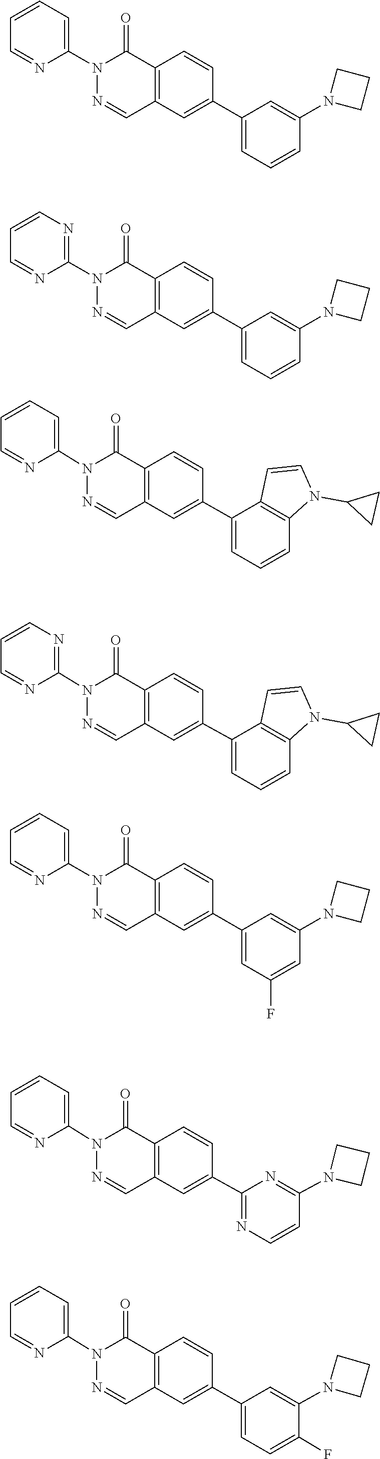 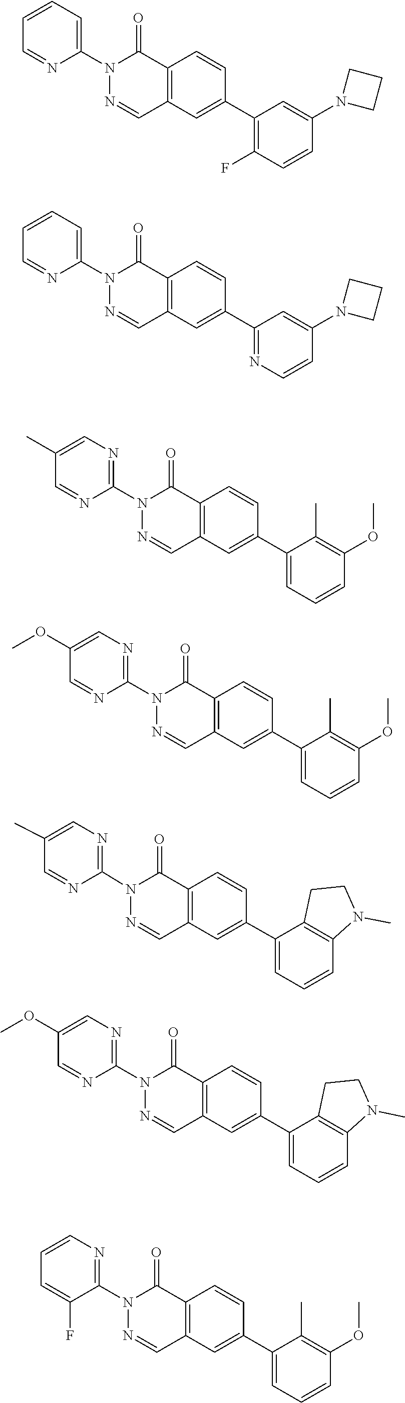 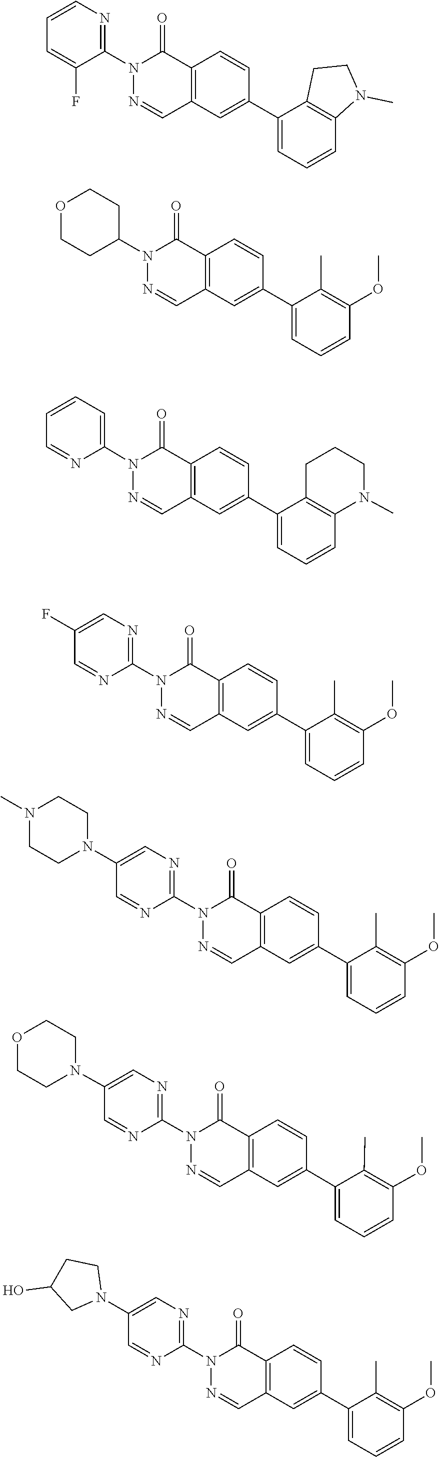 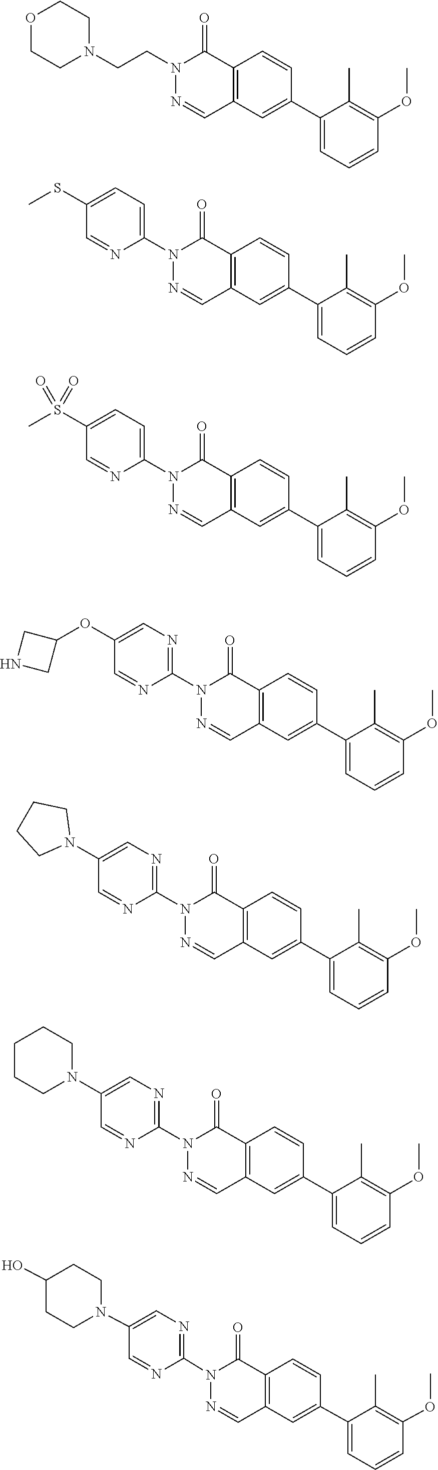 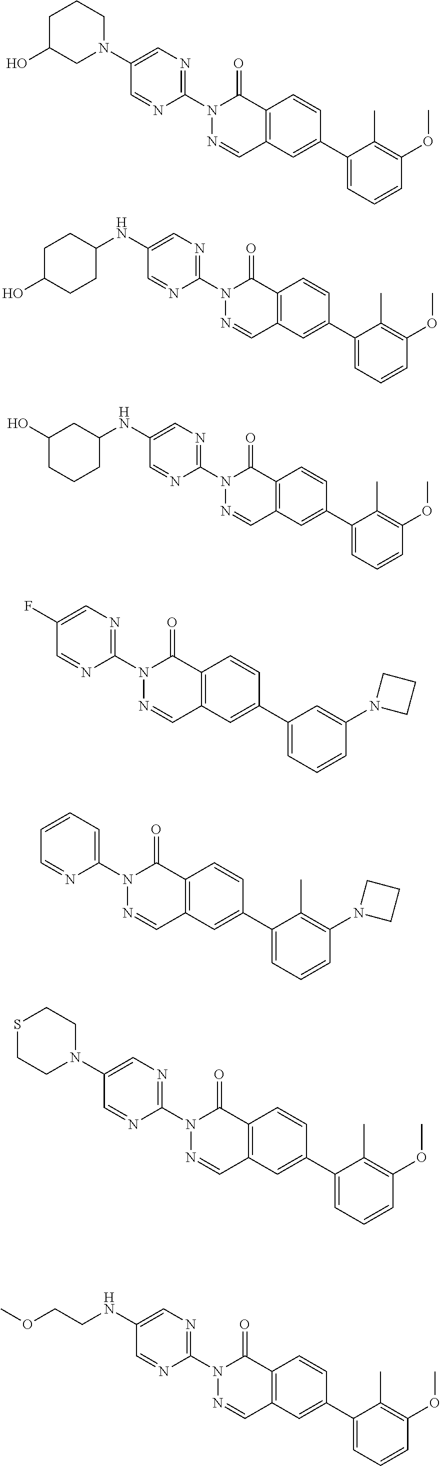 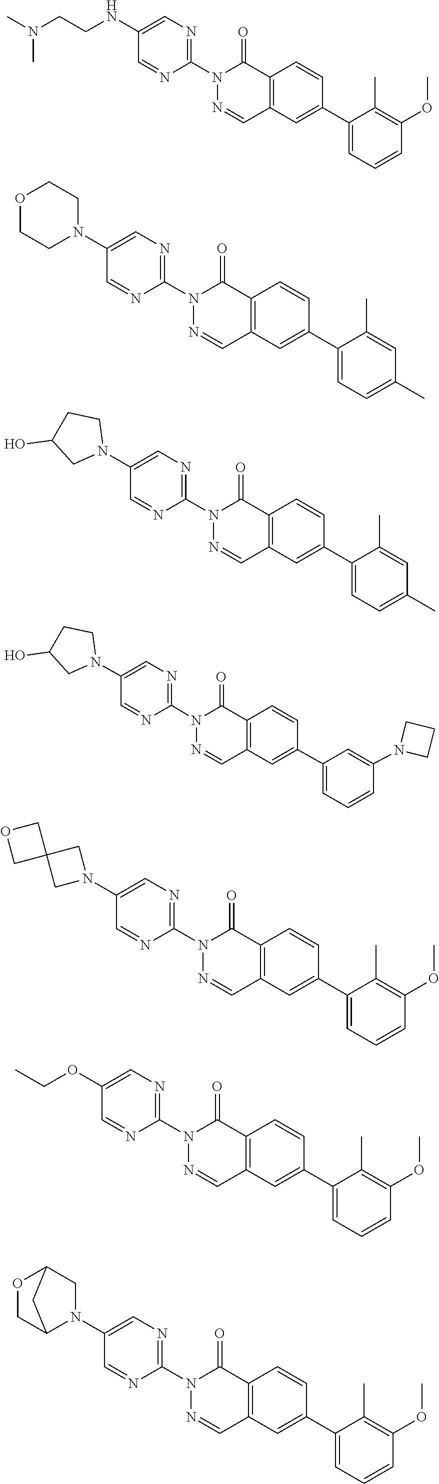 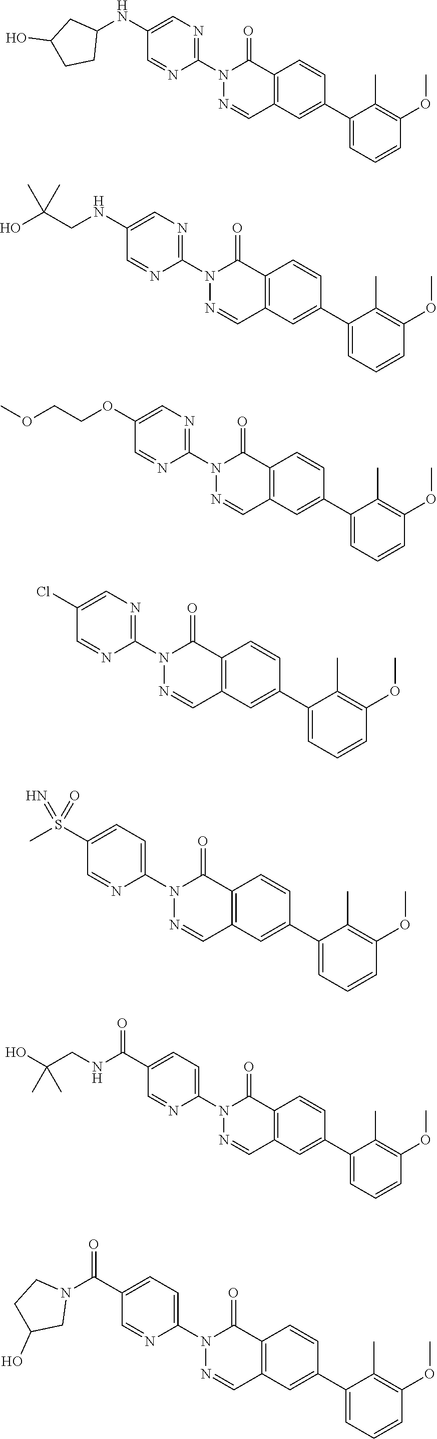 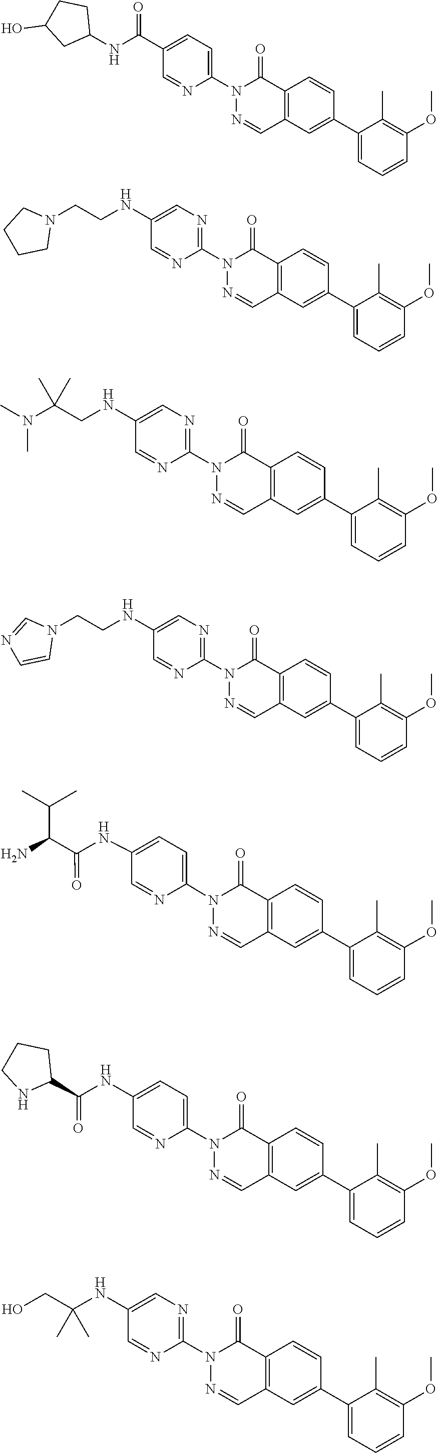 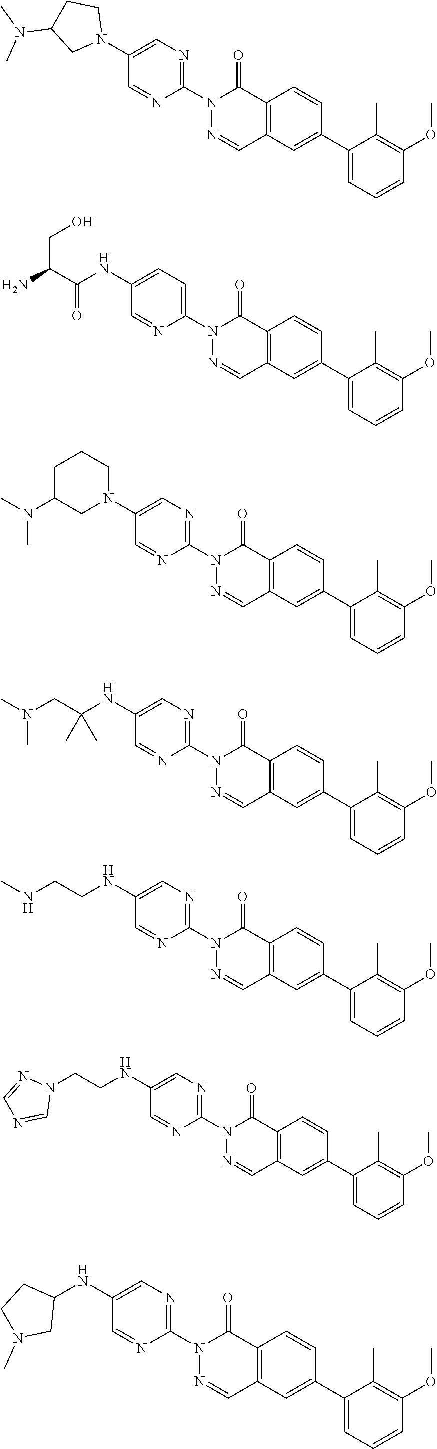 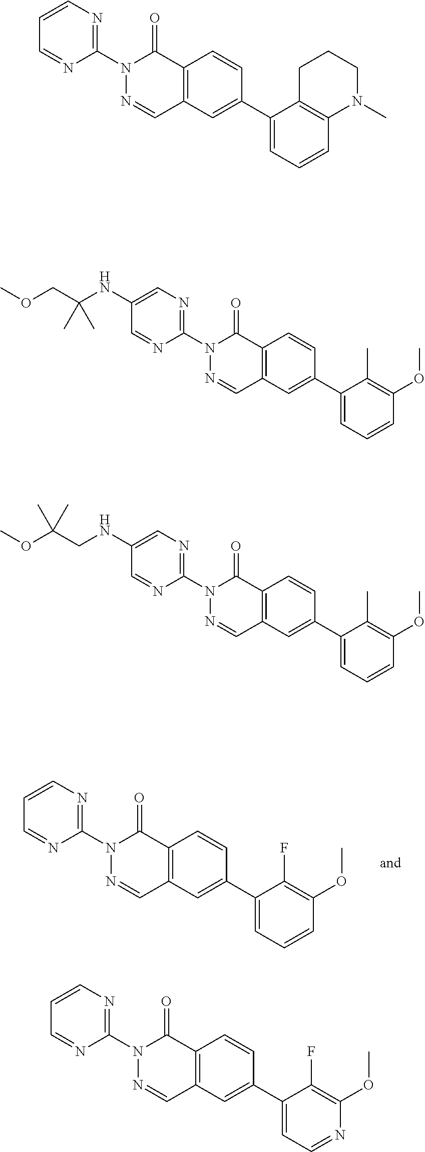
and a pharmaceutically acceptable acid or base addition salt thereof, a stereochemically isomeric form thereof or an N-oxide form thereof.

24. The compound according to claim 23, wherein said compound is one or more selected from 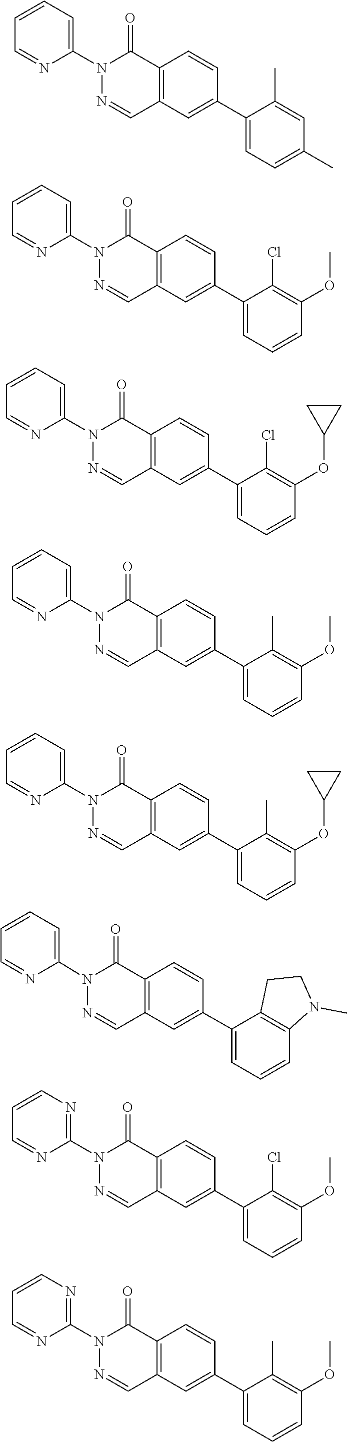 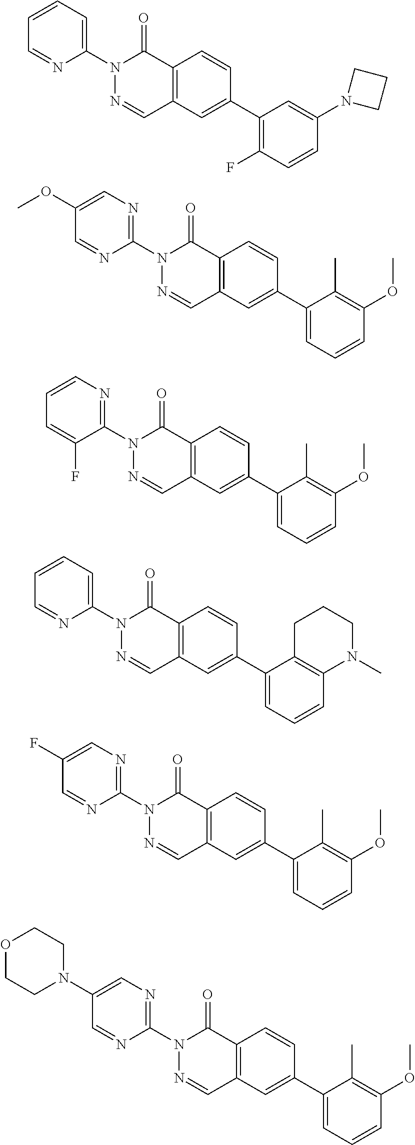 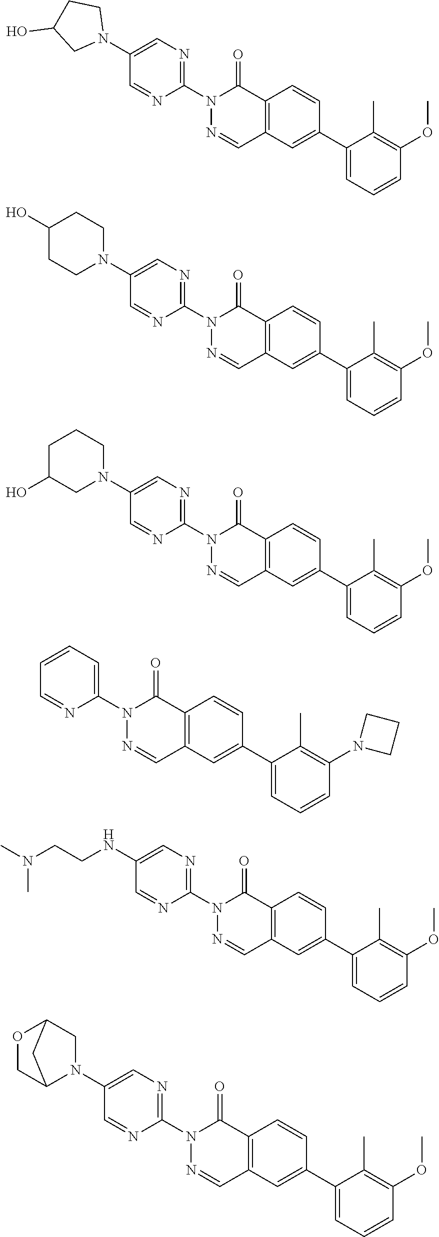 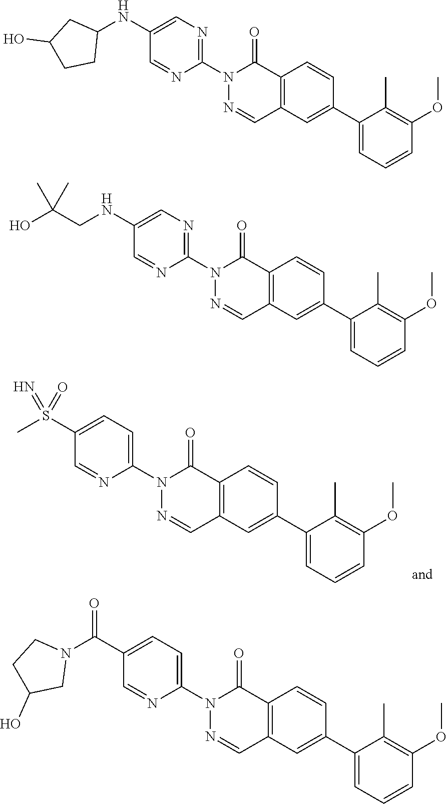
and a pharmaceutically acceptable acid or base addition salt thereof, a stereochemically isomeric form thereof or an N-oxide form thereof.

25. The compound according to claim 24, wherein said compound is one or more selected from 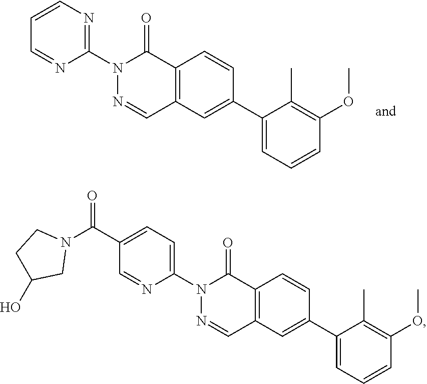 [SMILES: COc1cccc(-c2ccc3c(=O)n(-c4ncccn4)ncc3c2)c1C ; COc1cccc(-c2ccc3c(=O)n(-c4ccc(C(=O)N5CCC(O)C5)cn4)ncc3c2)c1C]
and a pharmaceutically acceptable acid or base addition salt thereof, a stereochemically isomeric form thereof or an N-oxide form thereof.

26. A pharmaceutical composition comprising a therapeutically effective amount of a compound according to claim 1 and a pharmaceutically acceptable carrier and/or excipient.

27. A method of treating or preventing a condition in a mammal, comprising administering to a mammal in need of such treatment or prevention, an effective amount of a compound/composition according to claim 1.

28. A method according to claim 27 wherein the treatment or prevention is affected or facilitated by the modulatory effect of a mGlu7 allosteric modulator such as a mGlu7 negative allosteric modulator.

29. A method of treating, preventing, ameliorating, controlling or reducing the risk of various neurological and psychiatric disorders associated with glutamate dysfunction in a mammal, comprising administering to a mammal in need of such treatment or prevention, an effective amount of a compound/composition according to claim 1.

30. A method according to claim 29, wherein the treatment or prevention is affected or facilitated by the modulatory effect of a mGlu7 negative allosteric modulator.

31. A method according to claim 27 wherein the condition is one or more of a central nervous system disorder, an otic disease or disorder or a pain disorder.

32. A method according to claim 31, wherein the central nervous system disorder is one or more of: an anxiety disorder such as agoraphobia, generalized anxiety disorder (GAD), obsessive-compulsive disorder (OCD), panic disorder, post-traumatic stress disorder (PTSD).

33. A method according to claim 31, wherein the otic disease and disorder is one or more of an inner ear impairment, age-related hearing impairment (presbycusis), Meniere's disease, sudden hearing loss, noise induced hearing loss, otitis media, autoimmune inner ear disease, acute tinnitus, chronic tinnitus, drug-induced hearing loss, hidden hearing loss, cisplatin-induced hearing loss, aminoglycosides-induced hearing loss, ototoxicity, central auditory processing disorder or vestibular disorder.

34. A method according to claim 31, wherein the pain disorder is one or more of neuropathic pain, inflammatory pain, visceral pain, acute pain, chronic pain, severe pain, intractable pain, post-traumatic pain, post-operative pain, headache pain or cancer pain.

35. A compound or composition according to claim 1 for use as a medicament.

36. A compound or composition according to claim 1 for use in a method of treatment or prevention as defined in any one of claims 27, 28, 31, 32, 33 or 34.

37. A compound or composition according to claim 1 for a use in a method as defined in claim 29 or 30.

38. Use of a compound according to claim 1 in the manufacture of a medicament for the treatment or prevention as defined in any one of claims 27, 28, 31, 32, 33 or 34.

39. Use of a compound according to claim 1 in the manufacture of a medicament for a treatment or prevention as defined in claim 29 or 30.
